(19) United States
(12) Patent Application Publication
(10) Pub. No.: US 2024/0246941 A1
(43) Pub. Date: Jul. 25, 2024

(54) SUBSTITUTED 4-(3-AMINOPROP-1-YL)AMINOQUINOLINE ANALOGS AS MODULATORS OF MELANOMA-ASSOCIATED ANTIGEN 11 UBIQUITIN LIGASE

(71) Applicant: ST. JUDE CHILDREN'S RESEARCH HOSPITAL, INC., Memphis, TN (US)

(72) Inventors: Ryan POTTS, Thousand Oaks, CA (US); Taosheng CHEN, Memphis, TN (US); Zoran RANKOVIC, Memphis, TN (US); Jaeki MIN, Memphis, TN (US); Wenwei LIN, Memphis, TN (US); Seung Wook YANG, Thousand Oaks, CA (US); Anand MAYASUNDARI, Memphis, TN (US)

(21) Appl. No.: 18/015,657

(22) PCT Filed: Jul. 15, 2021
(86) PCT No.: PCT/US2021/041816

§ 371 Date: Jan. 11, 2023

Related U.S. Application Data
(60) Provisional application No. 63/052,409, filed on Jul. 15, 2020.

(51) Int. Cl. C07D 405/12; A61K 31/4706; A61K 31/4709; A61K 45/06; A61P 35/00; C07D 215/46; C07D 401/12; C07K 7/08

(52) U.S. Cl. CPC C07D 405/12; A61K 31/4706; A61K 31/4709; A61K 45/06; A61P 35/00; C07D 215/46; C07D 401/12; C07K 7/08

(57) ABSTRACT

Testis-restricted melanoma antigen (MAGE) proteins are frequently hijacked in cancer and play a critical role in tumorigenesis. These proteins assemble with E3 ubiquitin ligases and function as substrate adaptors that direct the ubiquitination of novel targets, including key tumor suppressors. However, the development of MAGE-directed therapeutics heretofore has been extremely limited. In one aspect, the disclosure relates to compounds and peptides useful as inhibitors of MAGE-All: substrate interaction, methods of making same, pharmaceutical compositions comprising same, and methods of treating a disorder associated with a MAGE-All dysfunction. e.g., a cancer, using same. This abstract is intended as a scanning tool for purposes of searching in the particular art and is not intended to be limiting of the present disclosure.

### CROSS-REFERENCE TO RELATED APPLICATIONS

This application claims priority to and the benefit of U.S. Provisional Patent Application No. 63/052,409, filed Jul. 15, 2020 entitled “SUBSTITUTED 4-(3-AMINOPROP-1-YL)AMINOQUINOLINE ANALOGS AS MODULATORS OF OF MELANOMA-ASSOCIATED ANTIGEN 11 UBIQUITIN LIGASE” (Attorney Docket No. STJUDE-00004-U-USPRV-01), which is incorporated by reference herein in its entirety.

### BACKGROUND

Melanoma antigens (MAGEs) are a large (>40 in humans) and highly conserved family of proteins present in all eukaryotes (Ref. 1, 2). MAGEs can be classified into two categories based on their tissue expression pattern (Ref. 2, 3). Two-thirds of MAGEs, including MAGE-A, -B and -C subfamilies are considered type I MAGEs due to their restricted expression in the testis and other reproductive tissues (Refs. 3-6). Conversely, MAGE-D, -E, -F, -G, -H, -L and Necdin subfamilies are type II MAGEs that have broad expression in many tissues (Ref. 1, 2). Both type I and II MAGEs share a conserved domain known as the MAGE homology domain (MHD) involved in protein-protein interactions. MHDs consist of tandem winged-helix motifs (WH-A and WH-B) that feature a helix-turn-helix packed against a three-stranded antiparallel beta-sheet (Refs. 7-9). The overall structures of MHDs are similar between MAGEs, but the relative orientation of WH-A to WH-B can differ (Ref. 8,9).

Although type I MAGEs are typically restricted to expression in the testis, they are often aberrantly expressed in many cancer types and are often referred to as cancer-testis antigens (Ref. 1, 3). The aberrant expression of MAGEs in tumors is not simply an inert consequence of genomic dysregulation, but instead MAGEs play active roles in driving tumorigenesis (Refs. 1, 5, and 10-13). For example, MAGE-A3, -A6 and -A11 are aberrantly expressed in many cancer types, including melanoma, breast, colon and lung cancers, where they are required for cancer cell viability and promote multiple hallmarks of cancer, such as anchorage-independent growth and xenograft tumor growth (Ref. 1, 5, 10, and 14). Recent studies have highlighted an important role of MAGEs in controlling core oncogenic and tumor suppressor pathways in cells, including metabolic rewiring by MAGE-A3/6 through modulation of AMPK and autophagy and promotion of alternative polyadenylation by MAGE-A11 (Ref. 5, 10). In addition, MAGE-F1 is highly amplified in multiple cancer types leading to reduced DNA repair capacity and increased mutational burden in tumors through downregulation of the cytosolic iron-sulfur assembly pathway (Ref. 15). Thus, given the prominent, cancer selective expression of MAGEs and their oncogenic functions, MAGEs have emerged as prime therapeutic targets. However, this effort has been impeded by our limited understanding of how to target these enigmatic proteins.

Growing evidence suggests that MAGEs function as substrate adaptors for E3 ubiquitin ligases (reviewed in Ref. 2). MAGEs bind to specific single-subunit E3 ubiquitin ligases, both RING and HECT type ligases, including MAGE-A1-TRIM31, MAGE-A3-TRIM28, MAGE-A11-HUWE1, MAGE-B18-LNX1, MAGE-D1-PRAJA1, MAGE-F1-NSE1, MAGE-L2-TRIM27 and MAGE-G1-NSE1 (Ref. 8, 10, and 16-18). Importantly, MAGEs alter the function of these ligases by recruiting novel substrates to the ligases for ubiquitination, including MAGE-A3 mediating the ubiquitination of AMPK by TRIM28, MAGE-A11 mediating ubiquitination of PCF11 by HUWE1 and MAGE-F1 mediating ubiquitination of MMS19 by NSE1 (Ref. 5, 10, and 15). In each of these cases, the MAGE functions as ‘protein glue’ that mediates recruitment of the substrate to the ligase complex for ubiquitination. Thus, MAGEs reprogram single-subunit ubiquitin ligases. Notably, the specific MAGE bound to a ligase can dictate distinct substrates and functions. For example, both MAGE-F1 and MAGE-G1 bind the NSE1 ligase. However, these MAGEs have distinct substrates with opposing cellular functions in modulating DNA repair pathways (Ref. 8, 15, and 19). In many ways MAGEs have similar properties to Cullin-RING Ligase (CRL) adaptor protein families, such as F-box, BTB, DCAF and SOCS box proteins, which allow modularity, functional diversification and adaptability of CRLs20. However, unlike CRL adaptors, the molecular and structural basis of how MAGEs recognize substrates has been unknown.

Thus, although testis-restricted melanoma antigen (MAGE) proteins are frequently hijacked in cancer and play a critical role in tumorigenesis, the mechanism by which MAGEs recognize their targets is not previously known in the prior art and has impeded the development of MAGE-directed therapeutics.

Despite advances in cancer research and research directed to a better understanding of MAGE proteins, there is still a scarcity of compounds that are both potent, efficacious, and selective inhibitors of MAGE proteins, in particular, MAGE-A11:substrate interactions, and also effective in the treatment of disorders associated with MAGE proteins, e.g., a cancer. These needs and other needs are satisfied by the present disclosure.

### SUMMARY

In accordance with the purpose(s), as embodied and broadly described herein, the present disclosure provides compounds that are useful as inhibitors of MAGE-A11:substrate interaction, methods of making same, pharmaceutical compositions including the same, and methods of treating a disorder associated with a MAGE-A11 dysfunction, e.g., a cancer, using same. The present disclosure further relates to peptides useful as inhibitors of MAGE-A11:substrate interaction, methods of making same, pharmaceutical compositions including same, and methods of treating a disorder associated with a MAGE-A11 dysfunction, e.g., a cancer, using same.

Disclosed are compounds having a structure represented by a formula:

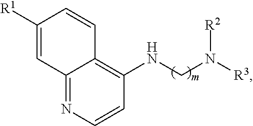

wherein m is selected from 2, 3, and 4; wherein R¹ is selected from hydrogen, halogen, C1-C10 haloalkyl, C1-C10 alkoxy, C1-C10 aminoalkyl, C1-C10 alkylamino, C1-C10 hydoxyalkyl, C1-C10 alkyl, -Q-Ar², -Q-Cy¹, —Ar², and —Cy¹; wherein Q is selected from —(CH₂)ₙ— and —O—; wherein n is selected from 1 and 2; wherein the Ar¹ is selected from phenyl, benzyl, indolyl, benzo[d][1,3]dioxolyl naphthyl, and anthracenyl; wherein the Ar¹ is substituted with 0, 1, or 2 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; wherein the Cy¹ is C3-C8 cycloalkyl; and wherein the Cy¹ is substituted with 0, 1, 2, 3, 4, or 5 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; wherein R² is selected from H and C1-C3 alkyl; wherein R³ is selected from H, C1-C3 alkyl, and —(CH₂)ₚ—Ar²; wherein p is selected from 1 and 2; wherein Ar² is selected from a phenyl, indolyl, benzo[d][1,3]dioxolyl, naphthyl, and anthracenyl; wherein the Ar² is substituted with 0, 1, or 2 groups independently selected from halogen, —SF₅, —CN, —N₃, —NO₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; or a pharmaceutically acceptable salt thereof.

Also disclosed are peptides having a structure given by the sequence:

FLVVVHQIRQLXQ,

wherein X is a substituted phenylalanine analog having the structure given by the formula:

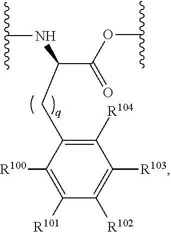

wherein q is selected from 0, 1, and 2; wherein each of R¹⁰⁰, R¹⁰¹, R¹⁰², R¹⁰³, and R¹⁰⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.

Also disclosed are pharmaceutical compositions comprising a therapeutically effective amount of one or more disclosed compounds, or pharmaceutically acceptable salt thereof; a therapeutically effective amount of one or more disclosed peptides, or pharmaceutically acceptable salt thereof; or combinations thereof; and a pharmaceutically acceptable carrier.

Also disclosed are methods for the treatment of a disorder associated with MAGE-A11 dysfunction in a mammal comprising the step of administering to the mammal a therapeutically effective amount of one or more disclosed compounds, or pharmaceutically acceptable salt thereof; a therapeutically effective amount of one or more disclosed peptides, or pharmaceutically acceptable salt thereof; or combinations thereof.

Also disclosed are methods for inhibiting a MAGE-A11:substrate interaction in a mammal comprising the step of administering to the mammal a therapeutically effective amount of one or more disclosed compounds, or pharmaceutically acceptable salt thereof; a therapeutically effective amount of one or more disclosed peptides, or pharmaceutically acceptable salt thereof; or combinations thereof.

Also disclosed are methods for inhibiting MAGE-A11 activity in at least one cell, comprising the step of contacting the cell with an effective amount of one or more disclosed compounds, or pharmaceutically acceptable salt thereof; an effective amount of one or more disclosed peptides, or pharmaceutically acceptable salt thereof; or combinations thereof.

Also disclosed are uses of a disclosed compound, or a pharmaceutically acceptable salt thereof; a disclosed peptide, or a pharmaceutically acceptable salt thereofl; a disclosed product of making a disclosed compound and/or a product of making a disclosed peptide, or a pharmaceutically acceptable salt thereof; or a disclosed pharmaceutical composition including a therapeutically effective amount of one or more disclosed compounds, or pharmaceutically acceptable salt thereof; a therapeutically effective amount of one or more disclosed peptides, or pharmaceutically acceptable salt thereof; or combinations thereof.

Also disclosed are uses of a one or more disclosed compounds, or pharmaceutically acceptable salt thereof; one or more disclosed peptides, or pharmaceutically acceptable salt thereof; or combinations thereof; in the manufacture of a medicament for the treatment of a disorder associated with a MAGE-A11 dysfunction in a mammal.

Also disclosed are methods for the manufacture of a medicament to inhibit a MAGE-A11:substrate interaction in a mammal including combining a pharmaceutically acceptable carrier or diluent and one or more disclosed compounds, or pharmaceutically acceptable salt thereof; one or more disclosed peptides, or pharmaceutically acceptable salt thereof; or combinations thereof.

Also disclosed are kits including at least one disclosed compound, or a pharmaceutically acceptable salt, hydrate, solvate, or polymorph thereof, and one or more of: (a) at least one agent known to increase MAGE-A11 activity; (b) at least one agent known to decrease MAGE-A11 activity; (c) at least one agent known to treat a disorder of uncontrolled cellular proliferation; (d) instructions for treating a disorder of uncontrolled cellular proliferation; or (e) instructions for treating a disorder associated with MAGE-11A dysfunction.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1H show representative data pertaining to characterization of MAGE-A11 interaction with substrates. FIG. 1A shows the MAGE-A11 recognition sequence in PCF11 is within amino acids 653-702. HEK293FT cells stably expressing FLAG-vector or FLAG-MAGE-A11 were transfected with PCF11 wild-type or PCF11 653-702 deletion construct for 48 hr followed by IP with anti-FLAG, SDS-PAGE and immunoblotting for anti-Myc. FIGS. 1B and 1C show data pertaining to mapping of MAGE-A11-interacting motif in PCF11 by TR-FRET. Sequences of PCF11 degron motif and tested eight PCF11 peptides are shown FIG. 1B. The binding activity of each peptide to GST-MAGE-A11 by TR-FRET is shown FIG. 1C. FIG. 1D shows data pertaining to validation of PCF11 peptide binding to MAGE-A11 using a thermal stability assay. Thermal unfolding of MAGE-A11 was monitored with increasing concentrations of PCF11_6 peptide. FIG. 1E shows data pertaining to alanine scanning mutagenesis of PCF11_6 peptide binding to GST-MAGE-A11 as determined using a TR-FRET assay as described herein.

FIG. 1F show data pertaining to relative TR-FRET affinity measurements of PCF11 degron peptide variants library (248 peptides). Shading indicates relative binding affinity normalized at each position. The circles indicate the sequence of the natural PCF11 degron peptide. FIG. 1G show a comparison of 16 predicted MAGE-A11-binding motifs. FIG. 1H show data pertaining to binding of recombinant GST-MAGE-A11 to in vitro translated Myc-RACGAP1, ANKRD65 and ZBTB7C.

FIGS. 2A-2G show data pertaining to the crystal structure of MAGE-A11:PCF11 and identification of the MAGE substrate binding cleft. FIG. 2A show representative data pertaining to MAGE-A11 directly interacting with PCF11 through its MHD. In vitro translated Myc-MAGE-A11 MHD, but not N-terminal fragment, binds to recombinant GST-PCF11. FIG. 2B show a representation of a model of the overall structure of MAGE-A11 MHD with PCF11 degron peptide. The alpha-helical PCF11 degron peptide binds into SBC at interface of WH-A and WH-B motifs of MAGE-A11 MHD. PCF11 peptide is shown in darker grey and MAGE-A11 in lighter grey. FIG. 2C show a representative space filling model of MAGE-A11 atoms within 5 Å of PCF11 are shown in darker grey to indicate the SBC region. Residues in the SBC that are mutated in this study are labeled and shown in a lighter grey. FIG. 2D shows a structural model of substrate interaction in which interaction is achieved by hydrophobic interactions along the alpha-helical interface of PCF11 with MAGE-A11 SBC. FIG. 2E shows representative data obtained using myc-tagged MAGE-A11 wild-type or mutants that were in vitro translated and followed by an in vitro binding assay with recombinant GST-PCF11 637-702 amino acids, SDS-PAGE and immunoblotting for anti-Myc. FIG. 2F shows representative data suggesting that MAGE-A11 SBC is crucial for PCF11 substrate binding, but not HUWE1 E3 ligase binding, in cells. HEK293FT cells stably expressing FLAG-vector, FLAG-MAGE-A11 wild-type or mutants were subjected to pull-down with anti-FLAG followed by SDS-PAGE and immunoblotting for anti-HUWE1 and anti-PCF11. FIG. 2G show representative data obtained using TR-FRET binding assays of GST-MAGE-A11 with PCF11 peptide (FLVVVHQIRQLF*Q) containing phenylalanine, 3-methyl-phenylalanine or 4-cyano-phenylalanine at the indicated position.

FIGS. 3A-3G show representative data indicating that mutation of MAGE-A11 SBC disrupts its oncogenic activity. FIG. 3A shows representative data suggesting that MAGE-A11-induced ubiquitination of PCF11 depends on its substrate recognition by MAGE-A11 SBC. Ubiquitinated proteins from FLAG-vector, FLAG-MAGE-A11 wild-type or SBC mutants stably expressing HEK293FT cells were treated with 10 μM MG132 for 4 hr and isolated with tandem ubiquitin binding entity-agarose followed by SDS-PAGE and immunoblotting for endogenous PCF11. FIG. 3B shows representative data suggesting that MAGE-A11 SBC mutants fail to promote proliferation of MAGE-A11 knockout DAOY cells. Wild-type, MAGE-A11-knockout DAOY cells or those reconstituted with MAGE-A11 were counted for cell proliferation at the indicated time points. FIGS. 3C, 3D, and 3E show representative data suggesting that MAGE-A11 knockout DAOY cells reconstituted with MAGE-A11 SBC mutants reduced clonogenic growth and xenograft tumor growth of MAGE-A11 knockout DAOY cells. Stable expression of wild-type MAGE-A11 or SBC mutants in MAGE-A11-knockout DAOY cells were assayed for clonogenic growth (FIG. 3C) and xenograft tumor growth in mice (n=6 per group). Tumor burden (FIG. 3D) and tumor weight (FIG. 3E) are shown. Data are mean±SD. Asterisks indicate p<0.05. FIG. 3F show representative data suggesting that MAGE-A11 M341R mutant fails to promote mRNA 3′UTR shortening. Transcriptome analysis of tumors (n=3 per group) from MAGE-A11 knockout DAOY cells stably expressing FLAG-MAGE-A11 wild-type or M341R mutant was determined and scatterplot of Percentage of Distal polyA site Usage Index (PDUI) is shown. False discovery rate ≤0.05 and ΔPDUIs≥0.2 are colored. The shift towards distal polyadenylation site is significant (p=5.9×10⁻¹⁶, binomial test). FIG. 3G show representative RNA-seq density plots.

FIGS. 4A-4G show representative data indicating that the SBC region mediates substrate recognition and specificity across the MAGE family. FIG. 4A shows a representative space filling structural model indicating sequence conservation of MAGE-A genes mapped onto MAGE-A11:PCF11 structure. FIG. 4B show a representative structural model showing comparison of MAGE-A3 and -A11 SBC that reveals key sequence differences in critical substrate binding residues. FIG. 4C shows representative obtained using in vitro GST-pulldown assays. The data reveal that GST-PCF11 binds in vitro translated Myc-MAGE-A11, but no other Myc-MAGE-A protein. FIGS. 4D, 4E, 4F, and 4G show data indicating that mutation of MAGE-A3 or -F1 SBC disrupts binding to their respective substrates AMPK and MMS19, not the E3 ligases TRIM28 and NSE1. Surface representations of MAGE-A3 (protomer model formed by PDB ID 4V0P for residues 1-296 and MAGE-A11 based homology model for C-terminal residues 297-311) (FIG. 4D) and MAGE-F1 (SWISS-MODEL based on PDB:4V0P) (FIG. 4F) show positioning of SBC residues mutated. Consistent with FIG. 2c, MAGE atoms within 5 Å of modeled PCF11 (based on superimposition of MAGE-A11:PCF11) are colored cyan. Residues in the SBC that are mutated are labeled and colored yellow. Co-IP of MAGEs with substrates and ligases were determined in Hela cells (FIG. 4F) or HEK293FT cells (FIG. 4G) upon transfection of the indicated constructs for 48 hr, IP with anti-Myc, SDS-PAGE and immunoblotting for indicated proteins.

FIGS. 5A-5G show representative data pertaining to disclosed small molecule inhibitors targeting MAGE-A11. FIG. 5A show representative obtained from 500 analogs from 9 scaffolds that were tested in dose response TR-FRET assays for disruption of MAGE-A11 binding to PCF11 identified the quinoline scaffold. FIG. 5B show a chemical structure of a representative disclosed compound, SJ521054. FIG. 5C show representative data obtained using a TR-FRET competition assay using indicated concentrations of quinoline series compounds for disruption of MAGE-A11:PCF11 binding. FIG. 5D show representative data indicating that SJ521054 inhibits MAGE-A11 binding to PCF11 in cell lysate. FLAG-MAGE-A11 stably expressing HEK293FT cell lysates were incubated with 100 μM SJ521054 for 24 hours and then subjected to pull-down with anti-FLAG followed by SDS-PAGE and immunoblotting for endogenous PCF11, and quantitated (n=4). *=p<0.05 (student's t-test). FIG. 5E show representative data indicating that SJ521054 blocks PCF11 degradation by MAGE-A11. HEK293FT cells stably expressing FLAG-vector or FLAG-MAGE-A11 were treated with indicated concentrations of SJ521054 for 24 hours before immunoblotting. FIG. 5F show representative data indicating that SJ521054 reduces viability of MAGE-A11 expressing DAOY cancer cells. MAGE-A11-knockout DAOY cells or those reconstituted with MAGE-A11 were treated with indicated concentrations of SJ521054 and cell viability was measured by alamarBlue assay 24 hours later. FIG. 5G show representative data indicating derivatives of SJ521054 that were synthesized and tested by TR-FRET competition assays with SJ1008066 had improved activity.

FIGS. 6A-6E show representative data pertaining to identification of the degron motif in PCF11 required for MAGE-A11 binding. FIG. 6A shows a representative schematic illustration of a disclosed TR-FRET MAGE-A11:PCF11 peptide binding assay. To monitor the MAGE-A11 and PCF11 interaction, recombinant GST-tagged MAGE-A11 MHD was incubated with FAM-labeled and unlabeled series of PCF11 peptides and TR-FRET signal was quantified. FIG. 6B show representative analysis of the Tm-shift in shown in FIG. 1D obtained by plotting the temperature at 50% relative fluorescence. FIGS. 6C and 6D show a schematic representation of alanine scanning mutations in PCF11 degron peptide. The amino acid sequences of PCF11 degron are listed with alanine substitutions for each residue. IC₅₀ values for each mutant by TR-FRET (FIG. 6C) and relative binding (FIG. 6D) are shown. FIG. 6E shows a representation of the MAGE-A11 binding motif sequence and weighted position matrix.

FIGS. 7A-7D show pertaining to the characterization of the MAGE-A11 SBC in recognition of PCF11. FIGS. 7A-7B show representative electron-density maps of PCF11 peptide bound to MAGE-A11. Shown are simulated annealing 2Fₒ-F_{c} composite omit maps contoured at 1σ. FIG. 7C shows a 2D representation of MAGE-A11-PCF11 interactions by Ligplot⁺(Ref. 44). FIG. 7D shows representative IC₅₀ results obtained using TR-FRET binding assay of GST-MAGE-A11 with PCF11 peptide (FLVVVHQIRQLF*Q) containing the indicated phenylalanine substitutions at the indicated position.

FIG. 8 shows a representative sequence alignment of MAGE-A3, -A11 and -F1 MHDs. Sequence alignment and secondary structure of MHDs from MAGE-A11, -A3 and -F1. Key conserved interaction residues within the SBC chosen for mutagenesis are shown. Residues in blue highlight substitutions within the SBC that likely contribute to substrate binding selectivity.

FIGS. 9A-9G show representative data obtained from a chemical screen of disclosed small molecular inhibitors of MAGE-A11:PCF11 interaction. FIG. 9A shows a summary of high-throughput screening approach disclosed herein. FIG. 9B representative results obtained from a primary screen for all 31,407 compounds screened at a single concentration (15 μM) using a TR-FRET assay. Positive controls (data shown having about 100% activity); negative control (DMSO, lighter grey centered on about 0% activity), and screening compounds (black). FIG. 9C shows the Z′-factor in the order of plates screened in the primary screen. FIG. 9D shows a representative percentage activity scatterplot for 797 analogs from five quinoline series screened at single concentration (15 μM) as determined using a TR-FRET assay. FIG. 9E shows representative data obtained using FLAG-MAGE-A11 stably expressing HEK293FT cell lysates that were incubated with 100 μM of the indicated quinoline compounds for 24 hours and then subjected to pull-down with anti-FLAG followed by SDS-PAGE and immunoblotting for endogenous PCF11. Note: SJ521054 (0.40 μM), SJ311286 (1.16 μM) and SJ311270 (0.58 μM) are active inhibitors by TR-FRET and SJ311280 (4.07 μM), SJ311281 (5.58 μM) and SJ311284 (20.60 μM) show low activity by TR-FRET. FIG. 9F shows representative data indicating that treatment of SJ521054 reduces viability of MAGE-A11 expressing HEK293FT cells. HEK293FT cells stably expressing FLAG-vector or FLAG-MAGE-A11 were treated with the indicated concentrations of SJ521054 and cell viability was measured by alamarBlue assay 24 hours later. FIG. 9G shows representative chemical structures of disclosed SJ521054 derivative compounds. IC₅₀, solubility, Caco-2 permeability and c Log P values of each compound are shown.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects of the present disclosure will employ, unless otherwise indicated, techniques of molecular biology, microbiology, organic chemistry, biochemistry, physiology, cell biology, blood vessel biology, and the like, which are within the skill of the art. Such techniques are explained fully in the literature.

Prior to describing the various aspects of the present disclosure, the following definitions are provided and should be used unless otherwise indicated. Additional terms may be defined elsewhere in the present disclosure.

### Definitions

As used herein, terms such as “by”, “including,” “includes,” “included,” “involving,” “involves,” “involved,” and “such as” should be interpreted to included examples and aspects encompassed by the terms “consisting essentially of” and “consisting of” as well as examples and aspects that include the presence of the stated features, integers, steps, or components as referred to, but does not preclude the presence or addition of one or more features, integers, steps, or components, or groups thereof as would commonly be encompassed by terms such as “comprising,” “comprises”, and “comprised of.”

As used herein, the term “and/or” includes any and all combinations of one or more of the associated listed items. Expressions such as “at least one of,” when preceding a list of elements, modify the entire list of elements and do not modify the individual elements of the list.

As used in the specification and the appended claims, the singular forms “a,” “an” and “the” include plural referents unless the context clearly dictates otherwise. Thus, for example, reference to “a compound,” “a peptide,” or “a cell,” including, but not limited to, two or more such compounds, peptides, or cells

Reference to “a/an” chemical compound, peptide, and cell each refers to one or more molecules of the chemical compound, peptide, and cell rather than being limited to a single molecule of the chemical compound, peptide, and cell. Furthermore, the one or more molecules may or may not be identical, so long as they fall under the category of the chemical compound, peptide, and cell. Thus, for example, “a” peptide is interpreted to include one or more peptide molecules of the peptide, where the peptide molecules may or may not be identical.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y′”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of’ the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refer to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “melanoma antigen, family A, 11,” “MAGEA11,” and “MAGE-A11” can be used interchangeably and refer to an enzyme encoded by a gene in humans with a cytogenetic location of Xq28 and a genomic coordinate of X:149,688, 192-149,717,267 (Homo sapiens Annotation Release 109, GRCh38). It has an intracellular location within the nucleus and cytoplasm. MAGE-A11 has also been referred to as Cancer/testis antigen 1.11 and MAGE-11 antigen. It is believed to be expressed in tumors such as melanoma, head and neck squamous cell carcinoma, lung carcinoma and breast carcinoma. Moreover, it is believed to be expressed in testis, ovary, prostate, cancerous prostate, breast and adrenal tissue.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit including an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can include one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, TT-TT interactions, cation-IT interactions, anion-IT interactions, polar IT-interactions, and hydrophobic effects.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and juvenile subjects, whether male or female, are intended to be covered. In one aspect, the subject is a mammal. A patient refers to a subject afflicted with a disease or disorder. The term “patient” includes human and veterinary subjects.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a cancer. The effect can be therapeutic in terms of a partial or complete cure of a disease, condition, symptom or adverse effect attributed to the disease, disorder, or condition. The term “treatment” as used herein can include any treatment of a cancer in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1 -to-C 6 alkyl esters and C 5 -to-C 7 cycloalkyl esters, although C 1 -to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1 -to-C 6 alkyl amines and secondary C 1 -to-C 6 dialkyl amines. In the case of secondary amines, the amine can also be in the form of a 5- or 6-membered heterocycle containing one nitrogen atom. Amides derived from ammonia, C 1 -to-C 3 alkyl primary amides and C 1 -to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

It is understood, that unless otherwise specified, temperatures referred to herein are based on atmospheric pressure (i.e. one atmosphere).

Described herein are compounds and peptides that have therapeutic or clinical utility. Also described herein are methods of synthesizing the disclosed compounds and peptides. Also described herein are methods of administering the disclosed compounds and peptides to a subject in need thereof. In some aspects, the subject can have a cancer. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Compounds.

In various aspects, the present disclosure relates to compounds useful as inhibitors of MAGE-A11:substrate interaction. Disclosed are compounds having a structure represented by a formula:

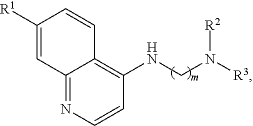

wherein m is selected from 2, 3, and 4; wherein R¹ is selected from hydrogen, halogen, C1-C10 haloalkyl, C1-C10 alkoxy, C1-C10 aminoalkyl, C1-C10 alkylamino, C1-C10 hydoxyalkyl, C1-C10 alkyl, -Q-Ar², -Q-Cy¹, —Ar², and —Cy¹; wherein Q is selected from —(CH₂)ₙ— and —O—; wherein n is selected from 1 and 2; wherein the Ar¹ is selected from phenyl, benzyl, indolyl, benzo[d][1,3]dioxolyl naphthyl, and anthracenyl; wherein the Ar¹ is substituted with 0, 1, or 2 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; wherein the Cy¹ is C3-C8 cycloalkyl; and wherein the Cy¹ is substituted with 0, 1, 2, 3, 4, or 5 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; wherein R² is selected from H and C1-C3 alkyl; wherein R³ is selected from H, C1-C3 alkyl, and —(CH₂)ₚ—Ar²; wherein p is selected from 1 and 2; wherein Ar² is selected from a phenyl, indolyl, benzo[d][1,3]dioxolyl, naphthyl, and anthracenyl; wherein the Ar² is substituted with 0, 1, or 2 groups independently selected from halogen, —SF₅, —CN, —N₃, —NO₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; or a pharmaceutically acceptable salt thereof.

In a further aspect, m is selected from 2 and 3. In a still further aspect, m is 1. In a yet further aspect, m is 2. In an even further aspect, m is 3. In yet further aspect, m is selected from 1 and 3.

In a further aspect, n is 1. In a still further aspect, n is 2.

In a further aspect, p is 1. In a still further aspect, p is 2.

In a further aspect, the disclosed compound has a structure represented by a formula:

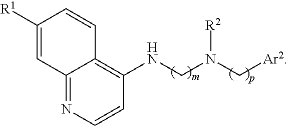

In a further aspect, the disclosed compound has a structure represented by a formula:

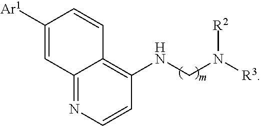

In a further aspect, the disclosed compound has a structure represented by a formula:

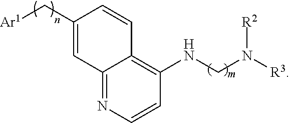

In a further aspect, the disclosed compound has a structure represented by a formula:

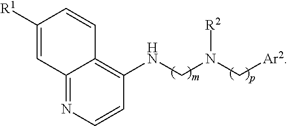

In a further aspect, the disclosed compound has a structure represented by a formula:

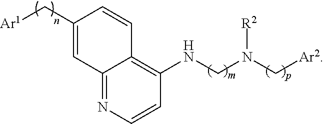

In a further aspect, the disclosed compound has a structure represented by a formula:

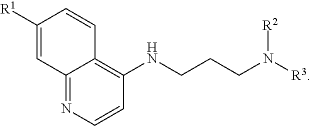

In a further aspect, the disclosed compound has a structure represented by a formula:

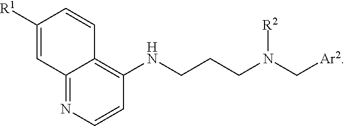

In a further aspect, the disclosed compound has a structure represented by a formula:

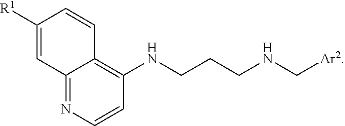

In a further aspect, the disclosed compound has a structure represented by a formula:

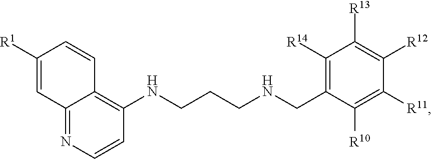

wherein each of R¹⁰, R¹¹, R¹², R¹³, and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R¹⁰, R¹¹, R¹², R¹³, and R¹⁴ are hydrogen.

In a further aspect, the disclosed compound has a structure represented by a formula:

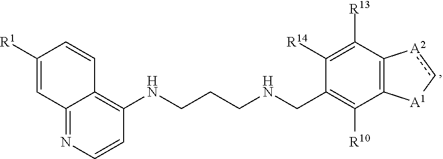

wherein each of R¹⁰, R¹³, and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; wherein A1 is selected from —O— and —NH—; and wherein A2 is selected from —O— and —CR¹¹═; and wherein the dashed line represents a bond as required to maintain appropriate valency.

In a further aspect, the disclosed compound has a structure represented by a formula:

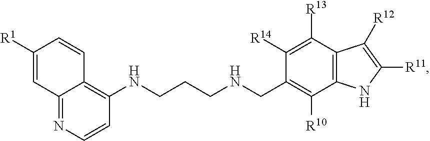

wherein each of R¹⁰, R¹¹, R¹², R¹³, and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R¹⁰, R¹¹, R¹², R¹³, and R¹⁴ are hydrogen.

In a further aspect, the disclosed compound has a structure represented by a formula:

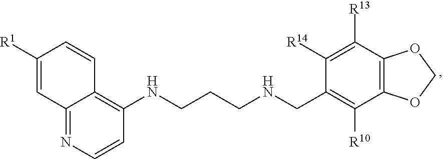

wherein each of R¹⁰, R¹³, and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.

In a further aspect, the disclosed compound has a structure represented by a formula:

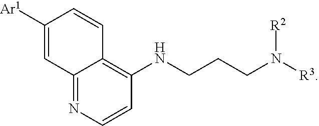

In a further aspect, the disclosed compound has a structure represented by a formula:

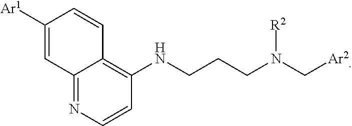

In a further aspect, the disclosed compound has a structure represented by a formula:

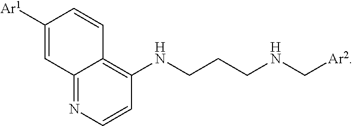

In a further aspect, the disclosed compound has a structure represented by a formula:

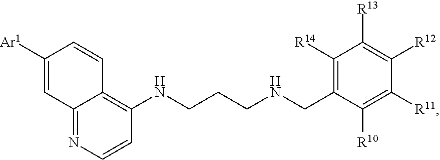

wherein each of R¹⁰, R¹¹, R¹², R¹³, and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R¹⁰, R¹¹, R¹², R¹³, and R¹⁴ are hydrogen.

In a further aspect, the disclosed compound has a structure represented by a formula:

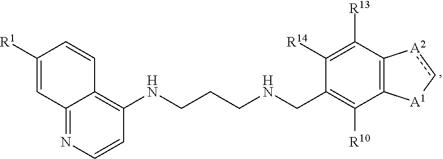

wherein each of R¹⁰, R¹³, and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; wherein A1 is selected from —O— and —NH—; and wherein A2 is selected from —O— and —CR¹¹═; and wherein the dashed line represents a bond as required to maintain appropriate valency.

In a further aspect, the disclosed compound has a structure represented by a formula:

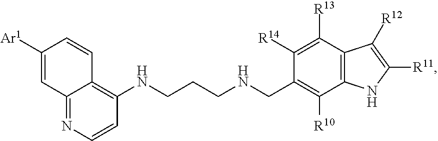

wherein each of R¹⁰, R¹¹, R¹², R¹³, and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R¹⁰, R¹¹, R¹², R¹³, and R¹⁴ are hydrogen.

In a further aspect, the disclosed compound has a structure represented by a formula:

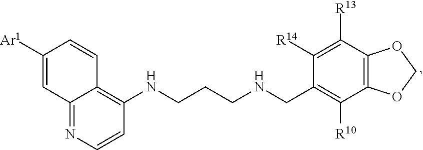

wherein each of R¹⁰, R¹³, and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.

In a further aspect, the disclosed compound has a structure represented by a formula:

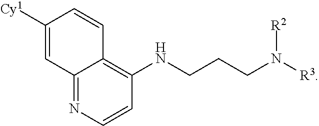

In a further aspect, the disclosed compound has a structure represented by a formula:

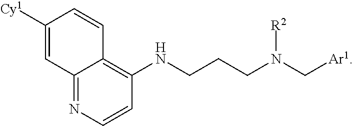

In a further aspect, the disclosed compound has a structure represented by a formula:

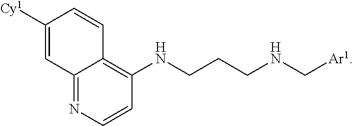

In a further aspect, the disclosed compound has a structure represented by a formula:

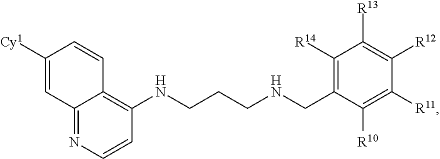

wherein each of R¹⁰, R¹¹, R¹², R¹³, and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R¹⁰, R¹¹, R¹², R¹³, and R¹⁴ are hydrogen.

In a further aspect, the disclosed compound has a structure represented by a formula:

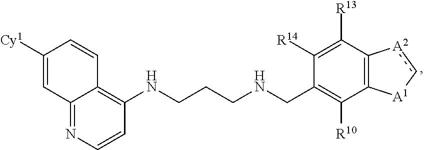

wherein each of R¹⁰, R¹³, and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; wherein A1 is selected from —O— and —NH—; and wherein A2 is selected from —O— and —CR¹¹═; and wherein the dashed line represents a bond as required to maintain appropriate valency.

In a further aspect, the disclosed compound has a structure represented by a formula:

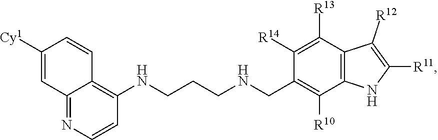

wherein each of R¹⁰, R¹¹, R¹², R¹³, and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R¹⁰, R¹¹, R¹², R¹³, and R¹⁴ are hydrogen.

In a further aspect, the disclosed compound has a structure represented by a formula:

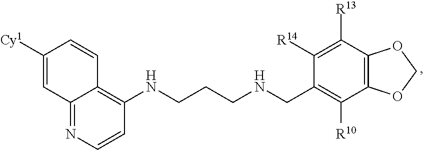

wherein each of R¹⁰, R¹³, and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.

In a further aspect, the disclosed compound has a structure represented by a formula:

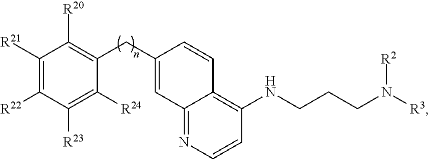

wherein n is selected from 1 and 2; and wherein each of R²⁰, R²¹, R²², R²³, and R²⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R²⁰, R²¹, R²², R²³, and R²⁴ are hydrogen.

In a further aspect, the disclosed compound has a structure represented by a formula:

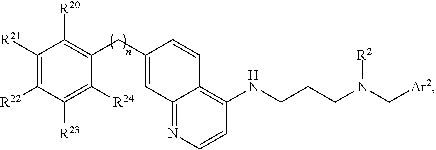

wherein n is selected from 1 and 2; and wherein each of R²⁰, R²¹, R²², R²³, and R²⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R²⁰, R²¹, R²², R²³, and R²⁴ are hydrogen.

In a further aspect, the disclosed compound has a structure represented by a formula:

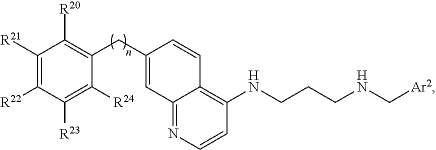

wherein n is selected from 1 and 2; and wherein each of R²⁰, R²¹, R²², R²³, and R²⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R²⁰, R²¹, R²², R²³, and R²⁴ are hydrogen.

In a further aspect, the disclosed compound has a structure represented by a formula:

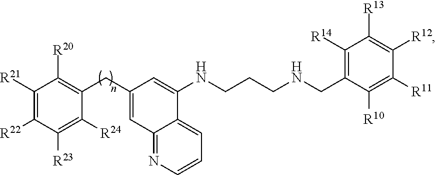

wherein n is selected from 1 and 2; wherein each of R¹⁰, R¹¹, R¹², R¹², and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R¹⁰, R¹¹, R¹², R¹², and R¹⁴ are hydrogen; and wherein each of R²⁰, R²¹, R²², R²³, and R²⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R²⁰, R²¹, R²², R²³, and R²⁴ are hydrogen.

In a further aspect, the disclosed compound has a structure represented by a formula:

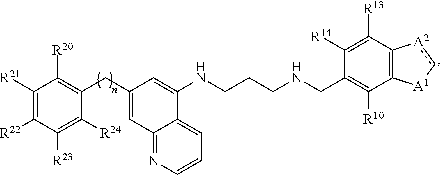

wherein n is selected from 1 and 2; wherein each of R¹⁰, R¹², and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; wherein A1 is selected from —O— and —NH—; and wherein A2 is selected from —O— and —CR¹¹═, and wherein the dashed line represents a bond as required to maintain appropriate valency; and wherein each of R²⁰, R²¹, R²², R²³, and R²⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R²⁰, R²¹, R²², R²³, and R²⁴ are hydrogen.

In a further aspect, the disclosed compound has a structure represented by a formula:

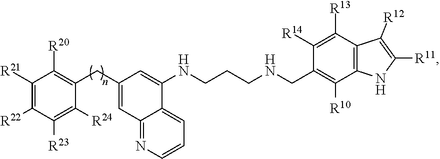

wherein n is selected from 1 and 2; wherein each of R¹⁰, R¹¹, R¹², R¹², and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R¹⁰, R¹¹, R¹², R¹², and R¹⁴ are hydrogen; and wherein each of R²⁰, R²¹, R²², R²³, and R²⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R²⁰, R²¹, R²², R²³, and R²⁴ are hydrogen.

In a further aspect, the disclosed compound has a structure represented by a formula:

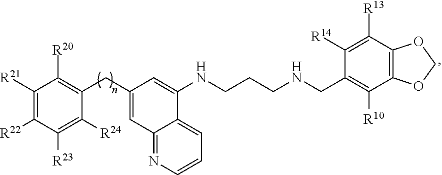

wherein n is selected from 1 and 2; wherein each of R¹⁰, R¹², and R¹⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; and wherein each of R²⁰, R²¹, R²², R²³, and R²⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that at least three of R²⁰, R²¹, R²², R²³, and R²⁴ are hydrogen.

In a further aspect, R¹ is selected from halogen, C1-C10 haloalkyl, C1-C10 alkoxy, C1-C10 aminoalkyl, C1-C10 alkylamino, C1-C10 hydoxyalkyl, C1-C10 alkyl, —Ar¹, —Cy¹, —O—Ar¹, —O—Cy¹, —(C1-C2 alkanediyl)-Ar¹, and —(C1-C2 alkanediyl)-Cy¹. In a still further aspect, R¹ is selected from halogen, C1-C6 haloalkyl, C1-C6 alkoxy, C1-C6 aminoalkyl, C1-C6 alkylamino, C1-C6 hydoxyalkyl, C1-C6 alkyl, -cyclohexyl, —(C1-C2 alkanediyl)-cyclohexyl, —O—Ar¹, and —(C1-C2 alkanediyl)-Ar¹. In a yet further aspect, R¹ is selected from chloro, C1-C3 haloalkyl, C1-C6 alkyl, -cyclohexyl, —(CH₂)-cyclohexyl, —O—Ar¹, and —(CH₂)—Ar¹.

In a further aspect, R² is selected from hydrogen and methyl. In a still further aspect, R² is hydrogen.

In a further aspect, R³ is selected from hydrogen and methyl. In a still further aspect, R³ is hydrogen. In an even further aspect, R³ is —(CH₂)ₙ—Ar².

In a further aspect, Ar¹ is phenyl substituted with 0, 1, or 2 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl. In a still further aspect, Ar¹ is phenyl substituted with 0, 1, or 2 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.

In a further aspect, Ar² is selected from indolyl, benzo[d][1,3]dioxolyl and phenyl substituted with 0, 1, or 2 groups independently selected from halogen, —SF₅, —CN, —N₃, —NO₂, —OH, —CN, C1-C3 alkoxy, C1-C3 haloalkyl, C1-C3 aminoalkyl, C1-C3 alkylamino, C1-C3 hydoxyalkyl, and C1-C3 alkyl. In a still further aspect, Ar² is indolyl. In a yet further aspect, indolyl is unsubstituted. In an even further aspect, Ar² is benzo[d][1,3]dioxolyl. In a still further aspect, benzo[d][1,3]dioxolyl is unsubstituted. In a yet further aspect, Ar² is phenyl substituted with 0, 1, or 2 groups independently selected from halogen, —SF₅, —CN, —N₃, —NO₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.

In various aspects, the disclosed has a structure represented by a formula:

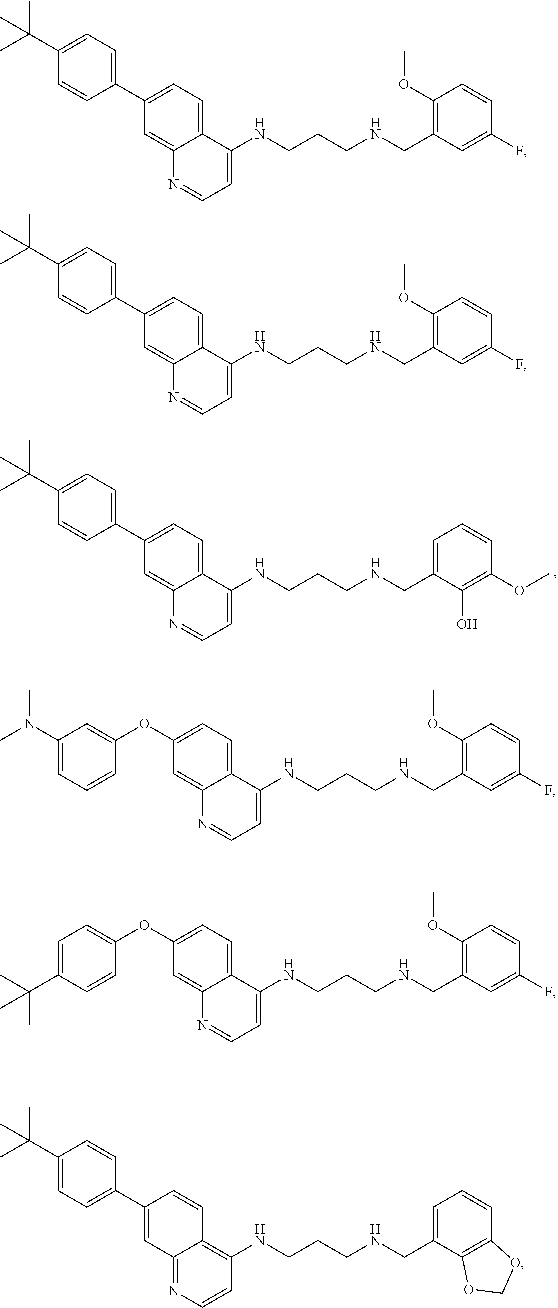

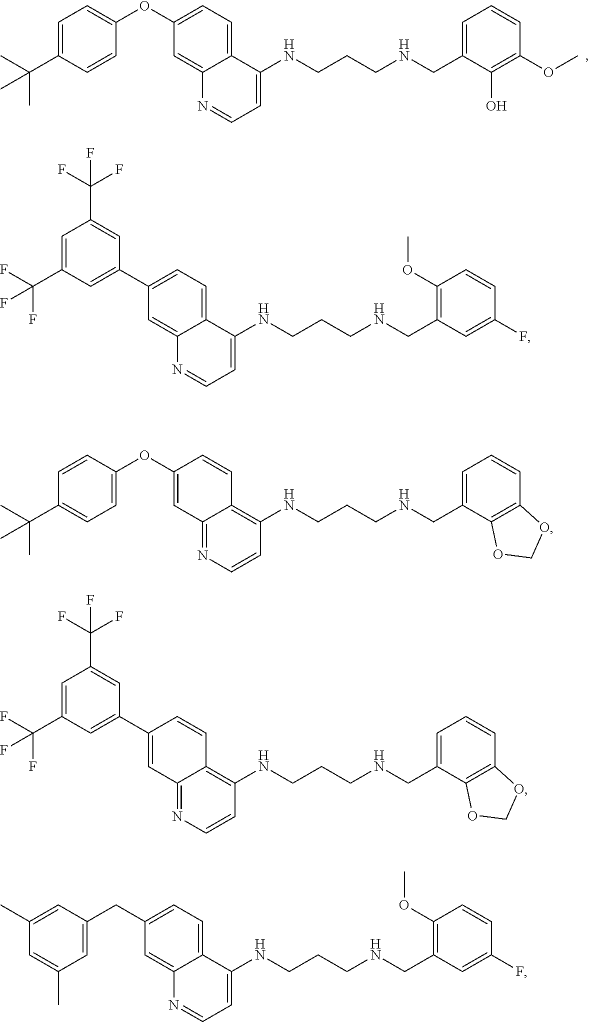

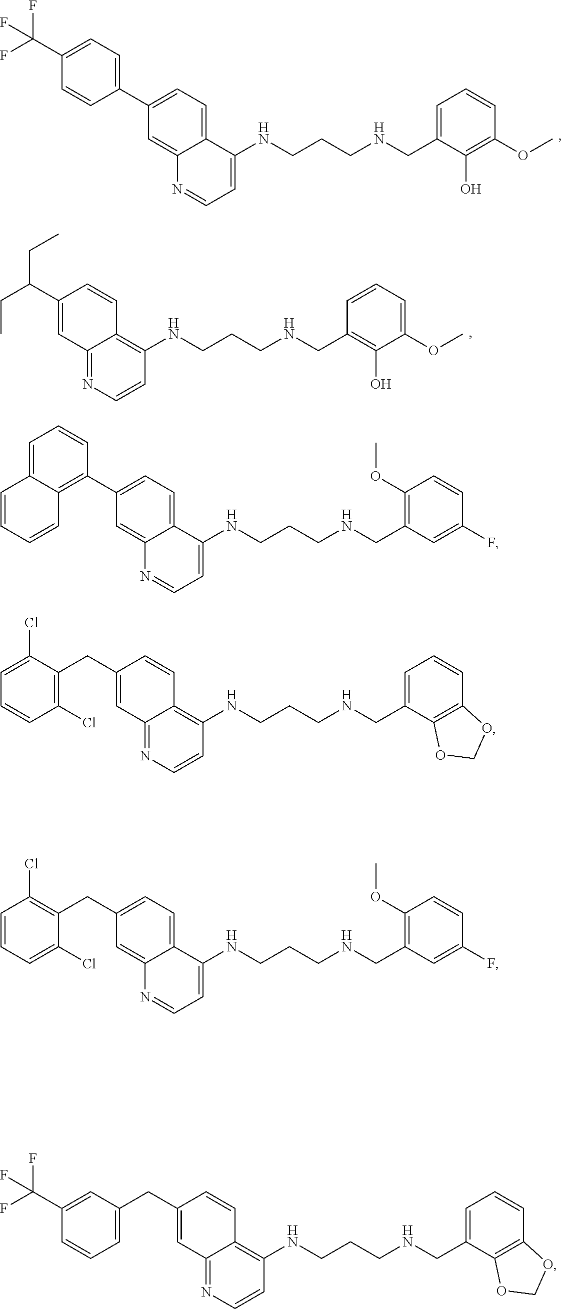 [SMILES: COc1cccc(CNCCCNc2ccnc3cc(-c4ccc(C(F)(F)F)cc4)ccc23)c1O ; CCC(CC)c1ccc2c(NCCCNCc3cccc(OC)c3O)ccnc2c1 ; COc1ccc(F)cc1CNCCCNc1ccnc2cc(-c3cccc4ccccc34)ccc12 ; Clc1cccc(Cl)c1Cc1ccc2c(NCCCNCc3cccc4c3OCO4)ccnc2c1 ; COc1ccc(F)cc1CNCCCNc1ccnc2cc(Cc3c(Cl)cccc3Cl)ccc12 ; FC(F)(F)c1cccc(Cc2ccc3c(NCCCNCc4cccc5c4OCO5)ccnc3c2)c1]

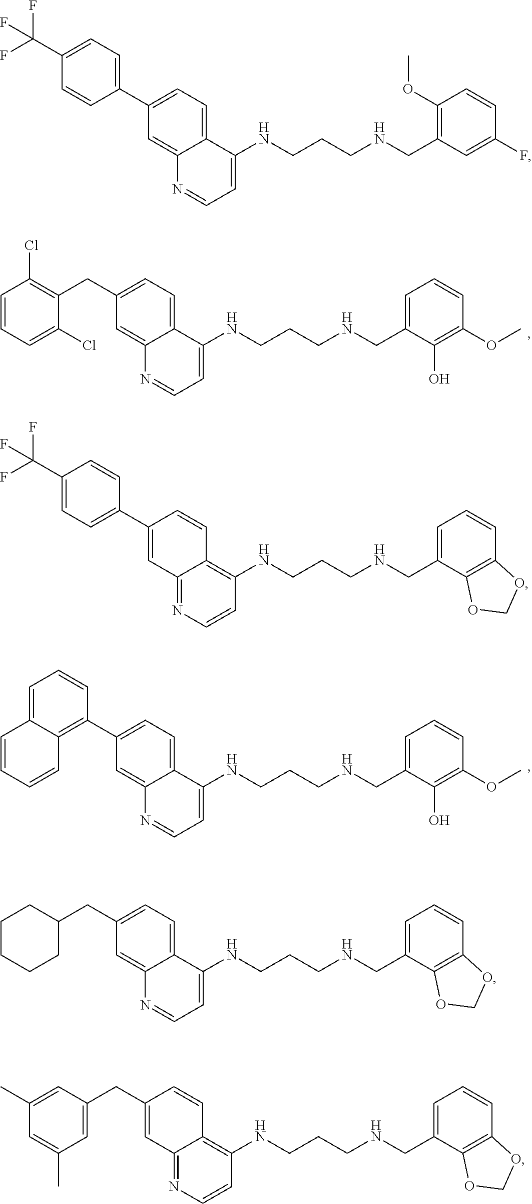

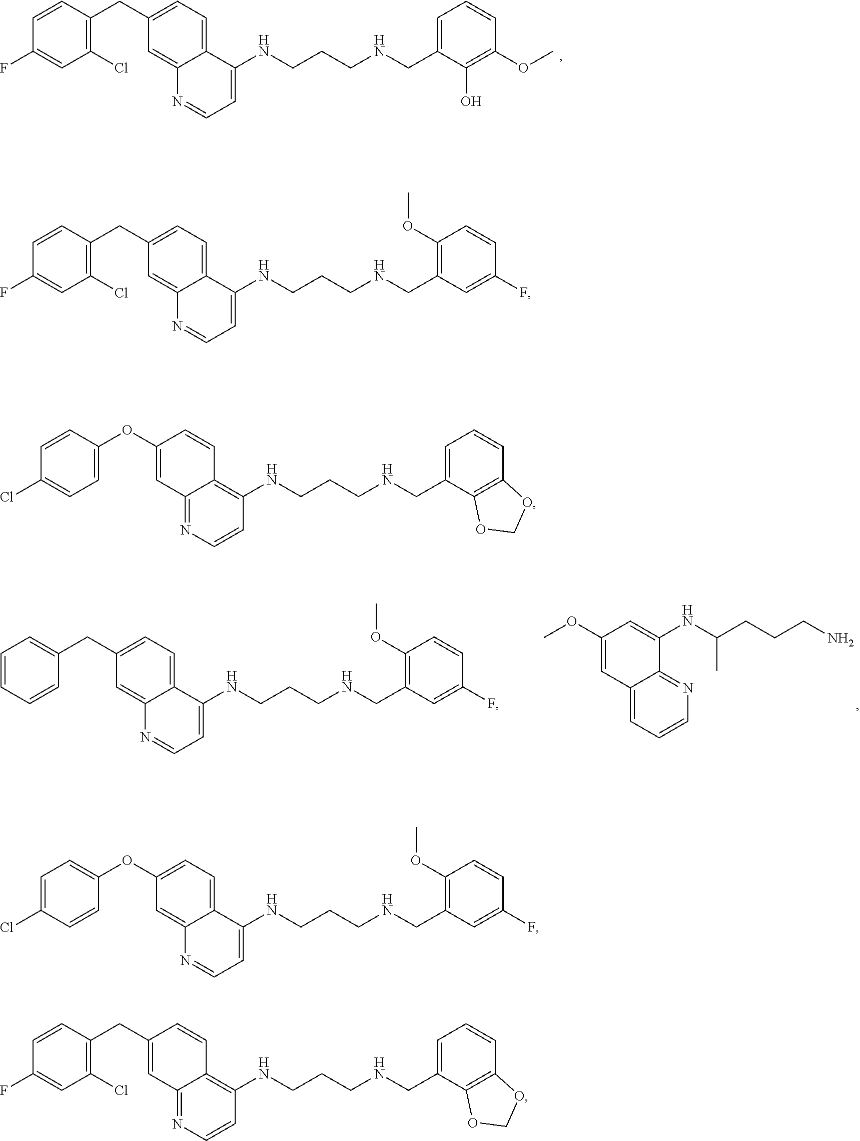

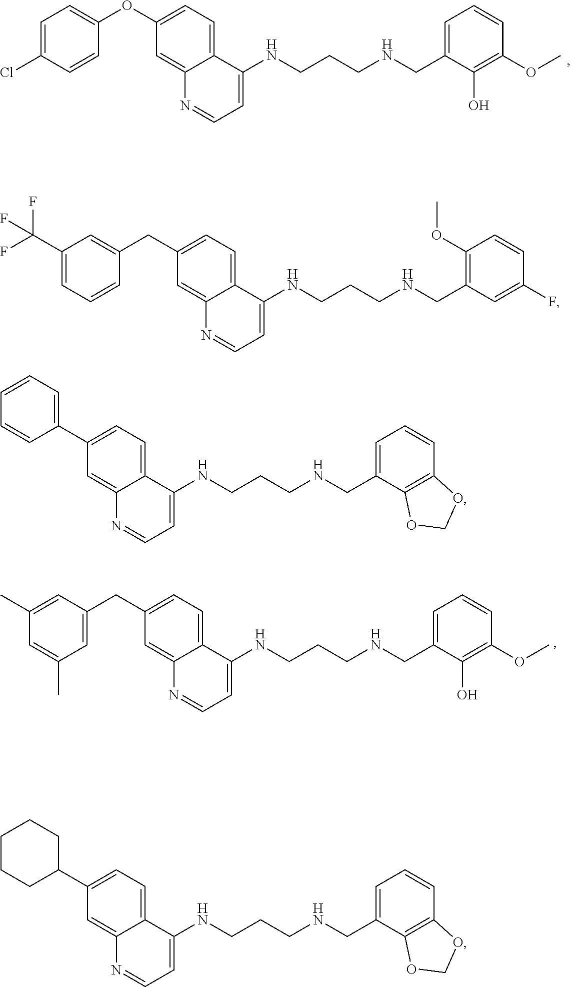

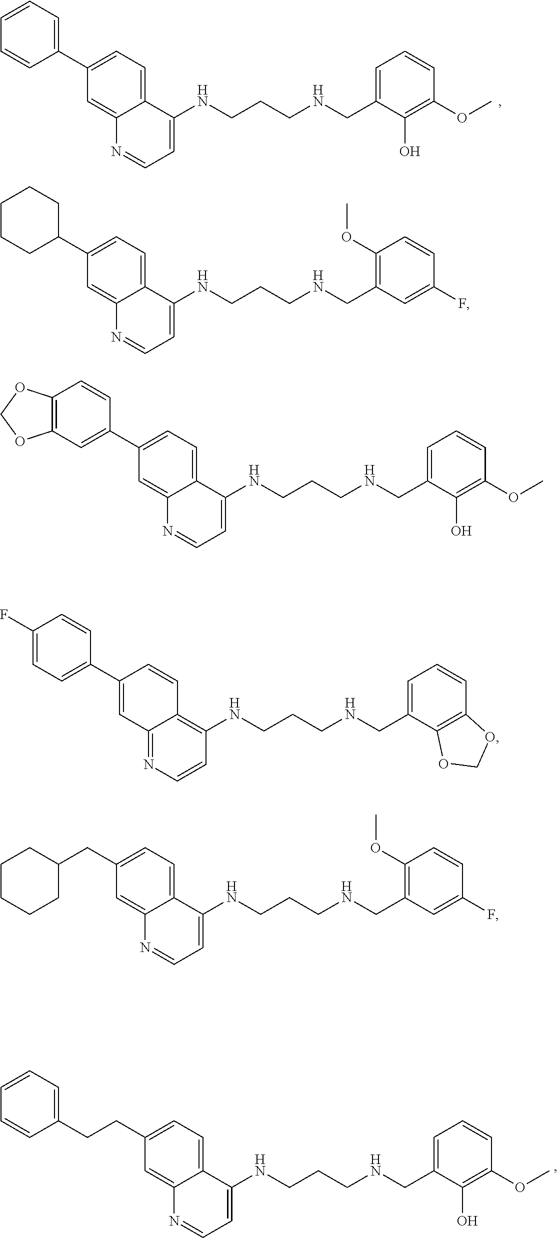 [SMILES: COc1cccc(CNCCCNc2ccnc3cc(-c4ccccc4)ccc23)c1O ; COc1ccc(F)cc1CNCCCNc1ccnc2cc(C3CCCCC3)ccc12 ; COc1cccc(CNCCCNc2ccnc3cc(-c4ccc5c(c4)OCO5)ccc23)c1O ; Fc1ccc(-c2ccc3c(NCCCNCc4cccc5c4OCO5)ccnc3c2)cc1 ; COc1ccc(F)cc1CNCCCNc1ccnc2cc(CC3CCCCC3)ccc12 ; COc1cccc(CNCCCNc2ccnc3cc(CCc4ccccc4)ccc23)c1O]

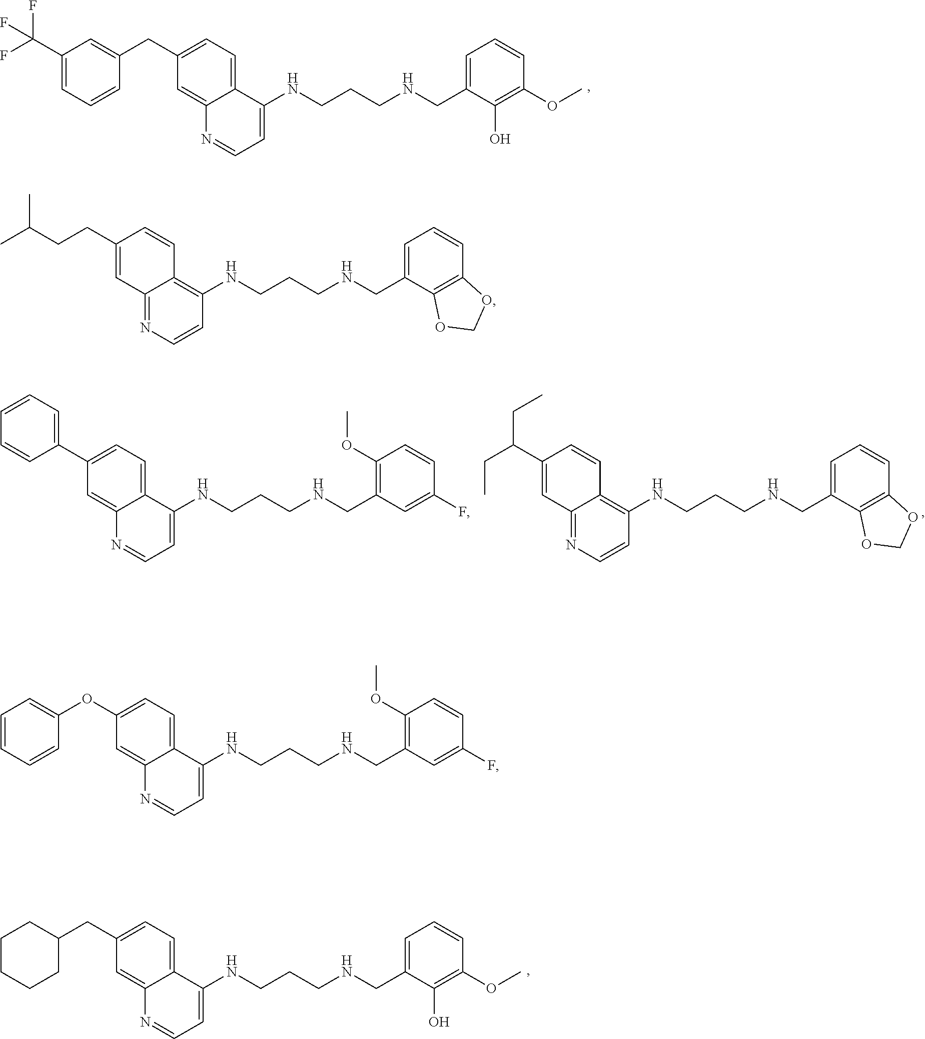

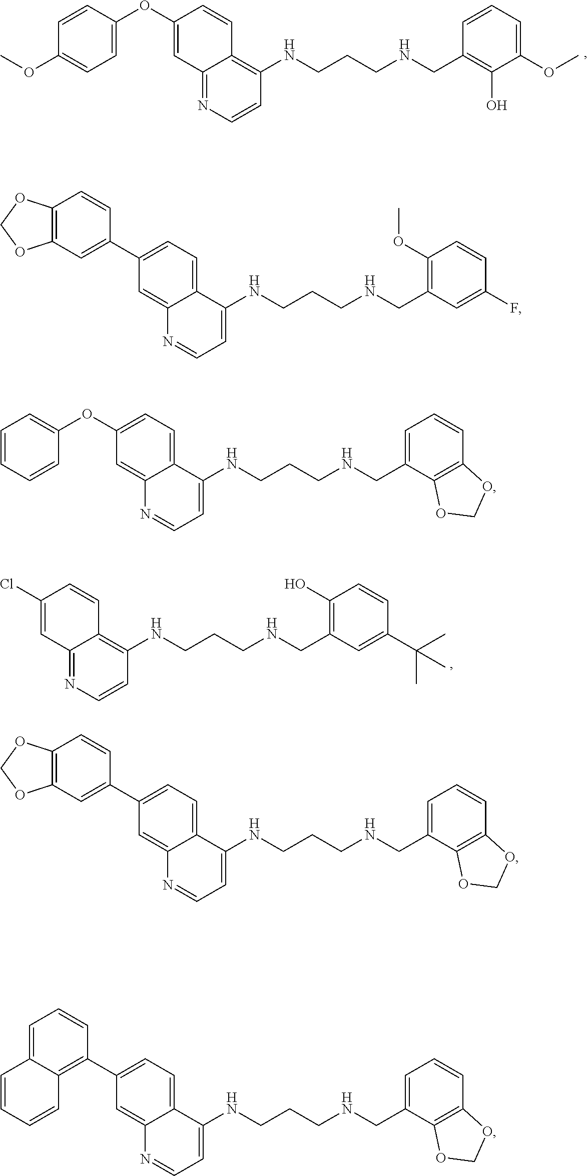

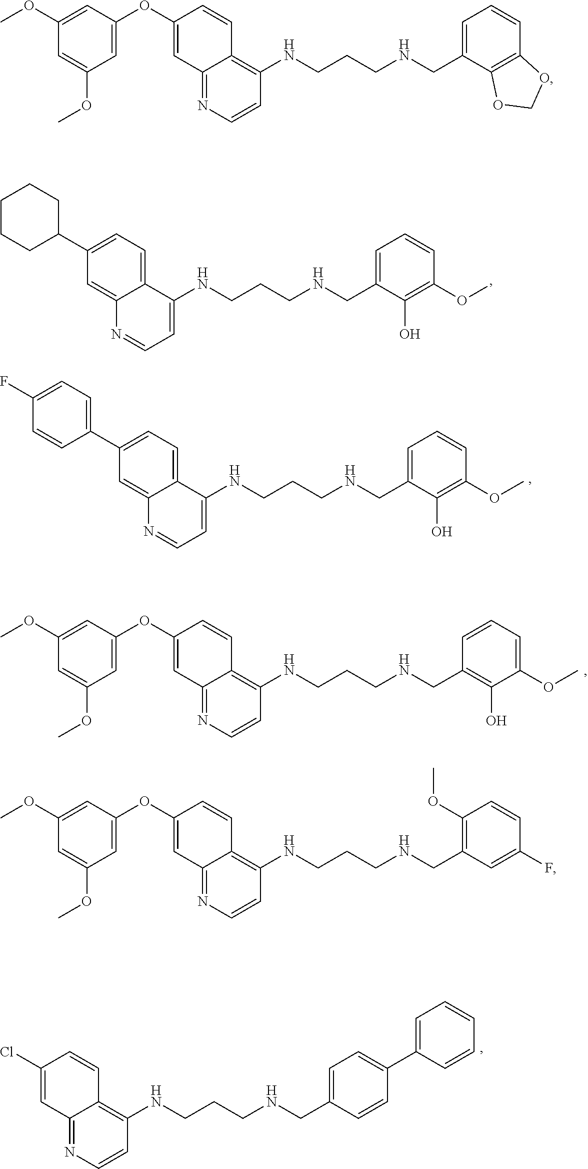

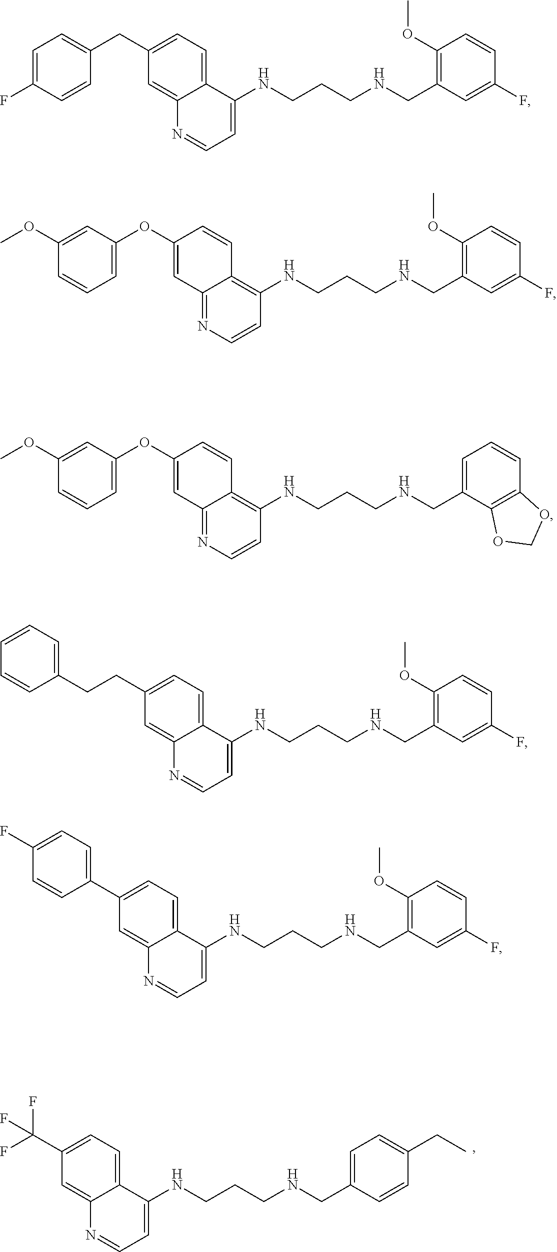 [SMILES: COc1ccc(F)cc1CNCCCNc1ccnc2cc(Cc3ccc(F)cc3)ccc12 ; COc1cccc(Oc2ccc3c(NCCCNCc4cc(F)ccc4OC)ccnc3c2)c1 ; COc1cccc(Oc2ccc3c(NCCCNCc4cccc5c4OCO5)ccnc3c2)c1 ; COc1ccc(F)cc1CNCCCNc1ccnc2cc(CCc3ccccc3)ccc12 ; COc1ccc(F)cc1CNCCCNc1ccnc2cc(-c3ccc(F)cc3)ccc12 ; CCc1ccc(CNCCCNc2ccnc3cc(C(F)(F)F)ccc23)cc1]

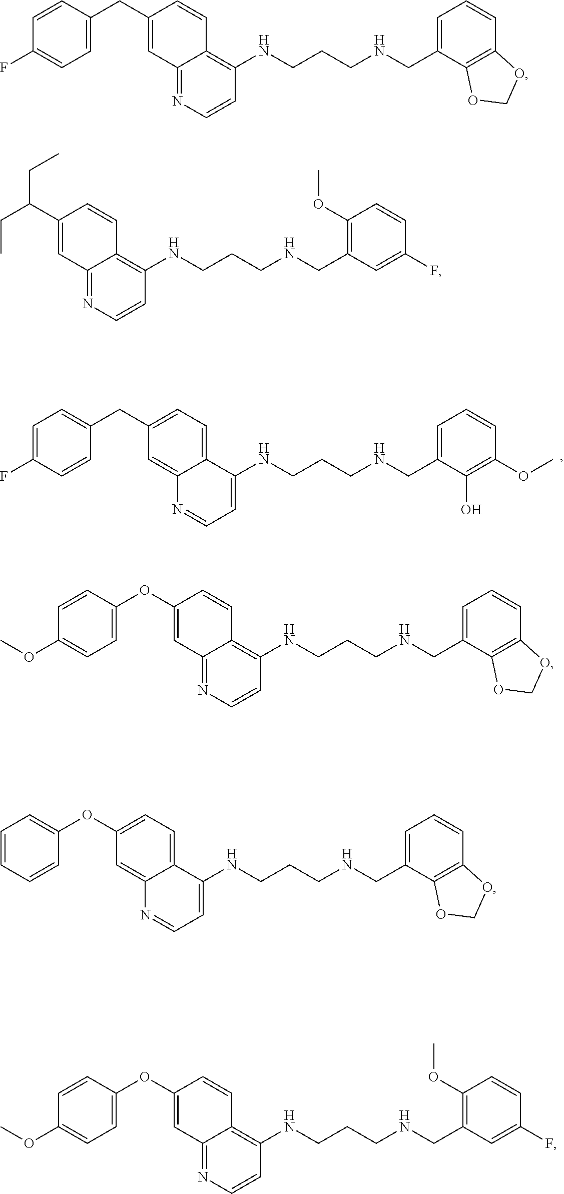

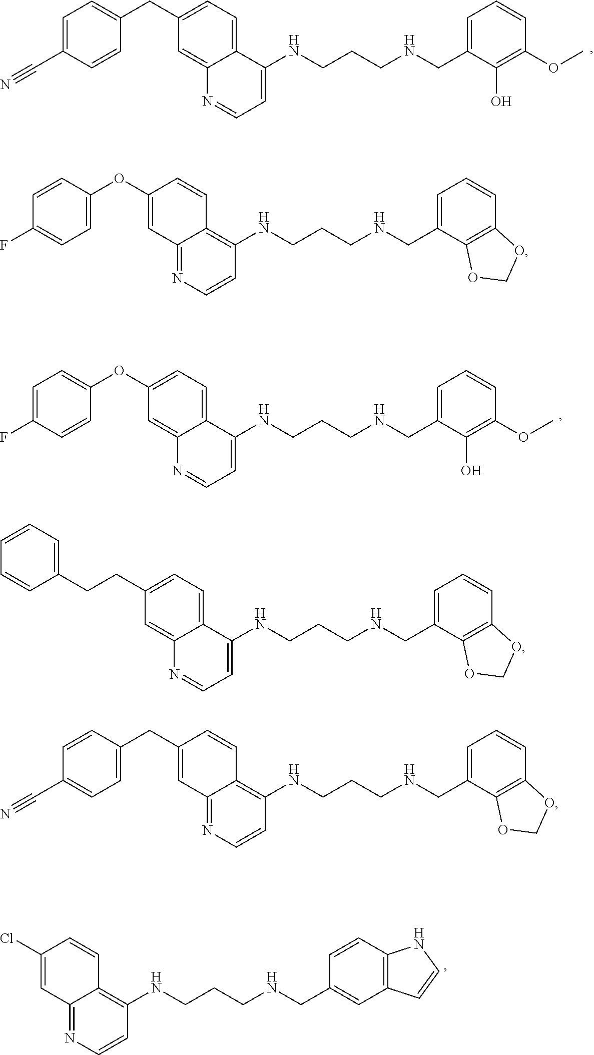

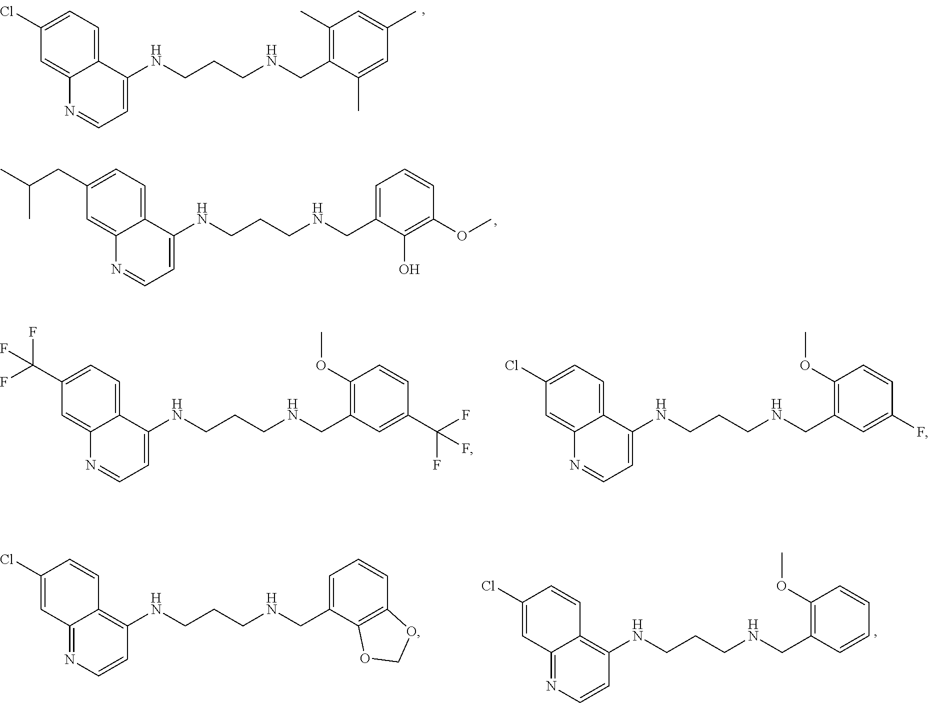

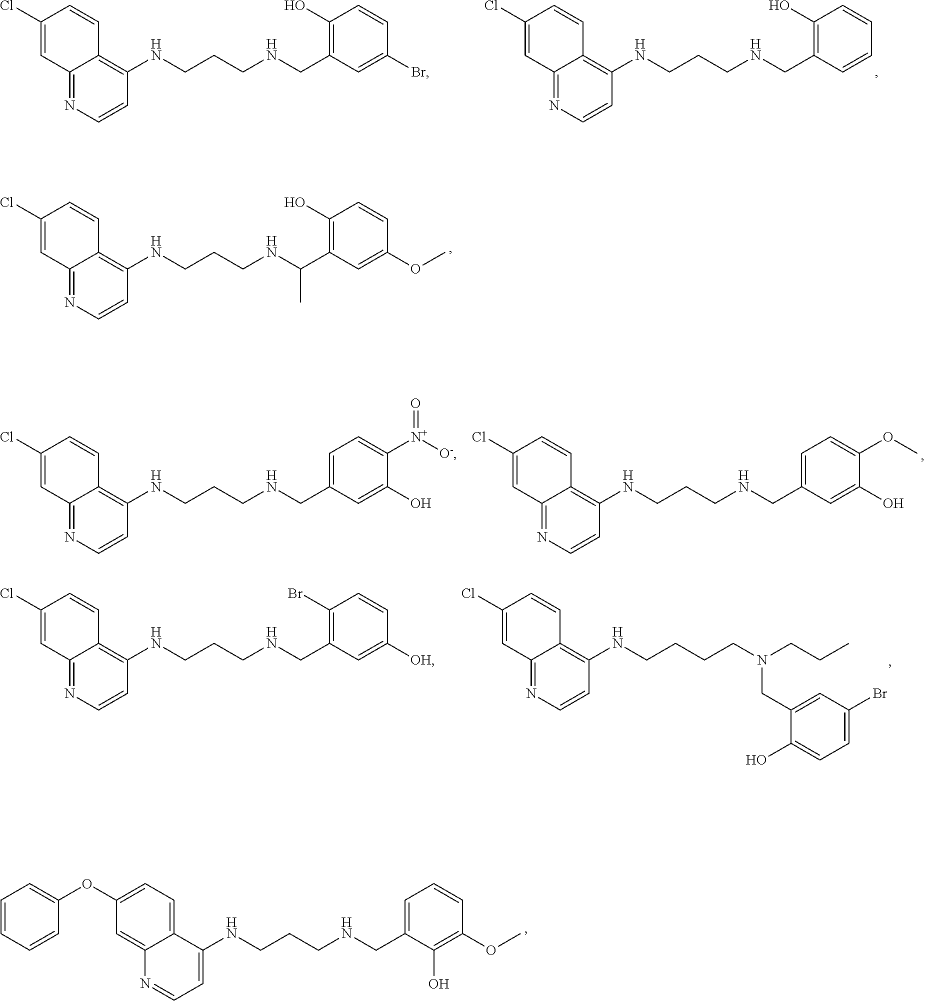

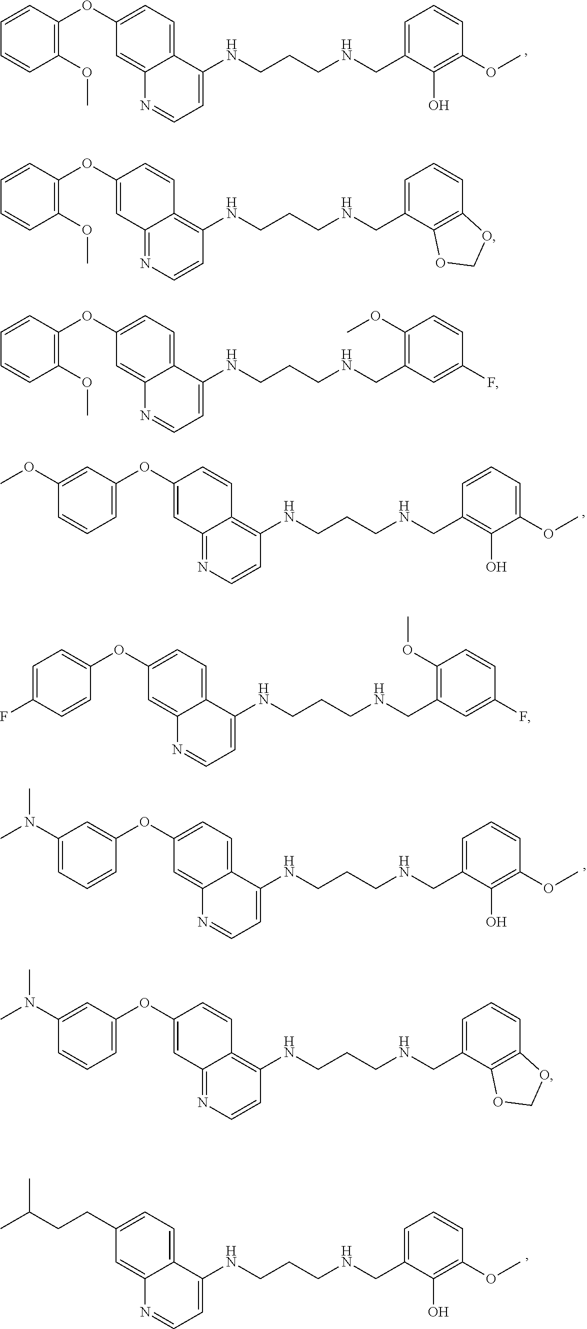

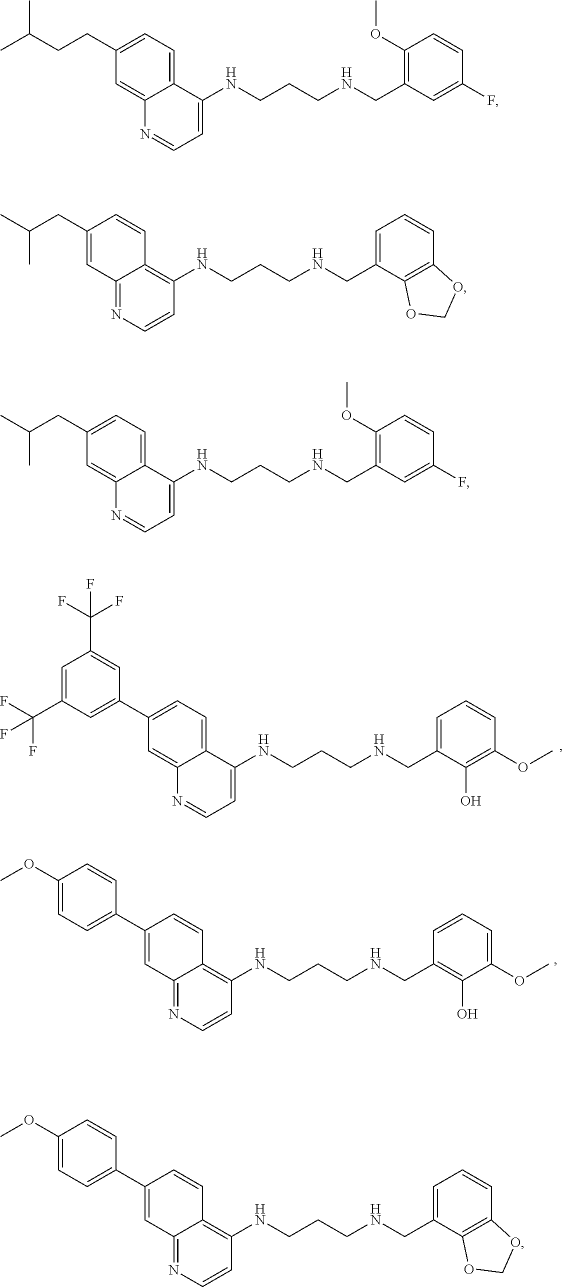

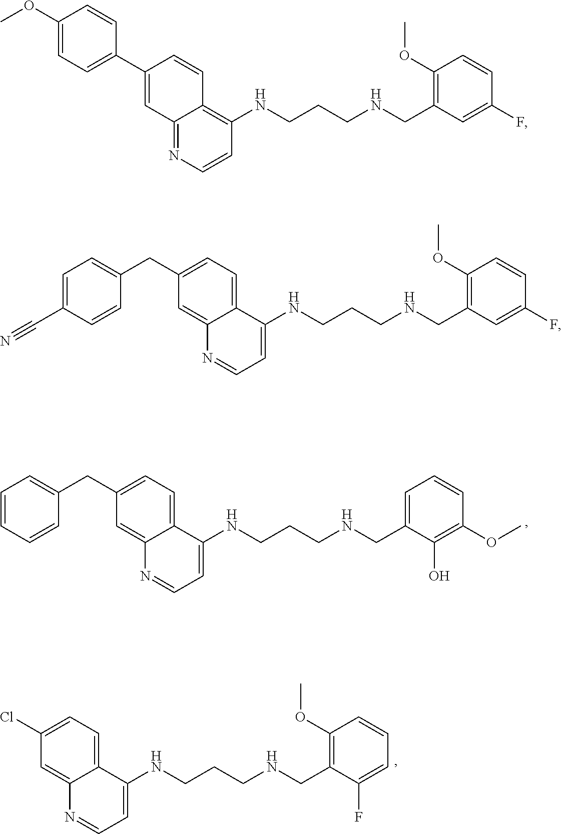 [SMILES: COc1ccc(-c2ccc3c(NCCCNCc4cc(F)ccc4OC)ccnc3c2)cc1 ; COc1ccc(F)cc1CNCCCNc1ccnc2cc(Cc3ccc(C#N)cc3)ccc12 ; COc1cccc(CNCCCNc2ccnc3cc(Cc4ccccc4)ccc23)c1O ; COc1cccc(F)c1CNCCCNc1ccnc2cc(Cl)ccc12]

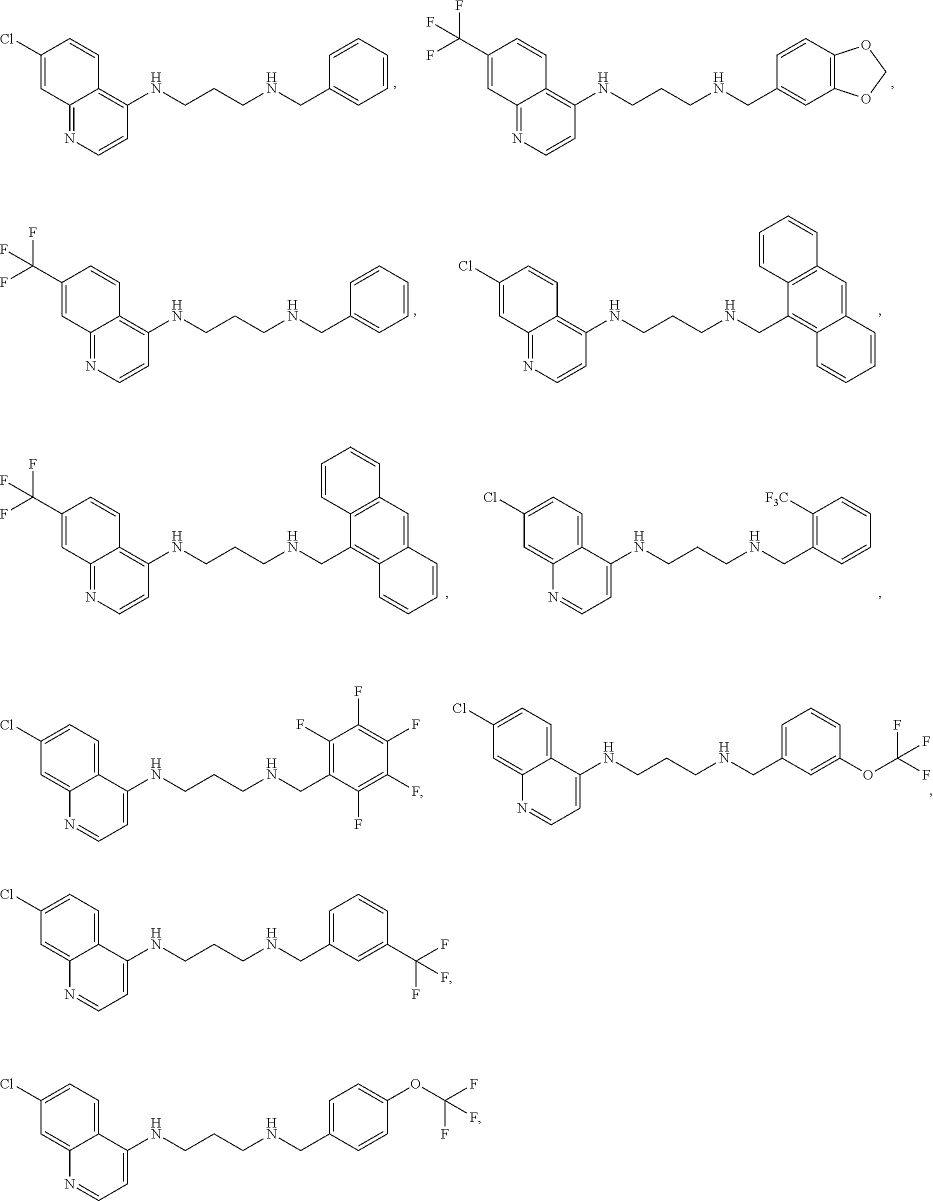

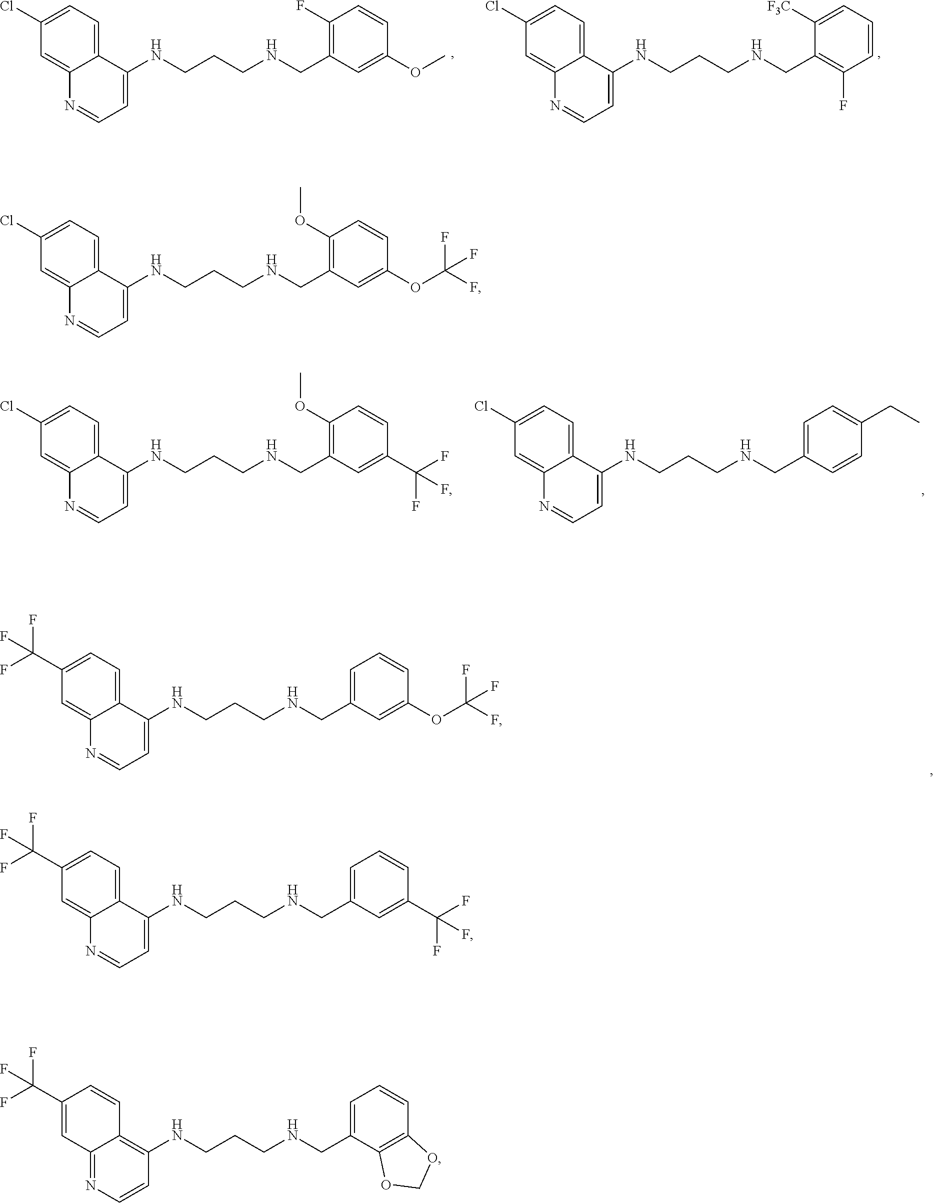

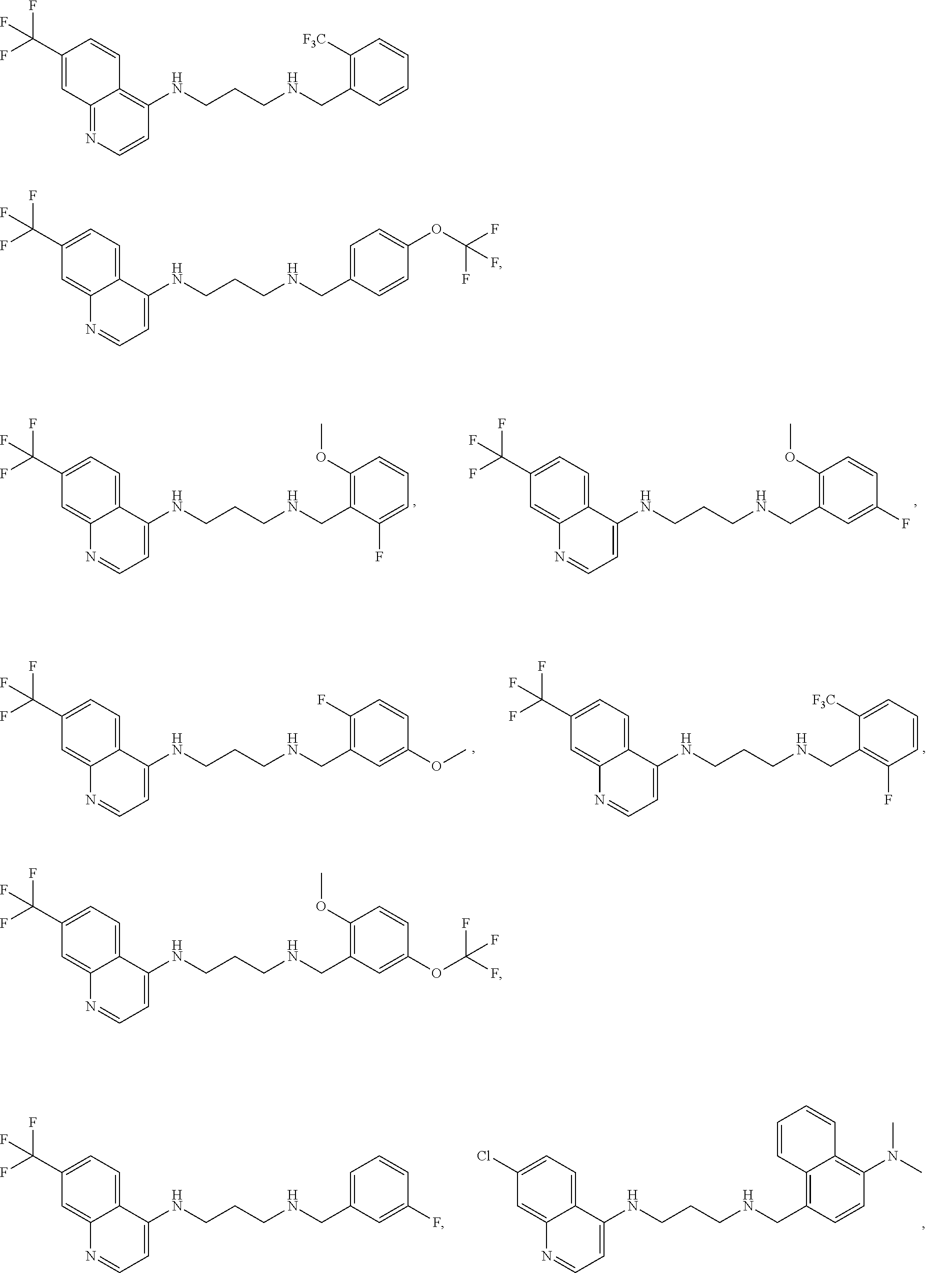

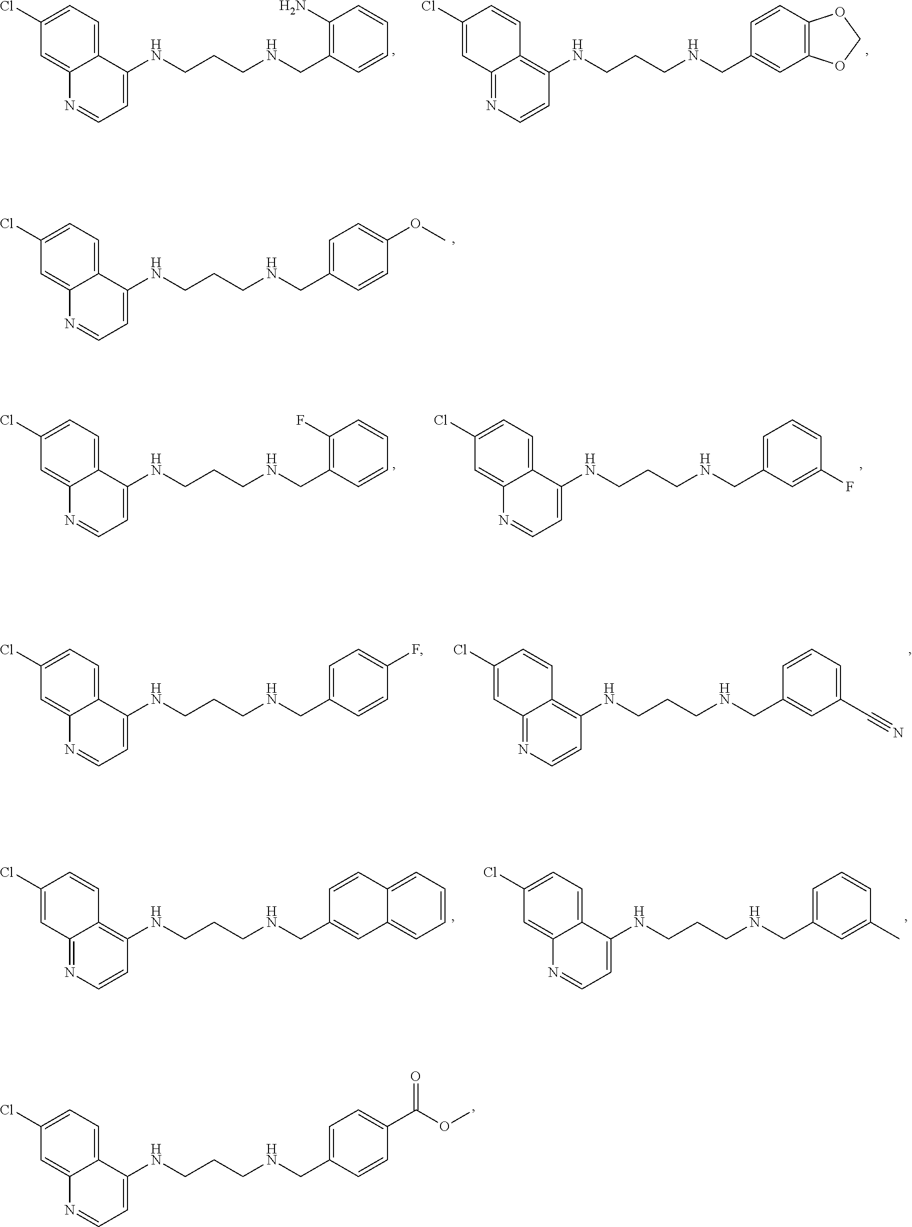

or a subgroup thereof.

In various aspects, the disclosed has a structure represented by a formula:

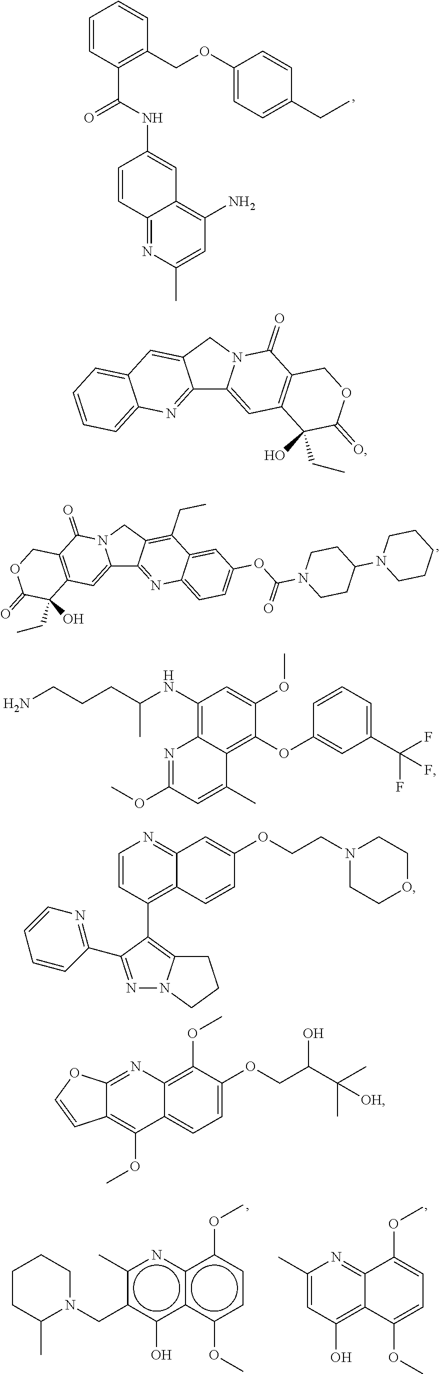

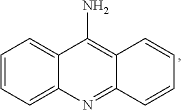 [SMILES: Nc1c2ccccc2nc2ccccc12]

or a subgroup thereof.

In various aspects, it is contemplated herein that the disclosed compounds further include their biosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prog. Drug Res. 1991, 37, 287-371; (iii) Burger A, “Isosterism and bioanalogy in drug design”, Med. Chem. Res. 1994, 4, 89-92; (iv) Clark R D, Ferguson A M, Cramer R D, “Bioisosterism and molecular diversity”, Perspect. Drug Discovery Des. 1998, 9/10/11, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thornber C W, “Isosterism and molecular modification in drug design”, Chem. Soc. Rev. 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one embodiment, the ester, amide or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, it is contemplated herein that the disclosed compounds further include their isotopically-labelled or isotopically-substituted variants, i.e., compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further include prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties including one or more nitrogen groups with a suitable acid. In various aspects, acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quatemized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### Peptides.

In various aspects, the present disclosure relates to compounds useful as inhibitors of MAGE-A11:substrate interaction. Disclosed are peptides having a structure given by the sequence:

FLVVVHQIRQLXQ,

wherein X is a substituted phenylalanine analog having the structure given by the formula:

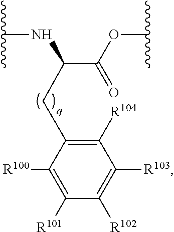

wherein q is selected from 0, 1, and 2; wherein each of R¹⁰⁰, R¹⁰¹, R¹⁰², R¹⁰³, and R¹⁰⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.

In a further aspect, q is selected from 2 and 3. In a still further aspect, q is 1. In a yet further aspect, q is 2. In an even further aspect, q is 3. In yet further aspect, q is selected from 1 and 3.

In a further aspect, each of R¹⁰⁰, R¹⁰¹, R¹⁰², R¹⁰³, and R¹⁰⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that four of R¹⁰⁰, R¹⁰¹, R¹⁰², R¹⁰³, and R¹⁰⁴ are hydrogen. In a still further aspect, each of R¹⁰⁰, R¹⁰¹, R¹⁰², R¹⁰³, and R¹⁰⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that three of R¹⁰⁰, R¹⁰¹, R¹⁰², R¹⁰³, and R¹⁰⁴ are hydrogen. In a yet further aspect, each of R¹⁰⁰, R¹⁰¹, R¹⁰², R¹⁰³, and R¹⁰⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that two of R¹⁰⁰, R¹⁰¹, R¹⁰², R¹⁰³, and R¹⁰⁴ are hydrogen. In an even further aspect, each of R¹⁰⁰, R¹⁰¹, R¹⁰², R¹⁰³, and R¹⁰⁴ is independently selected from hydrogen, fluoro, chloro, —SF₅, —CN, —N₃, —NH₂, —OH, —CN, C1-C2 alkoxy, C1-C2 haloalkyl, C1-C2 aminoalkyl, C1-C2 alkylamino, C1-C2 hydoxyalkyl, and C1-C3 alkyl. In a still further aspect, each of R¹⁰⁰, R¹⁰¹, R¹⁰², R¹⁰³, and R¹⁰⁴ is independently selected from hydrogen, fluoro, chloro, —NH₂, —OH, methyl, methoxy, trifluoromethyl, trichloromethyl, methylamino, and dimethylamino. In a yet further aspect, each of R¹⁰⁰, R¹⁰¹, R¹⁰², R¹⁰³, and R¹⁰⁴ is independently selected from hydrogen, fluoro, chloro, methyl, methoxy, and trifluoromethyl. In an even further aspect, each of R¹⁰⁰, R¹⁰¹, R¹⁰², R¹⁰³, and R¹⁰⁴ is hydrogen.

In a further aspect, X is a substituted phenylalanine analog having the structure given by the formula:

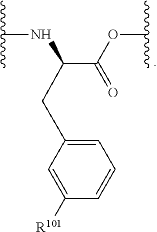

In a further aspect, X is a substituted phenylalanine analog having the structure given by the formula:

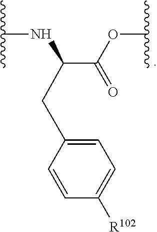

In a further aspect, X is a substituted phenylalanine analog having the structure given by the formula:

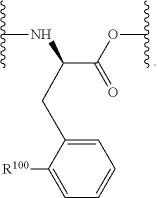

### Pharmaceutical Compositions.

In various aspects, the present disclosure relates to pharmaceutical compositions including a therapeutically effective amount of one or more disclosed compounds, or pharmaceutically acceptable salt thereof; a therapeutically effective amount of one or more disclosed peptides, or pharmaceutically acceptable salt thereof; or combinations thereof; and a pharmaceutically acceptable carrier. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In various aspects, the disclosed pharmaceutical composition further includes at least one agent known to treat a cancer. In a further aspect, the at least one agent known to treat a cancer is a hormone therapy agent; an alkylating agent, an antimetabolite agent, an antineoplastic antibiotic agent, a mitotic inhibitor agent, a mTor inhibitor agent, other chemotherapeutic agent, or combinations thereof.

In a further aspect, the at least one agent known to treat a cancer is a hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt thereof.

In a further aspect, the at least one agent known to treat a cancer is a antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In a further aspect, the at least one agent known to treat a cancer is an antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In a further aspect, the at least one agent known to treat a cancer is an alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt.

In a further aspect, the at least one agent known to treat a cancer is a mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt.

In a further aspect, the at least one agent known to treat a cancer is a mTor inhibitor agent is selected from one or more of the group consisting of everolimus, siroliumus, and temsirolimus, or a pharmaceutically acceptable salt thereof.

In a further aspect, the at least one agent known to treat a cancer is selected from uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17?-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.

In a further aspect, the disclosed pharmaceutical compositions include a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition including a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to include the therapeutically active non-toxic acid and base addition salt forms which the disclosed compounds are able to form.

In various aspects, a disclosed compound including an acidic group or moiety, e.g., a carboxylic acid group, can be used to prepare a pharmaceutically acceptable salt. For example, such a disclosed compound may include an isolation step including treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound including a protonatable group or moiety, e.g., an amino group, can be used to prepare a pharmaceutically acceptable salt. For example, such a disclosed compound may include an isolation step including treatment with a suitable inorganic or organic acid. In some cases, it may be desirable in practice to initially isolate a compound from the reaction mixture as a pharmaceutically unacceptable salt and then simply convert the latter back to the free base compound by treatment with an basoc reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt includes an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein include a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can include one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can include a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may include hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can include solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further include preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further include liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier includes saline solution, glucose solution or a mixture of saline and glucose solution. Injectable suspensions may also be prepared in which case appropriate liquid carriers, suspending agents and the like may be employed. In some aspects, a disclosed parenteral formulation can include about 0.01-0.1 M, e.g. about 0.05 M, phosphate buffer. In a further aspect, a disclosed parenteral formulation can include about 0.9% saline.

In various aspects, a disclosed parenteral pharmaceutical composition can include pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can include may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally includes a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes include a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally included of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically include a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further include a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, include a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions including the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example include metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions including a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will include from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require of inhibition of MAGE-A11 interaction with a cellular or physiological substrate an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for inhibiting MAGE-A11 interaction with a cellular or physiological substrate (e.g., treatment of one or more disorders associated with MAGE-A11 dysfunction) in mammals (e.g., humans) including combining one or more disclosed compounds, products, or compositions with a pharmaceutically acceptable carrier or diluent. Thus, in one aspect, the present disclosure further relates to a method for manufacturing a medicament including combining at least one disclosed compound or at least one disclosed product with a pharmaceutically acceptable carrier or diluent.

The disclosed pharmaceutical compositions can further include other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition including a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

### Methods of Using the Compounds.

In a further aspect, the present disclosure provides methods of treatment including administration of a therapeutically effective amount of a disclosed compound or pharmaceutical composition as disclosed herein above to a subject in need thereof.

In various aspects, the present disclosure relates to methods for the treatment of a disorder associated with MAGE-A11 dysfunction in a mammal including the step of administering to the mammal a therapeutically effective amount of one or more disclosed compounds, or pharmaceutically acceptable salt thereof; a therapeutically effective amount of one or more disclosed peptides, or pharmaceutically acceptable salt thereof; or combinations thereof.

In a further aspect, the present disclosure relates to methods for inhibiting a MAGE-A11:substrate interaction in a mammal including the step of administering to the mammal a therapeutically effective amount of one or more disclosed compounds, or pharmaceutically acceptable salt thereof; a therapeutically effective amount of one or more disclosed peptides, or pharmaceutically acceptable salt thereof; or combinations thereof.

In a further aspect, the present disclosure relates to methods for inhibiting MAGE-A11 activity in at least one cell, including the step of contacting the cell with an effective amount of one or more disclosed compounds, or pharmaceutically acceptable salt thereof; an effective amount of one or more disclosed peptides, or pharmaceutically acceptable salt thereof; or combinations thereof.

In a further aspect, the present disclosure relates to methods for the treatment of a disorder of uncontrolled cellular proliferation associated with a MAGE-A11 dysfunction in a mammal including the step of administering to the mammal a therapeutically effective amount of at least one disclosed compound, or a pharmaceutically acceptable salt thereof; at least one disclosed peptide; a disclosed pharmaceutical composition; or combinations thereof.

In a further aspect, the mammal is a human. In a still further aspect, the mammal has been diagnosed with a need for treatment of the disorder of uncontrolled cellular proliferation associated with a MAGE-A11 dysfunction prior to the administering step.

In a further aspect, the method further includes the step of identifying a mammal in need of treatment of the disorder of uncontrolled cellular proliferation associated with a MAGE-A11 dysfunction.

In a further aspect, the MAGE-A11 dysfunction is associated with aberrant expression of MAGE-A11. In a still further aspect, the disorder of uncontrolled cellular proliferation is a cancer. In a yet further aspect, the cancer is selected from the cancer is selected from a brain cancer, lung cancer, hematological cancer, bladder cancer, colon cancer, cervical cancer, ovarian cancer, squamous cell cancer, kidney cancer, peritoneal cancer, breast cancer, gastric cancer, colorectal cancer, prostate cancer, pancreatic cancer, genitourinary tract cancer, lymphatic system cancer, stomach cancer, larynx cancer, malignant melanoma, colorectal cancer, endometrial carcinoma, thyroid cancer, rhabdosarcoma, and combinations thereof. In a yet further aspect, the cancer is selected from lung cancer, ovarian cancer, and brain cancer. The lung cancer can be selected from small-cell lung cancer, non-small cell lung cancer, and combinations thereof. In some instances, the kidney cancer is a kidney clear cell carcinoma. The brain cancer can include, but is not limited to, a glioblastoma, medullablastoma, glioma, and combinations thereof. An exemplary, but non-limiting, bladder cancer is a bladder urothelial carcinoma; and an exemplary, but non-limiting, liver cancer is a hepatic carcinoma.

In a further aspect, the hematological cancer is selected from chronic myeloid leukemia (CML), acute myeloid leukemia (AML), chronic lymphoid leukemia (CLL), acute lymphoid leukemia (ALL), hairy cell leukemia, chronic myelomonocytic leukemia (CMML), juvenile myelomonocyte leukemia (JMML), large granular lymphocytic leukemia (LGL), acute lymphocytic leukemia, acute lymphoblastic leukemia, B-cell lymphoma, T-cell-lymphoma, Hodgkin's lymphoma, non-Hodgkin's lymphoma, hairy cell lymphoma, Burkett's lymphoma, Hodgkin lymphoma, non-Hodgkin lymphoma, and combinations thereof.

In a further aspect, the method further includes the step of administering a therapeutically effective amount of at least one agent known to treat a cancer. In a still further aspect, the at least one agent is selected from uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, and vasostatin. In a yet further aspect, the at least one agent is a DNA methyltransferase inhibitor, an HDAC-inhibitor, a glucocorticoid, an mTOR inhibitor, a cytotoxic agent, or combinations thereof. Suitable DNA methyltransferase inhibitors include, but are not limited to, 5-aza-2′-deoxycytidine, 5-azacytidine, zebularin, epigallocatechin-3-gallate, procaine, or combinations thereof. Exemplary, but non-limiting, HDAC-inhibitors include vorinostat, entinostat, panbinostat, trichostatin A, mocetinostat, belinostat, dacinostat, givinostat, tubastatin A, pracinostat, droxinostat, quisinostat, romidepsin, valproic acid, AR-42 (OSU-HDAC42), tacedinaline, rocilinostat, apicidin, or combinations thereof. A suitable glucocorticoid can be dexamethasone, prednisolone, methylprednisolone, betamethasone, triamicinolone, fludrocortisone, beclomethasone, or combinations thereof.

In a further aspect, the mTor inhibitor is BEZ235, everolimus, sirolimus, temsirolimus, rapamycin, AZD8055, or combinations thereof. In a still further aspect, the cytotoxic agent is an alkylating agent, an antimetabolite agent, an antineoplastic antibiotic agent, a mitotic inhibitor agent, a mTor inhibitor agent or other chemotherapeutic agent. In a yet further aspect, the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof. In an even further aspect, the antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof.

In various aspects, the alkylating agent can be selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof. In a further aspect, a suitable mitotic inhibitor agent can be, but is not limited to, irinotecan, topotecan, rubitecan, cabazitaxel, docetaxel, paclitaxel, etopside, vincristine, ixabepilone, vinorelbine, vinblastine, and teniposide, or a pharmaceutically acceptable salt, hydrate, solvate, or polymorph thereof.

In some instances, the other chemotherapeutic agent can be an agent such as an anthracycline, cytarabine, a purine analog, sorafenib, gemtuzumab ozogamicin, rituximab, or combinations thereof. Exemplary anthracyclines include, but are not limited to, daunorubicin, idarubicin, or combinations thereof. A suitable purine analog useful in the disclosed methods is cladribine, fludarabine, clofarabine, or combinations thereof.

In a further aspect, the at least one compound and the at least one agent are administered sequentially.

In a further aspect, the at least one compound and the at least one agent are administered simultaneously.

In a further aspect, the at least one compound, the at least one peptide, and the at least one agent are co-formulated. Alternatively, the at least one compound, the at least one peptide, and the at least one agent are co-packaged.

In a further aspect, the present disclosure relates to methods for modulating of MAGE-A11 activity in a mammal including the step of administering to the mammal a therapeutically effective amount of at least one disclosed compound, or a pharmaceutically acceptable salt thereof; at least one disclosed peptide; a disclosed pharmaceutical composition; or combinations thereof.

In a further aspect, the present disclosure relates to methods for modulating of MAGE-A11 activity in at least one cell, including the step of contacting the at least one cell with an effective amount of at least one disclosed compound, or a pharmaceutically acceptable salt thereof; at least one disclosed peptide; a disclosed pharmaceutical composition; or combinations thereof. The cell conteacted can be a mammalian cell, e.g., a human cell. In some instances, the cell has been isolated from a mammal prior to the contacting step.

Modulation of MAGE-A11 activity can be inhibition of the binding MAGE-A11 with a substrate, e.g., a cellular or physiological substrate. In some instances, contacting the cell can be administration to a mammal.

In a further aspect, the mammal has been diagnosed with a need for modulating of MAGE-A11 activity prior to the administering step. In a still further aspect, the mammal has been diagnosed with a need for treatment of a disorder related to MAGE-11A activity prior to the administering step.

In various aspects, the compound or peptide exhibits in a TR-FRET-based assay inhibition of MAGE-11A binding to a PCF-11 substrate with an IC₅₀ of less than about 10,000 nM; less than about 5,000 nM; less than about 1,000 nM; less than about 750 nM; less than about 500 nM; less than about 250 nM; less than about 200 nM; or less than about 150 nM.

### Kits.

In a further aspect, the present disclosure relates to kits including at least one disclosed compound, or a pharmaceutically acceptable salt, hydrate, solvate, or polymorph thereof, and one or more of: (a) at least one agent known to increase MAGE-A11 activity; (b) at least one agent known to decrease MAGE-A11 activity; (c) at least one agent known to treat a disorder of uncontrolled cellular proliferation; (d) instructions for treating a disorder of uncontrolled cellular proliferation; or (e) instructions for treating a disorder associated with MAGE-11A dysfunction.

The disclosed compounds and/or pharmaceutical compositions including the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit including one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also include compounds and/or products co-packaged, co-formulated, and/or co-delivered with other components. For example, a drug manufacturer, a drug reseller, a physician, a compounding shop, or a pharmacist can provide a kit including a disclosed compound and/or product and another component for delivery to a patient.

It is contemplated that the disclosed kits can be used in connection with the disclosed methods of making, the disclosed methods of using or treating, and/or the disclosed compositions.

### Research Tools.

The disclosed compounds and pharmaceutical compositions have activity as inhibitors of MAGE-A11 interaction with a cellular or physiological substrate. As such, the disclosed compounds are also useful as research tools. Accordingly, one aspect of the present disclosure relates to a method of using a compound of the invention as a research tool, the method including conducting a biological assay using a compound of the invention. Compounds of the invention can also be used to evaluate new chemical compounds. Thus another aspect of the invention relates to a method of evaluating a test compound in a biological assay, including: (a) conducting a biological assay with a test compound to provide a first assay value; (b) conducting the biological assay with a compound of the invention to provide a second assay value; wherein step (a) is conducted either before, after or concurrently with step (b); and (c) comparing the first assay value from step (a) with the second assay value from step (b). Exemplary biological assays include a TR-FRET assay as disclosed herein that can be conducted in vitro system. Still another aspect of the invention relates to a method of studying a biological system, e.g., a model animal for a clinical condition, or biological sample including a MAGE-A11 protein, the method including: (a) contacting the biological system or sample with a compound of the invention; and (b) determining the effects caused by the compound on the biological system or sample.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### REFERENCES

References are cited herein throughout using the format of reference number(s) enclosed by parentheses corresponding to one or more of the following numbered references. For example, citation of references numbers 1 and 2 immediately herein below may be indicated in the disclosure as (Refs. 1 and 2), (Ref. 1, 2), or the like.
1. Weon, J. L. & Potts, P. R. The MAGE protein family and cancer. Curr Opin Cell Biol 37, 1-8 (2015).
2. Lee, A. K. & Potts, P. R. A Comprehensive Guide to the MAGE Family of Ubiquitin Ligases. J Mol Biol 429, 1114-1142 (2017).
3. Fon Tacer, K. et al. MAGE cancer-testis antigens protect the mammalian germline under environmental stress. Sci Adv 5, eaav4832 (2019).
4. Tacer, K. F. & Potts, P. R. Cellular and disease functions of the Prader-Willi Syndrome gene MAGEL2. Biochem J 474, 2177-2190 (2017).
5. Pineda, C. T. et al. Degradation of AMPK by a cancer-specific ubiquitin ligase. Cell 160, 715-728 (2015).
6. Simpson, A. J., Caballero, O. L., Jungbluth, A., Chen, Y. T. & Old, L. J. Cancer/testis antigens, gametogenesis and cancer. Nat Rev Cancer 5, 615-25 (2005).
7. Barker, P. A. & Salehi, A. The MAGE proteins: emerging roles in cell cycle progression, apoptosis, and neurogenetic disease. J Neurosci Res 67, 705-12 (2002).
8. Doyle, J. M., Gao, J., Wang, J., Yang, M. & Potts, P. R. MAGE-RING protein complexes comprise a family of E3 ubiquitin ligases. Mol Cell 39, 963-74 (2010).
9. Newman, J. A. et al. Structures of Two Melanoma-Associated Antigens Suggest Allosteric Regulation of Effector Binding. PLOS One 11, e0148762 (2016).
10. Yang, S. W. et al. A Cancer-Specific Ubiquitin Ligase Drives mRNA Alternative Polyadenylation by Ubiquitinating the mRNA 3′ End Processing Complex. Mol Cell (2020).
11. Yang, B. et al. Select cancer testes antigens of the MAGE-A, -B, and -C families are expressed in mast cell lines and promote cell viability in vitro and in vivo. J Invest Dermatol 127, 267-75 (2007).
12. Yang, B. et al. MAGE-A, mMage-b, and MAGE-C proteins form complexes with KAP1 and suppress p53-dependent apoptosis in MAGE-positive cell lines. Cancer Res 67, 9954-62 (2007).
13. Atanackovic, D. et al. Cancer-testis antigens MAGE-C1/CT7 and MAGE-A3 promote the survival of multiple myeloma cells. Haematologica 95, 785-93 (2010).
14. Su, S. et al. Proto-oncogene activity of melanoma antigen-A11 (MAGE-A11) regulates retinoblastoma-related p107 and E2F1 proteins. J Biol Chem 288, 24809-24 (2013).
15. Weon, J. L., Yang, S. W. & Potts, P. R. Cytosolic Iron-Sulfur Assembly Is Evolutionarily Tuned by a Cancer-Amplified Ubiquitin Ligase. Mol Cell 69, 113-125 e6 (2018).
16. Hao, Y. H. et al. Regulation of WASH-dependent actin polymerization and protein trafficking by ubiquitination. Cell 152, 1051-64 (2013).
17. Sasaki, A., Masuda, Y., Iwai, K., Ikeda, K. & Watanabe, K. A RING finger protein Praja1 regulates DIx5-dependent transcription through its ubiquitin ligase activity for the DIx/Msx-interacting MAGE/Necdin family protein, DIxin-1. J Biol Chem 277, 22541-6 (2002).
18. Kozakova, L. et al. The melanoma-associated antigen 1 (MAGEA1) protein stimulates the E3 ubiquitin-ligase activity of TRIM31 within a TRIM31-MAGEA1-NSE4 complex. Cell Cycle 14, 920-30 (2015).
19. Potts, P. R., Porteus, M. H. & Yu, H. Human SMC5/6 complex promotes sister chromatid homologous recombination by recruiting the SMC1/3 cohesin complex to double-strand breaks. EMBO J 25, 3377-88 (2006).
20. Petroski, M. D. & Deshaies, R. J. Function and regulation of cullin-RING ubiquitin ligases. Nat Rev Mol Cell Biol 6, 9-20 (2005).
21. Bai, S., He, B. & Wilson, E. M. Melanoma antigen gene protein MAGE-11 regulates androgen receptor function by modulating the interdomain interaction. Mol Cell Biol 25, 1238-57 (2005).
22. Lian, Y. et al. Expressions of MAGE-A10 and MAGE-A11 in breast cancers and their prognostic significance: a retrospective clinical study. J Cancer Res Clin Oncol 138, 519-27 (2012).
23. Xia, L. P., Xu, M., Chen, Y. & Shao, W. W. Expression of MAGE-A11 in breast cancer tissues and its effects on the proliferation of breast cancer cells. Mol Med Rep 7, 254-8 (2013).
24. Braisted, A. C. et al. Discovery of a potent small molecule IL-2 inhibitor through fragment assembly. J Am Chem Soc 125, 3714-5 (2003).
25. Oltersdorf, T. et al. An inhibitor of Bcl-2 family proteins induces regression of solid tumours. Nature 435, 677-81 (2005).
26. Grasberger, B. L. et al. Discovery and cocrystal structure of benzodiazepinedione HDM2 antagonists that activate p53 in cells. J Med Chem 48, 909-12 (2005).
27. Zhang, J. H., Chung, T. D. & Oldenburg, K. R. A Simple Statistical Parameter for Use in Evaluation and Validation of High Throughput Screening Assays. J Biomol Screen 4, 67-73 (1999).
28. van der Bruggen, P. et al. A gene encoding an antigen recognized by cytolytic T lymphocytes on a human melanoma. Science 254, 1643-7 (1991).
29. Jager, E. & Knuth, A. The discovery of cancer/testis antigens by autologous typing with T cell clones and the evolution of cancer vaccines. Cancer Immun 12, 6 (2012).
30. Pitre, A. et al. An unexpected protein interaction promotes drug resistance in leukemia. Nat Commun 8, 1547 (2017).
31. Otwinowski, Z. & Minor, W. Processing of X-ray diffraction data collected in oscillation mode. Methods Enzymol 276, 307-26 (1997).
32. McCoy, A. J. et al. Phaser crystallographic software. J Appl Crystallogr 40, 658-674 (2007).
33. Emsley, P., Lohkamp, B., Scott, W. G. & Cowtan, K. Features and development of Coot. Acta Crystallogr D Biol Crystallogr 66, 486-501 (2010).
34. Murshudov, G. N., Vagin, A. A. & Dodson, E. J. Refinement of macromolecular structures by the maximum-likelihood method. Acta Crystallogr D Biol Crystallogr 53, 240-55 (1997).
35. Delano, W. L. The PyMOL Molecular Graphics System, (DeLano Scientific, San Carlos, 2002).
36. Dobin, A. et al. STAR: ultrafast universal RNA-seq aligner. Bioinformatics 29, 15-21 (2013).
37. Quinlan, A. R. & Hall, I. M. BEDTools: a flexible suite of utilities for comparing genomic features. Bioinformatics 26, 841-2 (2010).
38. Goldman, M. et al. The UCSC Cancer Genomics Browser: update 2015. Nucleic Acids Res 43, D812-7 (2015).
39. Feng, X., Li, L., Wagner, E. J. & Li, W. TC3A: The Cancer 3′ UTR Atlas. Nucleic Acids Res 46, D1027-D1030 (2018).
40. Natarajan, J. K. et al. 4-N-, 4-S-, and 4-O-chloroquine analogues: influence of side chain length and quinolyl nitrogen pKa on activity vs chloroquine resistant malaria. J Med Chem 51, 3466-79 (2008).
41. Hwang, J. Y. et al. Synthesis and evaluation of 7-substituted 4-aminoquinoline analogues for antimalarial activity. J Med Chem 54, 7084-93 (2011).
42. Jacobs, L. et al. Design, Synthesis, and Evaluation of Novel Ferroquine and Phenylequine Analogues as Potential Antiplasmodial Agents. ChemMedChem 10, 2099-110 (2015).
43. Ghose, A. K. & Crippen, G. M. Atomic physicochemical parameters for three-dimensional-structure-directed quantitative structure-activity relationships. 2. Modeling dispersive and hydrophobic interactions. J Chem Inf Comput Sci 27, 21-35 (1987).
44. Laskowski, R. A. & Swindells, M. B. LigPlot+: multiple ligand-protein interaction diagrams for drug discovery. J Chem Inf Model 51, 2778-86 (2011).

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Methods.

Cell culture and stable cell lines. HEK293FT, HeLa and DAOY cells were grown in DMEM (Gibco) supplemented with 10% (v/v) FBS (Hyclone), 2 mM L-glutamine, 100 units/ml penicillin, 100 units/ml streptomycin, and 0.25 mg/ml amphotericin B (Invitrogen). HEK293FT and DAOY cells were transfected with either HA-FLAG-vector, FLAG-MAGE-A11 wild-type or SBC mutants using Effectene (QIAGEN) according to the manufacturer's protocol in 6 cm² plates. After 48 hours, cells were selected with 1 μg/ml puromycin (Sigma) over 2 weeks.

Immunoprecipitation. HEK293FT cells were plated in 6 cm² plates and transfected 24 hours later with Effectene (QIAGEN) according to the manufacturer's protocol. After 48 hours, cells were washed and scraped in cold PBS, spun down and resuspended in lysis buffer (50 mM Tris pH 7.4, 150 mM NaCl, 1 mM DTT, 0.1% (v/v) Triton X-100, 10 mM N-Ethylmaleimide and 1× protease inhibitor cocktail (Sigma)). Cell lysates were incubated with appropriate antibodies overnight at 4° C. and then with protein A beads for 2 hours at 4° C. Beads were then washed with lysis buffer three times and eluted with 2×SDS sample buffer.

Western Blotting and Antibodies. For western blotting, samples in SDS sample buffer were resolved on SDS-PAGE gels and then transferred to nitrocellulose membranes prior to blocking in TBST with 5% (w/v) milk powder or 3% (w/v) bovine serum albumin and probing with primary and secondary antibodies (GE Healthcare). Primary antibodies were: anti-Myc (Sigma, #C3956), anti-FLAG (Sigma, #F3165), anti-HUWE1 (Novus Biologicals, #NB 100-652), anti-PCF11 (Bethyl Laboratories, #A303-706A), anti-GAPDH (Cell Signaling Technology, #2118), anti-TRIM28 (Abcam, #ab22553), anti-AMPKa1 (Cell Signaling Technology, #2795) and anti-MMS19 (Proteintech, #16015-1-AP). Protein signal was visualized after addition of ECL detection reagent (GE Healthcare) according to manufacturer's instructions.

Peptide Synthesis. N,N-dimethylformamide (DMF), N-methylpyrrolidone (NMP), trifluoroacetic acid (TFA), 2-(6-Chloro-1-H-benzotriazole-1-yl)-1,1,3,3-tetramethylaminium hexafluorophosphate (HCTU) and N-methylmorpholine (NMM), were purchased from GyrosProteinTechnologies (Tucson, AZ). O-(7-Azabenzotriazol-1-yl)-N,N,N′,N′-tetramethyluronium hexafluorophosphate (HATU), 1-Hydroxy-7-azabenzotriazole (HOAt), and substituted FMOC-phenylalanine analogs were purchased from ChemImpex (Wood Dale, IL). Piperidine, N,N-Diisopropylethylamine (DIPEA), acetic anhydride, thioanisole, triisopropylsilane (TIS), phenol, 1,2-ethanedithiol (EDT), Fmoc-Lys(Ac)-OH, diethyl ether and Hoveyda-Grubbs 2nd generation catalyst were purchased from Sigma-Aldrich (St Louis, MO). Cyanine5 NHS ester was purchased from Lumiprobe (Hunt Valley, MD). Fmoc-(S)-2-(4-pentenyl)alanine and Fmoc-(R)-2-(4-pentenyl)alanine were purchased from Aapptec (Louisville, KY). All standard Fmoc-amino acids and preloaded Wang resins were purchased Millipore-Sigma (Burlington, MA). Dichloromethane (DCM) was purchased from Fisher Scientific (Waltham, MA). N,N-Diisopropylcarbodiimide (DIC) and fluorescein-5-carboxylic acid (5-FAM) were purchased from Anaspec (Fremont, CA).

For standard peptide synthesis, peptides were synthesized at a 25 μmol scale on a SymphonyX peptide synthesizer (GyrosProteinTechnologies, Tucson, AZ) using preloaded Wang resins. Deprotection was performed with 20% piperidine in NMP for 3×5 minutes at room temperature. Coupling was performed with 0.2 M amino acid with a ratio of 1/5/5 amino acid /HCTU/NMM in NMP for 2×60 minutes at room temperature. Capping was performed after each amino acid cycle using 5% acetic anhydride in NMP at room temperature. After synthesis, peptides were cleaved from the resin using 82.5/5/5/2.5/2.5/2.5-TFA/water/thioanisole/TIS/phenol/EDT for 2 hours at room temperature. After filtration peptides were precipitated using ice-cold diethyl ether followed by centrifugation. Peptides were dissolved in water and lyophilized on a Freezemobile (SP Scientific, Warminster, PA). Peptides were HPLC purified using a Waters Preparative HPLC system (Waters, Milford, MA). Peptide purity was checked using an Analytical HPLC system (Waters, Milford, MA). All peptides synthesized had the expected molecular weights and were analyzed using a MicroFlex MALDI mass spectrophotometer (Bruker, Billerica, MA).

For peptides labeled with 5-FAM, peptides were synthesized at a 25 μmol scale in the same way as described above. After the synthesis was complete, the peptide resin was treated with 5-FAM/HOAt/DIC in the ratio 1/3.25/4.20 in NMP at room temperature overnight. Peptides were cleaved, purified and analyzed as described above.

For library peptide synthesis, a peptide library of 250 peptides was synthesized at a 10 μmol scale on an Overture robotic peptide synthesizer (GyrosProteinTechnologies, Tucson, AZ) using preloaded Wang resins. Deprotection was performed with 20% piperidine in NMP for 3×5 minutes at room temperature. Coupling was performed with 0.2 M amino acid with a ratio of 1/5/5 amino acid/HCTU/NMM in NMP for 2×60 minutes at room temperature. Capping was performed after each amino acid cycle using 5% acetic anhydride in NMM at room temperature. After synthesis, peptides were cleaved from the resin using 82.5/5/5/2.5/2.5/2.5-TFA/water/thioanisole/TIS/phenol/EDT for 2 hours at room temperature. After filtration peptides were precipitated using ice-cold diethyl ether followed by centrifugation. All peptides synthesized had the expected molecular weights and were analyzed using a MicroFlex MALDI mass spectrophotometer (Bruker, Billerica, MA).

For synthesis of peptides containing modified phenylalanine, peptides were synthesized at a 25 μmol scale in the same way as described above. The modified phenylalanines were introduced by manually activating the corresponding Fmoc-phenylalanine derivatives and coupling using a ratio of 1/5/5 amino acid/HATU/DIPEA in NMP for 2×60 minutes at room temperature. Peptides were cleaved, purified and analyzed as described above.

TR-FRET Assays and High-throughput Screen. Terbium-anti-GST antibody, 1 M Tris pH 7.5 1 M MgCl₂ and 1 M DTT were purchased from Invitrogen (Carlsbad, CA), BSA was purchased from Sigma (St. Louis, MO). Dimethyl sulfoxide (DMSO) was purchased from Fisher Scientific (Pittsburgh, PA). Black 384-well low volume plates and 384-well polypropylene compound plates were purchased from Corning Incorporated (Tewksbury, MA). The St. Jude FDA drug and Bioactive library (11,293 chemicals), Drug-like library (10,073 chemicals) and Lead-like library (10,041 chemicals) were custom assembled and purchased from commercial sources including Sigma, Microsource (Gaylordsville, CT), selleckchem (Houston, TX), ChemDiv (San Diego, CA), ChemBridge (San Diego, CA), Enamine (Monmouth Jct., NJ), and Life Chemicals (Niagara-on-the-Lake, ON, Canada). They were arrayed (10 mM in DMSO) in 384-well polypropylene compound plates with columns 1, 2, 13 and 14 empty which were reserved for controls.

The TR-FRET assay buffer with a formula of 50 mM Tris pH 7.5, 20 mM MgCl₂, 0.1 mg/ml BSA and 1 mM DTT was freshly prepared before each experiment. All TR-FRET assays were performed in black 384-well plates with 20 μl/well assay volume in the TR-FRET assay buffer at room temperature (approximately 25° C.). All peptides or compounds were solubilized in DMSO. To test the competitive activity of peptides or compounds against the interaction between GST-MAGEA11 MHD and FAM-PCF11_4 peptide, 10 μl/well peptide or compound at specified concentration was dispensed into a black 384-well low volume plate, followed by 5 μl/well FAM-PCF11_4 (800 nM), 5 μl/well Tb-anti-GST (20 nM) and GST-MAGEA11 MHD (20 nM). The assay components in each well was then mixed by shaking the plate on an IKA MTS 2/4 digital microtiter plate shaker (IKA Works; Wilmington, NC, USA) at 900 RPM for 1 minute. The plate was further centrifuged in an Eppendorf 5810 centrifuge with an A-4-62 swing-bucket rotor (Eppendorf AG, Hamburg, Germany) at 201 g (1000 rpm) for 30 seconds. The plate was then incubated for 90 min with a lid to avoid light exposure to the assay components. After incubation, the fluorescent emission signals at 520 nm and 490 nm channels of individual wells were measured with a PHERAstar FS plate reader (BMG Labtech; Durham, NC, USA) by using a 340 nm excitation filter, 100 μs delay time, and 200 μs integration time. The TR-FRET fluorescence emission ratio (TR-FRET signal) for each well was presented as 10 000×520 nm/490 nm, which was used directly for curve fitting, or further converted to % Inhibition based on the respective positive and negative controls. The graphic software GraphPad Prism 4.81 (GraphPad Software; La Jolla, CA, USA) was used to generate curves and derive IC₅₀ values of tested peptides or compounds, if applicable.

TR-FRET inhibitory activity characterization of a panel of PCF11 peptides against the interaction between GST-MAGEA11 MHD and FAM-PCF11_4 was performed as follows. In a 20 μl/well assay volume, dilutions of PCF11_1 to PCF11_8 peptides (concentrations ranged from 3.1 nM to 100 μM for peptides PCF11_4, PCF11_5, PCF11_6 and PCF11_8 in a 1-to-2 dilution pattern for 16 concentration levels, and ranged from 48.9 nM to 100 μM for peptides of PCF11_1, PCF11_2, PCF11_3 and PCF11_7 in a 1-to-2 dilution pattern for 12 concentration levels), 200 nM FAM-PCF11_4, 5 nM Tb-anti-GST and 5 nM GST-MAGEA11 were mixed together and incubated for 90 min before the TR-FRET signal for each well was measured. The final DMSO concentration was 1.1% (v/v). In each group, a group of wells with 1.1% (v/v) DMSO along with 200 nM FAM-PCF11_4, 5 nM Tb-anti-GST and 5 nM GST-MAGEA11 MHD (DMSO group) was included as the negative control (SignalpMso, 0% inhibition). A group of wells with 1.1% (v/v) DMSO along with 200 nM FAM-PCF11_4 and 5 nM Tb-anti-GST (DMSO without GST-MAGEA11 group) was also included as the positive control (SignalDMSO without GST-MAGEA11, 100% inhibition). The signal of each peptide-treated well (Signalpeptide) was normalized to the positive and negative controls to derive % inhibition of the peptide at a specific concentration with equation 1. Where applicable, the % Inhibition data were fit into a Sigmoidal dose response equation to derive IC₅₀ values.

$$\% Inhibition = 100 \% - 100 \% \times \frac{( {Signal}_{peptide} - {Signal}_{DMSO without GST - MAGEA 11} )}{( {Signal}_{DMSO} - {Signal}_{DMSO without GST - M G E A 11} )} . \qquad Equation 1$$

TR-FRET screen for identifying MAGEA11 small molecule inhibitors was performed as follows. The primary one-concentration screening and subsequent 10 or 20-concentration dose response tests were accomplished with an automated High Throughput Screening system (HighRes Biosolutions, Beverly, MA)³⁰. In the primary screening, 15 μl/well FAM-PCF11_4 (267 nM) was first dispensed with a Multidrop Combi Reagent Dispenser (Thermo Fisher Scientific, Waltham, MA) to black 384-well low volume assay plates. The plates were spun down with a Velocity11 VSpin microplate centrifuge (Agilent Technologies, Santa Clara, CA). 30 nl/well of 10 mM compound/DMSO solutions, or DMSO alone, were transferred from compound plates (the St. Jude FDA drug and bioactive library, Drug-like library and Lead-like library) or a control plate to the assay plates with a Pin Tool (V&P Scientific, San Diego, CA). The compounds were arrayed in columns 3 to 12 and columns 15 to 24 of compound plates. The controls were arrayed in columns 1, 2, 13 and 14 of a compound plate. 5 μl/well Tb-anti-GST (20 nM) and GST-MAGEA11 (20 nM) or 5 μl/well Tb-anti-GST (20 nM) alone was then dispensed to selected wells with a Multidrop Combi Reagent Dispenser. The final assay volume was 20 μl/well. The final DMSO concentration was 0.25% (v/v) (0.1% (v/v) from the FAM-PCF11_4 DMSO stock solution and 0.15% (v/v) from the compound-DMSO solution or the DMSO for the control wells). The final tested compound concentration was 15 μM. The final FAM-PCF11_4 concentration was 200 nM and the final Tb-anti-GST concentration was 5 nM. The final GST-MAGEA11 concentration was 5 nM except for those selected positive control wells in which GST-MAGEA11 was not presented. The plates were then shaken at 900 RPM with a big bear shaker (Big Bear Automation, Santa Clara, California) and spun down with the Velocity11 VSpin microplate centrifuge for 20 seconds. The plates were then incubated at room temperature in a Liconic incubator (Liconic US, Woburn, MA) for 90 min. The TR-FRET signals for individual wells were measured with a PHERAstar FS plate reader. The % Inhibition of each tested inhibitor was calculated by using equation 1. Those inhibitors with % Inhibition >30% in the primary screening were selected for further dose response test, along with additional newly synthesized compounds.

The Z′-factor of each screening plate was calculated by using equation 2 (Ref. 27) with 16 data points in both the negative control (the DMSO group) and the positive control (DMSO without GST-MAGEA11 group) included.

$${Z}^{′} = 1 - \frac{3 {σ}^{+} + 3 {σ}^{-}}{{Mean}^{+} - {Mean}^{-}} . \qquad Equation 2$$

where σ+ is the standard deviation of the negative control group (DMSO group), σ− is the standard deviation of the positive control group (DMSO without GST-MAGEA11 group), mean+ is the mean of the negative control group (DMSO group), and mean− is the mean of the positive control group (DMSO without GST-MAGEA11 group).

In the TR-FRET dose response test, a protocol similar to the primary screening was followed except that the final DMSO concentration was 0.8% (v/v) with 0.1% (v/v) from the FAM-PCF11_4 DMSO stock solution and 0.7% (v/v) from the compound-DMSO solution or the DMSO for the control wells (Pin Tool was used to transfer 140 nl of compound-DMSO solutions or DMSO). The inhibitors were arrayed in columns 1 to 24 and the controls were arrayed in columns 21 to 24 of 384-well compound plates. The final tested compound concentrations ranged from 3.6 nM to 70 μM in a 1-to-3 dilution pattern for 10 concentration levels or ranged from 0.13 nM to 70 μM in a 1-to-2 dilution pattern for 20 concentration levels. The % Inhibitions of each tested inhibitor at individual concentrations were calculated by using equation 1 and were fit into a Sigmoidal dose response equation to derive IC₅₀ values with the GraphPad Prism 4.81 software, if applicable.

Thermal Stability Measurements. The thermal stability of the MAGE-A11 in the presence of ligand was measured using differential scanning fluorimetry. 200 n of ligand PCF11_6, diluted to achieve the desired final concentration in DMSO, was transferred to an Applied Biosystems MicroAmp Optical reaction plate using acoustic transfer. To each well, 20 μl thermal stability assay solution (10 μM His-SUMO-MAGE-A11 and 5× SyproOrange in 50 mM HEPES, 100 mM NaCl, 50 mM MgCl₂, pH 7.5) was added, and the plate was sealed with MicroAmp optical adhesive film. The plate was exposed to a range of temperatures (25-99° ° C., ramp rate 0.1° C./s) while measuring the fluorescence of SyproOrange in an Applied Biosystems QuantStudio 5 real-time PCR instrument. Resulting data were then analyzed using GraphPad Prism (v8.3.1), and the melting temperatures of each condition calculated using the Boltzmann Equation. All experiments were performed in triplicate.

Protein Expression and Purification. GST-PCF11 (full-length or amino acids 637-702), GST-MAGE-A11 MHD (218-429) or GST tag alone were induced in BL21(DE3) cells at 16° C. with 0.5 mM isopropyl β-D-1-thiogalactopyranoside (IPTG). GST-tagged proteins were purified from bacterial lysates in lysis buffer (50 mM Tris pH 7.7, 150 mM KCl, 0.1% (v/v) Triton X-100, 1 mM DTT, 1 mg/ml lysozyme) using GSTrap Hp 5 ml column (GE Amersham) followed by Resource Q ion exchange chromatography (GE Amersham) and was concentrated to 15 mg/ml in 20 mM Tris pH 7.4, 150 mM NaCl, 1 mM DTT and 5% (v/v) glycerol.

For crystallization, PCF11 peptide (677-701) is fused at the N-terminus of MAGE-A11 MHD (218-429) with linker sequence GGSGRP. The fusion protein was expressed in BL21(DE3) E. coli using the pSUMO3 vector. The protein complex was purified using a HisTrap Hp 5 ml column (GE Amersham) followed by Resource Q ion exchange chromatography (GE Amersham). The His-SUMO tag was subsequently cleaved off by addition of SENP protease followed by secondary HisTrap Hp 5 ml column (GE Amersham). The resulting fusion protein complex was concentrated to 15 mg/ml in 50 mM Tris, PH 7.4, 200 mM NaCl and 5 mM DTT.

Crystallization, Data Collection, Structure Determination, and Model Quality. Crystals were grown by the sitting-drop vapor diffusion method at 20° C. The well solution contained 0.2 M Na acetate, 0.1 M Bis Tris propane pH 7.5 and 20% (w/v) PEG 3350. The drop contained a 1:1 volume ratio of well solution to protein (15 mg/ml in 50 mM Tris, pH 7.4, 200 mM NaCl and 5 mM DTT). Crystals appeared overnight and matured in about one week. For cryo-preservation, the crystals were incubated in reservoir solution with 10% (v/v) glycerol prior to flash-cooling in liquid nitrogen. Data were collected at the Southeast Regional Collaborative Access Team (SER-CAT) Sector 22-BM beamline at 1 Å. Data were integrated and scaled using HKL2000 (Ref. 31) to 2.2 Å. The structure was solved by molecular replacement with Phaser (Ref. 32) using MAGE-A3 (PDB ID 4V0P) as the search model. Iterative rounds of model building and refinement were performed with COOT (Ref. 33) (Emsley et al, 2010) and Refmac (Ref. 34), respectively. Processing and refinement statistics are summarized in Table 1.

The final model is of high quality, with four fusion protein protomers in the P 1 cell. Although PCF11 peptide residues were readily identified in all protomers, the MAGE-A11 linker was disordered. All protomers are remarkably similar, with a C-alpha RMSD of <0.4 Å relative to protomer A (chain B 0.141, chain C 0.351, chain D 0.349). Detailed descriptions and figures refer to protomer A, where PCF11 is solvent exposed and unperturbed by crystal packing. The coordinates and structure factors have been deposited in the Protein Data Bank with accession number 6WJH. Figures were made using PyMOL (Ref. 35).

GST-Pulldown In Vitro Binding Assays. Myc-tagged proteins were in vitro translated using the SP6-TNT Quick rabbit reticulocyte lysate system (Promega) according to manufacturer's instructions. In vitro binding assays were performed by incubating 15 mg of purified GST-tagged protein PCF11 (637-702) or MAGE-A11 (218-429) with glutathione Sepharose beads (GE, Amersham) for 1 hr in binding buffer (25 mM Tris pH 8.0, 2.7 mM KCl, 137 mM NaCl, 0.05% (v/v) Tween-20, and 10 mM 2-mercaptoethanol). Bound beads were blocked for 1 hr in binding buffer containing 5% (w/v) milk powder. In vitro translated proteins (5 μl; Promega TNT rabbit reticulocyte lysate quick SP6 system) were then incubated with the bound beads for 1 hr in binding buffer containing 5% (w/v) milk powder. After four washes in binding buffer, the proteins were eluted in SDS-sample buffer, boiled, subjected to SDS-PAGE, and blotted with anti-Myc.

Tandem Ubiquitin-Binding Entity (TUBE) Ubiquitination Assay. One 10 cm² plates of HEK293FT cells stably expressing FLAG-vector, FLAG-MAGE-A11 wild-type or SBC mutants were lysed with TUBE lysis buffer (50 mM Tris pH 7.5, 150 mM NaCl, 1 mM EDTA, 1% (v/v) NP-40, 10% (v/v) glycerol, 20 mM N-Ethylmaleimide and 1× protease inhibitor cocktail), and the lysates were bound to TUBE-agarose (LifeSensors) overnight at 4° C. Beads were subsequently washed three times in wash buffer (20 mM Tris pH 8.0, 150 mM NaCl, 0.1% (v/v) Tween 20) and then the ubiquitinated proteins were eluted in SDS sample buffer, boiled, and subjected to SDS-PAGE and western blotting.

Clonogenic Growth Assay. For clonogenic growth assays on plates, wild-type MAGE-A11 or SBC mutants reconstituted MAGE-A11-knockout DAOY cells were plated in 6-well plates in triplicate. After 2-3 weeks, cells were fixed and stained with 0.05% (w/v) crystal violet and counted (colonies ≥100 μm in size).

Cell viability Assay. To assess cell viability after SJ521054 treatment, 1×10⁴ cells/ml were treated with SJ521054 (0-40 μM) and incubated for 24 hours prior to changing the media and adding alamarBlue (Thermo Fisher Scientific) and incubating for 4 hours at 37° C. Plates were read by measuring the fluorescence with excitation wavelength at 540 nm and emission wavelength at 590 nm on an Enspire plate reader.

Xenograt Tumor Growth Assays. 3×10⁶ MAGE-A11-knockout DAOY cells reconstituted with wild-type MAGE-A11 or SBC mutants were mixed with matrigel (Corning) before injection into the flank of NOD scid gamma mice (Jackson Lab) (n=6 for each group). Tumor size was measured 2-3 times a week during the duration of the experiment.

RNA Sequencing and 3′-UTR Analysis. Total RNA was extracted from xenograft tumors using RNeasy kit (Qiagen) according to manufacturer's instructions. RNA quality was assessed by 2100 Bioanalyzer RNA 6000 Nano assay (Agilent). Libraries were prepared using TruSeq Stranded mRNA kits (Illumina) and subjected to 100 cycle paired-end sequencing on the Illumina HiSeq platform. Low quality reads were filtered out using Trim Galore and then aligned to the human genome (hg19/GRCh37) using STAR version 2.5.2b (Ref. 36). The mapped BAM files were converted into bedgraph format using bedtools version 2.17.0 (Ref. 37). The RNA-seq read coverage was visualized at UCSC Genome Browser (Ref. 38). Data was deposited to NCBI gene expression omnibus as accession GSE148458. DaPars (Ref. 39) was used to identify the most significant APA events between MAGE-A11 WT and M341R conditions. RNA-seq coverage for 3′UTR region had to be greater than 20 to be included for analysis. The significant APA events were defined as 1) the adjusted P value was controlled at 5%. 2) the absolute mean difference of PDUI must be no less than 0.2. 3) the mean PDUI fold change must be no less than 1.5.

### Chemical Synthesis.

Representative synthetic routes used to explore the structure-activity relationship of disclosed quinoline analogs are shown in Scheme 1-3 below. Chloroquinoline 1a-1d were condensed with 1,3-propyldiamine (2) to afford the diamine compounds 3a-3d (Scheme 1; see Ref. 40). Reaction of compounds 3a-3d with aldehydes 4 followed by reduction with sodium borohdride provided the quinoline analogs 5a-5g (Ref 41). Suzuki coupling of compound 5d with aryl boronic acids 6 generated substituted quionlines 5h and 5i (Scheme 2). Acylation of compound 5a with acryloyl chloride (7) provided the substituted quinoline 5j with a covalent warhead (Scheme 3; see Ref. 42). Analogs 5a-5j were converted to the HCl salts by treatment with methanolic solution of hydrochloric acid.

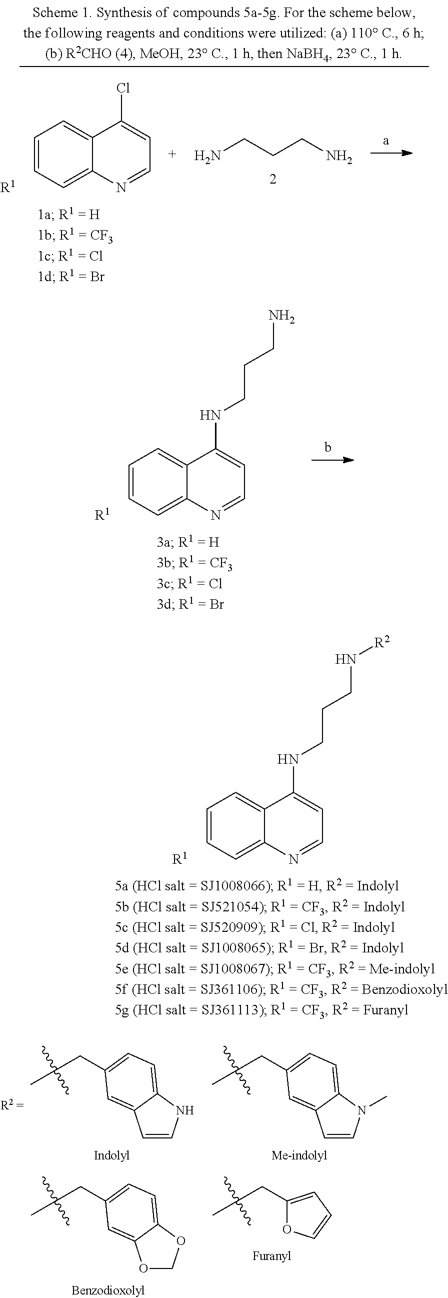

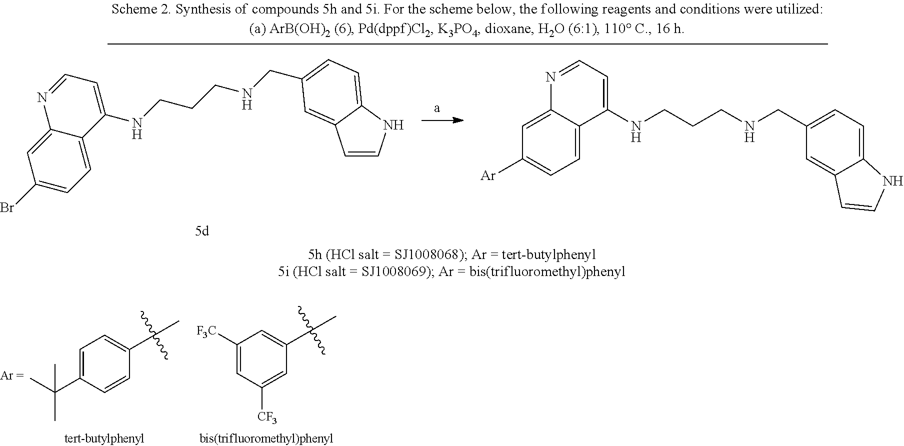

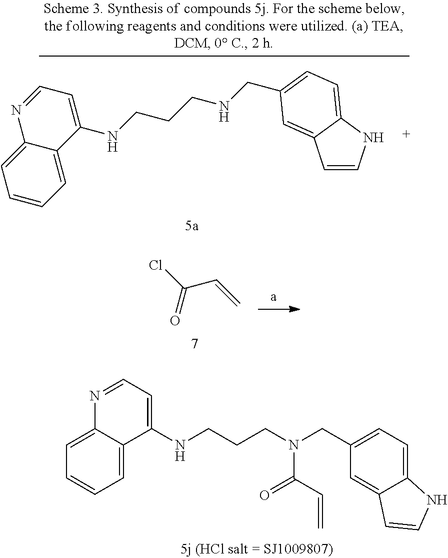

Further detailed protocols for the synthesis of disclosed quinoline analogs are described herein below.

### ADME Studies on Disclosed Compounds.

Solubility assays were carried out on a Biomek FX lab automation workstation (Beckman Coulter, Inc., Fullerton, CA) using SOL Evolution software (pION Inc., Woburn, MA). The detailed method is described as follows. 10 μl of 10 mM compound stock (in DMSO) was added to 190 μl 1-propanol to make a reference stock plate. 5 μl from this reference stock plate were mixed with 70 μl 1-propanol and 75 μl citrate phosphate-buffered saline (PBS; isotonic) to make the reference plate, and the UV spectrum (250-500 nm) of the reference plate was read. 6 μl of 10 mM test compound stock was added to 594 μl buffer in a 96-well storage plate and mixed. The storage plate was sealed and incubated at RT for 18 h. The suspension was then filtered through a 96-well filter plate (pION Inc., Woburn, MA). 75 μl of filtrate was mixed with 75 μl 1-propanol to make the sample plate, and the UV spectrum of the sample plate was read. Calculations were carried out with μSOL Evolution software based on the area under the curve (AUC) of the UV spectrum of the sample and reference plates. All compounds were tested in triplicate.

High throughput Caco-2 permeability was performed in the TranswellR 0.4 μm polycarbonate membrane 96-well system with modified methods. Caco-2 cells were maintained at 37° C. in a humidified incubator with an atmosphere of 5% CO₂. The cells were cultured in 75 cm² flasks with Dulbecco's Modified Eagle's Medium (DMEM) containing 10% (v/v) FBS, 1% (v/v) non-essential amino acids (NEAA), 100 units/ml of penicillin, and 100 μg/ml of streptomycin. The Caco-2 cells were seeded onto inserts at a density of 2×10⁴ cells/insert separately. The medium in the wells was exchanged each other day, and the trans epithelial electrical resistance (TEER) value was measured using an epithelial voltohm meter (Millipore, Billerica, Massachusetts). Caco2 cells were grown for 7 days to reach consistent TEER values (typically 2000 ohms greater than initial value when cells are first seeded into transwells), indicating that the cells had formed a confluent polarized monolayer. For transport experiments, each cultured monolayer on the 96-well plate was washed twice with a transport buffer (HBSS containing 25 mM HEPES, pH 7.4). The permeability assay was initiated by the addition of each compound solution (10 μmol/l) into inserts (apical side, A) or receivers (basolateral side, B). The Caco-2 cell monolayers were incubated for 2 h at 37° C. Fractions were collected from receivers (if apical to basal permeability) or inserts (if basal to apical permeability), and concentrations were assessed by UPLC/MS (Waters; Milford, MA). All compounds were tested in triplicates. The A→B (or B→A) apparent permeability coefficients (Papp) of each compound were calculated using the equation, Papp=dQ/dt×1/AC0, where dQ/dt equals the flux of a drug across the monolayer, A equals the total insert well surface area, and C0 is the initial concentration of substrate in the donor compartment. The efflux ratio was determined by dividing the Papp in the B-A direction by the Papp in the A-B direction. An efflux ratio >2 suggested that a given substrate was actively transported across the membrane.

c Log P and tPSA values provided in the Table 6 were estimated using the PerkinElmer ChemDraw Professional, version 17.1.0.105 software (Ref. 43).

Characterization of MAGE-A11 Interaction with the PCF11 Substrate.

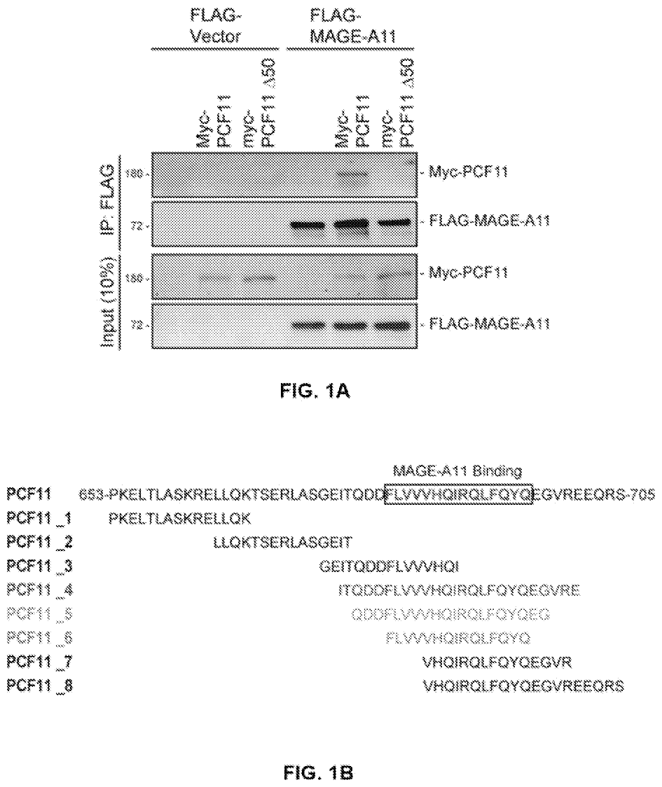

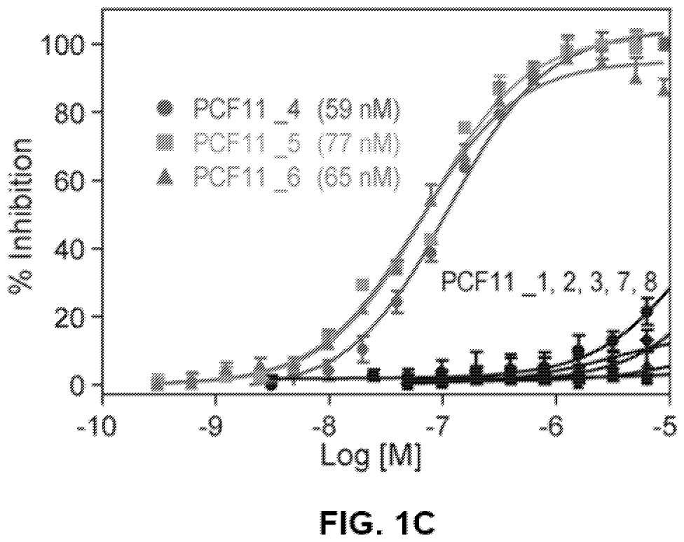

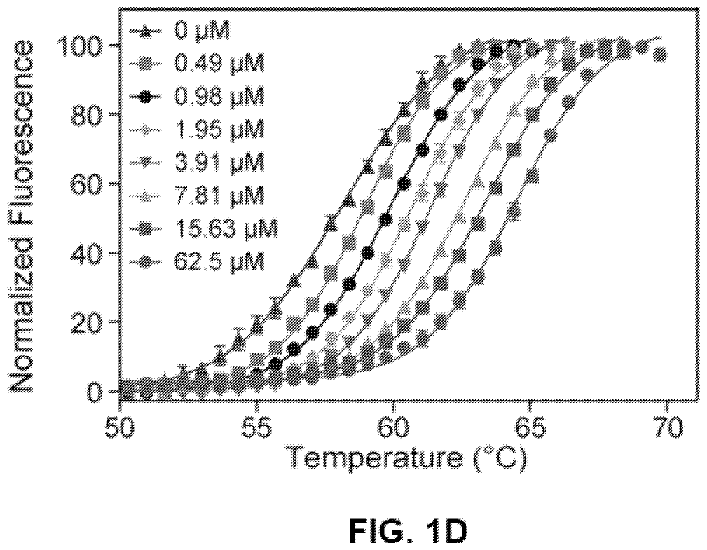

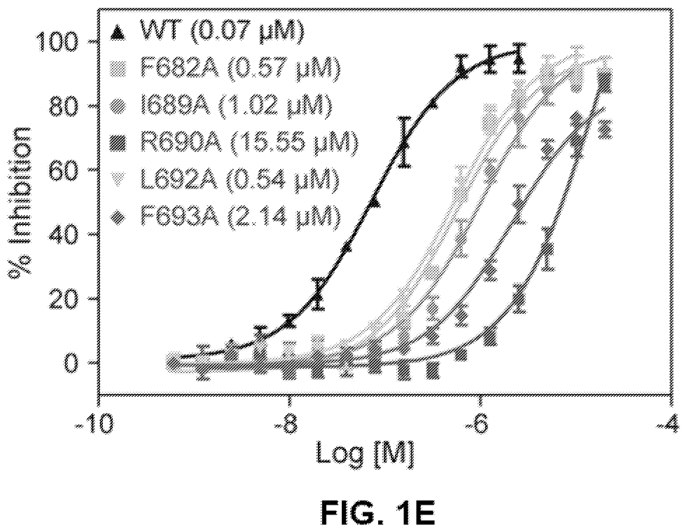

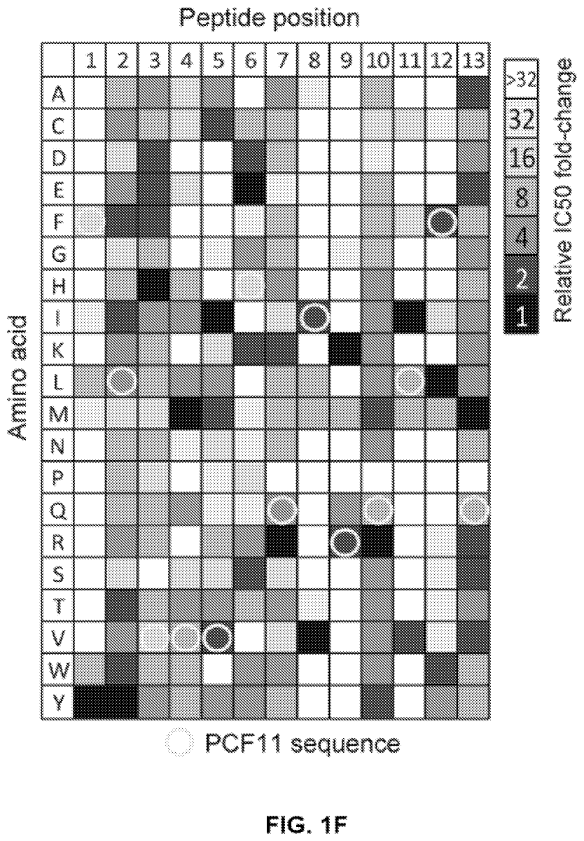

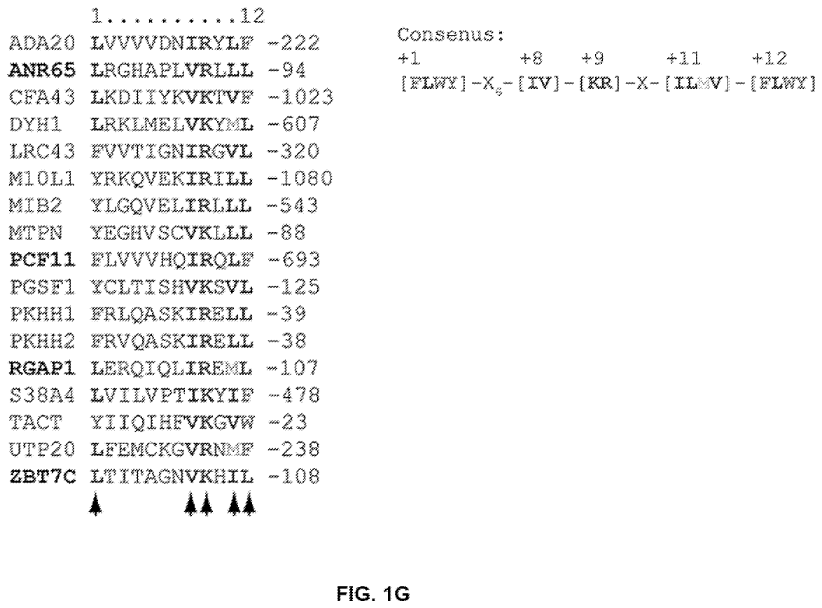

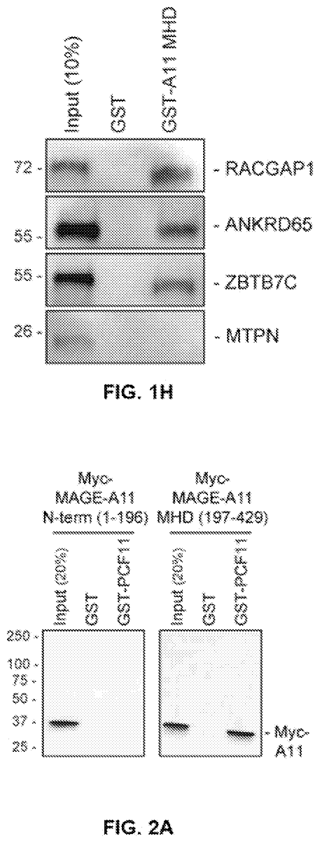

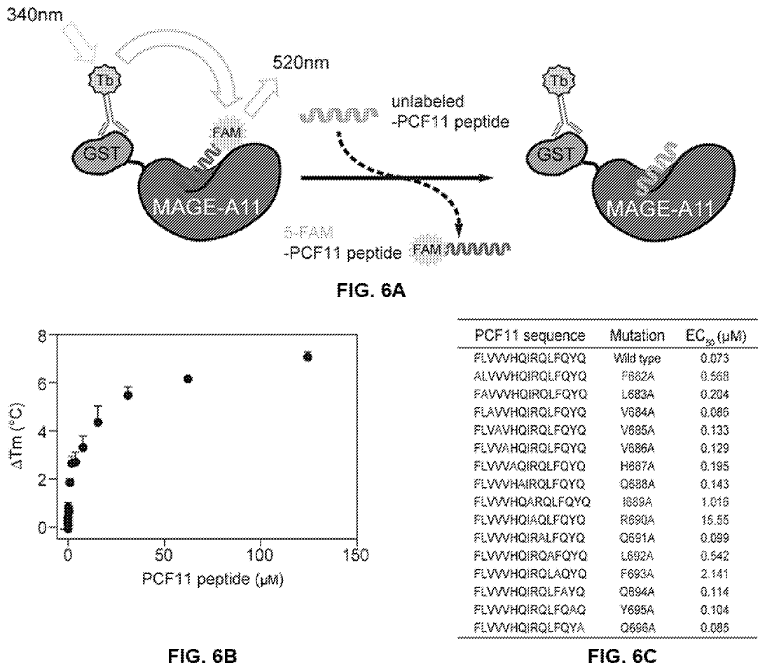

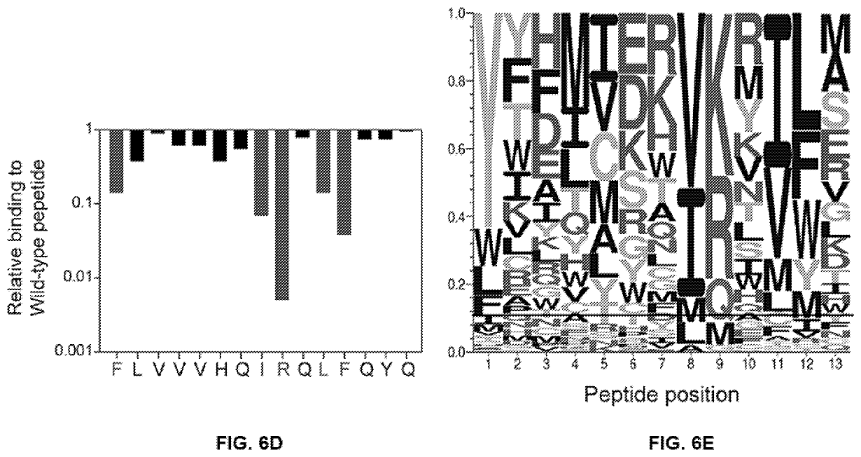

It has been previously reported that MAGE-A11 drives tumorigenesis through ubiquitination of the 3′-mRNA processing factor PCF11 resulting in alternative polyadenylation (APA) of transcripts and 3′-UTR shortening (3′-US; see Ref. 10). To understand how MAGE-A11 recognizes its substrate, the degron motif in PCF11 required for MAGE-A11 binding was mapped. It was found that deletion of PCF11 amino acids 653-702 completely blocked MAGE-A11 binding by co-immunoprecipitation (co-IP) (FIG. 1A). To narrow down the minimal degron motif within these 50 amino acids, a time-resolved fluorescence resonance energy transfer (TR-FRET) assay was developed to monitor binding of a series of PCF11 peptides to GST-MAGE-A11 in vitro (FIG. 1B and FIG. 6A). Notably, of the eight PCF11 peptides tested, three overlapping peptides centered on PCF11 amino acids 682-696 showed robust binding with IC₅₀ of 59-77 nM (FIG. 1C). These findings were confirmed by an orthogonal assay showing changes in the thermal stability of MAGE-A11 by the PCF11 degron peptide (FIG. 1D and FIG. 6B).

To identify the specific residues in the PCF11 degron responsible for interaction with MAGE-A11, alanine scanning mutagenesis was carried out for the PCF11 degron peptide and binding to MAGE-A11 was monitored by TR-FRET (FIG. 6C). Mutation of F682, I689, R690, L692 or F693 to alanine dramatically decreased binding 10-200 fold, with other positions having minimal effects (FIG. 1E and FIG. 6D). These findings are consistent with previous reports that mutation of PCF11 I689A blocked PCF11 interaction with MAGE-A11 in cells and prevented MAGE-A11-induced xenografts tumor growth (Ref. 10).

Given the success of the binding assay to map specific amino acid sequences competent for MAGE-A11 binding, the study was expanded to perform TR-FRET affinity measurements on a peptide array consisting of 248 peptides in which each position within the model 13 amino acid PCF11 peptide degron was mutated to all possible amino acids. Consistent with the alanine scanning mutagenesis, the same five amino acid positions were again identified as being the most important (FIG. 1F). Position +1 (PCF11 F682) preferred an aromatic residue, position +8 (PCF11 I689) required a hydrophobic residue, position +9 (PCF11 R690) only tolerated a basic residue, position +11 (PCF11 L692) preferred a large hydrophobic residue and position +12 (PCF11 F693) preferred aromatic residues. Overall, the degron sequence conforms to [FLWY]-X₆-[IV]-[KR]-X-[ILMV]-[FLWY], where residues denoted as X being tolerable to several amino acids. Notably, the PCF11 sequence is not fully optimized with several substitutions yielding higher affinity (FIG. 1F). These insights provided for a consensus MAGE-A11 binding motif weighted matrix (FIG. 6E). Search of the proteome using this matrix revealed 17 possible MAGE-A11 targets with the consensus binding motif (FIG. 1G). To validate this prediction, the ability of MAGE-A11 to bind four of these newly predicted targets was examined in vitro, and it was found that RACGAP1, ANKRD65 and ZBTB7C are robust binding partners (FIG. 1H). These results provide detailed understanding of the sequences recognized by MAGE-A11.

### Crystal Structure of MAGE-A11 Bound to PCF11.

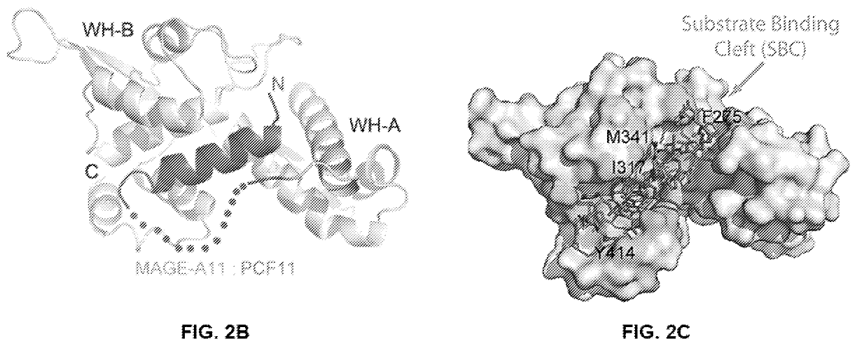

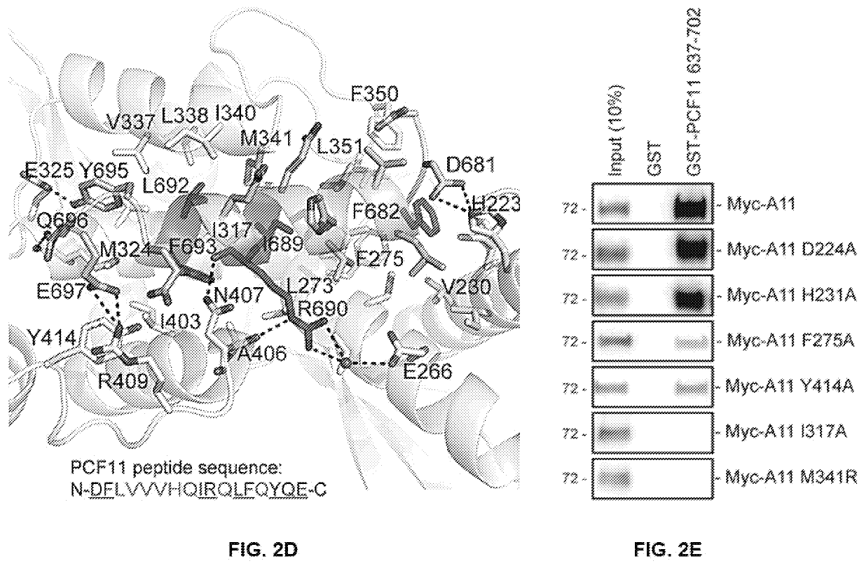

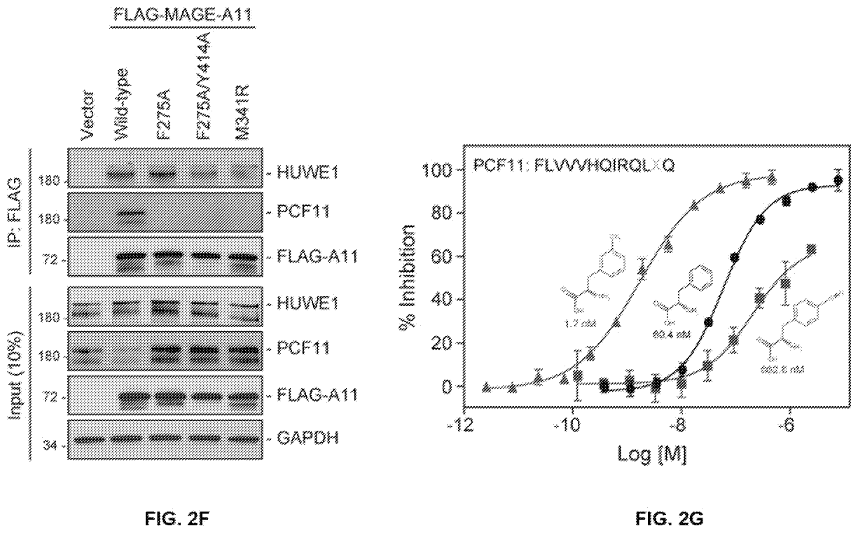

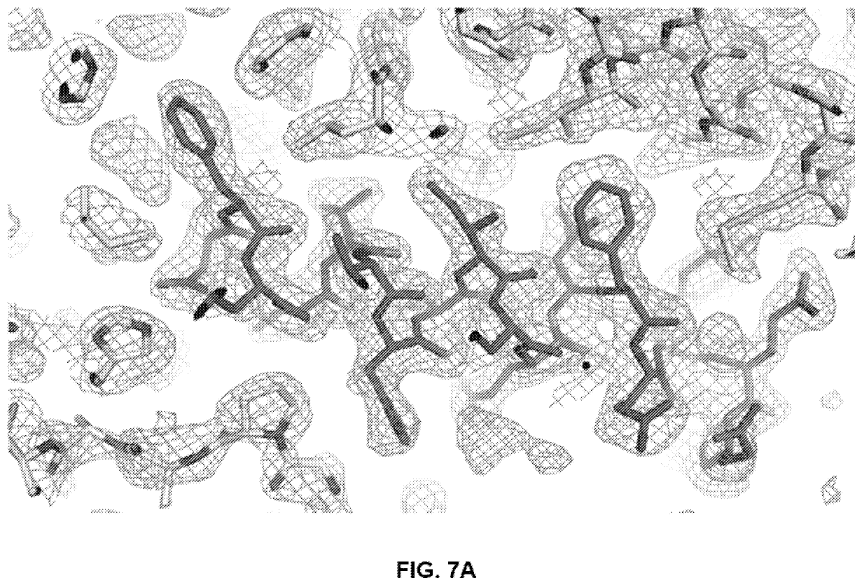

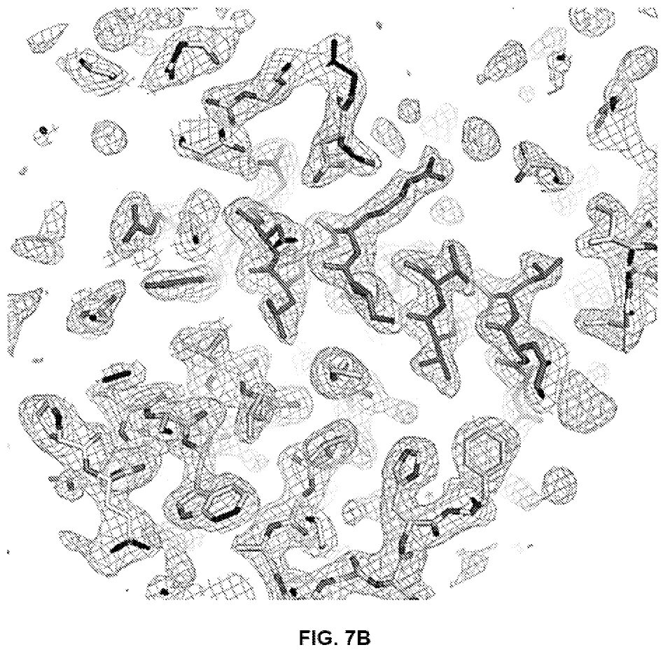

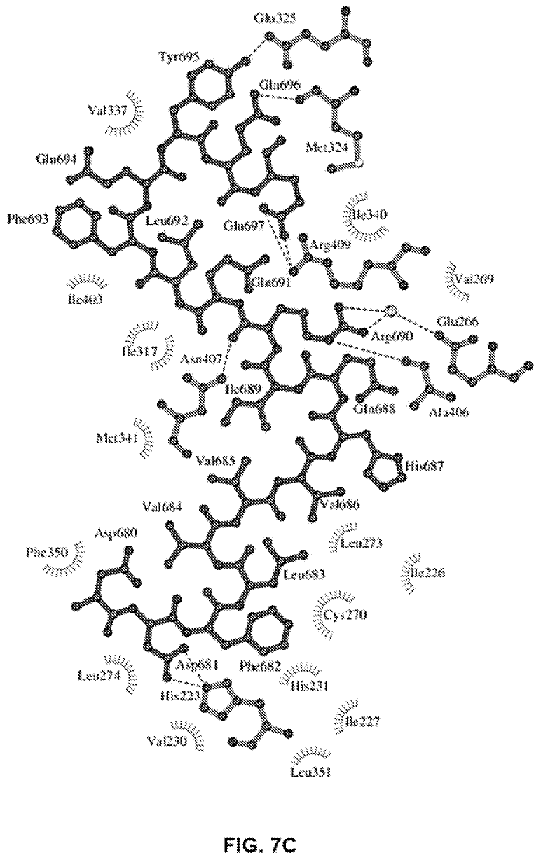

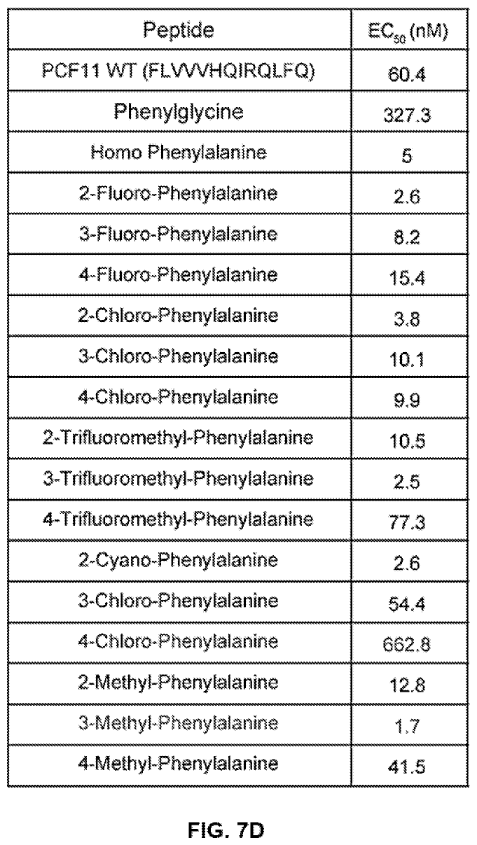

MAGE genes are defined by a common conserved MAGE homology domain (MHD), which is involved in protein-protein interactions (Ref. 2 and 8-9). Similar to other MAGE:substrate interactions (Ref. 5, 8), PCF11 binding to MAGE-A11 is mediated through the MAGE-A11 MHD (197-429) (FIG. 2A). To understand how a substrate interacts with MAGEs, the crystal structure of MAGE-A11 bound to the PCF11 degron was determined. Specifically, the PCF11 degron sequence fused to the MAGE-A11 MHD (S218 to C-terminus) from bacteria was purified and the crystal structure solved to 2.2 Å (Table 1). MAGE-A11 MHD formed two winged-helix motifs (WH-A and WH-B) conjugated by a linker loop, where each WH motif contains three alpha-helices and two beta-sheets. The overall MAGE-A11 structure is similar to the other two reported MAGE-A family protein structures (MAGE-A3-MHD PDB: 4V0P and MAGE-A4-MHD PDB: 2WA0). However, the extreme C-terminal residues of MAGE-A11 formed three alpha-helices, whereas these residues are unstructured in MAGE-A3-MHD and MAGE-A4-MHD structures (FIG. 2B; see also Ref. 9). Of particular interest, the PCF11 degron forms a short alpha-helix (FIGS. 2B-2C and FIGS. 7A-7B) that binds into a shallow cleft (FIG. 2C) formed at the interface of the WH-A and WH-B motifs. This region on MAGE-A11 is referred to herein as the substrate binding cleft (SBC).

TABLE 1
Data collection and refinement statistics.

| Parameter | MAGE-A11:PCF11 |
|---|---|
| Data collection | |
| Space group | P 1 |
| Cell dimensions | |
| a, b, c (Å) | 55.50 78.12 72.91 |
| α, β, γ (°) | 77.44 77.34 89.59 |
| Resolution (Å) | 38.79-2.19 (2.24-2.19)* |
| R_{sym} | 0.120 (0.372) |
| I/σ (I) | 19.9 (2.4) |
| CC_{1/2} | 0.952 (0.757) |
| Completeness (%) | 96.6 (94.7) |
| Redundancy | 2.5 (2.3) |
| Refinement | |
| Resolution (Å) | 38.79-2.19 |
| No. reflections | 53723 |
| R_{work} / R_{free} | 0.205 / 0.246 |
| No. atoms | |
| Protein | 7085 |
| Water | 208 |
| B factors (Å²) | |
| Protein | 40.12 |
| Water | 45.27 |
| R.M.S. deviations | |
| Bond lengths (Å) | 0.008 |
| Bond angles (°) | 1.48 |
| Ramachandran statistics (%) | |
| Favored | 96.38 |
| Allowed | 3.62 |
| Outliers | 0 |

*Values in parentheses are for the highest resolution shell.

PCF11 binding to the MAGE-A11 SBC appears to be largely mediated by numerous hydrophobic interactions that extend along its helical core and capping residues (FIG. 2D and FIG. 7C). I689 and L692 of PCF11 appear to form a hydrophobic core with L273, L274, I317, M341, V337, I340 and L338 of MAGE-A11. Notably, the N-terminal capping residue F682 of PCF11 interacts with F275, F350, L351, H231 and V230, whereas F693 at the C-terminus interacts with I317, I403 and A406 in MAGE-A11. In addition, a key polar interaction network enhances binding; R690 in PCF11 appears to form a water-mediated salt bridge with E266 in MAGE-A11 and is further stabilized by forming a hydrogen bond between the NE2 moiety and the backbone carbonyl of A406. Taken together, these structural observations are highly consistent with the TR-FRET mutagenesis data disclosed herein. A detailed description of polar and hydrophobic PCF11 interactions with MAGE-A11 is provided below in Table 2.

TABLE 2
Polar and hydrophobic PCF11 interactions with MAGE-A11

| PCF11 | MAGE-A11 Residue | MAGE-A11 Residue |
|---|---|---|
| Residue* | (Hydrophobic Contacts) | (Polar, Hydrogen Bond) |
| D681 | | H223 NE2 |
| F682 | F275, H231, C270, F350, | |
| | L351, V230 | |
| L683 | I226, I227, C270 | |
| V684 | | |
| V685 | L274, F350 | |
| V686 | L273, L274, F275, C270 | |
| H687 | | |
| Q688 | | |
| I689 | L273, L274, I317, M341 | |
| R690 | L269, L273 | E266 (water-mediated), A406 |
| | | backbone carbonyl-R690 NE, |
| | | N407 - R690 backbone |
| | | carbonyl |
| Q691 | | |
| L692 | I317, V337, L338, I340, | |
| | M341 | |
| F693 | I317, A406, I403 | |
| Q694 | | |
| Y695 | | E325 |
| Q696 | | M324 backbone carbonyl |
| E697 | | R409 |

*Residues observed in the electron density map for PCF11 protomer A are listed.

Next, validation of the MAGE-A11:PCF11 structure was assessed through mutation of the MAGE-A11 SBC and measuring PCF11 binding in vitro and in cells. Mutation of MAGE-A11 SBC residues F275 or Y414 to alanine significantly decreased MAGE-A11 interaction with PCF11 in vitro (FIG. 2E). These findings were corroborated by co-IP studies in cells (FIG. 2F). Furthermore, disruption of the hydrophobic core of the MAGE-A11 SBC by I317R or M341R ablated PCF11 interaction in vitro (FIG. 2E) and in cells (FIG. 2F). In comparison, mutation of MAGE-A11 D224 that is not involved in PCF11 recognition had no effect on PCF11 binding in vitro (FIG. 2E). These results are consistent with the MAGE-A11:PCF11 crystal structure suggesting that the MAGE-A11 SBC serves as a critical region for substrate recognition. Importantly, mutation of the MAGE-A11 SBC did not disrupt binding to the MAGE-A11 associated E3 ligase HUWE1 (FIG. 2F). Together, these results suggest that the MAGE-A11 SBC mediates substrate interaction and E3 ligase binding occurs independent of the MAGE-A11 SBC.

Given the importance of the C-terminal end of the PCF11 motif for MAGE-A11 binding and that MAGE-A11:PCF11 interaction affinity is not fully optimized, several peptides were synthesized with non-natural phenylalanine derivatives at PCF11 F693. This included F, Cl, CF₃, CN or CH₃ substitutions at the 2, 3 or 4 position of the phenylalanine benzene ring (FIG. 2G and FIG. 7D). One variant, 3-methyl-phenylanaline, significantly increased PCF11 binding to MAGE-A11 (FIG. 2G; IC₅₀=1.7 nM), likely due to increased hydrophobic and Van der Waals contacts. However, substitutions at the 4th position on the benzene ring, including 4-cyano-phenylalaninnne (FIG. 2G; IC₅₀=663 nM), were not tolerated well, likely due to steric hindrance. Overall, these findings provide structural basis of MAGE-A11 binding to PCF11 and suggest mechanisms to improve MAGE-A11:PCF11 substrate binding affinity.

### Mutation of MAGE-A11 SBC Disrupts its Oncogenic Activity.

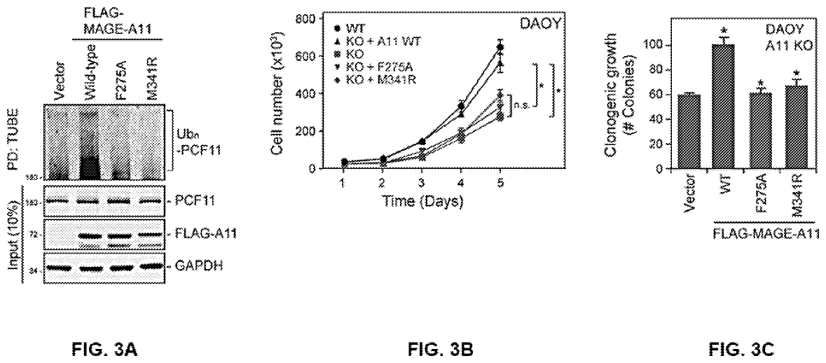

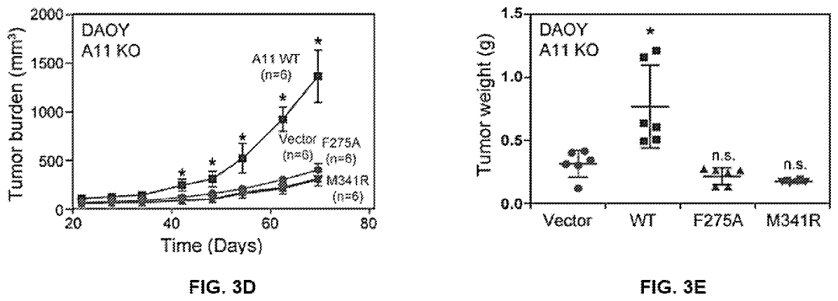

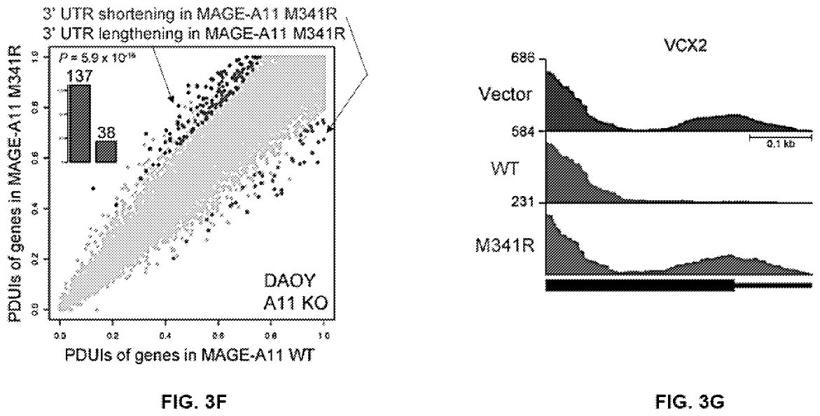

To determine whether mutation of the MAGE-A11 SBC disrupts its ability to ubiquitinate PCF11 and control tumor cell growth, MAGE-A11 knockout DAOY cells were stably reconstituted with MAGE-A11 SBC mutants F275A or M341R and assessed PCF11 ubiquitination and subsequent effects on cell growth and tumor formation. Consistent with disruption of PCF11 binding by MAGE-A11 SBC mutation (FIG. 2F), MAGE-A11 F275A and M341R failed to promote PCF11 ubiquitination in cells (FIG. 3A). Moreover, unlike wild-type MAGE-A11, MAGE-A11 F275A and M341R failed to promote proliferation of MAGE-A11 knockout DAOY cells (FIG. 3B). Compared to wild-type MAGE-A11, re-expression of MAGE-A11 F275A or M341R reduced clonogenic growth (FIG. 3C). To test the importance of the MAGE-A11 SBC for MAGE-A11 oncogenic activity in vivo, xenograft tumor growth assays were performed using the MAGE-A11 knockout DAOY cells reconstituted with wild-type, F275A or M341R MAGE-A11. Consistent with MAGE-A11 oncogenic activity, wild-type MAGE-A11 promoted increased tumor growth (FIGS. 3D and 3E). However, DAOY cells reconstituted with MAGE-A11 SBC mutants failed to promote tumor growth and were indistinguishable from vector control cells (FIGS. 3D and 3E). To determine if MAGE-A11 M341R fails to promote 3′-UTR shortening, tumors were isolated and transcriptome analysis was performed. Consistent with MAGE-A11 M341R failing to bind to and ubiquitinate PCF11, MAGE-A11 M341R tumors had significantly lengthened 3′-UTRs compared to wild-type MAGE-A11 (FIGS. 3F and 3G). Together, these results suggest that the MAGE-A11 SBC is required for its ability to regulate PCF11, promote alternative polyadenylation and drive tumor growth.

### Substrate Recognition and Specificity Across the MAGE Family is Mediated by the SBC Region.

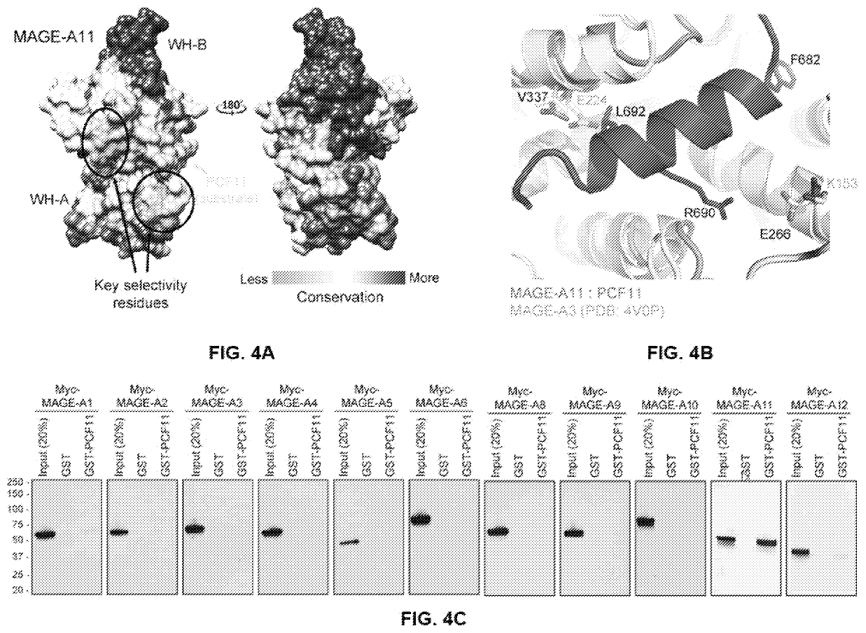

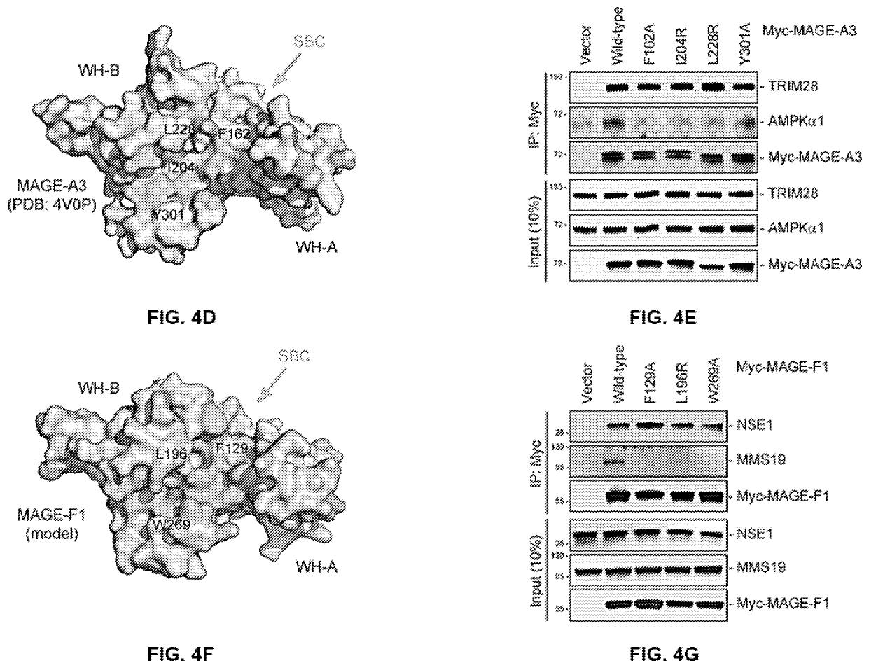

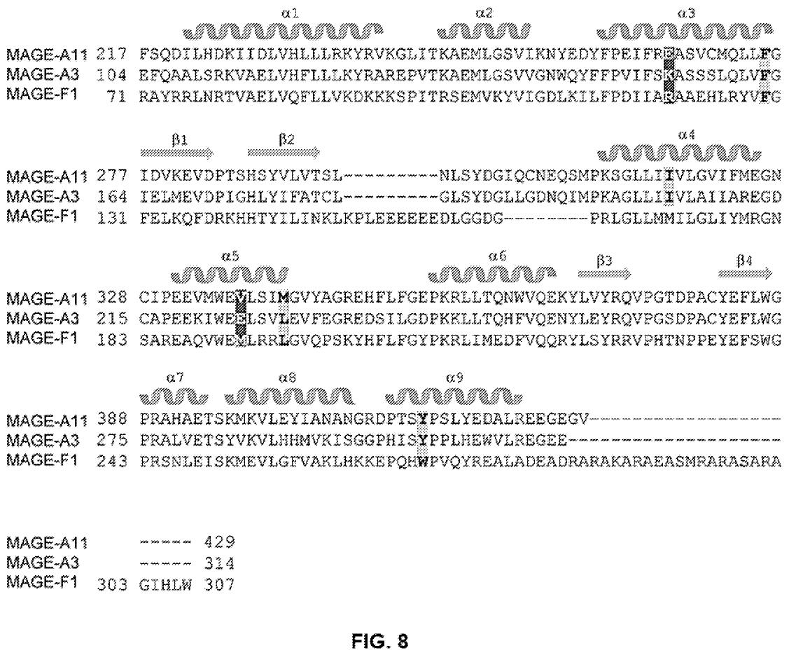

In humans there are at least 40 distinct MAGE genes that each contain at least one broadly conserved MHD, with some MHDs being >90% identical. However, not all MAGEs are redundant and their MHDs have been shown to recognize distinct substrates, including MAGE-A11:PCF11, MAGE-A3:AMPK and MAGE-F1:MMS19 (Ref. 5, 10, and 15). Thus, specific sequence elements in the MHD likely dictate substrate specificity. Mapping sequence conservation of MAGEs to the MAGE-A11:PCF11 structure revealed that the majority of residues that are highly conserved across MAGEs are on the ‘MHD backside’ away from PCF11 binding site FIG. 4A). However, the most variable residues are concentrated around the MAGE SBC, suggesting that these are likely key positive and negative selection filters that dictate substrate specificity across the MAGE protein family. This is exemplified in comparing the SBC of MAGE-A11 with -A3 (FIG. 4B). Although the MHD structures are highly similar (RMSD 1.07; MAGE-A3 (108-293) to MAGE-A11 (221-406)), detailed examination of the SBCs revealed important differences. For example, PCF11 R690 forms a salt bridge with E266 in MAGE-A11 SBC (FIG. 4B). However, in the MAGE-A3 SBC this residue has undergone a charge swap to lysine (FIG. 4B and FIG. 8). This along with other sequence variation in the SBC likely dictate substrate specificity for MAGE ligases. Consistent with these observations, the ability to bind PCF11 is highly specific to MAGE-A11 with none of the other ten MAGE-A proteins having detectable binding (FIG. 4C), despite overall sequence conservation between these MHDs being quite high (>80% similarity).

To determine whether other MAGEs utilize a similar SBC formed at the interface of WH-A and WH-B to mediate substrate binding, this region in MAGE-A3 and -F1 was mutated, and the ability to bind their respective substrates, AMPK and MMS19, was determined. The mutation of key interaction residues in MAGE-A3 SBC (F162A, I204R, L228R or Y301A) and MAGE-F1 SBC (F129A, L196R or W269A) that correspond to those mutated in the MAGE-A11 SBC (FIG. 4D, FIG. 4F and FIG. 8) disrupted their ability to bind AMPK and MMS19 substrates, respectively (FIGS. 4E and 4G). Additionally and consistent with MAGE-A11, mutation of MAGE-A3 SBC or MAGE-F1 SBC did not impair interaction with their cognate E3 ligase binding partners, TRIM28 and NSE1, respectively (FIGS. 4D and 4E). Together, these results suggest that the mechanism for substrate recognition is conserved across MAGE proteins and is mediated the MHD SBC.

### Small Molecule Inhibitors Targeting MAGE-A11.

MAGE-A11 expression is typically restricted to reproductive tissues, but is aberrantly expressed in many tumor types where its expression correlates with poor patient prognosis (Refs. 10, 14, and 21-23). Moreover, MAGE-A11 functions as an oncogene and its genetic ablation inhibits tumorigenesis (Ref. 10, 14). Thus, the cancer-specific MAGE-A11 E3 ubiquitin ligase is an ideal therapeutic target. Our findings suggest that disruption of MAGE-A11 interaction with PCF11 may be a viable strategy to design cancer-specific therapeutics. Although disruption of protein-protein interactions are difficult, several examples have emerged, including small molecule agonist that inhibit T-cell proliferation through the inhibition of IL-2 binding to its receptor IL2Rα (Ref. 24), small molecule inhibitors of the anti-apoptotic proteins Bcl-2, Bcl-XL and Bcl-w (Ref. 25) and small molecule antagonists block HDM2 binding to p53 (Ref. 26).

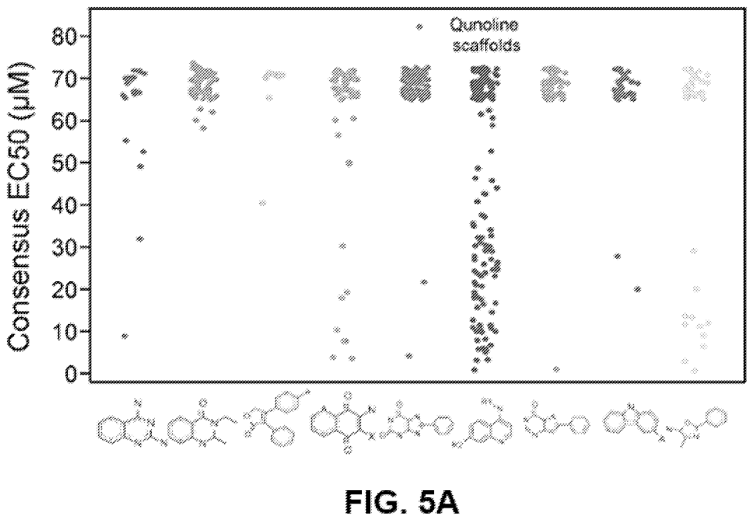

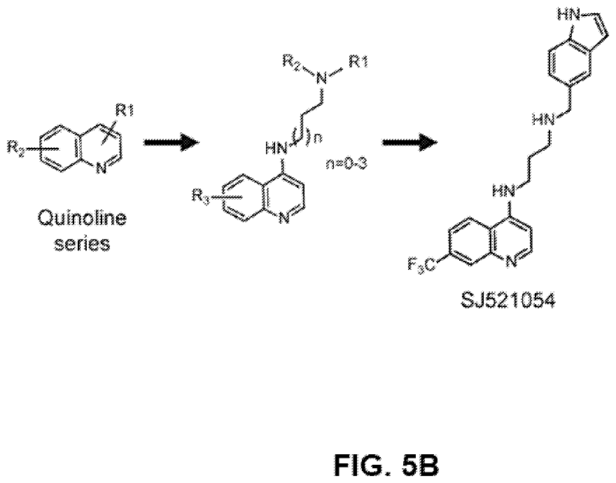

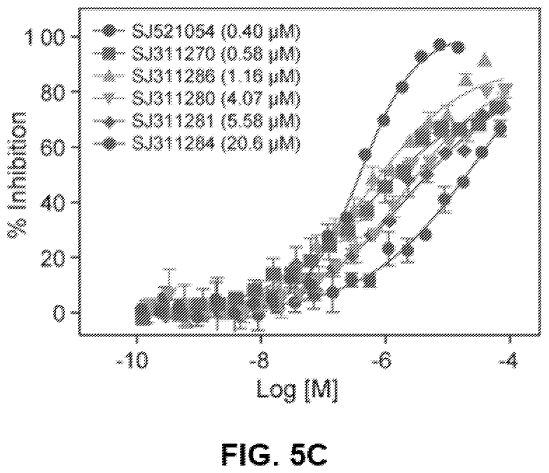

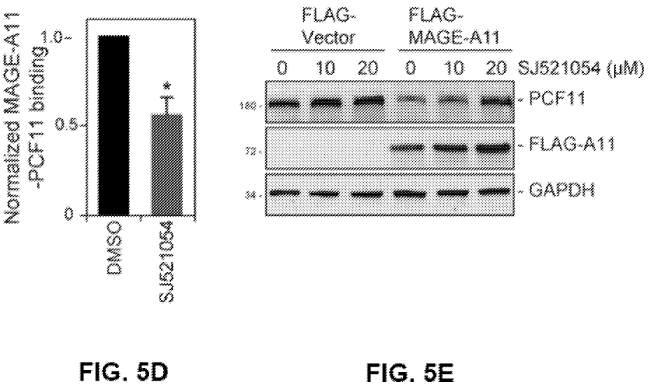

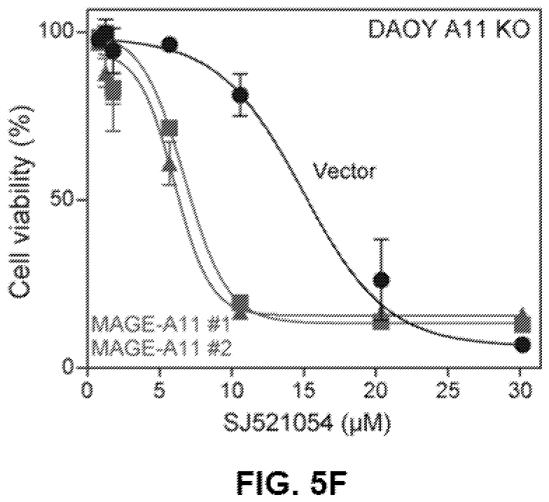

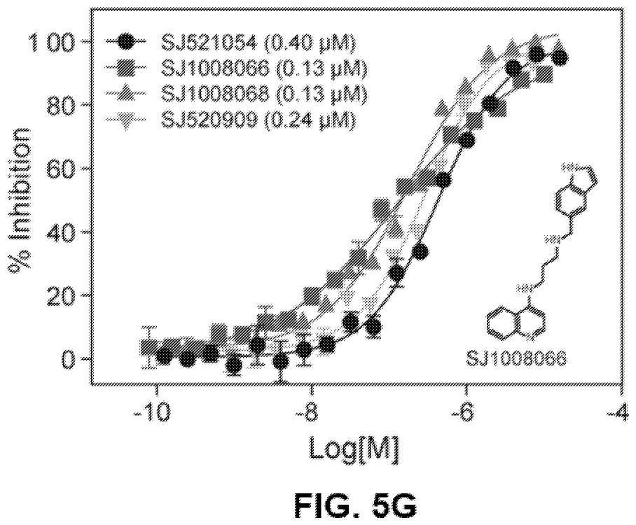

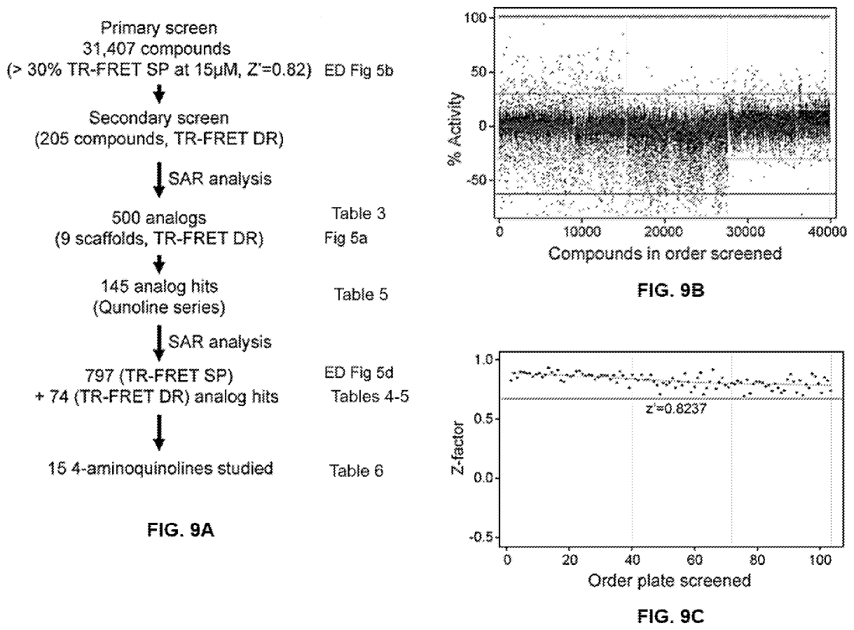

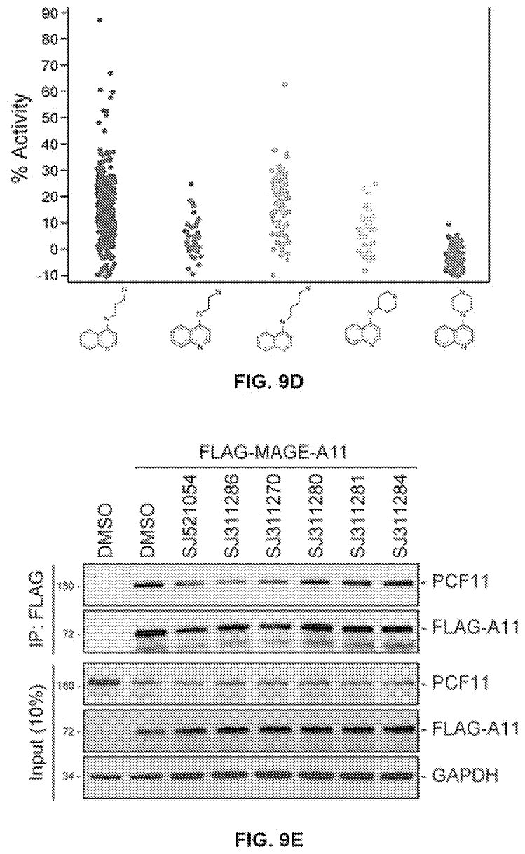

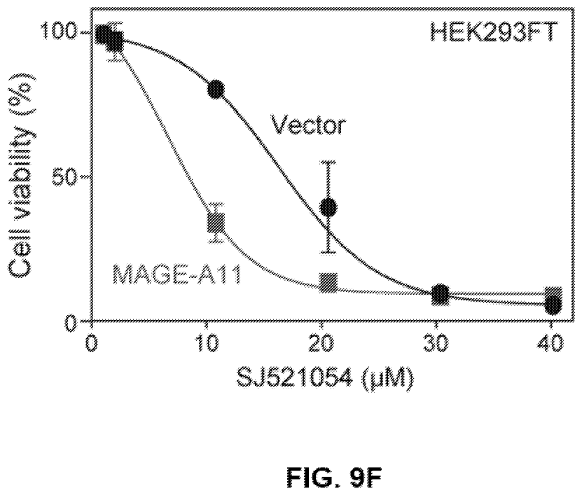

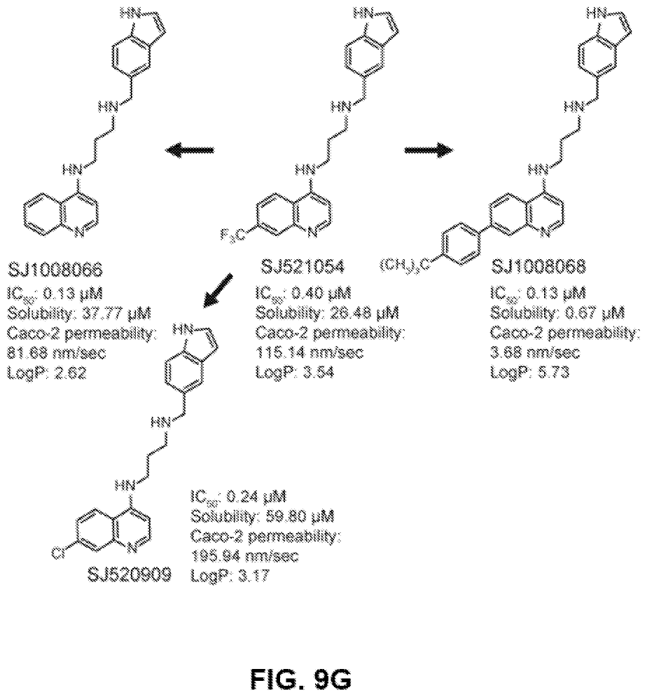

To identify inhibitors of MAGE-A11, a TR-FRET-based high-throughput screen was performed on 31,407 compounds (summarized in FIG. 9A) to identify small molecules that block MAGE-A11 binding to PCF11 in vitro. From this primary screen, 205 compounds (0.65% hit rate, Z′=0.82) that had greater than 30% inhibition (FIGS. 9B and 9C) were cherry-picked for follow-up dose response screening. A Z′-factor value >0.5 indicated that the screen is robust (Ref. 27). After hit clustering and structure-activity relationship (SAR) analysis from our primary and secondary screening results, an additional 500 analogs of nine scaffold groups obtained by substructure search of an in-house library collection were assayed (FIG. 5A and Table 3; note that Tables 3-6 follow the text of the Examples herein below). Of these scaffolds, the 145 quinoline analogs (Scaffold-4 in Table 3) showed the most significant effects on MAGE-A11:PCF11 binding with reasonable SAR and range of activity (FIG. 5A). To expand SAR of the quinoline series, we extended diversity around the scaffold and conducted a single point TR-FRET screen of 797 additional quinoline analogs followed by dose response experiments for 59 compounds (>30% inhibition at 15 μM) (FIG. 9D, Table 4, and Table 5). From these primary and secondary screens, one of the 4-Aminoquinoline analogs, SJ521054, emerged as a promising hit with robust activity in TR-FRET for disrupting MAGE-A11:PCF11 interaction (IC₅₀ 0.40 μM; see FIGS. 5B and 5C).

To validate the activity of SJ521054 in orthogonal assays, its ability to disrupt MAGE-A11:PCF11 interaction in cell lysates was examined. Indeed, SJ521054 inhibited MAGE-A11 binding to PCF11 in cell lysates as determined by co-IP, with much less potent compounds having minimal effects (FIG. 5D and FIG. 9E). If SJ521054 disrupts MAGE-A11:PCF11 interaction in cells, it was expected that: 1) SJ521054 should prevent MAGE-A11-induced degradation of PCF11 and 2) should show preferred cytotoxicity towards MAGE-A11-positive cells compared to isogenic MAGE-A11-negative cells. Consistent with this, SJ521054 stabilized PCF11 protein levels in MAGE-A11 expressing HEK293FT cells but had no effect on vector control cells (FIG. 5E). Strikingly, SJ521054 decreased the viability of MAGE-A11 expressing DAOY and HEK293FT cells to a much greater extent than vector control MAGE-A11-negative cells (FIGS. 5F and 9F).

To extend upon the discovery of SJ521054, 9 additional derivatives were designed, synthesized and evaluated by the TR-FRET assay for their ability to disrupt MAGE-A11:PCF11 interaction and their physicochemical properties were determined along with selected previous hits for comparison (Table 6). From these data, SJ1008066 was identified as a potent compound that is cell permeable and has improved activity in the TR-FRET assay (IC₅₀ 0.13 μM) and solubility (FIGS. 5G and 9G; Table 6). Taken together, these results identify tool compounds that provide proof-of-principle that disruption of MAGE-A11:substrate interaction may be a means to discover cancer-specific therapeutics.

### Synthesis of Representative Inhibitors of MAGE-A11:Substrate Interactions.

Disclosed herein are syntheses of representative compounds that inhibit MAGE-A11:substrate (PCF11) interaction. Table 7 lists the various representative compounds described herein. The table makes reference to groups Ra and Rb, which are shown in the compound having the structure shown below.

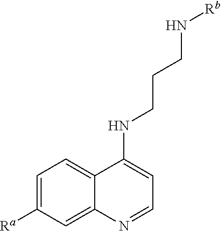

TABLE 7
List of quinoline analogs

| Compound | | | |
|---|---|---|---|
| Ref. No.^{a} | SJ Number | R^{a} | R^{b} |
| 5a | SJ1008066 | H | 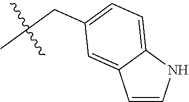 [SMILES: *Cc1ccc2[nH]ccc2c1] |
| 5b | SJ521054 | CF₃ | 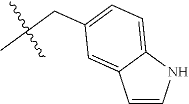 [SMILES: *Cc1ccc2[nH]ccc2c1] |
| 5c | SJ520909 | Cl | 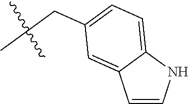 [SMILES: *Cc1ccc2[nH]ccc2c1] |
| 5d | SJ1008065 | Br | 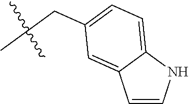 [SMILES: *Cc1ccc2[nH]ccc2c1] |
| 5e | SJ1008067 | CF₃ | 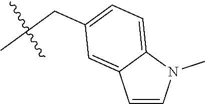 [SMILES: *Cc1ccc2c(ccn2C)c1] |
| 5f | SJ361106 | CF₃ | 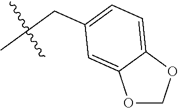 [SMILES: *Cc1ccc2c(c1)OCO2] |
| 5g | SJ361113 | CF₃ | 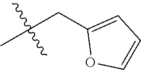 [SMILES: *Cc1ccco1] |
| 5h | SJ1008068 | 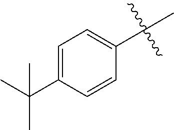 [SMILES: *c1ccc(C(C)(C)C)cc1] | 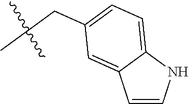 [SMILES: *Cc1ccc2[nH]ccc2c1] |
| 5i | SJ1008069 | 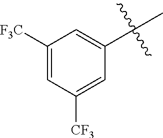 [SMILES: *c1cc(C(F)(F)F)cc(C(F)(F)F)c1] | 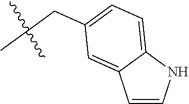 [SMILES: *Cc1ccc2[nH]ccc2c1] |

| | | |
|---|---|---|
| 5j | SJ1009807 | 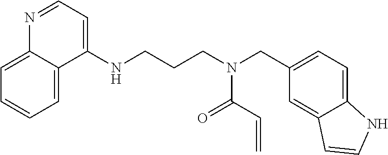 [SMILES: C=CC(=O)N(CCCNc1ccnc2ccccc12)Cc1ccc2[nH]ccc2c1] |

^{a}All compounds prepared as HCl salt form.

General Synthetic Procedures. All commercial reagents were used without further purification and the solvents were dried using the Glass Contour Solvent Systems by SG Water USA. All reactions were monitored by UPLC and thin-layer chromatography (TLC) carried out on silica gel coated glass plates. Flash-chromatography was performed on a Biotage Isolera One chromatography system using Biotage KP SIL or Biotage KP NH pre-packed columns and the solvent mixture in brackets was used as eluent. Evaporations were carried out in vacuo in a Buchi rotary evaporator. Nuclear magnetic resonance (NMR) spectra were obtained on a Bruker Avance II NMR spectrometer at 500 MHz for ¹H-NMR spectra and 126 MHz for ¹³C-NMR spectra. Chemical shifts (ppm) are reported relative to TMS or the solvent peak. Signals are designated as follows: s, singlet; d, doublet; dd, doublet of doublet; t, triplet; q, quadruplet; m, multiplet. Coupling constants (J) are expressed in Hertz.

### Synthesis of N¹-(quinolin-4-yl)propane-1,3-diamine (3a)

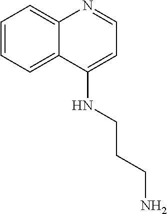 [SMILES: NCCCNc1ccnc2ccccc12]

4-chloroquinoline (0.500 g, 3.06 mmol) and propane-1,3-diamine (1.276 ml, 15.28 mmol) were stirred at 110° C. for 6 h and then cooled at room temperature overnight. Added 1M NaOH (10 mL) and water (5 mL) and extracted the aqueous layer with DCM. The DCM layer washed washed with water, brine and dried over anhydrous Na₂SO₄, filtered and concentrated to obtain a pale white solid (0.49 g, 80%). The crude product was pure by NMR and used directly for the next step. ¹H NMR (500 MHZ, Chloroform-d) δ 8.54 (d, J=5.3 Hz, 1H), 7.98-7.93 (m, 1H), 7.81-7.76 (m, 1H), 7.63-7.57 (m, 1H), 7.42-7.35 (m, 1H), 7.09 (s, 1H), 6.36 (d, J=5.3 Hz, 1H), 3.47-3.37 (m, 2H), 3.08-2.97 (m, 2H), 1.96-1.84 (m, 2H), 1.57 (s, 2H). ¹³C NMR (126 MHZ, CDCl₃) δ 151.15, 150.34, 148.46, 129.73, 128.85, 124.32, 120.17, 119.06, 98.12, 43.42, 41.38, 30.39. LR MS (ESI) Exact mass calcd for C₁₂H₁₆N₃ [M+H]⁺ 202.13, found 202.38.

### Synthesis of N¹-((1H-Indol-5-yl)methyl)-N³-(quinolin-4-yl)propane-1,3-diamine (5a)

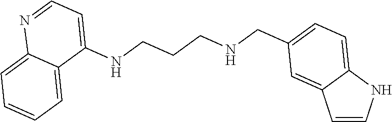 [SMILES: c1ccc2c(NCCCNCc3ccc4[nH]ccc4c3)ccnc2c1]

N¹-(quinolin-4-yl)propane-1,3-diamine (270 mg, 1.341 mmol) and 1H-indole-5-carbaldehyde (195 mg, 1.341 mmol) in MeOH (13 ml) were stirred at 23° C. for 1 h, then sodium borohydride (25.4 mg, 0.671 mmol) was added and continued stirring for 1 h more The reaction mixture was concentrated to remove the solvent, water added and the aqueous layer extracted with ethyl acetate. The ethyl acetate layer was washed with brine, dried over anhydrous Na₂SO₄, filtered and concentrated. The crude product was purified on Biotage NH column using a hexanes-ethyl actate (0-100%) gradient with MeOH (2%) as additive to provide the product (285 mg, 64%). ¹H NMR (500 MHZ, Chloroform-d) δ 8.51 (t, J=7.5 Hz, 2H), 7.93 (d, J=8.4 Hz, 1H), 7.67 (d, J=8.0 Hz, 2H), 7.63 (s, 1H), 7.55 (t, J=7.6 Hz, 1H), 7.38 (d, J=8.3 Hz, 1H), 7.27-7.24 (m, 1H), 7.20 (dd, J=8.3, 1.2 Hz, 1H), 7.14 (t, J=7.4 Hz, 1H), 6.54 (s, 1H), 6.32 (d, J=5.3 Hz, 1H), 3.94 (s, 2H), 3.41 (q, J=5.7 Hz, 2H), 3.05-2.96 (m, 2H), 1.95 (p, J=5.8 Hz, 2H), 1.74 (s, 1H). ¹³C NMR (126 MHZ, CDCl₃) δ 151.11, 150.58, 148.37, 135.21, 131.13, 129.43, 128.76, 128.07, 124.77, 124.22, 122.91, 120.80, 120.50, 119.16, 111.18, 102.53, 97.95, 54.96, 49.30, 44.10, 27.53. LRMS (ESI) Exact mass calcd for C₂₁H₂₃N₄ [M+H]⁺ 331.19, found 331.51. The product was resuspended in 1.25 M HCl in methanol solution (2 mL) and stirred for 1 h at 23° C. The solvent and excess HCl were then evaporated in vacuo to give the corresponding HCl salt. (SJ1008066).

### Synthesis of N¹-(7-(Trifluoromethyl)quinolin-4-yl)propane-1,3-diamine (3b)

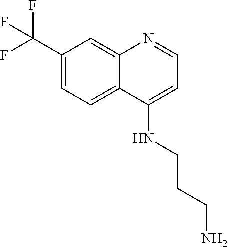 [SMILES: NCCCNc1ccnc2cc(C(F)(F)F)ccc12]

4-chloro-7-(trifluoromethyl)quinoline (1.3 g, 5.61 mmol) and propane-1,3-diamine (2.343 ml, 28.1 mmol) were stirred at 110° C. for 6 h and then cooled at room temperature overnight. Added 1M NaOH (10 mL) and water (5 mL) and extracted the aqueous layer with DCM. The DCM layer was washed with water, brine and dried over anhydrous Na₂SO₄, filtered and concentrated to obtain a pale yellow solid. (0.9 g, 60%). The crude product was pure by NMR and used directly for the next step. ¹H NMR (500 MHz, Chloroform-d) δ 8.58 (d, J=5.3 Hz, 1H), 8.23 (s, 1H), 7.90 (d, J=8.7 Hz, 1H), 7.72 (s, 1H), 7.53 (dd, J=8.7, 1.6 Hz, 1H), 6.40 (d, J=5.4 Hz, 1H), 3.43 (q, J=6.0 Hz, 2H), 3.13-3.01 (m, 2H), 1.97-1.84 (m, 2H), 1.49 (s, 2H). ¹³C NMR (126 MHZ, CDCl₃) δ 152.39, 150.31, 147.74, 131.00, 130.74, 130.48, 130.22, 127.44, 127.41, 127.37, 127.34, 125.21, 123.05, 121.87, 120.88, 119.76, 119.74, 119.71, 119.69, 99.20, 44.00, 41.64, 29.71. LRMS (ESI) C₁₃H₁₅F₃N₃ [M+H]⁺ 270.12, found 270.40.

### Synthesis of N¹-((1H-Indol-5-yl)methyl)-N³-(7-(trifluoromethyl)quinolin-4-yl)propane-1,3-diamine (5b)

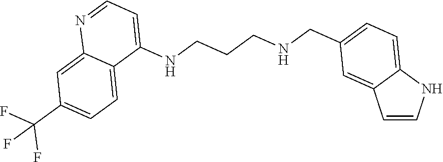 [SMILES: FC(F)(F)c1ccc2c(NCCCNCc3ccc4[nH]ccc4c3)ccnc2c1]

N¹-(7-(trifluoromethyl)quinolin-4-yl)propane-1,3-diamine (135 mg, 0.500 mmol) and 1H-indole-5-carbaldehyde (72.6 mg, 0.500 mmol) in MeOH (5 ml) were stirred at 23° C. for 1 h, then sodium borohydride (9.46 mg, 0.250 mmol) was added and continued stirring for 1 h more. The reaction mixture was concentrated to remove the solvent, water added and the aqueous layer extracted with ethyl acetate. The ethyl acetate layer was washed with brine, dried over anhydrous Na₂SO₄, filtered and concentrated. The crude product was purified on Biotage NH column using a DCM-MeOH (0-10%) gradient to give the desired product (83 mg, 42%). ¹H NMR (500 MHZ, DMSO-d6) δ 11.00 (s, 1H), 8.47 (d, J=5.4 Hz, 1H), 8.25 (d, J=8.8 Hz, 1H), 8.05 (s, 1H), 7.85 (s, 1H), 7.49-7.45 (m, 2H), 7.34-7.28 (m, 2H), 7.09 (dd, J=8.3, 1.5 Hz, 1H), 6.55 (d, J=5.5 Hz, 1H), 6.36-6.32 (m, 1H), 3.77 (s, 2H), 3.38-3.31 (m, 2H), 2.70 (t, J=6.4 Hz, 2H), 2.31 (s, 1H), 1.85 (p, J=6.5 Hz, 2H). ¹³C NMR (126 MHZ, DMSO-d6) δ 152.32, 149.98, 147.41, 134.98, 131.03, 129.34, 129.09, 128.84, 128.58, 127.56, 127.42, 126.34, 126.30, 126.27, 126.24, 125.30, 125.26, 123.77, 123.09, 121.78, 120.93, 120.85, 119.31, 118.77, 118.75, 118.72, 118.70, 111.00, 100.77, 99.56, 53.81, 46.97, 41.61, 27.59. LRMS (ESI) C₂₂H₂₂F₃N₄ [M+H]⁺ 399.18, found 399.53. The product was resuspended in 1.25 M HCl in methanol solution (2 mL) and stirred for 1 h at 23° C. The solvent and excess HCl were then evaporated in vacuo to give the corresponding HCl salt (SJ521054).

### Synthesis of N¹-(7-chloroquinolin-4-yl)propane-1,3-diamine (3c)

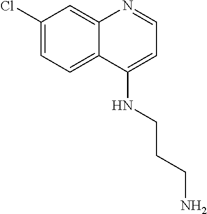 [SMILES: NCCCNc1ccnc2cc(Cl)ccc12]

4,7-dichloroquinoline (1.000 g, 5.05 mmol) and propane-1,3-diamine (2.107 ml, 25.2 mmol) were stirred at 110° C. for 6 h and then cooled at room temperature overnight. Added 1M NaOH (10 mL) and water (5 mL) and extracted the aqueous layer with DCM. The organic layer was washed with water, brine and dried over anhydrous Na₂SO₄, filtered and concentrated to obtain a pale yellow solid (0.85 g, 71%). The crude product was pure by NMR and used directly for the next step. ¹H NMR (500 MHZ, Chloroform-d) δ 8.51 (d, J=5.4 Hz, 1H), 7.93 (d, J=2.1 Hz, 1H), 7.72 (d, J=8.9 Hz, 1H), 7.45 (s, 1H), 7.32 (dd, J=8.9, 2.2 Hz, 1H), 6.33 (d, J=5.4 Hz, 1H), 3.45-3.38 (m, 2H), 3.08-3.03 (m, 2H), 1.94-1.85 (m, 2H), 1.51 (s, 2H). ¹³C NMR (126 MHz, CDCl₃) δ 152.17, 150.41, 149.21, 134.61, 128.60, 124.94, 121.95, 117.53, 98.29, 43.81, 41.57, 29.92. LRMS (ESI) Exact mass calcd for C₁₂H₁₅ClN₃ [M+H]⁺ 236.10, found 236.27.

### Synthesis of N¹-((1H-Indol-5-yl)methyl)-N³-(7-chloroquinolin-4-yl)propane-1,3-diamine (5c)

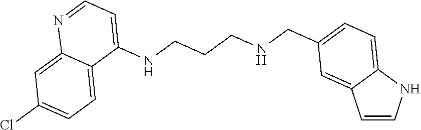 [SMILES: Clc1ccc2c(NCCCNCc3ccc4[nH]ccc4c3)ccnc2c1]

N¹-(7-chloroquinolin-4-yl)propane-1,3-diamine (200 mg, 0.848 mmol) and 1H-indole-5-carbaldehyde (123 mg, 0.848 mmol) in MeOH (8 ml) were stirred at 23° C. for 1 h, then sodium borohydride (16.05 mg, 0.424 mmol) was added and continued stirring for 1 h more The reaction mixture was concentrated to remove the solvent, water added and the aqueous layer extracted with ethyl acetate. The ethyl acetate layer was washed with brine, dried over anhydrous Na₂SO₄, filtered and concentrated. The crude product was purified on Biotage NH column using a hexanes-ethyl actate (0-100%) gradient with MeOH (2%) as additive to provide the product (158 mg, 51%). ¹H NMR (500 MHz, Chloroform-d) δ 8.52 (s, 1H), 8.47 (d, J=5.4 Hz, 1H), 7.97 (s, 1H), 7.88 (d, J=2.1 Hz, 1H), 7.61 (s, 1H), 7.48 (d, J=8.9 Hz, 1H), 7.39 (d, J=8.3 Hz, 1H), 7.28 (t, J=2.8 Hz, 1H), 7.18 (dd, J=8.3, 1.4 Hz, 1H), 6.92 (dd, J=8.9, 2.2 Hz, 1H), 6.56-6.52 (m, 1H), 6.27 (d, J=5.4 Hz, 1H), 3.92 (s, 2H), 3.42-3.36 (m, 2H), 3.06-3.00 (m, 2H), 1.98-1.91 (m, 2H), 1.74 (s, 1H). ¹³C NMR (126 MHZ, CDCl₃) δ 152.03, 150.63, 149.06, 135.23, 134.48, 131.01, 128.20, 128.12, 124.93, 124.85, 122.84, 122.57, 120.56, 117.59, 111.25, 102.53, 98.12, 54.97, 49.56, 44.30, 27.23. LRMS (ESI) Exact mass calcd for C₂₁H₂₂ClN₄ [M+H]⁺ 365.15, found 365.40. The product was resuspended in 1.25 M HCl in methanol solution (2 mL) and stirred for 1 h at 23° C. The solvent and excess HCl were then evaporated in vacuo to give the corresponding HCl salt (SJ520909).

### Synthesis of N¹-(7-Bromoquinolin-4-yl)propane-1,3-diamine (3d)

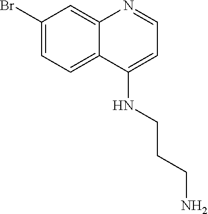 [SMILES: NCCCNc1ccnc2cc(Br)ccc12]

7-bromo-4-chloroquinoline (1.000 g, 4.12 mmol) and propane-1,3-diamine (1.721 ml, 20.62 mmol) were stirred at 110° C. for 6 h and then cooled at room temperature overnight. Added 1M NaOH (10 mL) and water (5 mL) and extracted the aqueous layer with DCM. The DCM layer washed washed with water, brine and dried over anhydrous Na₂SO₄, filtered and concentrated to obtain a pale white solid (0.93 g, 80%). The crude product was pure by NMR and used directly for the next step. ¹H NMR (500 MHZ, Chloroform-d) δ 8.49 (d, J=5.4 Hz, 1H), 8.11 (d, J=2.0 Hz, 1H), 7.64 (d, J=8.9 Hz, 1H), 7.50-7.41 (m, 2H), 6.33 (d, J=5.4 Hz, 1H), 3.41 (q, J=6.0 Hz, 2H), 3.08-3.00 (m, 2H), 1.93-1.85 (m, 2H), 1.59 (s, 2H). ¹³C NMR (126 MHZ, CDCl₃) δ 152.06, 150.48, 149.42, 131.85, 127.46, 122.86, 122.05, 117.83, 98.36, 43.75, 41.53, 29.90. LRMS (ESI) Exact mass calcd for C₁₂H₁₅BrN₃ [M+H]⁺ 280.04, found 280.20.

### Synthesis of N¹-((1H-Indol-5-yl)methyl)-N³-(7-bromoquinolin-4-yl)propane-1,3-diamine (5d)

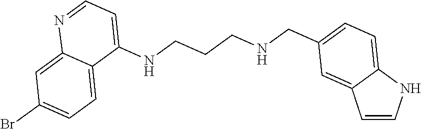 [SMILES: Brc1ccc2c(NCCCNCc3ccc4[nH]ccc4c3)ccnc2c1]

N¹-(7-bromoquinolin-4-yl)propane-1,3-diamine (406 mg, 1.449 mmol) and 1H-indole-5-carbaldehyde (210 mg, 1.449 mmol) in MeOH (14 ml) were stirred at 23° ° C. for 1 h, then sodium borohydride (27.4 mg, 0.725 mmol) was added and continued stirring for 1 h more The reaction mixture was concentrated to remove the solvent, water added and the aqueous layer extracted with ethyl acetate. The ethyl acetate layer was washed with brine, dried over anhydrous Na₂SO₄, filtered and concentrated. The crude product was purified on Biotage NH column using a hexanes-ethyl actate (0-100%) gradient with MeOH (2%) as additive to provide the product (452 mg, 76%). ¹H NMR (500 MHZ, Chloroform-d) δ 8.47 (d, J=5.3 Hz, 1H), 8.35 (s, 1H), 8.06 (d, J=2.0 Hz, 1H), 7.98 (s, 1H), 7.61 (s, 1H), 7.40 (dd, J=8.6, 4.3 Hz, 2H), 7.29 (t, J=2.8 Hz, 1H), 7.18 (d, J=8.3 Hz, 1H), 7.05 (dd, J=8.9, 2.0 Hz, 1H), 6.56-6.53 (m, 1H), 6.29 (d, J=5.4 Hz, 1H), 3.92 (s, 2H), 3.40 (q, J=5.5 Hz, 2H), 3.08-2.99 (m, 2H), 1.99-1.92 (m, 2H), 1.64 (s, 1H). ¹³C NMR (126 MHZ, CDCl₃) δ 152.03, 150.66, 149.39, 135.20, 131.58, 131.06, 128.12, 127.38, 124.90, 122.86, 122.75, 122.61, 120.59, 117.91, 111.23, 102.59, 98.20, 54.98, 49.59, 44.33, 27.25. LRMS (ESI) Exact mass calcd for C₂₁H₂₂BrN₄ [M+H]⁺ 409.10, found 409.33. The product was resuspended in 1.25 M HCl in methanol solution (2 mL) and stirred for 1 h at 23° C. The solvent and excess HCl were then evaporated in vacuo to give the corresponding HCl salt (SJ1008065).

### Synthesis of N¹-((1-Methyl-1H-indol-5-yl)methyl)-N³-(7-(trifluoromethyl)quinolin-4-yl)propane-1,3-diamine (5e)

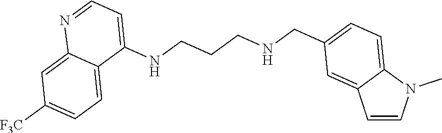 [SMILES: Cn1ccc2cc(CNCCCNc3ccnc4cc(C(F)(F)F)ccc34)ccc21]

N¹-(7-(trifluoromethyl)quinolin-4-yl)propane-1,3-diamine (100 mg, 0.371 mmol) and 1-methyl-1H-indole-5-carbaldehyde (59.1 mg, 0.371 mmol) in MeOH (3.5 ml) were stirred at 23° C. for 1 h, then sodium borohydride (7.02 mg, 0.186 mmol) was added and continued stirring for 1 h more The reaction mixture was concentrated to remove the solvent, water added and the aqueous layer extracted with ethyl acetate. The ethyl acetate layer was washed with brine, dried over anhydrous Na₂SO₄, filtered and concentrated. The crude product was purified on Biotage NH column using a hexanes-ethyl actate (0-100%) gradient to provide the product (87 mg, 57%). 1H NMR (500 MHZ, Chloroform-d) δ 8.53 (d, J=5.3 Hz, 1H), 8.22 (s, 1H), 8.16 (s, 1H), 7.60 (s, 1H), 7.56 (d, J=8.7 Hz, 1H), 7.31 (d, J=8.3 Hz, 1H), 7.20 (dd, J=8.3, 1.5 Hz, 1H), 7.12 (d, J=3.1 Hz, 1H), 7.03 (dd, J=8.7, 1.7 Hz, 1H), 6.46 (d, J=3.0 Hz, 1H), 6.34 (d, J=5.4 Hz, 1H), 3.93 (s, 2H), 3.83 (s, 3H), 3.41 (q, J=5.5 Hz, 2H), 3.09-3.04 (m, 2H), 2.00-1.93 (m, 2H), 1.67 (s, 1H). ¹³C NMR (126 MHZ, CDCl₃) δ 152.31, 150.44, 147.61, 136.14, 130.77, 130.50, 130.25, 130.00, 129.56, 128.70, 127.07, 127.04, 127.00, 126.97, 125.24, 123.08, 122.41, 122.40, 120.94, 120.75, 119.70, 119.68, 119.65, 119.62, 109.46, 100.84, 98.95, 54.96, 49.71, 44.44, 32.89, 27.15. LRMS (ESI) C₂₃H₂₄F₃N₄ [M+H]⁺ 413.20, found 413.44. The product was resuspended in 1.25 M HCl in methanol solution (2 mL) and stirred for 1 h at 23° C. The solvent and excess HCl were then evaporated in vacuo to give the corresponding HCl salt (SJ1008067).

### Synthesis of N¹-(Benzo[d][1,3]dioxol-5-ylmethyl)-N³-(7-(trifluoromethyl)quinolin-4-yl)propane-1,3-diamine (5f)

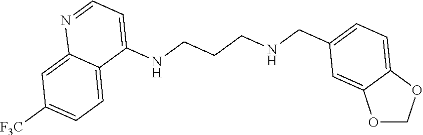 [SMILES: FC(F)(F)c1ccc2c(NCCCNCc3ccc4c(c3)OCO4)ccnc2c1]

N¹-(7-(trifluoromethyl)quinolin-4-yl)propane-1,3-diamine (100 mg, 0.371 mmol) and benzo[d][1,3]dioxole-5-carbaldehyde (55.8 mg, 0.371 mmol) in MeOH (3.5 ml) were stirred at 23 ºC for 1 h, then sodium borohydride (7.02 mg, 0.186 mmol) was added and continued stirring for 1 h more. The reaction mixture was concentrated to remove the solvent, water added and the aqueous layer extracted with ethyl acetate. The ethyl acetate layer was washed with brine, dried over anhydrous Na₂SO₄, filtered and concentrated. The crude product was purified on Biotage NH column using a hexanes-ethyl actate (0-100%) gradient to obtain the product (63.5 mg, 42%). ¹H NMR (500 MHZ, Chloroform-d) δ 8.58 (d, J=5.3 Hz, 1H), 8.22 (s, 1H), 7.83 (s, 1H), 7.73 (d, J=8.7 Hz, 1H), 7.42 (d, J=8.7 Hz, 1H), 6.85 (s, 1H), 6.82-6.77 (m, 2H), 6.38 (d, J=5.3 Hz, 1H), 5.99 (s, 2H), 3.75 (s, 2H), 3.42 (q, J=5.0 Hz, 2H), 3.03-2.96 (m, 2H), 1.99-1.92 (m, 2H), 1.68 (s, 1H). ¹³C NMR (126 MHZ, CDCl₃) δ 152.40, 150.29, 147.92, 147.71, 146.95, 133.45, 130.98, 130.72, 130.46, 130.20, 127.39, 127.37, 127.34, 127.31, 125.23, 123.06, 122.04, 121.58, 120.89, 119.75, 119.73, 119.70, 119.68, 109.02, 108.32, 101.13, 99.20, 54.32, 49.43, 44.24, 27.32. LRMS (ESI) C₂₁H₂₁F₃N₃O₂ [M+H]⁺ 404.16, found 404.39. The product was resuspended in 1.25 M HCl in methanol solution (2 mL) and stirred for 1 h at 23° C. The solvent and excess HCl were then evaporated in vacuo to give the corresponding HCl salt (SJ361106).

### Synthesis of N¹-(Furan-2-ylmethyl)-N³-(7-(trifluoromethyl)quinolin-4-yl)propane-1,3-diamine (5g)

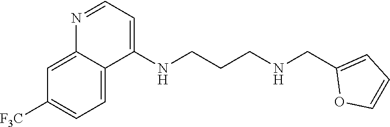 [SMILES: FC(F)(F)c1ccc2c(NCCCNCc3ccco3)ccnc2c1]

N¹-(7-(trifluoromethyl)quinolin-4-yl)propane-1,3-diamine (100 mg, 0.371 mmol) and furan-2-carbaldehyde (35.7 mg, 0.371 mmol) in MeOH (3.5 ml) were stirred at 23° ° C. for 1 h then sodium borohydride (7.02 mg, 0.186 mmol) was added and continued stirring for 1 h more. The reaction mixture was concentrated to remove the solvent, water added and the aqueous layer extracted with ethyl acetate. The ethyl acetate layer was washed with brine, dried over anhydrous Na₂SO₄, filtered and concentrated. The crude product was purified on Biotage NH column using a hexanes-ethyl actate (0-100%) gradient to obtain the product (107 mg, 82%). ¹H NMR (500 MHz, Chloroform-d) δ 8.58 (d, J=5.3 Hz, 1H), 8.22 (s, 1H), 7.91 (s, 1H), 7.82 (d, J=8.7 Hz, 1H), 7.44 (dd, J=8.7, 1.5 Hz, 1H), 7.41-7.38 (m, 1H), 6.40-6.35 (m, 2H), 6.24 (d, J=3.1 Hz, 1H), 3.87 (s, 2H), 3.42 (q, J=5.7 Hz, 2H), 3.01-2.96 (m, 2H), 1.99-1.92 (m, 2H), 1.70 (s, 1H). ¹³C NMR (126 MHZ, CDCl₃) δ 153.08, 152.40, 150.37, 147.74, 142.21, 130.96, 130.70, 130.44, 130.18, 127.40, 127.37, 127.33, 127.30, 125.24, 123.07, 122.14, 120.93, 119.68, 119.65, 119.63, 119.60, 110.33, 107.37, 99.15, 49.05, 46.23, 44.28, 27.02. LRMS (ESI) C₁₈H₁₉F₃N₃O [M+H]⁺ 350.15, found 350.43. The product was resuspended in 1.25 M HCl in methanol solution (2 mL) and stirred for 1 h at 23° C. The solvent and excess HCl were then evaporated in vacuo to give the corresponding HCl salt (SJ361113).

### Synthesis of N¹-((1H-Indol-5-yl)methyl)-N³-(7-(4-(tert-butyl)phenyl)quinolin-4-yl)propane-1,3-diamine (5h)

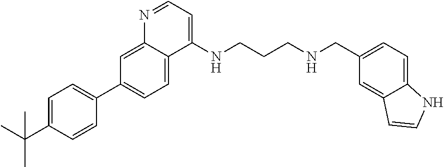 [SMILES: CC(C)(C)c1ccc(-c2ccc3c(NCCCNCc4ccc5[nH]ccc5c4)ccnc3c2)cc1]

N¹-((1H-indol-5-yl)methyl)-N³-(7-bromoquinolin-4-yl)propane-1,3-diamine (55 mg, 0.134 mmol), (4-(tert-butyl)phenyl)boronic acid (47.8 mg, 0.269 mmol) and potassium phosphate (62.7 mg, 0.296 mmol) in dioxane (1.5 ml) and water (0.250 ml) were degassed under N₂ and then Pd(dppf)Cl₂ (19.66 mg, 0.027 mmol) was added. The reaction mixture was heated at 110° C. under N₂ for 16 h and then cooled to room temperature. Water was added, extracted with DCM and the DCM layer was washed with brine, dried over anhydrous Na₂SO₄, filtered and concentrated to obtain a dark brown product. The crude product was purified on a Biotage 11 g NH column using a DCM-MOH (0-8% MeoH) gradient to provide the desired product (30 mg, 48%). ¹H NMR (500 MHZ, Chloroform-d) δ 8.52 (d, J=5.3 Hz, 1H), 8.36 (s, 1H), 8.16 (s, 1H), 7.71 (s, 1H), 7.68-7.61 (m, 4H), 7.51 (d, J=8.3 Hz, 2H), 7.40 (d, J=8.3 Hz, 1H), 7.34 (dd, J=8.6, 1.6 Hz, 1H), 7.27 (t, J=2.7 Hz, 1H), 7.22 (d, J=8.3 Hz, 1H), 6.56 (s, 1H), 6.31 (d, J=5.3 Hz, 1H), 3.96 (s, 2H), 3.43 (q, J=5.3 Hz, 2H), 3.10-2.97 (m, 2H), 2.02-1.90 (m, 2H), 1.65 (s, 1H), 1.39 (s, 9H). ¹³C NMR (126 MHZ, CDCl₃) δ 151.53, 150.62, 150.50, 148.79, 141.01, 137.57, 135.22, 131.26, 128.12, 126.91, 126.82, 125.82, 124.74, 123.52, 122.94, 121.31, 120.58, 118.13, 111.22, 102.68, 97.90, 54.95, 49.37, 44.14, 34.61, 31.40, 27.57. LRMS (ESI) C₃₁H₃₅N₄ [M+H]⁺ 463.29, found 463.45. The product was resuspended in 1.25 M HCl in methanol solution (2 mL) and stirred for 1 h at 23° C. The solvent and excess HCl were then evaporated in vacuo to give the corresponding HCl salt (SJ1008068).

### Synthesis of N¹_((1H-Indol-5-yl)methyl)-N³-(7-(3,5-bis(trifluoromethyl)phenyl)quinolin-4-yl)propane-1,3-diamine (5i)

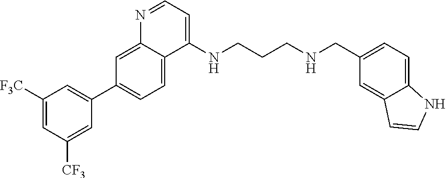 [SMILES: FC(F)(F)c1cc(-c2ccc3c(NCCCNCc4ccc5[nH]ccc5c4)ccnc3c2)cc(C(F)(F)F)c1]

N¹-((1H-indol-5-yl)methyl)-N³-(7-bromoquinolin-4-yl)propane-1,3-diamine (55 mg, 0.134 mmol), (3,5-bis(trifluoromethyl)phenyl)boronic acid (69.3 mg, 0.269 mmol) and potassium phosphate (62.7 mg, 0.296 mmol) in dioxane (1.5 ml) and water (0.250 ml) were degassed under N₂ and then Pd(dppf)Cl₂ (19.66 mg, 0.027 mmol) was added and the reaction mixture was heated at 110° C. under N₂ for 16 h and then cooled to room temperature. Water was added, extracted with DCM and the DCM layer was washed with brine, dried over anhydrous Na₂SO₄, filtered and concentrated to obtain a dark brown product. The crude product was purified on a Biotage 11 g NH column using a DCM-MOH (0-8% MeoH) gradient to provide the desired product (32 mg, 44%). ¹H NMR (500 MHZ, Chloroform-d) δ 8.53 (d, J=5.3 Hz, 1H), 8.29 (s, 2H), 8.15 (d, J=1.7 Hz, 1H), 8.09 (s, 2H), 7.88 (s, 1H), 7.70 (s, 1H), 7.54 (d, J=8.6 Hz, 1H), 7.42 (d, J=8.3 Hz, 1H), 7.29 (t, J=2.7 Hz, 1H), 7.20 (d, J=8.3 Hz, 1H), 6.96 (dd, J=8.6, 1.8 Hz, 1H), 6.56 (s, 1H), 6.32 (d, J=5.4 Hz, 1H), 3.96 (s, 2H), 3.46 (t, J=5.6 Hz, 2H), 3.16-3.08 (m, 2H), 2.04-1.96 (m, 2H), 1.84 (s, 1H). ¹³C NMR (126 MHz, CDCl₃) δ 151.97, 150.58, 148.48, 142.57, 137.76, 135.24, 132.48, 132.21, 131.95, 131.69, 131.22, 128.24, 127.97, 127.51, 127.26, 127.24, 126.73, 124.98, 124.72, 124.57, 122.85, 122.61, 122.52, 122.40, 122.01, 121.12, 121.09, 121.06, 121.03, 121.00, 120.69, 120.23, 119.72, 119.20, 111.30, 102.68, 98.26, 54.90, 50.00, 44.50, 27.19. LRMS (ESI) C₂₉H₂₅F₆N₄ [M+H]⁺ 543.20, found 543.55. The product was resuspended in 1.25 M HCl in methanol solution (2 mL) and stirred for 1 h at 23° C. The solvent and excess HCl were then evaporated in vacuo to give the corresponding HCl salt (SJ1008069).

### Synthesis of N-((1H-Indol-5-yl)methyl)-N-(3-(quinolin-4-ylamino)propyl)acrylamide (5j)

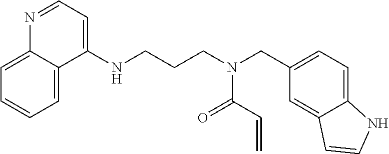 [SMILES: C=CC(=O)N(CCCNc1ccnc2ccccc12)Cc1ccc2[nH]ccc2c1]

To N¹-((1H-indol-5-yl)methyl)-N³-(quinolin-4-yl)propane-1,3-diamine (50 mg, 0.151 mmol) in DCM (1.5 ml) was added triethylamine (0.042 ml, 0.303 mmol). The reaction mixture was cooled to 0° C. and acryloyl chloride (0.012 ml, 0.151 mmol) was added and stirred under N₂ for 2 h at 0° C. Added water to the reaction mixture and extracted the aqueous layer with DCM. The DCM layer was washed with brine, dried over anhydrous Na₂SO₄, filtered and concentrated. The crude product was purified on a Biotage 11 g NH column using a DCM-MeOH (0-8% MeOH) gradient to obtain the desired product (6.2 mg, 11%). ¹H NMR (500 MHZ, Chloroform-d) δ 8.49 (d, J=5.3 Hz, 2H), 8.04 (d, J=8.2 Hz, 1H), 7.94 (d, J=8.4 Hz, 1H), 7.64-7.58 (m, 1H), 7.49-7.41 (m, 2H), 7.36 (d, J=8.4 Hz, 1H), 7.26-7.24 (m, 1H), 7.00 (dd, J=8.4, 1.4 Hz, 1H), 6.72 (dd, J=16.7, 10.4 Hz, 1H), 6.61 (t, J=5.7 Hz, 1H), 6.52 (d, J=2.0 Hz, 1H), 6.35 (d, J=5.4 Hz, 1H), 5.77 (dd, J=10.4, 1.9 Hz, 1H), 4.72 (s, 2H), 3.63 (t, J=6.2 Hz, 2H), 3.35 (q, J=6.0 Hz, 2H), 1.90-1.81 (m, 2H), 1.73 (s, 1H). ¹³C NMR (126 MHZ, CDCl₃) δ 167.97, 150.93, 149.93, 148.63, 135.39, 129.55, 129.07, 128.99, 128.19, 127.88, 127.46, 125.22, 124.61, 120.59, 120.38, 119.26, 118.66, 111.69, 102.56, 98.05, 51.76, 43.22, 39.29, 25.95. LRMS (ESI) C₂₄H₂₅N₄O [M+H]⁺ 385.20, found 385.47. The product was resuspended in 1.25 M HCl in methanol solution (2 mL) and stirred for 1 h at 23° C. The solvent and excess HCl were then evaporated in vacuo to give the corresponding HCl salt (SJ1009807).

### Representative Disclosed Compounds

Representative compounds were prepared by the chemical syntheses described herein above, activity measured by the TR-FRET assay described herein above, and IC₅₀ values calculated. The compound structures and IC₅₀ values are provided in Table 8 below. It should be noted that the compound structure is provided in SMILES (Simplified Molecular Input Line Entry System) format. SMILES was developed through funding from the U.S. Environmental Protection Agency, Mid-Continent Ecology Division-Duluth, (MED-Duluth) Duluth, MN to the Medicinal Chemistry Project at Pomona College, Claremont, CA and the Computer Sciences Corporation, Duluth, MN. Several publications discuss SMILES in more detail, including Anderson et al. 1987, Weininger 1988, Weininger et al. 1989, and Hunter et al., 1987.

### Further Discussion

MAGE genes were discovered almost three decades ago as reproductive tissue restricted genes that when aberrantly expressed in cancer elicit a cytotoxic immune response due to their antigenicity (Ref. 28). This discovery set off a flurry of research on the potential of MAGEs to prime the immune systems of cancer patients to target tumor cells (Ref. 29). However, the underlying function and biochemical activity of MAGE genes were largely understudied. More recently, greater understanding into the normal physiological and disease functions of MAGEs has been elucidated (Ref. 1-3). Importantly, the MAGE family of proteins were biochemically defined as scaffolding proteins that diversify the function of E3 ubiquitin ligases through the recruitment of novel substrates for ubiquitination (Ref., 2, 8). However, many questions remained unanswered that have hindered the understanding of how MAGEs work and therapeutically targeting the many MAGE cancer-testis antigens. Chiefly, the molecular and structural basis of how MAGEs identify substrates was unknown. The specific degron sequences recognized by any given MAGE was unclear and the sequence and structural features on MAGEs that mediate recognition were unknown. These questions have important implications into MAGE biology, including understanding how relatively similar MAGE proteins can have distinct targets that impinge on a variety of cellular pathways. Answering these questions will facilitate the design of MAGE chemical inhibitors. Herein are provided the first evidence of how MAGEs recognize substrates and identify chemical inhibitors of the cancer-selective, oncogenic MAGE-A11.

The studies disclosed herein described the crystal structure of MAGE-A11 bound to its substrate PCF11. Strikingly, it was found that a small peptide of PCF11 forms short alpha-helix that binds into a hydrophobic cleft formed at the interface of WH-A and WH-B motifs within the MAGE-A11 MHD. Mutational analysis of this interface across three distinct MAGEs (MAGE-A3, -A11 and -F1) established that this region defines the substrate binding site on MAGEs. Thus, this region is referred to herein as the substrate binding cleft (SBC). Notably, in the previously solved structures of MAGE-A3 and -A4, the SBC was occupied by unstructured intermolecular MAGE protein or tag residues, respectively (Ref. 9). Specifically, MAGE-A3 was observed as a domain swapped dimer where the C-terminus was observed as a random coil threaded through the SBC of the crystallographic symmetry related protomer. However, in the MAGE-A11:PCF11 structure, these residues form the terminal helix of a helical bundle structure at the C-terminus that partly contributes to substrate binding. In MAGE-A4, the N-terminal purification tag threads through the SBC as part of a domain-swapped symmetry related dimer (Ref. 9). The data disclosed herein suggest that interaction of the unstructured C-termini of MAGE-A3 and N-terminal tag of MAGE-A4 with the SBC may be induced during crystallization due to the absence of a natural substrate. This could lead to the hydrophobic cleft being filled unnaturally to allow protein stabilization and crystallization. In either case, the SBC appears to be an important binding site for protein-protein interaction, including substrates.

The MAGE family is evolutionarily conserved in all eukaryotes and underwent dynamic expansion from a single gene in protozoa to a large multigene family in eutherian mammals (Ref. 2). Evolutionarily, type II MAGEs appeared earlier than type I MAGEs, but the MHD is a common feature to both type I and II MAGEs. MHDs are highly conserved (46% identical) in human, especially, the MHDs of MAGE-A subfamily share >80% protein sequence identity (Ref. 7, 8). However, distinct MHDs often recognize non-overlapping substrates (Ref. 5, 10. 15). The data and studies disclosed herein show that this substrate selectivity is likely imparted by sequence diversity around the SBC that may provide key positive and negative filtering. In addition to binding substrates, the MHD also mediates binding to RING or HECT ligases. For example, MAGE-C2 MHD bound the coiled-coil region on TRIM28, MAGE-B18 bound a basic region of LNX1 and MAGE-G1 bound the WH-A motif of NSE1 (Ref. 8). Although the MHD mediates ligase binding, it was determined herein that mutation of MAGE-A3, -A11 or -F1 SBC did not disrupt binding to their cognate E3 ligases. This suggests that: 1) SBC mutations do not cause global structural changes to the MHD and 2) ligase and substrate binding to the MHD occur independently on non-overlapping surfaces. Consistent with these findings, a previous co-crystal structure of MAGE-G1:NSE1 showed a distinct binding surface for NSE1 not restricted to the MHD SBC (Ref. 8). Notably, the relative orientation of WH-A and WH-B of MAGE-G1 MHD in the MAGE-G1:NSE1 structure is quite distinct compared to the structures of MAGE-A3, -A4 and -A11:PCF11 (Ref. 5). Therefore, E3 binding may induce structural dynamics within the MHD and the relative positioning of the WH motifs may contribute to selective ligase binding between MAGEs. Additional MHD structures with both ligase and substrate bound will provide important answers to these questions.

Finally. MAGEs and other cancer-testis antigens have been extensively proposed as cancer-specific therapeutic targets. Thus far there has been limited success. The present disclosure provides compounds and peptides targeting oncogenic MAGEs through blocking binding to substrates. It is believed that this approach will be of broad utility against the many cancer-specific MAGEs. As proof-of-principle, the studies disclosed herein target MAGE-A11 and identify a novel genus of sub-micromolar 4-aminoquinolines inhibitors of MAGE-A11 binding to PCF11.

### FURTHER ASPECTS OF THE DISCLOSURE

The current disclosure will be better appreciated upon reading the following aspects, which should not be confused with the claims. Each of the aspects described below can in some instances be provided in combination with other aspects, including other numbered aspects below or aspects described elsewhere in the disclosure, without departing from the spirit of the disclosure.
Aspect 1. A compound having a structure represented by a formula:

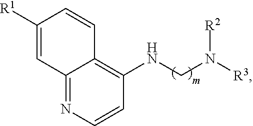

wherein m is selected from 2, 3, and 4; wherein R¹ is selected from hydrogen, halogen, C1-C10 haloalkyl, C1-C10 alkoxy, C1-C10 aminoalkyl, C1-C10 alkylamino, C1-C10 hydoxyalkyl, C1-C10 alkyl, -Q-Ar¹, -Q-Cy¹, —Ar¹, and —Cy¹; wherein Q is selected from —(CH₂)ₙ— and —O—; wherein n is selected from 1 and 2; wherein the Ar¹ is selected from phenyl, benzyl, indolyl, benzo[d][1,3]dioxolyl naphthyl, and anthracenyl; wherein the Ar¹ is substituted with 0, 1, or 2 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; wherein the Cy¹ is C3-C8 cycloalkyl; and wherein the Cy¹ is substituted with 0, 1, 2, 3, 4, or 5 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; wherein R² is selected from H and C1-C3 alkyl; wherein R³ is selected from H, C1-C3 alkyl, and —(CH₂)ₚ—Ar²; wherein p is selected from 1 and 2; wherein Ar² is selected from a phenyl, indolyl, benzo[d][1,3]dioxolyl, naphthyl, and anthracenyl wherein the Ar² is substituted with 0, 1, or 2 groups independently selected from halogen, —SF₅, —CN, —N₃, —NO₂, —OH, —CN, C1-C3 alkoxy, C1-C3 haloalkyl, C1-C3 aminoalkyl, C1-C3 alkylamino, C1-C3 hydoxyalkyl, and C1-C3 alkyl; or a pharmaceutically acceptable salt thereof.
Aspect 2. The compound according to any one of Aspects 1-26, wherein m is selected from 2 and 3.
Aspect 3. The compound according to any one of Aspects 1-26, wherein m is 3.
Aspect 4. The compound according to any one of Aspects 1-26, wherein R¹ is selected from halogen, C1-C10 haloalkyl, C1-C10 alkoxy, C1-C10 aminoalkyl, C1-C10 alkylamino, C1-C10 hydoxyalkyl, C1-C10 alkyl, —Ar¹, —Cy¹, —O—Ar¹, —O—Cy¹, —(C1-C2 alkanediyl)-Ar¹, and —(C1-C2 alkanediyl)-Cy¹.
Aspect 5. The compound according to any one of Aspects 1-26, wherein R¹ is selected from halogen, C1-C6 haloalkyl, C1-C6 alkoxy, C1-C6 aminoalkyl, C1-C6 alkylamino, C1-C6 hydoxyalkyl, C1-C6 alkyl, -cyclohexyl, —(C1-C2 alkanediyl)-cyclohexyl, —O—Ar¹, and —(C1-C2 alkanediyl)-Ar¹.
Aspect 6. The compound according to any one of Aspects 1-26, wherein R¹ is selected from chloro, C1-C3 haloalkyl, C1-C6 alkyl, -cyclohexyl, —(CH₂)-cyclohexyl, —O—Ar¹, and —(CH₂)—Ar¹.
Aspect 7. The compound according to any one of Aspects 1-26, wherein Ar¹ is phenyl substituted with 0, 1, or 2 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.
Aspect 8. The compound according to any one of Aspects 1-26, wherein Ar¹ is phenyl substituted with 0, 1, or 2 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.
Aspect 9. The compound according to any one of Aspects 1-26, wherein R² is selected from hydrogen and methyl.
Aspect 10. The compound according to any one of Aspects 1-26, wherein R² is hydrogen.
Aspect 11. The compound according to any one of Aspects 1-26, wherein R³ is selected from hydrogen and methyl.
Aspect 12. The compound according to any one of Aspects 1-26, wherein R³ is hydrogen.
Aspect 13. The compound according to any one of Aspects 1-26, wherein R³ is —(CH₂)ₙ—Ar².
Aspect 14. The compound according to any one of Aspects 1-26, wherein n is 1.
Aspect 15. The compound according to any one of Aspects 1-26, wherein Ar² is selected from indolyl, benzo[d][1,3]dioxolyl and phenyl substituted with 0, 1, or 2 groups independently selected from halogen, —SF₅, —CN, —N₃, —NO₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.
Aspect 16. The compound according to any one of Aspects 1-26, wherein Ar² is indolyl.
Aspect 17. The compound according to any one of Aspects 1-26, wherein indolyl is unsubstituted.
Aspect 18. The compound according to any one of Aspects 1-26, wherein Ar² is benzo[d][1,3]dioxolyl.
Aspect 19. The compound according to any one of Aspects 1-26, wherein benzo[d][1,3]dioxolyl is unsubstituted.
Aspect 20. The compound according to any one of Aspects 1-26, wherein Ar² is phenyl substituted with 0, 1, or 2 groups independently selected from halogen, —SF₅, —CN, —N₃, —NO₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.
Aspect 21. The compound according to any one of Aspects 1-26, wherein the compound has a structure represented by a formula:

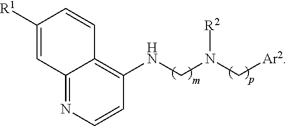

Aspect 22. The compound according to any one of Aspects 1-26, wherein the compound has a structure represented by a formula:

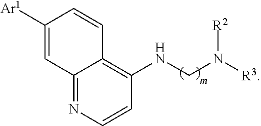

Aspect 23. The compound according to any one of Aspects 1-26, wherein the compound has a structure represented by a formula:

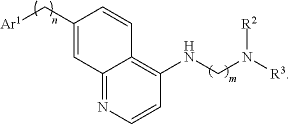

Aspect 24. The compound according to any one of Aspects 1-26, wherein the compound has a structure represented by a formula:

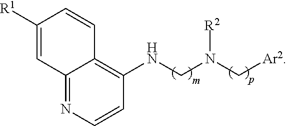

Aspect 25. The compound according to any one of Aspects 1-26, wherein the compound has a structure represented by a formula:

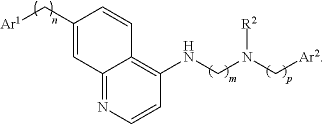

Aspect 26. The compound according to any one of Aspects 1-25, having a structure represented by a formula:

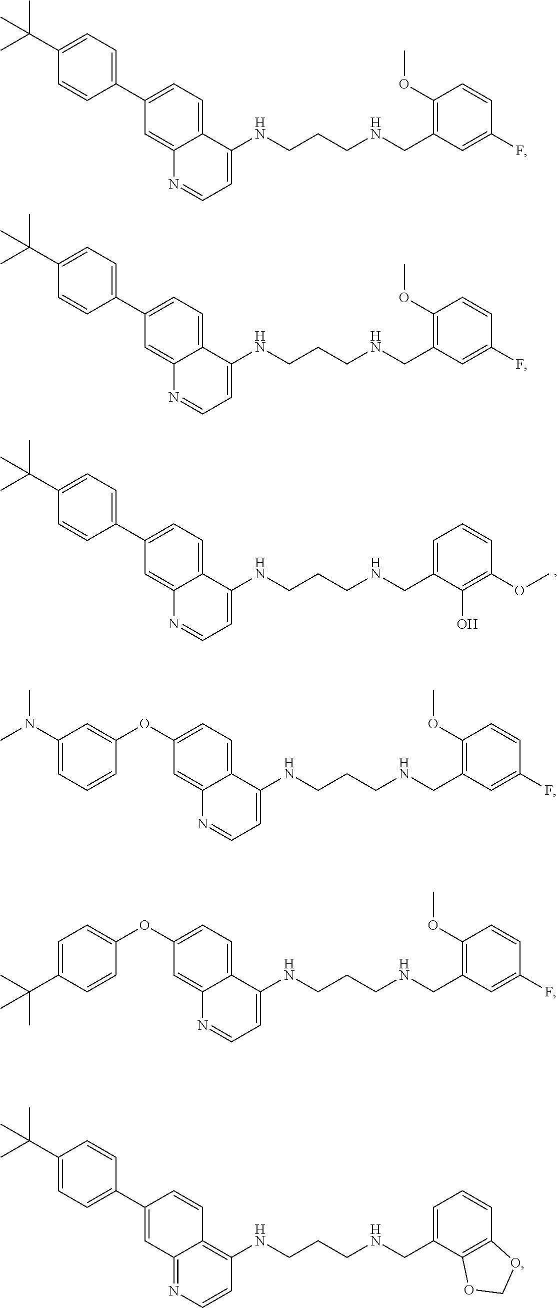

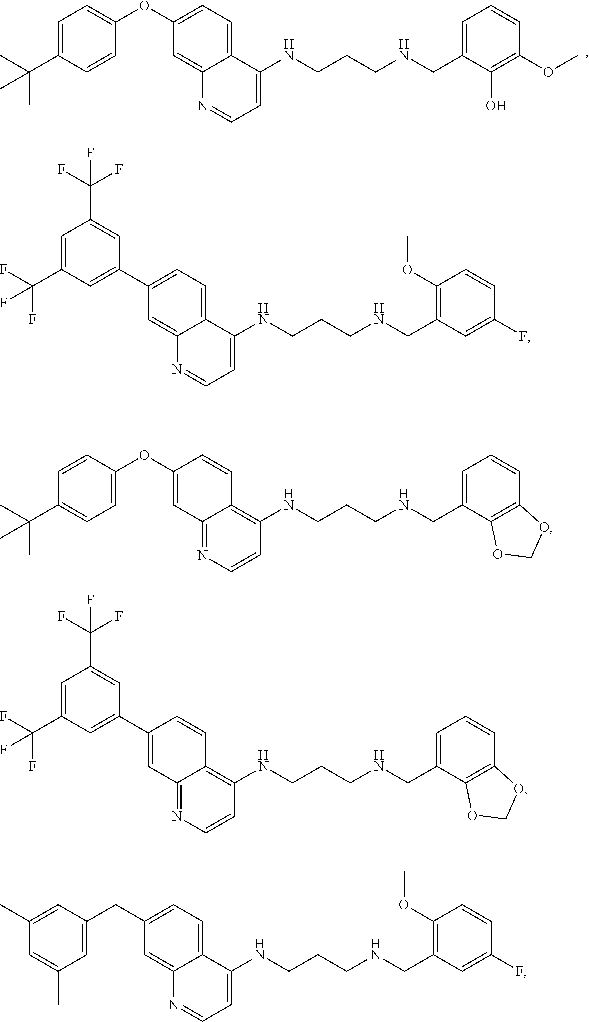

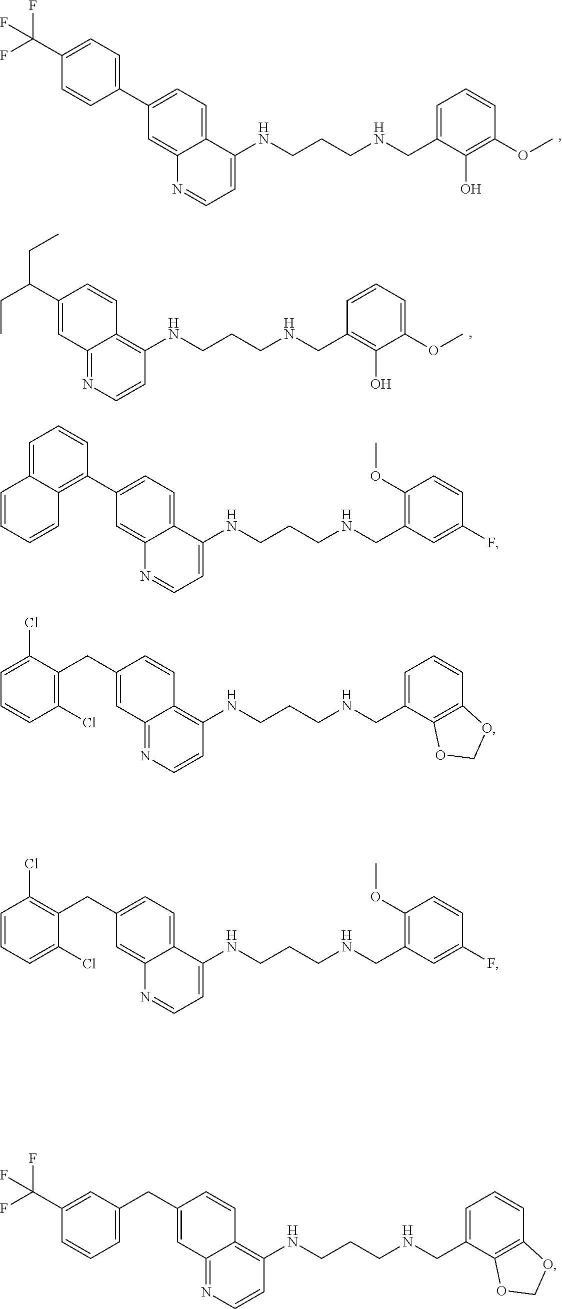 [SMILES: COc1cccc(CNCCCNc2ccnc3cc(-c4ccc(C(F)(F)F)cc4)ccc23)c1O ; CCC(CC)c1ccc2c(NCCCNCc3cccc(OC)c3O)ccnc2c1 ; COc1ccc(F)cc1CNCCCNc1ccnc2cc(-c3cccc4ccccc34)ccc12 ; Clc1cccc(Cl)c1Cc1ccc2c(NCCCNCc3cccc4c3OCO4)ccnc2c1 ; COc1ccc(F)cc1CNCCCNc1ccnc2cc(Cc3c(Cl)cccc3Cl)ccc12 ; FC(F)(F)c1cccc(Cc2ccc3c(NCCCNCc4cccc5c4OCO5)ccnc3c2)c1]

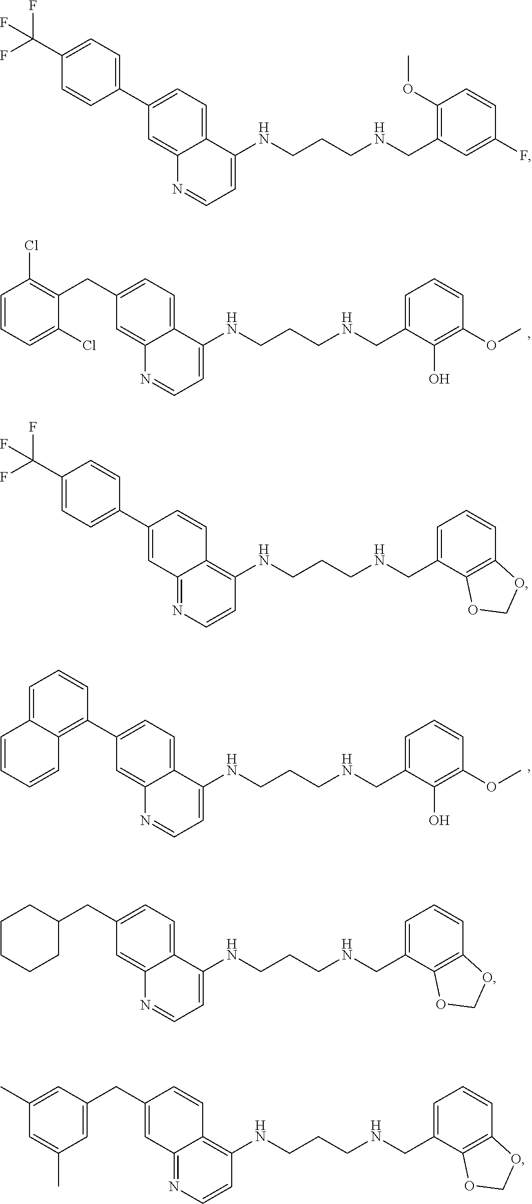

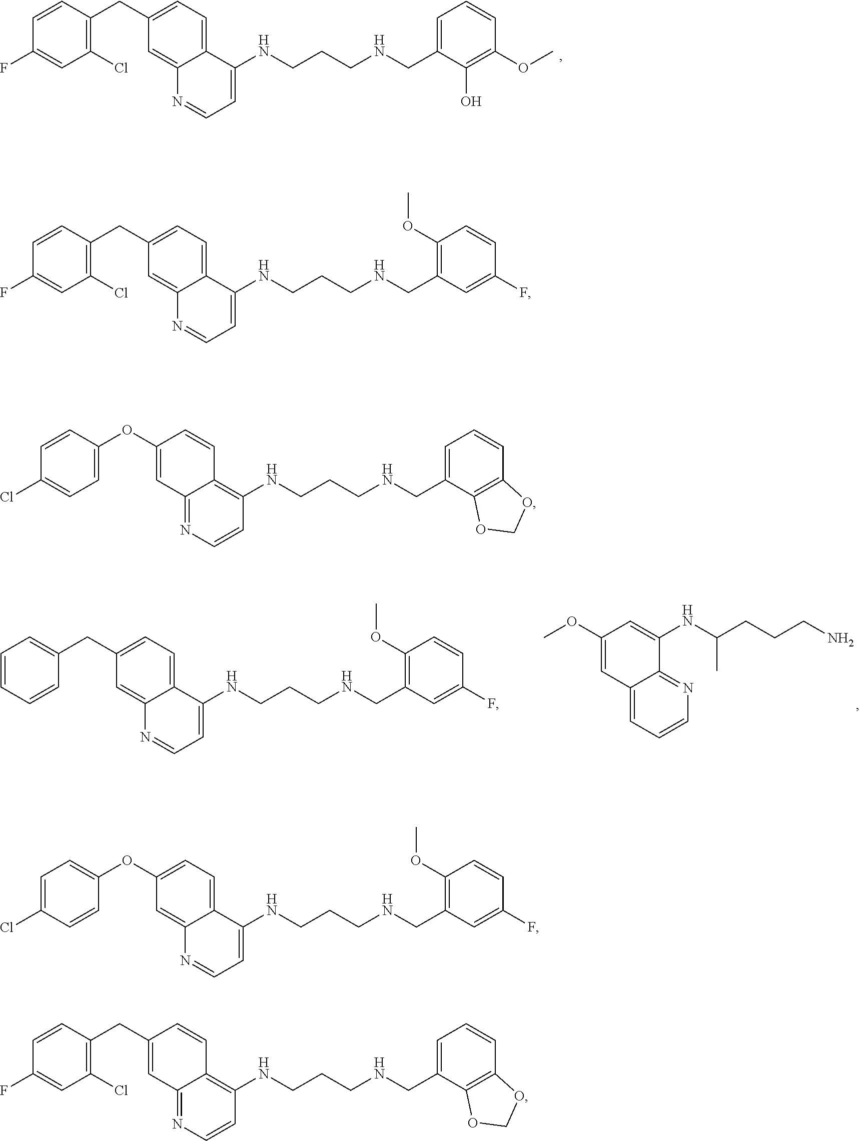

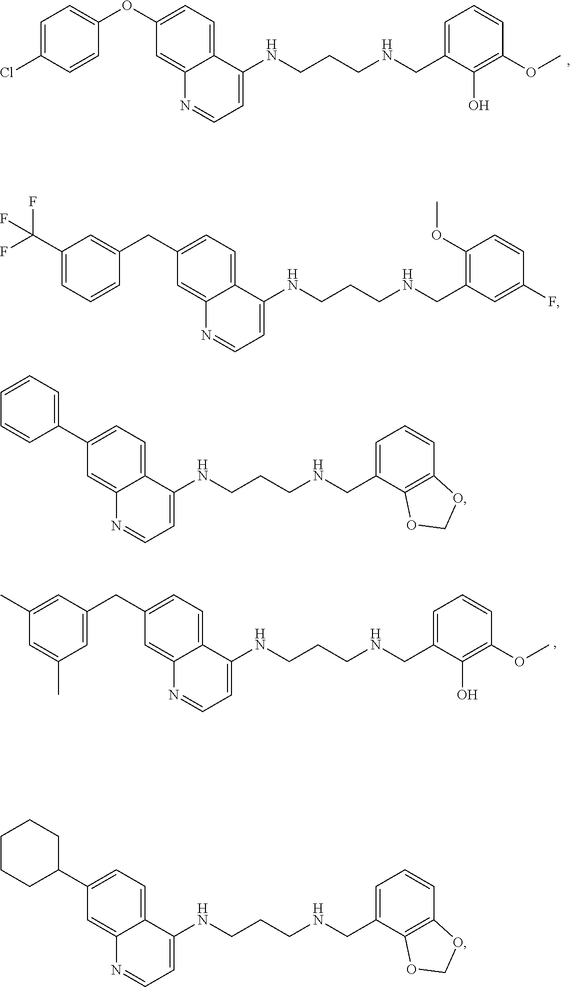

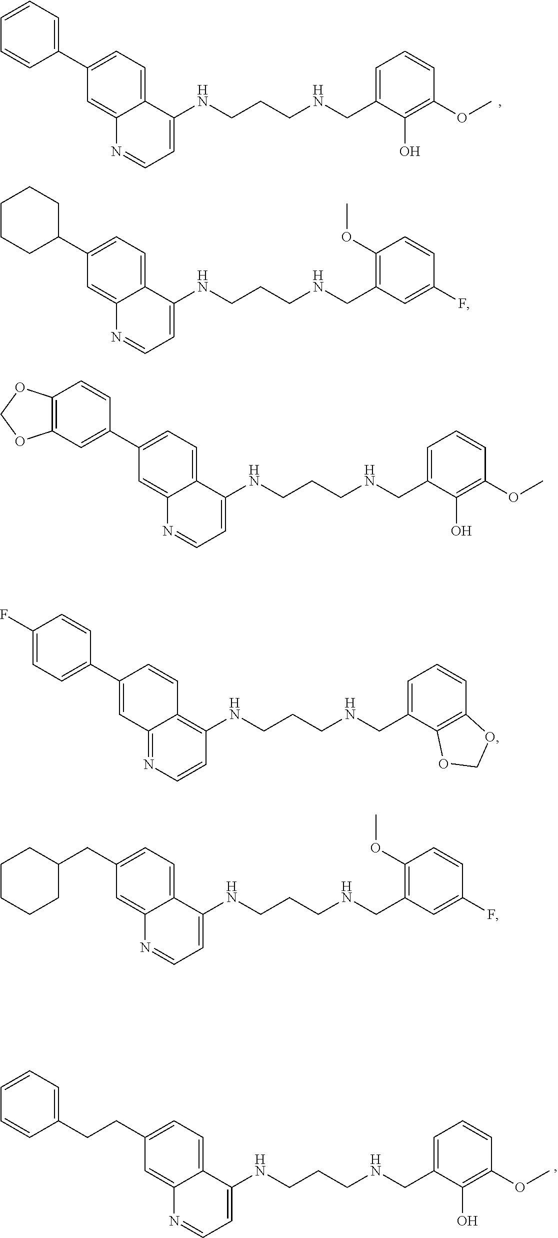 [SMILES: COc1cccc(CNCCCNc2ccnc3cc(-c4ccccc4)ccc23)c1O ; COc1ccc(F)cc1CNCCCNc1ccnc2cc(C3CCCCC3)ccc12 ; COc1cccc(CNCCCNc2ccnc3cc(-c4ccc5c(c4)OCO5)ccc23)c1O ; Fc1ccc(-c2ccc3c(NCCCNCc4cccc5c4OCO5)ccnc3c2)cc1 ; COc1ccc(F)cc1CNCCCNc1ccnc2cc(CC3CCCCC3)ccc12 ; COc1cccc(CNCCCNc2ccnc3cc(CCc4ccccc4)ccc23)c1O]

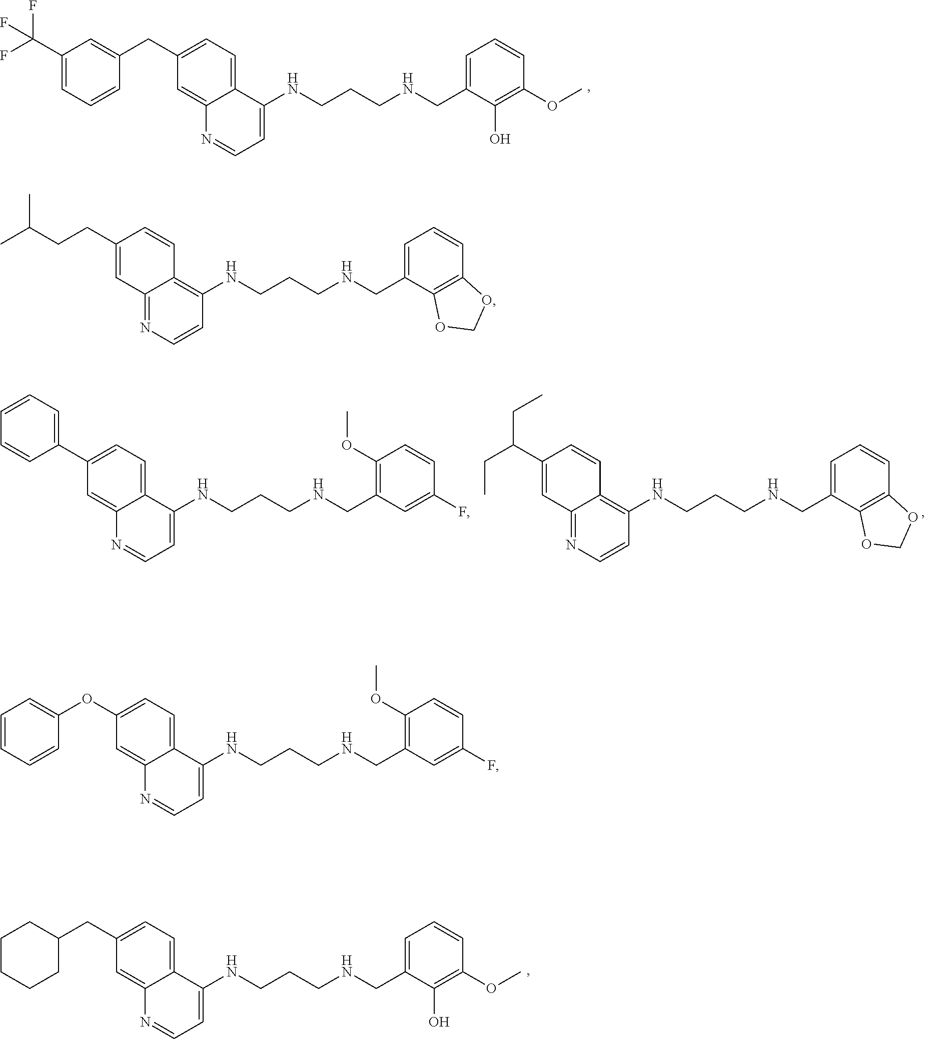

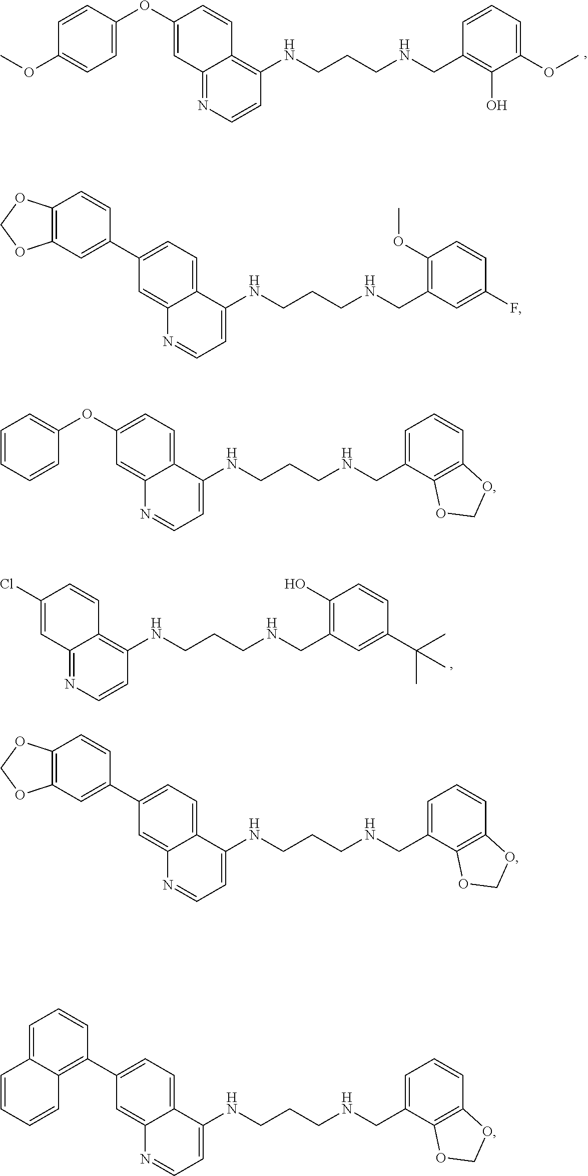

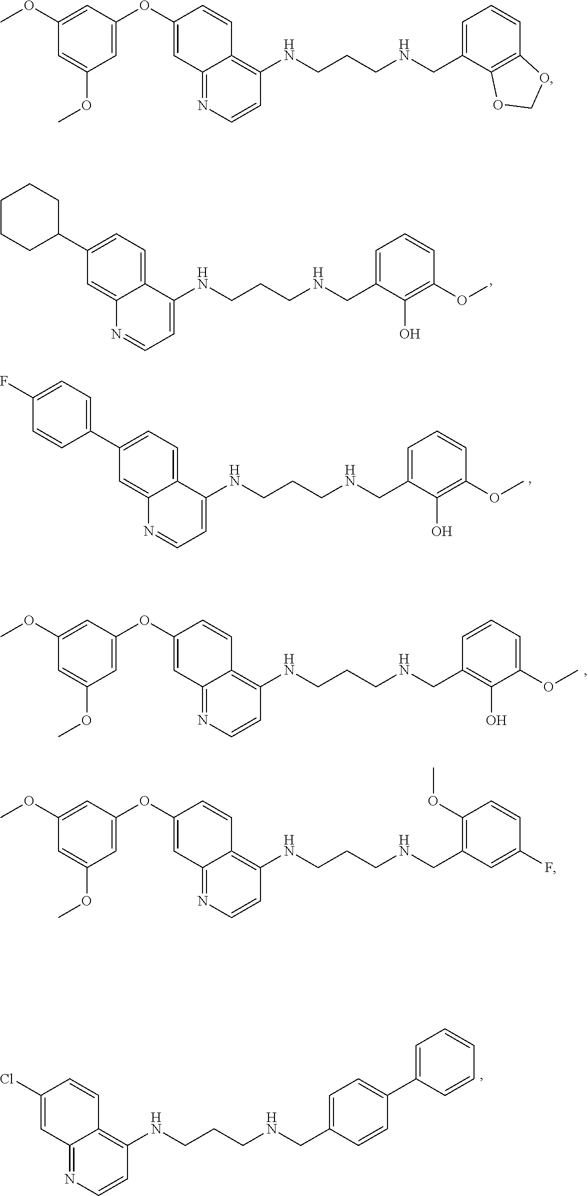

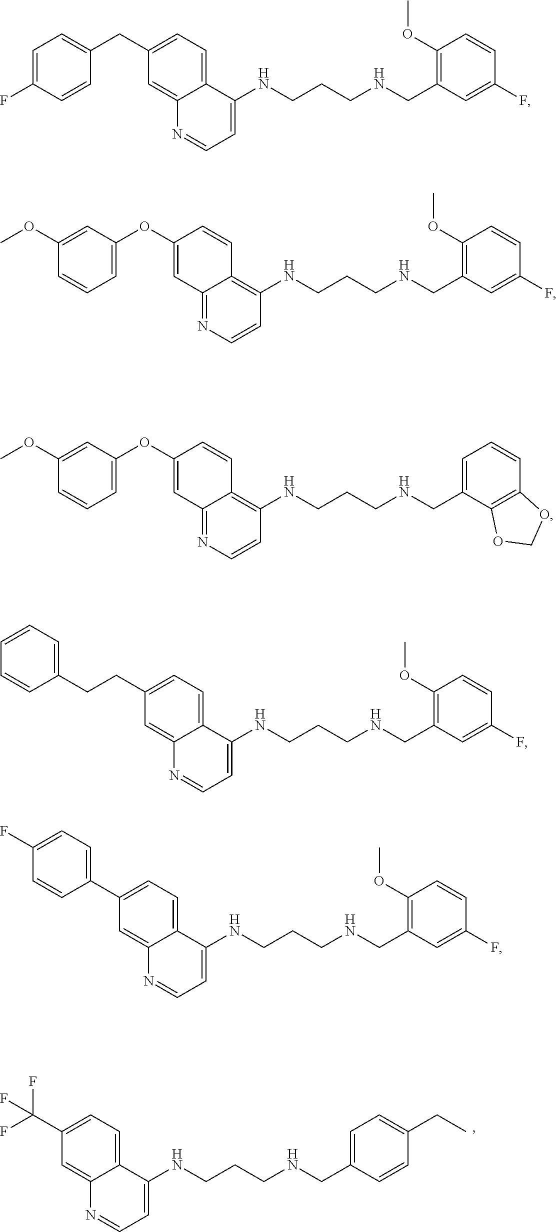 [SMILES: COc1ccc(F)cc1CNCCCNc1ccnc2cc(Cc3ccc(F)cc3)ccc12 ; COc1cccc(Oc2ccc3c(NCCCNCc4cc(F)ccc4OC)ccnc3c2)c1 ; COc1cccc(Oc2ccc3c(NCCCNCc4cccc5c4OCO5)ccnc3c2)c1 ; COc1ccc(F)cc1CNCCCNc1ccnc2cc(CCc3ccccc3)ccc12 ; COc1ccc(F)cc1CNCCCNc1ccnc2cc(-c3ccc(F)cc3)ccc12 ; CCc1ccc(CNCCCNc2ccnc3cc(C(F)(F)F)ccc23)cc1]

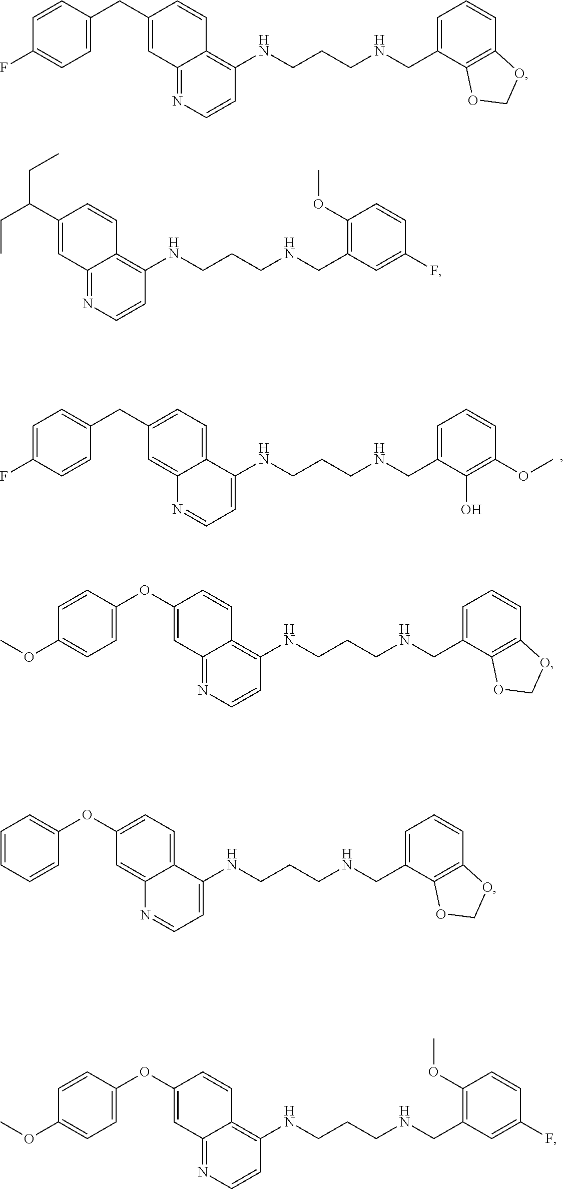

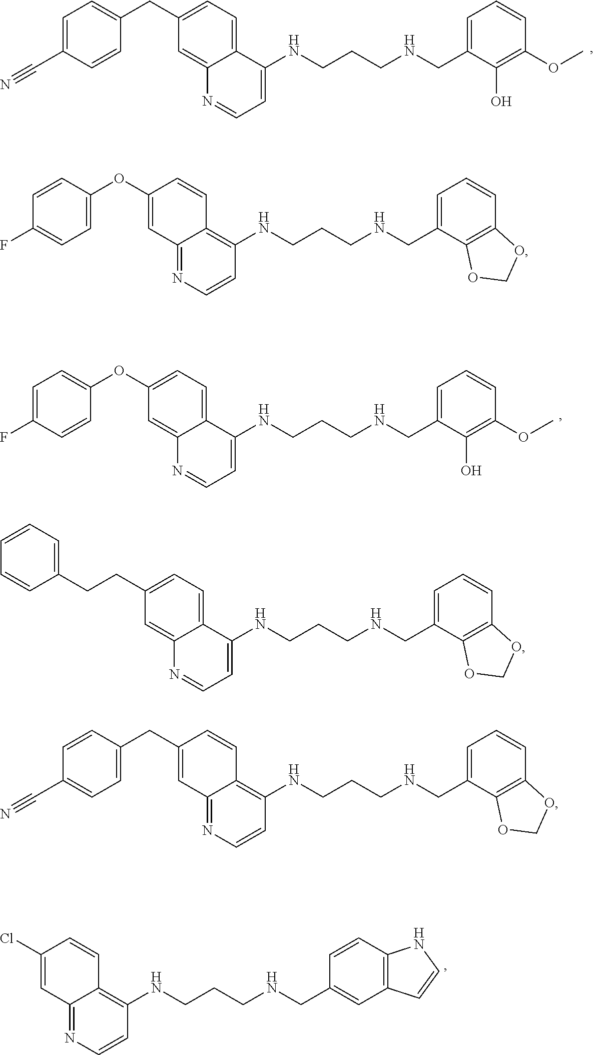

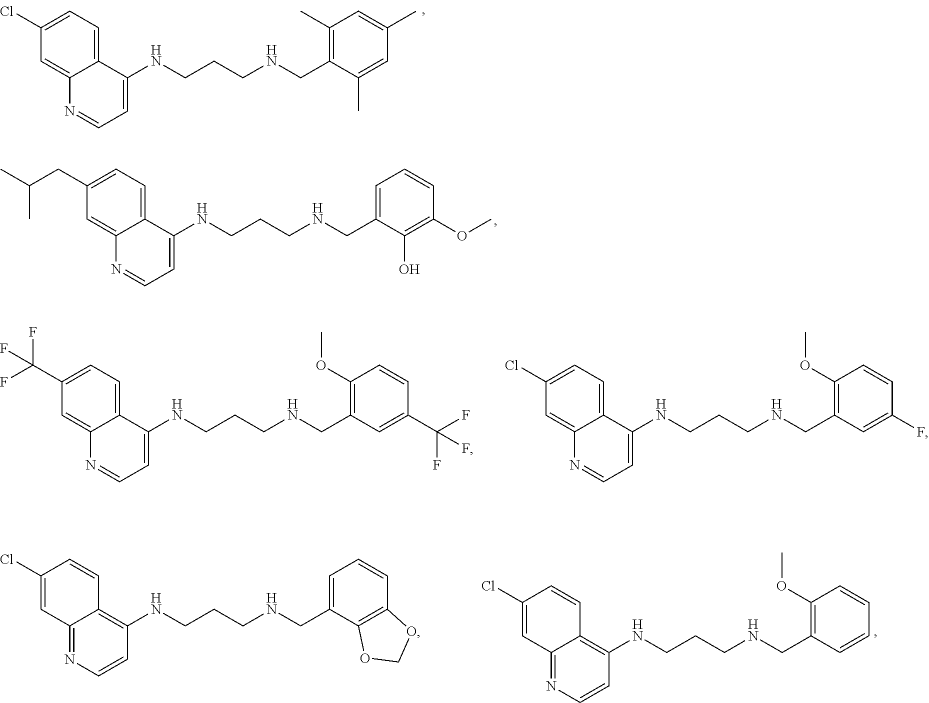

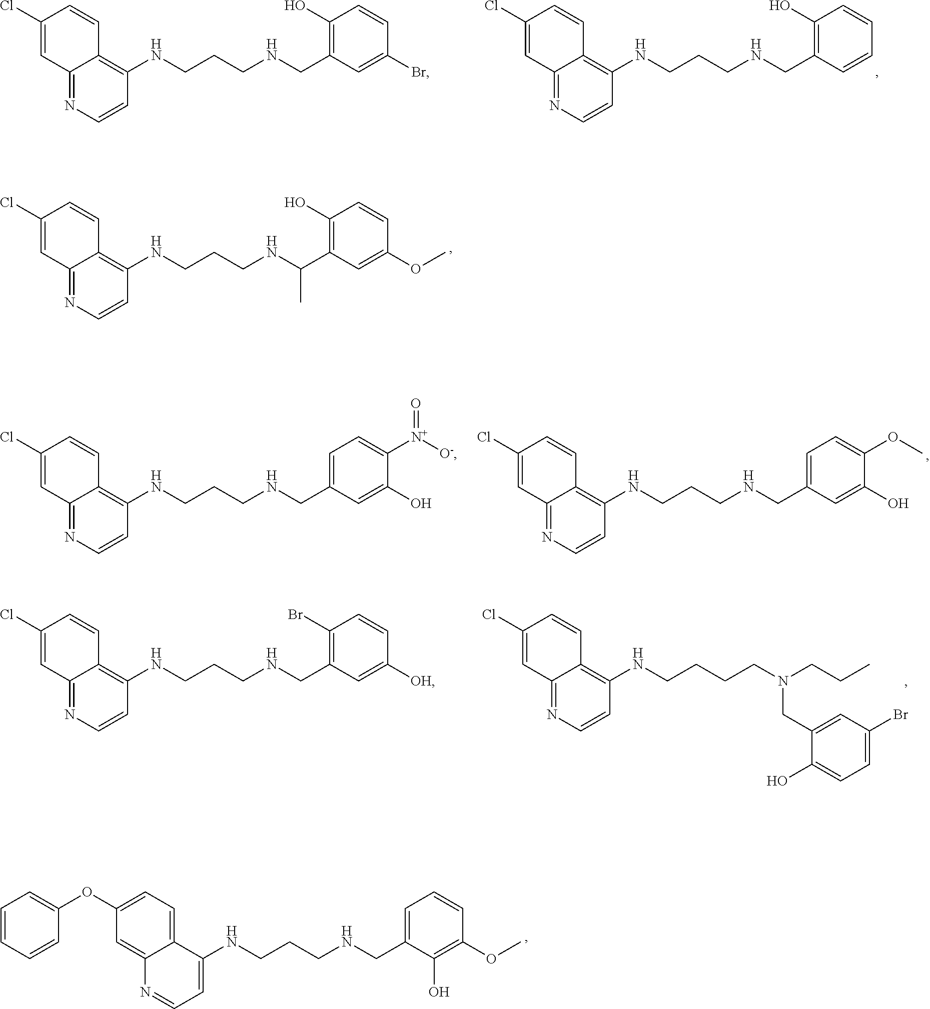

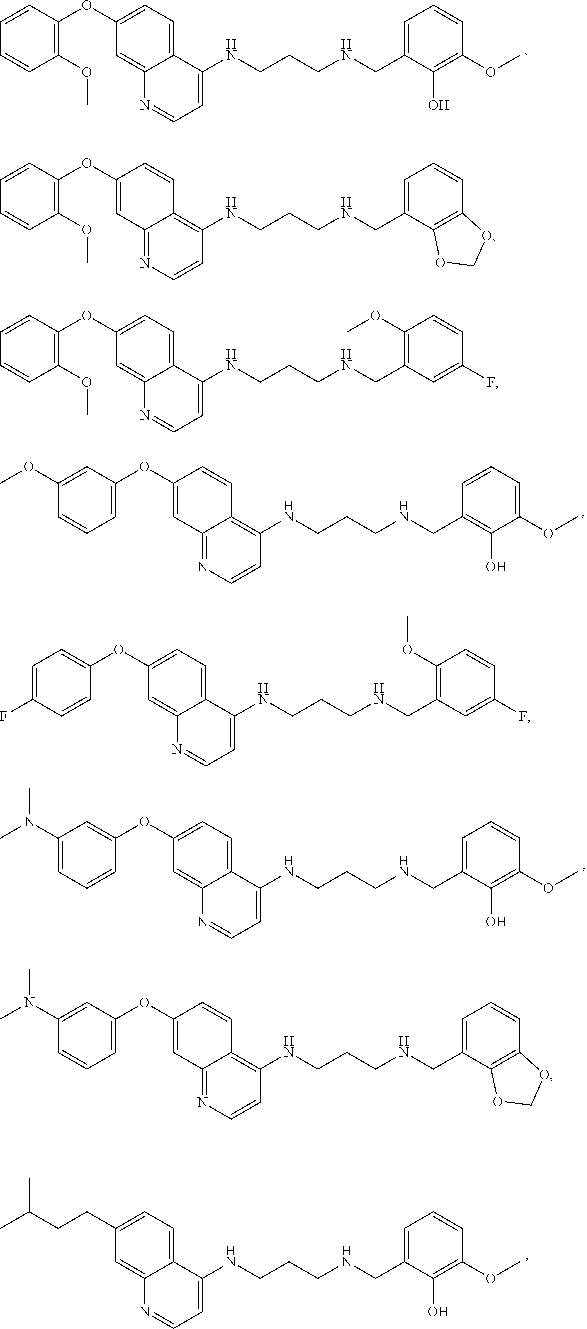

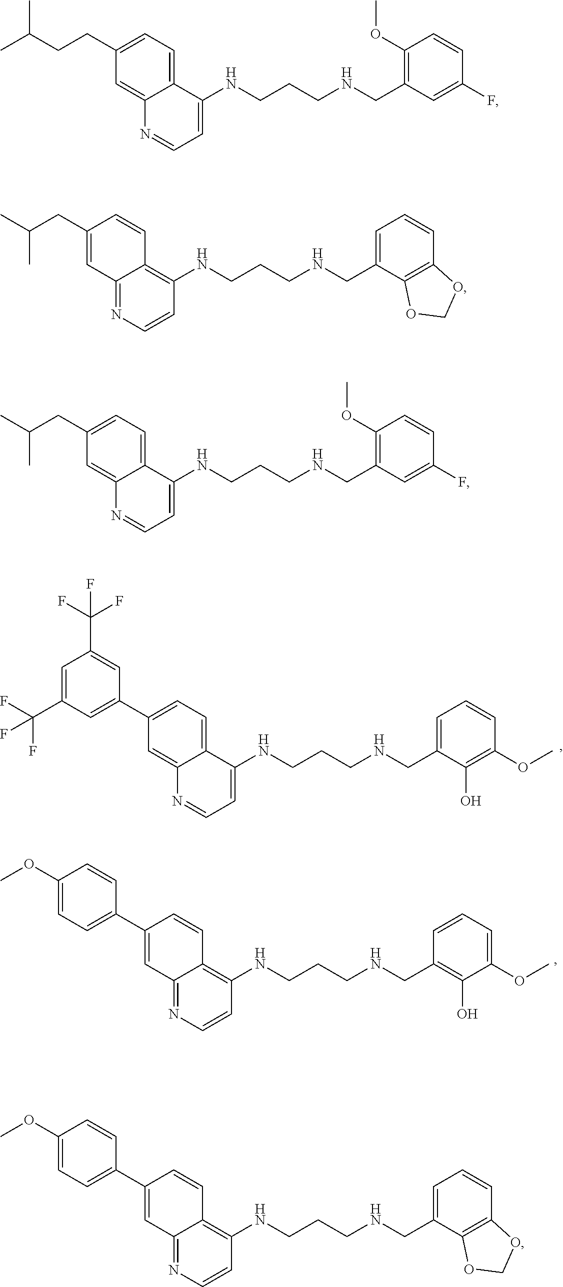

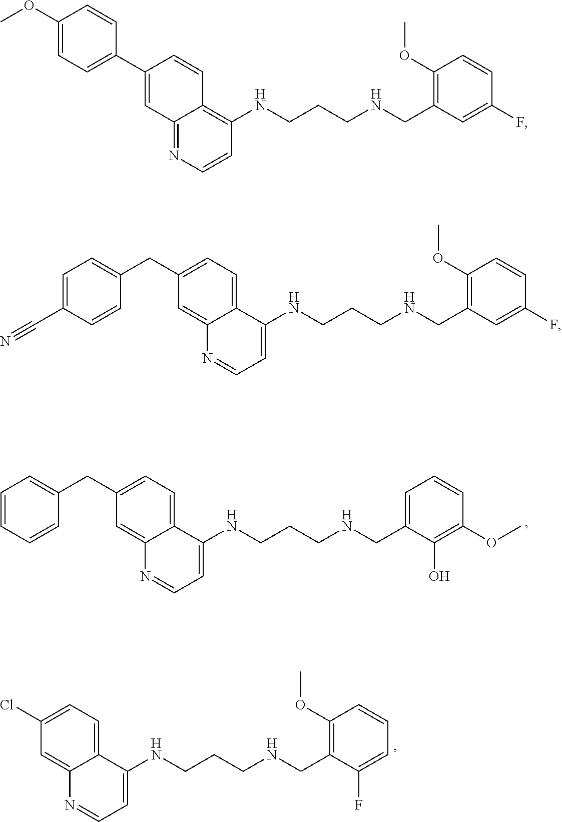 [SMILES: COc1ccc(-c2ccc3c(NCCCNCc4cc(F)ccc4OC)ccnc3c2)cc1 ; COc1ccc(F)cc1CNCCCNc1ccnc2cc(Cc3ccc(C#N)cc3)ccc12 ; COc1cccc(CNCCCNc2ccnc3cc(Cc4ccccc4)ccc23)c1O ; COc1cccc(F)c1CNCCCNc1ccnc2cc(Cl)ccc12]

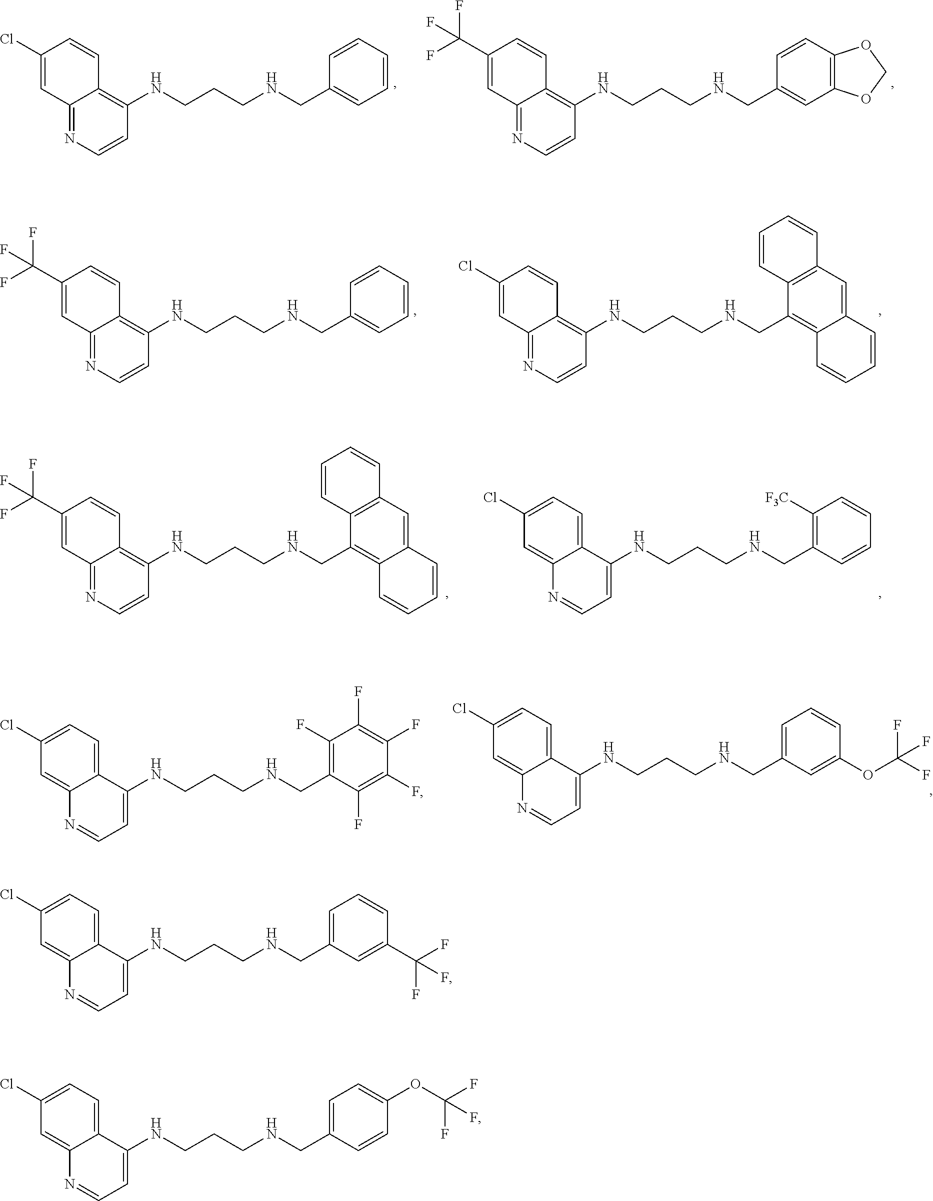

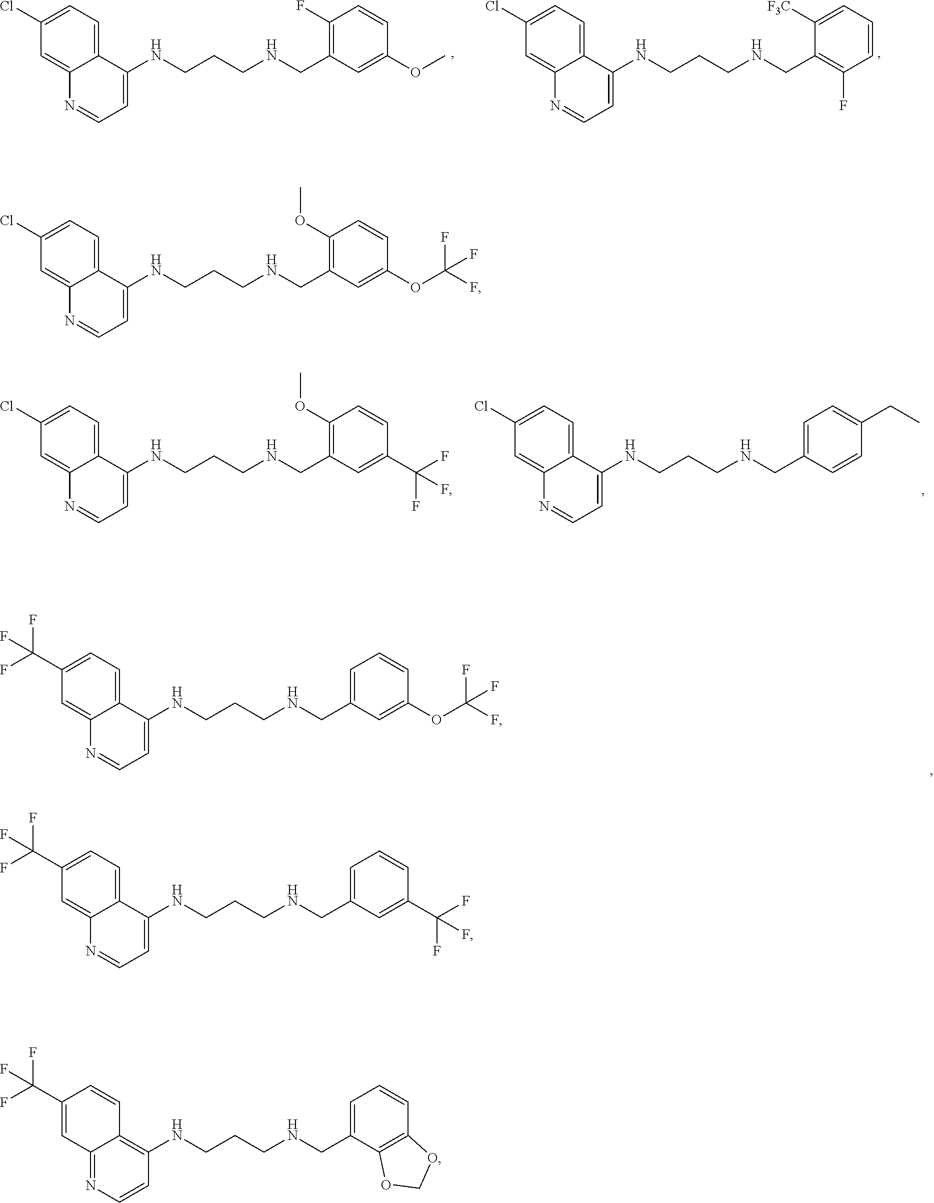

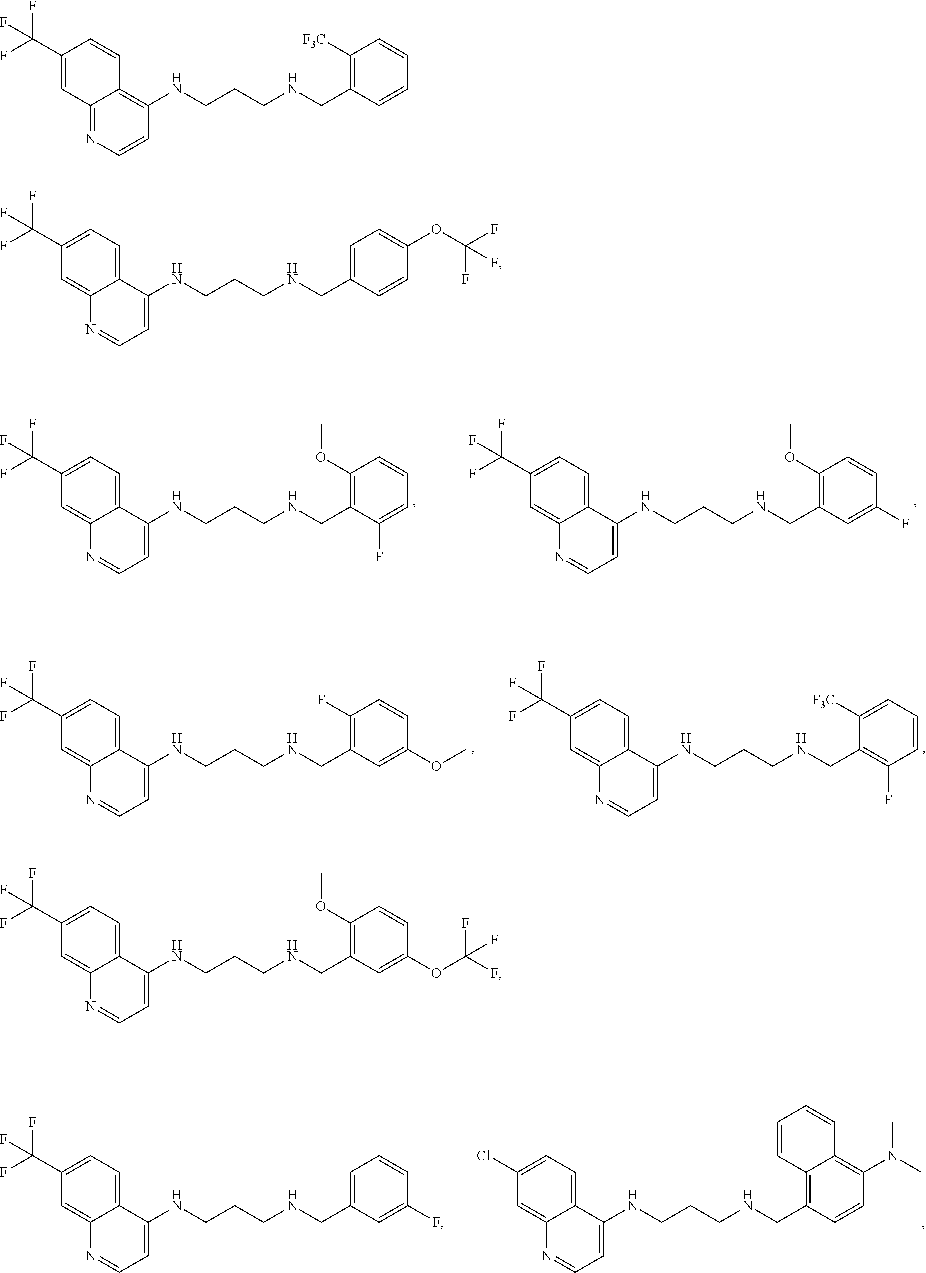

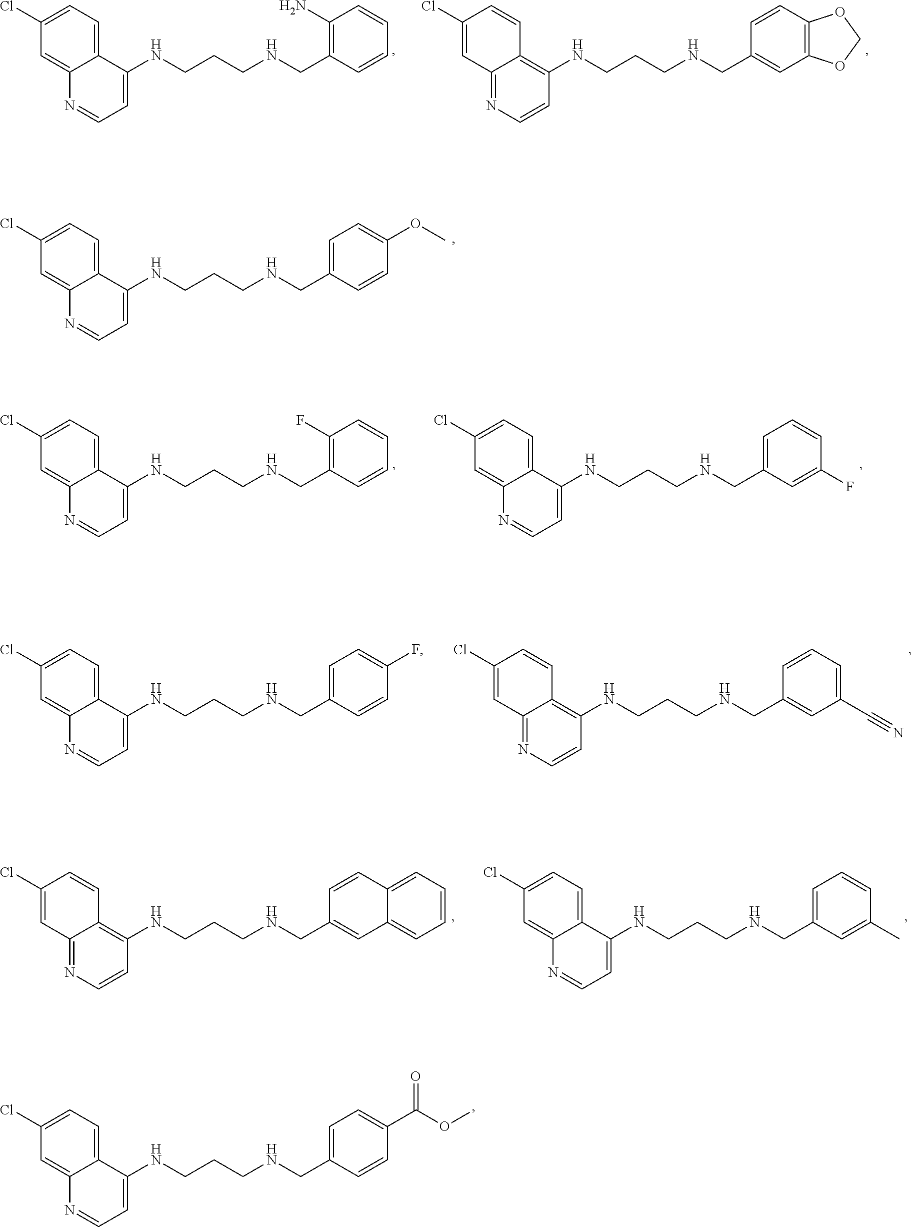

or a subgroup thereof.
Aspect 27. A peptide having a structure given by the sequence:

FLVVVHQIRQLXQ,

wherein X is a substituted phenylalanine analog having the structure given by the formula:

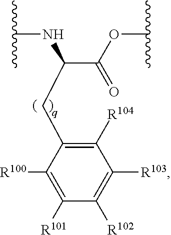

wherein q is selected from 0, 1, and 2; wherein each of R¹⁰⁰, R¹⁰¹, R¹⁰², R¹⁰³, and R¹⁰⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.
Aspect 28. The peptide according to any one of Aspects 27-41, wherein q is 1.
Aspect 29. The peptide according to any one of Aspects 27-41, wherein each of R¹⁰⁰, R¹⁰¹, R¹⁰², R¹⁰³, and R¹⁰⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that four of R¹⁰⁰, R¹⁰¹, R¹⁰², R¹⁰³, and R¹⁰⁴ are hydrogen.
Aspect 30. The peptide according to any one of Aspects 27-41, wherein each of R¹⁰⁰, R¹⁰¹, R¹⁰², R¹⁰³, and R¹⁰⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that three of R¹⁰⁰, R¹⁰¹, R¹⁰², R¹⁰³, and R¹⁰⁴ are hydrogen.
Aspect 31. The peptide according to any one of Aspects 27-41, wherein each of R¹⁰⁰, R¹⁰¹, R¹⁰², R¹⁰³, and R¹⁰⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl, provided that two of R¹⁰⁰, R¹⁰¹, R¹⁰², R¹⁰³, and R¹⁰⁴ are hydrogen.
Aspect 32. The peptide according to any one of Aspects 27-41, wherein each of R¹⁰⁰, R¹⁰¹, R¹⁰², R¹⁰³, and R¹⁰⁴ is independently selected from hydrogen, fluoro, chloro, —SF₅, —CN, —N₃, —NH₂, —OH, —CN, C1-C2 alkoxy, C1-C2 haloalkyl, C1-C2 aminoalkyl, C1-C2 alkylamino, C1-C2 hydoxyalkyl, and C1-C3 alkyl.
Aspect 33. The peptide according to any one of Aspects 27-41, wherein each of R¹⁰⁰, R¹⁰¹, R¹⁰², R¹⁰³, and R¹⁰⁴ is independently selected from hydrogen, fluoro, chloro, —SF₅, —CN, —N₃, —NH₂, —OH, —CN, C1-C2 alkoxy, C1-C2 haloalkyl, C1-C2 aminoalkyl, C1-C2 alkylamino, C1-C2 hydoxyalkyl, and C1-C3 alkyl.
Aspect 34. The peptide according to any one of Aspects 27-41, wherein each of R¹⁰⁰, R¹⁰¹, R¹⁰², R¹⁰³, and R¹⁰⁴ is independently selected from hydrogen, fluoro, chloro, —SF₅, —CN, —N₃, —NH₂, —OH, —CN, C1-C2 alkoxy, C1-C2 haloalkyl, C1-C2 aminoalkyl, C1-C2 alkylamino, C1-C2 hydoxyalkyl, and C1-C3 alkyl.
Aspect 35. The peptide according to any one of Aspects 27-41, wherein each of R¹⁰⁰, R¹⁰¹, R¹⁰², R¹⁰³, and R¹⁰⁴ is independently selected from hydrogen, fluoro, chloro, —SF₅, —CN, —N₃, —NH₂, —OH, —CN, C1-C2 alkoxy, C1-C2 haloalkyl, C1-C2 aminoalkyl, C1-C2 alkylamino, C1-C2 hydoxyalkyl, and C1-C3 alkyl.
Aspect 36. The peptide according to any one of Aspects 27-41, wherein each of R¹⁰⁰, R¹⁰¹, R¹⁰², R¹⁰³, and R¹⁰⁴ is independently selected from hydrogen, fluoro, chloro, —NH₂, —OH, methyl, methoxy, trifluoromethyl, trichloromethyl, methylamino, and dimethylamino.
Aspect 37. The peptide according to any one of Aspects 27-41, wherein each of R¹⁰⁰, R¹⁰¹, R¹⁰², R¹⁰³, and R¹⁰⁴ is independently selected from hydrogen, fluoro, chloro, methyl, methoxy, and trifluoromethyl.
Aspect 38. The peptide according to any one of Aspects 27-41, wherein each of R¹⁰⁰, R¹⁰¹, R¹⁰², R¹⁰³, and R¹⁰⁴ is hydrogen.
Aspect 39. The peptide according to any one of Aspects 27-41, wherein X is a substituted phenylalanine analog having the structure given by the formula:

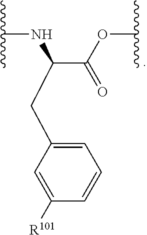

Aspect 40. The peptide according to any one of Aspects 27-41, wherein X is a substituted phenylalanine analog having the structure given by the formula:

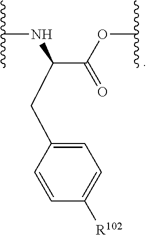

Aspect 41. The peptide according to any one of Aspects 27-40, wherein X is a substituted phenylalanine analog having the structure given by the formula:

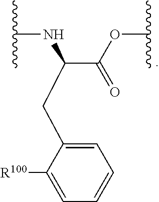

Aspect 42. A pharmaceutical composition comprising a therapeutically effective amount of at least one compound according to any one of Aspects 1-26, or a pharmaceutically acceptable salt, solvate, or polymorph thereof, and a pharmaceutically acceptable carrier; or a peptide according to any one of Aspects 27-41, or a pharmaceutically acceptable salt, solvate, or polymorph thereof.
Aspect 43. The pharmaceutical composition according to any one of Aspects 42-51, further comprising at least one agent known to treat a cancer.
Aspect 44. The pharmaceutical composition according to any one of Aspects 42-51, wherein the at least one agent known to treat a cancer is a hormone therapy agent; an alkylating agent, an antimetabolite agent, an antineoplastic antibiotic agent, a mitotic inhibitor agent, a mTor inhibitor agent, other chemotherapeutic agent, or combinations thereof.
Aspect 45. The pharmaceutical composition according to any one of Aspects 42-51, wherein the at least one agent known to treat a cancer is a hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt thereof.
Aspect 46. The pharmaceutical composition according to any one of Aspects 42-51, wherein the at least one agent known to treat a cancer is a antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.
Aspect 47. The pharmaceutical composition according to any one of Aspects 42-51, wherein the at least one agent known to treat a cancer is an antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.
Aspect 48. The pharmaceutical composition according to any one of Aspects 42-51, wherein the at least one agent known to treat a cancer is an alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt.
Aspect 49. The pharmaceutical composition according to any one of Aspects 42-51, wherein the at least one agent known to treat a cancer is a mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt.
Aspect 50. The pharmaceutical composition according to any one of Aspects 42-51, wherein the at least one agent known to treat a cancer is a mTor inhibitor agent is selected from one or more of the group consisting of everolimus, siroliumus, and temsirolimus, or a pharmaceutically acceptable salt thereof.
Aspect 51. The pharmaceutical composition according to any one of Aspects 42-50, wherein the at least one agent known to treat a cancer is selected from uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17?-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.
Aspect 52. A method for the treatment of a disorder of uncontrolled cellular proliferation associated with a MAGE-A11 dysfunction in a mammal comprising the step of administering to the mammal a therapeutically effective amount of at least one compound according to any one of Aspects 1-26, or a pharmaceutically acceptable salt thereof; at least one peptide of any one of Aspects 27-41, the pharmaceutical composition of any one of Aspects 42-51, or combinations thereof.
Aspect 53. The method according to any one of Aspects 52-85, wherein the mammal is a human.
Aspect 54. The method according to any one of Aspects 52-85, wherein the mammal has been diagnosed with a need for treatment of the disorder of uncontrolled cellular proliferation associated with a MAGE-A11 dysfunction prior to the administering step.
Aspect 55. The method according to any one of Aspects 52-85, further comprising the step of identifying a mammal in need of treatment of the disorder of uncontrolled cellular proliferation associated with a MAGE-A11 dysfunction.
Aspect 56. The method according to any one of Aspects 52-85, wherein the MAGE-A11 dysfunction is associated with aberrant expression of MAGE-A11.
Aspect 57. The method according to any one of Aspects 52-85, wherein the disorder of uncontrolled cellular proliferation is a cancer.
Aspect 58. The method according to any one of Aspects 52-85, wherein the cancer is selected from a brain cancer, lung cancer, hematological cancer, bladder cancer, colon cancer, cervical cancer, ovarian cancer, squamous cell cancer, kidney cancer, peritoneal cancer, breast cancer, gastric cancer, colorectal cancer, prostate cancer, pancreatic cancer, genitourinary tract cancer, lymphatic system cancer, stomach cancer, larynx cancer, malignant melanoma, colorectal cancer, endometrial carcinoma, thyroid cancer, rhabdosarcoma, and combinations thereof.
Aspect 59. The method according to any one of Aspects 52-85, wherein the cancer is selected from lung cancer, ovarian cancer, and brain cancer.
Aspect 60. The method according to any one of Aspects 52-85, wherein the cancer is lung cancer; and wherein the lung cancer is selected from small-cell lung cancer, non-small cell lung cancer, and combinations thereof.
Aspect 61. The method according to any one of Aspects 52-85, wherein cancer is kidney cancer; and wherein the kidney cancer is a kidney clear cell carcinoma.
Aspect 62. The method according to any one of Aspects 52-85, wherein the cancer is brain cancer; and wherein the brain cancer is selected from a glioblastoma, medullablastoma, glioma, and combinations thereof.
Aspect 63. The method according to any one of Aspects 52-85, wherein the cancer is bladder cancer; and wherein the bladder cancer is a bladder urothelial carcinoma.
Aspect 64. The method according to any one of Aspects 52-85, wherein the cancer is liver cancer; and wherein the liver cancer is a hepatic carcinoma.
Aspect 65. The method according to any one of Aspects 52-85, wherein the cancer is hematological cancer; and wherein the hematological cancer is selected from chronic myeloid leukemia (CML), acute myeloid leukemia (AML), chronic lymphoid leukemia (CLL), acute lymphoid leukemia (ALL), hairy cell leukemia, chronic myelomonocytic leukemia (CMML), juvenile myelomonocyte leukemia (JMML), large granular lymphocytic leukemia (LGL), acute lymphocytic leukemia, acute lymphoblastic leukemia, B-cell lymphoma, T-cell-lymphoma, Hodgkin's lymphoma, non-Hodgkin's lymphoma, hairy cell lymphoma, Burkett's lymphoma, Hodgkin lymphoma, non-Hodgkin lymphoma, and combinations thereof.
Aspect 66. The method according to any one of Aspects 52-85, further comprising the step of administering a therapeutically effective amount of at least one agent known to treat a cancer.
Aspect 67. The method according to any one of Aspects 52-85, wherein the at least one agent is selected from uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, and vasostatin.
Aspect 68. The method according to any one of Aspects 52-85, wherein the at least one agent is a DNA methyltransferase inhibitor, an HDAC-inhibitor, a glucocorticoid, an mTOR inhibitor, a cytotoxic agent, or combinations thereof.
Aspect 69. The method according to any one of Aspects 52-85, wherein the at least one agent is a DNA methyltransferase inhibitor; and wherein the DNA methyltransferase inhibitor is 5-aza-2′-deoxycytidine, 5-azacytidine, zebularin, epigallocatechin-3-gallate, procaine, or combinations thereof.
Aspect 70. The method according to any one of Aspects 52-85, wherein the at least one agent is a HDAC-inhibitor; and wherein the HDAC-inhibitor is vorinostat, entinostat, panbinostat, trichostatin A, mocetinostat, belinostat, dacinostat, givinostat, tubastatin A, pracinostat, droxinostat, quisinostat, romidepsin, valproic acid, AR-42 (OSU-HDAC42), tacedinaline, rocilinostat, apicidin, or combinations thereof.
Aspect 71. The method according to any one of Aspects 52-85, wherein the at least one agent is a glucocorticoid; and wherein the glucocorticoid is dexamethasone, prednisolone, methylprednisolone, betamethasone, triamicinolone, fludrocortisone, beclomethasone, or combinations thereof.
Aspect 72. The method according to any one of Aspects 52-85, wherein the at least one agent is a mTor inhibitor; and wherein the mTor inhibitor is BEZ235, everolimus, temsirolimus, rapamycin, AZD8055, or combinations thereof.
Aspect 73. The method according to any one of Aspects 52-85, wherein the at least one agent is a cytotoxic agent; and wherein the cytotoxic agent is an alkylating agent, an antimetabolite agent, an antineoplastic antibiotic agent, a mitotic inhibitor agent, a mTor inhibitor agent or other chemotherapeutic agent.
Aspect 74. The method according to any one of Aspects 52-85, wherein the cytotoxic agent is an antineoplastic antibiotic agent; and wherein the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof.
Aspect 75. The method according to any one of Aspects 52-85, wherein the cytotoxic agent is an antimetabolite agent; and wherein the antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof.
Aspect 76. The method according to any one of Aspects 52-85, wherein the cytotoxic agent is an alkylating agent; and wherein the alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof.
Aspect 77. The method according to any one of Aspects 52-85, wherein the cytotoxic agent is a mitotic inhibitor agent; and wherein the mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt, hydrate, solvate, or polymorph thereof.
Aspect 78. The method according to any one of Aspects 52-85, wherein the mTor inhibitor is everolimus, sirolimus, temsirolimus, or combinations thereof.
Aspect 79. The method according to any one of Aspects 52-85, wherein the cytotoxic agent is an other chemotherapeutic agent; and wherein the other chemotherapeutic agent is an anthracycline, cytarabine, a purine analog, sorafenib, gemtuzumab ozogamicin, rituximab, or combinations thereof.
Aspect 80. The method according to any one of Aspects 52-85, wherein the other chemotherapeutic agent is an anthracycline; and wherein the anthracycline is daunorubicin, idarubicin, or combinations thereof.
Aspect 81. The method according to any one of Aspects 52-85, wherein the other chemotherapeutic agent is a purine analog; and wherein the purine analog is cladribine, fludarabine, clofarabine, or combinations thereof.
Aspect 82. The method according to any one of Aspects 52-85, wherein the at least one compound and the at least one agent are administered sequentially.
Aspect 83. The method according to any one of Aspects 52-85, wherein the at least one compound and the at least one agent are administered simultaneously.
Aspect 84. The method according to any one of Aspects 52-85, wherein the at least one compound, the at least one peptide, and the at least one agent are co-formulated.
Aspect 85. The method according to any one of Aspects 52-84, wherein the at least one compound, the at least one peptide, and the at least one agent are co-packaged.
Aspect 86. A method for modulating of MAGE-A11 activity in a mammal comprising the step of administering to the mammal a therapeutically effective amount of at least one compound according to any one of Aspects 1-26, or a pharmaceutically acceptable salt thereof; at least one peptide according to any one of Aspects 27-41; the pharmaceutical composition according to any one of Aspects 42-51, or combinations thereof.
Aspect 87. The method according to any one of Aspects 86-90, wherein the mammal is a human.
Aspect 88. The method according to any one of Aspects 86-90, wherein the mammal has been diagnosed with a need for modulating of MAGE-A11 activity prior to the administering step.
Aspect 89. The method according to any one of Aspects 86-90, further comprising the step of identifying a mammal in need for modulating of MAGE-A11 activity.
Aspect 90. The method according to any one of Aspects 86-89, wherein the modulating is inhibiting.
Aspect 91. A method for modulating of MAGE-A11 activity in at least one cell, comprising the step of contacting the at least one cell with an effective amount of at least one compound according to any one of Aspects 1-26 or a pharmaceutically acceptable salt thereof; at least one peptide according to any one of Aspects 27-41, the pharmaceutical composition according to any one of Aspects 42-51, or combinations thereof.
Aspect 92. The method according to any one of Aspects 91-106, wherein the cell is mammalian.
Aspect 93. The method according to any one of Aspects 91-106, wherein the cell is human.
Aspect 94. The method according to any one of Aspects 91-106, wherein the cell has been isolated from a mammal prior to the contacting step.
Aspect 95. The method according to any one of Aspects 91-106, wherein the modulating is inhibiting.
Aspect 96. The method according to any one of Aspects 91-106, wherein contacting is via administration to a mammal.
Aspect 97. The method according to any one of Aspects 91-106, wherein the mammal has been diagnosed with a need for modulating of MAGE-A11 activity prior to the administering step.
Aspect 98. The method according to any one of Aspects 91-106, wherein the mammal has been diagnosed with a need for treatment of a disorder related to MAGE-11A activity prior to the administering step.
Aspect 99. The method according to any one of Aspects 91-106, wherein the compound exhibits in a TR-FRET-based assay inhibition of MAGE-11A binding to a PCF-11 substrate with an IC₅₀ of less than about 10,000 nM.
Aspect 100. The method according to any one of Aspects 91-106, wherein the compound exhibits in a TR-FRET-based assay inhibition of MAGE-11A binding to a PCF-11 substrate with an IC₅₀ of less than about 5,000 nM.
Aspect 101. The method according to any one of Aspects 91-106, wherein the compound exhibits in a TR-FRET-based assay inhibition of MAGE-11A binding to a PCF-11 substrate with an IC₅₀ of less than about 1,000 nM.
Aspect 102. The method according to any one of Aspects 91-106, wherein the compound exhibits in a TR-FRET-based assay inhibition of MAGE-11A binding to a PCF-11 substrate with an IC₅₀ of less than about 750 nM.
Aspect 103. The method according to any one of Aspects 91-106, wherein the compound exhibits in a TR-FRET-based assay inhibition of MAGE-11A binding to a PCF-11 substrate with an IC₅₀ of less than about 500 nM.
Aspect 104. The method according to any one of Aspects 91-106, wherein the compound exhibits in a TR-FRET-based assay inhibition of MAGE-11A binding to a PCF-11 substrate with an IC₅₀ of less than about 250 nM.
Aspect 105. The method according to any one of Aspects 91-106, wherein the compound exhibits in a TR-FRET-based assay inhibition of MAGE-11A binding to a PCF-11 substrate with an IC₅₀ of less than about 200 nM.
Aspect 106. The method according to any one of Aspects 91-105, wherein the compound exhibits in a TR-FRET-based assay inhibition of MAGE-11A binding to a PCF-11 substrate with an IC₅₀ of less than about 150 nM.
Aspect 107. A kit comprising at least one compound according to any one of Aspects 1-26, or a pharmaceutically acceptable salt thereof; at least one peptide according to any one of Aspects 27-41, the pharmaceutical composition according to any one of Aspects 42-51, or combinations thereof; and one or more of: (a) at least one agent known to increase MAGE-11A activity; (b) at least one agent known to decrease MAGE-11A activity; (c) at least one agent known to treat a disorder of uncontrolled cellular proliferation; (d) instructions for treating a disorder of uncontrolled cellular proliferation; or (e) instructions for treating a disorder associated with MAGE-11A dysfunction.
Aspect 108. The kit according to any one of Aspects 107-126, wherein the at least one compound or the at least one product and the at least one agent are co-formulated.
Aspect 109. The kit according to any one of Aspects 107-126, wherein the at least one compound or the at least one product and the at least one agent are co-packaged.
Aspect 110. The kit according to any one of Aspects 107-126, further comprising instructions to provide the compound in connection with surgery.
Aspect 111. The kit according to any one of Aspects 107-126, wherein the instructions provide that surgery is performed prior to the administering of at least one compound.
Aspect 112. The kit according to any one of Aspects 107-126, wherein the instructions provide that surgery is performed after the administering of at least one compound.
Aspect 113. The kit according to any one of Aspects 107-126, wherein the instructions provide that the administering of at least one compound is to effect presurgical debulking of a tumor.
Aspect 114. The kit according to any one of Aspects 107-126, wherein the instructions provide that surgery is performed at about the same time as the administering of at least one compound.
Aspect 115. The kit according to any one of Aspects 107-126, further comprising instructions to provide the at least one compound or the pharmaceutical composition in connection with radiotherapy.
Aspect 116. The kit according to any one of Aspects 107-126, wherein the instructions provide that radiotherapy is performed prior to the administering of at least one compound.
Aspect 117. The kit according to any one of Aspects 107-126, wherein the instructions provide that radiotherapy is performed after the step of the administering of at least one compound.
Aspect 118. The kit according to any one of Aspects 107-126, wherein the instructions provide that radiotherapy is performed at about the same time as the step of the administering of at least one compound.
Aspect 119. The kit according to any one of Aspects 107-126, further comprising a plurality of dosage forms, the plurality comprising one or more doses; wherein each dose comprises a therapeutically effective amount of the at least one compound or the pharmaceutical composition and the at least one agent.
Aspect 120. The kit according to any one of Aspects 107-126, wherein each dose of the at least one compound or the pharmaceutical composition and the at least one agent are co-formulated.
Aspect 121. The kit according to any one of Aspects 107-126, wherein each dose of the at least one compound or the pharmaceutical composition and the at least one agent are co-packaged.
Aspect 122. The kit according to any one of Aspects 107-126, wherein the dosage forms are formulated for oral administration and/or intravenous administration.
Aspect 123. The kit according to any one of Aspects 107-126, wherein the dosage form as are formulated for oral administration.
Aspect 124. The kit according to any one of Aspects 107-126, wherein the dosage forms are formulated for intravenous administration.
Aspect 125. The kit according to any one of Aspects 107-126, wherein the dosage form for the at least one compound or the pharmaceutical composition is formulated for oral administration and the dosage form for the at least one agent is formulated for intravenous administration.
Aspect 126. The kit according to any one of Aspects 107-125, wherein the dosage form for the at least one compound or the pharmaceutical composition is formulated for intravenous administration and the dosage form for the at least one agent is formulated for oral administration.
Aspect 127. Use of at least one compound according to any one of Aspects 1-26, or a pharmaceutically acceptable salt thereof; at least one peptide according to any one of Aspects 27-41, the pharmaceutical composition according to any one of Aspects 42-51, or combinations thereof in the manufacture of a medicament for the treatment of a disorder associated with a MAGE-11A dysfunction in a mammal.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

The following tables are referenced herein above in the Examples section and are included following the Examples to facilitate the formatting and flow of the disclosure. They constitute an integral part of the present disclosure.

TABLE 3
Dose response TR-FRET screening of nine scaffold groups against MAGE-A11:PCF11.

| SAMPLE | consensus IC50 | MOLSMILES | Clustering |
|---|---|---|---|
| SJ000574313-7 | 9.4561 | COc1cc(NCc2ccc3nc(N)nc(N)c3c2C)cc(OC)c1OC | Scaffold-10 |
| SJ000286094-9 | 36.1689 | COc1cc2nc(nc(N)c2cc1OC)N(C)CCCNC(═O)C3CCCO3 | Scaffold-10 |
| SJ000285540-13 | 53.1469 | COc1cc2nc(nc(N)c2cc1OC)N3CCN(CC3)C(═O)c4occc4 | Scaffold-10 |
| SJ000287388-10 | 54.1801 | COc1cc2nc(nc(N)c2cc1OC)N3CCN(CC3)C(═O)C4CCCO4 | Scaffold-10 |
| SJ000113084-10 | 57.1413 | COc1cc2nc(nc(N)c2cc1OC)N3CCN(CC3)C(═O)C4COc5ccccc5O4 | Scaffold-10 |
| SJ000045621-1 | 69.9301 | CCOc1ccc(Nc2nc(N)c3ccccc3n2)cc1 | Scaffold-10 |
| SJ000045622-1 | 69.9301 | Nc1nc(Nc2ccc(O)cc2)nc3ccccc13 | Scaffold-10 |
| SJ000067204-1 | 69.9301 | COc1ccc(Nc2nc(N)c3ccccc3n2)cc1 | Scaffold-10 |
| SJ000189299-1 | 69.9301 | Cc1cc(C)nc(Nc2nc(N)c3ccccc3n2)n1 | Scaffold-10 |
| SJ000192476-1 | 69.9301 | Cc1ccccc1Nc2nc(N)c3ccccc3n2 | Scaffold-10 |
| SJ000192505-1 | 69.9301 | COc1ccccc1Nc2nc(N)c3ccccc3n2 | Scaffold-10 |
| SJ000192933-1 | 69.9301 | Nc1nc(Nc2ccccc2O)nc3ccccc13 | Scaffold-10 |
| SJ000192944-1 | 69.9301 | Cc1ccc(C)c(Nc2nc(N)c3ccccc3n2)c1 | Scaffold-10 |
| SJ000200999-1 | 69.9301 | COc1ccc(Cl)cc1Nc2nc(N)c3ccccc3n2 | Scaffold-10 |
| SJ000201287-1 | 69.9301 | Cc1ccc(Nc2nc(N)c3ccccc3n2)cc1Cl | Scaffold-10 |
| SJ000201376-1 | 69.9301 | Nc1nc(Nc2ccc(Cl)c(Cl)c2)nc3ccccc13 | Scaffold-10 |
| SJ000201566-1 | 69.9301 | COc1ccc(C)cc1Nc2nc(N)c3ccccc3n2 | Scaffold-10 |
| SJ000202008-1 | 69.9301 | COc1ccc(OC)c(Nc2nc(N)c3ccccc3n2)c1 | Scaffold-10 |
| SJ000202381-1 | 69.9301 | Cc1ccc(Nc2nc(N)c3ccccc3n2)cc1C | Scaffold-10 |
| SJ000202571-1 | 69.9301 | CCN1CCN(CC1)c2nc(N)c3ccccc3n2 | Scaffold-10 |
| SJ000288233-2 | 69.9301 | COc1cc2nc(nc(N)c2cc1OC)N3CCN(C(C3)C(═O)NC(C)(C)C)C(═O)OCc4ccccc4 | Scaffold-10 |
| SJ000803757-1 | 58.4149 | Fc1ccc(cc1F)C2Nc3ccccc3C(═O)N2CCCN4CCOCC4 | Scaffold-13 |
| SJ000803761-1 | 62.331 | Cc1cc(C)c(cc1C)C2Nc3ccccc3C(═O)N2CCCN4CCOCC4 | Scaffold-13 |
| SJ000803768-1 | 65.8741 | Cc1ccc(cc1)C2Nc3ccccc3C(═O)N2CCCN4CCOCC4 | Scaffold-13 |
| SJ000803763-1 | 67.366 | COc1ccc2cc(ccc2c1)C3Nc4ccccc4C(═O)N3CCCN5CCOCC5 | Scaffold-13 |
| SJ000803759-1 | 67.7389 | CCOc1ccc(cc1)C2Nc3ccccc3C(═O)N2CCCN4CCOCC4 | Scaffold-13 |
| SJ000795275-1 | 67.8322 | Fc1ccc(C2Nc3ccccc3C(═O)N2CCCN4CCOCC4)c(F)c1 | Scaffold-13 |
| SJ000803764-1 | 67.972 | Fc1ccc(cc1Br)C2Nc3ccccc3C(═O)N2CCCN4CCOCC4 | Scaffold-13 |
| SJ000019510-1 | 69.9301 | CCCN1C(Nc2ccccc2C1═O)c3ccc(O)c(OCC)c3 | Scaffold-13 |
| SJ000019545-2 | 69.9301 | Cc1ccc(cc1)C2Nc3ccccc3C(═O)N2Cc4ccccc4 | Scaffold-13 |
| SJ000019546-2 | 69.9301 | Brc1ccc(cc1)C2Nc3ccccc3C(═O)N2Cc4ccccc4 | Scaffold-13 |
| SJ000019547-2 | 69.9301 | Clc1ccc(cc1)C2Nc3ccccc3C(═O)N2Cc4ccccc4 | Scaffold-13 |
| SJ000026516-2 | 69.9301 | CCCN1C(Nc2ccccc2C1═O)c3ccc(F)cc3 | Scaffold-13 |
| SJ000026517-1 | 69.9301 | CCCN1C(Nc2ccccc2C1═O)c3ccc(OC)c(OC)c3 | Scaffold-13 |
| SJ000026518-2 | 69.9301 | CCCN1C(Nc2ccccc2C1═O)c3ccc(OC)cc3 | Scaffold-13 |
| SJ000026596-1 | 69.9301 | Fc1ccc(cc1)C2Nc3ccccc3C(═O)N2Cc4ccccc4 | Scaffold-13 |
| SJ000051049-1 | 69.9301 | [O−][N+](═O)c1ccc(cc1)C2Nc3ccccc3C(═O)N2Cc4ccccc4 | Scaffold-13 |
| SJ000111312-2 | 69.9301 | COc1ccc(cc1CSc2ccccn2)C3Nc4ccccc4C(═O)N3Cc5ccccc5 | Scaffold-13 |
| SJ000288416-1 | 69.9301 | CCCN1C(Nc2ccccc2C1═O)c3cccc(F)c3 | Scaffold-13 |
| SJ000288419-1 | 69.9301 | COc1ccc(cc1Cn2nc(C)c(c2C)[N+](═O)[O−])C3Nc4ccccc4C(═O)N3Cc5ccccc5 | Scaffold-13 |
| SJ000288423-2 | 69.9301 | CCCN1C(Nc2ccccc2C1═O)c3ccc(C)cc3 | Scaffold-13 |
| SJ000288424-2 | 69.9301 | CCCN1C(Nc2ccccc2C1═O)c3ccc(Cl)cc3Cl | Scaffold-13 |
| SJ000288430-1 | 69.9301 | CCCN1C(Nc2ccccc2C1═O)c3ccc(OC)c(COc4ccc(cc4)[N+](═O)[O−])c3 | Scaffold-13 |
| SJ000288432-1 | 69.9301 | CCOc1cc(ccc1O)C2Nc3ccccc3C(═O)N2Cc4ccccc4 | Scaffold-13 |
| SJ000288433-1 | 69.9301 | COc1ccc(cc1Cn2cc(cn2)[N+](═O)[O−])C3Nc4ccccc4C(═O)N3Cc5ccccc5 | Scaffold-13 |
| SJ000288444-1 | 69.9301 | CCCN1C(Nc2ccccc2C1═O)c3ccc(OC)c(CSc4ccccn4)c3 | Scaffold-13 |
| SJ000288611-1 | 69.9301 | CCCN1C(Nc2ccccc2C1═O)c3ccccc3F | Scaffold-13 |
| SJ000288627-1 | 69.9301 | O═C1N(Cc2ccccc2)C(Nc3ccccc13)c4ccc5OCOc5c4 | Scaffold-13 |
| SJ000288637-1 | 69.9301 | COc1ccc(cc1Cn2nc(C)c(Cl)c2C)C3Nc4ccccc4C(═O)N3Cc5ccccc5 | Scaffold-13 |
| SJ000288667-1 | 69.9301 | CCCN1C(Nc2ccccc2C1═O)c3ccc4OCOc4c3 | Scaffold-13 |
| SJ000295321-1 | 69.9301 | CCCN1C(Nc2ccccc2C1═O)c3c(Cl)cccc3Cl | Scaffold-13 |
| SJ000295328-2 | 69.9301 | COc1ccc(cc1COc2ccccc2F)C3Nc4ccccc4C(═O)N3Cc5ccccc5 | Scaffold-13 |
| SJ000295698-1 | 69.9301 | COc1ccc(cc1CN2C(═O)c3ccccc3C2═O)C4Nc5ccccc5C(═O)N4Cc6ccccc6 | Scaffold-13 |
| SJ000295704-1 | 69.9301 | COc1cccc(OCc2cc(ccc2OC)C3Nc4ccccc4C(═O)N3Cc5ccccc5)c1 | Scaffold-13 |
| SJ000295722-1 | 69.9301 | CCCN1C(Nc2ccccc2C1═O)c3ccc(O)c(OC)c3 | Scaffold-13 |
| SJ000295736-1 | 69.9301 | CCCN1C(Nc2ccccc2C1═O)c3ccc(OC)c(COc4ccccc4F)c3 | Scaffold-13 |
| SJ000295745-1 | 69.9301 | CCCN1C(Nc2ccccc2C1═O)c3ccc(OC)c(COc4ccc(c(C)c4)[N+](═O)[O−])c3 | Scaffold-13 |
| SJ000296071-1 | 69.9301 | CCCN1C(Nc2ccccc2C1═O)c3ccc(Cl)c(Cl)c3 | Scaffold-13 |
| SJ000395233-1 | 69.9301 | CCCN1C(Nc2ccccc2C1═O)c3cccc(O)c3O | Scaffold-13 |
| SJ000413320-1 | 69.9301 | CCC(C)N1C(Nc2ccccc2C1═O)c3cccc(OC)c3O | Scaffold-13 |
| SJ000414199-1 | 69.9301 | CCCN1C(Nc2ccccc2C1═O)c3cc(Cl)c(O)c(OC)c3 | Scaffold-13 |
| SJ000508213-1 | 69.9301 | Clc1ccc(CN2C(Nc3ccccc3C2═O)c4ccc(Br)cc4)cc1 | Scaffold-13 |
| SJ000510740-1 | 69.9301 | COc1ccc(CN2C(Nc3ccccc3C2═O)c4ccc(F)cc4)cc1 | Scaffold-13 |
| SJ000510741-1 | 69.9301 | COc1ccc(CN2C(Nc3ccccc3C2═O)c4ccc(C)cc4)cc1 | Scaffold-13 |
| SJ000510742-1 | 69.9301 | COc1ccc(cc1)C2Nc3ccccc3C(═O)N2Cc4ccc(F)cc4 | Scaffold-13 |
| SJ000510743-1 | 69.9301 | COc1ccc(cc1)C2Nc3ccccc3C(═O)N2Cc4ccc(C)cc4 | Scaffold-13 |
| SJ000510744-1 | 69.9301 | COc1ccc(CN2C(Nc3ccccc3C2═O)c4ccc(OC)cc4)cc1 | Scaffold-13 |
| SJ000510745-1 | 69.9301 | COc1ccc(CN2C(Nc3ccccc3C2═O)c4ccc(cc4)[N+](═O)[O−])cc1 | Scaffold-13 |
| SJ000550608-1 | 69.9301 | CCCN1C(Nc2ccccc2C1═O)c3ccc(Cl)cc3 | Scaffold-13 |
| SJ000551164-1 | 69.9301 | Clc1ccc(C2Nc3ccccc3C(═O)N2Cc4ccccc4)c(Cl)c1 | Scaffold-13 |
| SJ000551165-1 | 69.9301 | CCCN1C(Nc2ccccc2C1═O)c3cc(OC)c(OC)c(OC)c3 | Scaffold-13 |
| SJ000551208-1 | 69.9301 | Clc1ccc(cc1Cl)C2Nc3ccccc3C(═O)N2Cc4ccccc4 | Scaffold-13 |
| SJ000551209-1 | 69.9301 | COc1ccc(cc1)C2Nc3ccccc3C(═O)N2Cc4ccccc4 | Scaffold-13 |
| SJ000803767-1 | 71.0839 | CN(C)c1ccc(cc1)C2Nc3ccccc3C(═O)N2CCCN4CCOCC4 | Scaffold-13 |
| SJ000803762-1 | 71.282 | CSc1ccc(cc1)C2Nc3ccccc3C(═O)N2CCCN4CCOCC4 | Scaffold-13 |
| SJ000803760-1 | 72.0746 | COc1ccc(cc1OC(F)F)C2Nc3ccccc3C(═O)N2CCCN4CCOCC4 | Scaffold-13 |
| SJ000803758-1 | 72.1212 | CCCOc1ccc(cc1)C2Nc3ccccc3C(═O)N2CCCN4CCOCC4 | Scaffold-13 |
| SJ000368145-1 | 40.769 | [O−][N+](═O)c1ccc(cc1)C2═C(COC2═O)c3ccc(OC(F)F)cc3 | Scaffold-14 |
| SJ000285362-5 | 67.5845 | CS(═O)(═O)c1ccc(cc1)C2═C(C(═O)OC2)c3ccccc3 | Scaffold-14 |
| SJ000367280-1 | 69.9301 | COc1ccc(cc1)C2═C(C(═O)OC2)c3ccc(cc3)[N+](═O)[O−] | Scaffold-14 |
| SJ000368097-1 | 69.9301 | CC(C)(C)c1ccc(cc1)C2═C(C(═O)OC2)c3ccc(cc3)[N+](═O)[O−] | Scaffold-14 |
| SJ000368117-1 | 69.9301 | CCCc1ccc(cc1)C2═C(C(═O)OC2)c3ccc(cc3)[N+](═O)[O−] | Scaffold-14 |
| SJ000368127-1 | 69.9301 | CCc1ccc(cc1)C2═C(C(═O)OC2)c3ccc(cc3)[N+](═O)[O−] | Scaffold-14 |
| SJ000368155-1 | 69.9301 | [O−][N+](═O)c1ccc(cc1)C2═C(COC2═O)c3ccc(F)cc3 | Scaffold-14 |
| SJ000368165-1 | 69.9301 | CCC(C)c1ccc(cc1)C2═C(C(═O)OC2)c3ccc(cc3)[N+](═O)[O−] | Scaffold-14 |
| SJ000413143-1 | 69.9301 | [O−][N+](═O)c1ccc(cc1)C2═C(COC2═O)c3ccc(Cl)cc3 | Scaffold-14 |
| SJ000312204-6 | 2.2509 | ClC1═C(NCCN2CCOCC2)C(═O)c3ncccc3C1═O | Scaffold-2 |
| SJ000109503-1 | 3.3 | CN(C)c1ccc(NC2═C(Cl)C(═O)c3ccccc3C2═O)cc1 | Scaffold-2 |
| SJ000032717-1 | 8.7103 | CCCCOc1ccc(cc1)N(C(═O)C)C2═C(Cl)C(═O)c3ccccc3C2═O | Scaffold-2 |
| SJ000024928-1 | 9.8694 | FC(F)(F)C(═O)NC1═C(Cl)C(═O)c2ccccc2C1═O | Scaffold-2 |
| SJ000021282-1 | 10.39 | Cc1ccc(cc1)S(═O)(═O)N2CCN(CC2)C3═C(Cl)C(═O)c4ccccc4C3═O | Scaffold-2 |
| SJ000149466-1 | 17.2484 | CCC(COC(═O)C)NC1═C(Cl)C(═O)c2ccccc2C1═O | Scaffold-2 |
| SJ000294373-2 | 20.0699 | NC1═C(N2CCCCC2)C(═O)c3ccccc3C1═O | Scaffold-2 |
| SJ000032724-1 | 28.2784 | CCCCCOc1ccc(cc1)N(C(═O)C)C2═C(Cl)C(═O)c3ccccc3C2═O | Scaffold-2 |
| SJ000562630-1 | 53.8462 | NC1═C(Nc2ccc(Cl)cc2)C(═O)c3ccccc3C1═O | Scaffold-2 |
| SJ000562629-1 | 54.7552 | CCCCCCCCOc1ccc(NC2═C(N)C(═O)c3ccccc3C2═O)cc1 | Scaffold-2 |
| SJ000562641-1 | 55.035 | Cc1cc(C)cc(NC2═C(N)C(═O)c3ccccc3C2═O)c1 | Scaffold-2 |
| SJ000562618-1 | 60.5594 | Cc1ccc(NC2═C(N)C(═O)c3ccccc3C2═O)cc1 | Scaffold-2 |
| SJ000562543-1 | 60.8392 | NC1═C(Nc2ccccc2)C(═O)c3ccccc3C1═O | Scaffold-2 |
| SJ000558312-2 | 65.6643 | CCOc1ccc(cc1)N(C(═O)C)C2═C(N)C(═O)c3ccccc3C2═O | Scaffold-2 |
| SJ000022282-2 | 69.9301 | OC(═O)c1cccc(NC2═C(Cl)C(═O)c3ccccc3C2═O)c1 | Scaffold-2 |
| SJ000024921-1 | 69.9301 | ClC1═C(Nc2ccc(Br)cc2)C(═O)c3ccccc3C1═O | Scaffold-2 |
| SJ000024922-1 | 69.9301 | CC(═O)Nc1ccc(NC2═C(Cl)C(═O)c3ccccc3C2═O)cc1 | Scaffold-2 |
| SJ000024923-1 | 69.9301 | CCCCOC(═O)c1ccc(NC2═C(Cl)C(═O)c3ccccc3C2═O)cc1 | Scaffold-2 |
| SJ000024924-2 | 69.9301 | FC(F)(F)c1cccc(NC2═C(Cl)C(═O)c3ccccc3C2═O)c1 | Scaffold-2 |
| SJ000024925-1 | 69.9301 | Cc1cccc(NC2═C(Cl)C(═O)c3ccccc3C2═O)c1 | Scaffold-2 |
| SJ000024926-1 | 69.9301 | COC(═O)c1cccc(NC2═C(Cl)C(═O)c3ccccc3C2═O)c1 | Scaffold-2 |
| SJ000024931-1 | 69.9301 | CC(═O)N(C1═C(Cl)C(═O)c2ccccc2C1═O)c3cccc(I)c3 | Scaffold-2 |
| SJ000024934-1 | 69.9301 | Cc1cccc(NC2═C(N)C(═O)c3ccccc3C2═O)c1 | Scaffold-2 |
| SJ000024935-1 | 69.9301 | CC(═O)N(C1═C(Cl)C(═O)c2ccccc2C1═O)c3ccc(cc3)C(═O)O | Scaffold-2 |
| SJ000024947-1 | 69.9301 | CC(═O)N(C1═C(Cl)C(═O)c2ccccc2C1═O)c3ccc(F)cc3 | Scaffold-2 |
| SJ000029651-1 | 69.9301 | ClC1═C(N2CCN(CC2)S(═O)(═O)c3ccc(Cl)cc3)C(═O)c4ccccc4C1═C | Scaffold-2 |
| SJ000030567-2 | 69.9301 | CCOc1ccc(cc1)N(C(═O)C)C2═C(Cl)C(═O)c3ccccc3C2═O | Scaffold-2 |
| SJ000032711-1 | 69.9301 | CCOC(═O)c1ccc(NC2═C(N)C(═O)c3ccccc3C2═O)cc1 | Scaffold-2 |
| SJ000032712-1 | 69.9301 | NC1═C(Nc2ccc(I)cc2)C(═O)c3ccccc3C1═O | Scaffold-2 |
| SJ000032713-1 | 69.9301 | NC1═C(Nc2ccc(Br)cc2)C(═O)c3ccccc3C1═O | Scaffold-2 |
| SJ000032723-1 | 69.9301 | CCCCCOc1ccc(NC2═C(N)C(═O)c3ccccc3C2═O)cc1 | Scaffold-2 |
| SJ000033014-1 | 69.9301 | ClC1═C(N2CCN(CC2)S(═O)(═O)c3ccc(Br)cc3)C(═O)c4ccccc4C1═O | Scaffold-2 |
| SJ000036320-1 | 69.9301 | CC(═O)Nc1ccc(cc1)S(═O)(═O)N2CCN(CC2)C3═C(Cl)C(═O)c4ccccc4C3═O | Scaffold-2 |
| SJ000038882-1 | 69.9301 | Fc1ccc(cc1)S(═O)(═O)N2CCN(CC2)C3═C(Cl)C(═O)c4ccccc4C3═O | Scaffold-2 |
| SJ000046178-1 | 69.9301 | CC(═O)N(C1═C(Cl)C(═O)c2ccccc2C1═O)c3cccc(O)c3 | Scaffold-2 |
| SJ000048680-1 | 69.9301 | ClC1═C(Nc2cccc(Br)c2)C(═O)c3ccccc3C1═O | Scaffold-2 |
| SJ000053101-1 | 69.9301 | CC(═O)NC1═C(N)C(═O)c2ccccc2C1═O | Scaffold-2 |
| SJ000067071-3 | 69.9301 | CC(═O)NC1═C(Cl)C(═O)c2ccccc2C1═O | Scaffold-2 |
| SJ000120750-1 | 69.9301 | Cc1ccc(NC2═C(Cl)C(═O)c3ccccc3C2═O)cc1 | Scaffold-2 |
| SJ000122936-1 | 69.9301 | CCOc1ccc(NC2═C(Cl)C(═O)c3ccccc3C2═O)cc1 | Scaffold-2 |
| SJ000141460-3 | 69.9301 | CCCNC1═C(Cl)C(═O)c2ccccc2C1═O | Scaffold-2 |
| SJ000141461-1 | 69.9301 | CNC1═C(Cl)C(═O)c2ccccc2C1═O | Scaffold-2 |
| SJ000147784-1 | 69.9301 | CC(C)NC1═C(Cl)C(═O)c2ccccc2C1═O | Scaffold-2 |
| SJ000148678-1 | 69.9301 | CCC(CO)NC1═C(Cl)C(═O)c2ccccc2C1═O | Scaffold-2 |
| SJ000157082-1 | 69.9301 | CCNC1═C(Cl)C(═O)c2ccccc2C1═O | Scaffold-2 |
| SJ000242799-1 | 69.9301 | ClC1═C(N2CCOCC2)C(═O)c3ccccc3C1═O | Scaffold-2 |
| SJ000244608-1 | 69.9301 | CC(NC1═C(Cl)C(═O)c2ccccc2C1═O)C(═O)O | Scaffold-2 |
| SJ000290718-2 | 69.9301 | CN(C(═O)C)C1═C(Cl)C(═O)c2ccccc2C1═O | Scaffold-2 |
| SJ000296883-2 | 69.9301 | CC(═O)N(C1═C(Cl)C(═O)c2ccccc2C1═O)c3ccc(Cl)cc3 | Scaffold-2 |
| SJ000297073-1 | 69.9301 | ClC1═C(Nc2ccc(Cl)cc2)C(═O)c3ccccc3C1═O | Scaffold-2 |
| SJ000299646-1 | 69.9301 | COC(═O)c1ccc(NC2═C(Cl)C(═O)c3ccccc3C2═O)cc1 | Scaffold-2 |
| SJ000299694-1 | 69.9301 | CC1CCCC(C)C1NC2═C(Cl)C(═O)c3ccccc3C2═O | Scaffold-2 |
| SJ000299699-2 | 69.9301 | CC(═O)N(C1═C(Cl)C(═O)c2ccccc2C1═O)c3ccc(Br)cc3 | Scaffold-2 |
| SJ000299708-1 | 69.9301 | ClC1═C(Nc2ccc(Nc3ccccc3)cc2)C(═O)c4ccccc4C1═O | Scaffold-2 |
| SJ000300361-1 | 69.9301 | NC1═C(Nc2cccc3ccccc23)C(═O)c4ccccc4C1═O | Scaffold-2 |
| SJ000562551-1 | 72.1678 | Cc1ccccc1NC2═C(N)C(═O)c3ccccc3C2═O | Scaffold-2 |
| SJ000244609-2 | 587.4126 | CC(C)C(NC1═C(Cl)C(═O)c2ccccc2C1═O)C(═O)O | Scaffold-2 |
| SJ000378900-1 | 2.6366 | CCCCCn1c(nc2N(CCCC)C(═O)NC(═O)c12)c3ccc(F)c(Br)c3 | Scaffold-3 |
| SJ000287642-4 | 21.7691 | CN1C(═O)N(C)c2[nH]c(nc2C1═O)c3ccc(cc3)S(═O)(═O)O | Scaffold-3 |
| SJ000140137-1 | 69.9301 | CCOC(═O)COc1ccc(cc1OC)c2nc3C(═O)N(C)C(═O)N(C)c3[nH]2 | Scaffold-3 |
| SJ000203904-1 | 69.9301 | CC(C)c1ccc(cc1)c2nc3C(═O)N(C)C(═O)N(C)c3[nH]2 | Scaffold-3 |
| SJ000205042-1 | 69.9301 | CCCOc1ccc(cc1)c2nc3C(═O)N(C)C(═O)N(C)c3[nH]2 | Scaffold-3 |
| SJ000287650-2 | 69.9301 | CCCN1C(═O)N(CCC)c2[nH]c(nc2C1═O)c3ccc(cc3)S(═O)(═O)O | Scaffold-3 |
| SJ000287997-2 | 69.9301 | CCCN1C(═O)N(CCC)c2[nH]c(nc2C1═O)c3ccc(OCC(═O)Nc4ccc(cc4)C#N)cc3 | Scaffold-3 |
| SJ000288094-2 | 69.9301 | CN1C(═O)N(C)c2[nH]c(nc2C1═O)c3ccccc3 | Scaffold-3 |
| SJ000288288-2 | 69.9301 | CCCN1C(═O)N(CCC)c2[nH]c(nc2C1═O)c3ccc(OCC(═O)NCCN)cc3 | Scaffold-3 |
| SJ000375541-1 | 69.9301 | CCCn1c(nc2N(Cc3ccccc3)C(═O)NC(═O)c12)c4ccc(Cl)cc4 | Scaffold-3 |
| SJ000375920-1 | 69.9301 | CCCn1c(nc2N(Cc3ccccc3)C(═O)NC(═O)c12)c4ccc(cc4)C(C)(C)C | Scaffold-3 |
| SJ000376601-1 | 69.9301 | CCCn1c(nc2N(Cc3ccccc3)C(═O)NC(═O)c12)c4cccc(c4)[N+](═O)[O−] | Scaffold-3 |
| SJ000376621-1 | 69.9301 | CCCn1c(nc2N(Cc3ccccc3)C(═O)NC(═O)c12)c4ccc(OC(F)F)cc4 | Scaffold-3 |
| SJ000377779-1 | 69.9301 | CCCN1C(═O)NC(═O)c2c1nc(c3ccc(OC(F)F)cc3)n2CCN4CCOCC4 | Scaffold-3 |
| SJ000377955-1 | 69.9301 | CCCN1C(═O)NC(═O)c2c1nc(c3ccc(cc3)C(C)(C)C)n2CCN4CCOCC4 | Scaffold-3 |
| SJ000378868-1 | 69.9301 | CCCCN1C(═O)NC(═O)c2c1nc(c3ccc(CC)c(c3)[N+](═O)[O−])n2C | Scaffold-3 |
| SJ000378878-1 | 69.9301 | CCCCn1c(nc2N(CCC)C(═O)NC(═O)c12)c3ccc(OCC═C)cc3 | Scaffold-3 |
| SJ000378880-1 | 69.9301 | COCCCn1c(nc2N(Cc3ccccc3)C(═O)NC(═O)c12)c4ccc(OCC═C)cc4 | Scaffold-3 |
| SJ000378893-1 | 69.9301 | CCCN1C(═O)NC(═O)c2c1nc(c3ccc(OC)c(Br)c3)n2CCOC | Scaffold-3 |
| SJ000378903-1 | 69.9301 | CCCCN1C(═O)NC(═O)c2c1nc(c3ccc(OCC(C)C)c(OCC)c3)n2CC | Scaffold-3 |
| SJ000378906-1 | 69.9301 | CCCCn1c(nc2N(CCC)C(═O)NC(═O)c12)c3ccc(OCCC)c(OCC)c3 | Scaffold-3 |
| SJ000378907-1 | 69.9301 | CCCCn1c(nc2N(Cc3ccccc3)C(═O)NC(═O)c12)c4ccc(Cl)cc4 | Scaffold-3 |
| SJ000378916-1 | 69.9301 | CCCCN1C(═O)NC(═O)c2c1nc(c3ccc(cc3)C(F)(F)F)n2C | Scaffold-3 |
| SJ000378917-1 | 69.9301 | COCCn1c(nc2N(CC(C)C)C(═O)NC(═O)c12)c3ccc(SC)cc3 | Scaffold-3 |
| SJ000378919-1 | 69.9301 | CCCCN1C(═O)NC(═O)c2c1nc(c3ccc(C)c(c3)[N+](═O)[O−])n2CC | Scaffold-3 |
| SJ000378928-1 | 69.9301 | CCCN1C(═O)NC(═O)c2c1nc(c3cccc(F)c3)n2CCOC | Scaffold-3 |
| SJ000378938-1 | 69.9301 | CCCCN1C(═O)NC(═O)c2c1nc(c3ccc(cc3)[N+](═O)[O−])n2CC | Scaffold-3 |
| SJ000378943-1 | 69.9301 | CCCCN1C(═O)NC(═O)c2c1nc(c3ccc(OC)c(Br)c3)n2CC(C)C | Scaffold-3 |
| SJ000378946-1 | 69.9301 | CCCCN1C(═O)NC(═O)c2c1nc(c3ccc(OCCC)c(OCC)c3)n2CCOC | Scaffold-3 |
| SJ000378966-1 | 69.9301 | CCCCN1C(═O)NC(═O)c2c1nc(c3ccc(OC(F)(F)C(F)C(F)(F)F)cc3)n2O | Scaffold-3 |
| SJ000379015-1 | 69.9301 | CC(C)CN1C(═O)NC(═O)c2c1nc(c3ccc(C)c(c3)[N+](═O)[O−])n2CC(C)C | Scaffold-3 |
| SJ000379301-1 | 69.9301 | CCCn1c(nc2N(Cc3ccccc3)C(═O)NC(═O)c12)c4ccc(OCC═C)c(Br)c4 | Scaffold-3 |
| SJ000379374-1 | 69.9301 | CCCN1C(═O)NC(═O)c2c1nc(c3ccc(OC(F)(F)C(F)C(F)(F)F)cc3)n2CCOC | Scaffold-3 |
| SJ000379462-1 | 69.9301 | CCCCN1C(═O)NC(═O)c2c1nc(c3ccc(OCC═C)c(Br)c3)n2C | Scaffold-3 |
| SJ000381119-1 | 69.9301 | CCCn1c(nc2N(Cc3ccccc3)C(═O)NC(═O)c12)c4ccc(SC)cc4 | Scaffold-3 |
| SJ000381474-1 | 69.9301 | CCCOc1ccc(cc1OCC)c2nc3N(Cc4ccccc4)C(═O)NC(═O)c3n2CCO | Scaffold-3 |
| SJ000382100-1 | 69.9301 | CCCCN1C(═O)NC(═O)c2c1nc(c3ccc(SC)cc3)n2C | Scaffold-3 |
| SJ000382136-1 | 69.9301 | COc1ccc(Br)cc1c2nc3N(Cc4ccccc4)C(═O)NC(═O)c3n2C | Scaffold-3 |
| SJ000382289-1 | 69.9301 | CCCn1c(nc2N(Cc3ccccc3)C(═O)NC(═O)c12)c4cccc(c4)C(F)(F)F | Scaffold-3 |
| SJ000382299-1 | 69.9301 | CCCn1c(nc2N(Cc3ccccc3)C(═O)NC(═O)c12)c4cccc(F)c4 | Scaffold-3 |
| SJ000383781-1 | 69.9301 | CCCn1c(nc2N(Cc3ccccc3)C(═O)NC(═O)c12)c4ccc(cc4)C#N | Scaffold-3 |
| SJ000383944-1 | 69.9301 | COCCCn1c(nc2N(Cc3ccccc3)C(═O)NC(═O)c12)c4cccc(Cl)c4 | Scaffold-3 |
| SJ000384618-1 | 69.9301 | Cn1c(nc2N(Cc3ccccc3)C(═O)NC(═O)c12)c4ccc(Cl)cc4 | Scaffold-3 |
| SJ000386224-1 | 69.9301 | Cn1c(nc2N(Cc3ccccc3)C(═O)NC(═O)c12)c4cc(Cl)ccc4O | Scaffold-3 |
| SJ000387665-1 | 69.9301 | Cn1c(nc2N(Cc3ccccc3)C(═O)NC(═O)c12)c4cccc(F)c4 | Scaffold-3 |
| SJ000389476-1 | 69.9301 | CCOc1ccc(cc1)c2nc3N(Cc4ccccc4)C(═O)NC(═O)c3n2C | Scaffold-3 |
| SJ000390112-1 | 69.9301 | CC(C)Cn1c(nc2N(Cc3ccccc3)C(═O)NC(═O)c12)c4cc(ccc4Cl)[N+](═O)[O−] | Scaffold-3 |
| SJ000394956-1 | 69.9301 | CSc1ccc(cc1)c2nc3N(CC(C)C)C(═O)NC(═O)c3n2CC(C)C | Scaffold-3 |
| SJ000396262-1 | 69.9301 | CN(C)c1ccc(cc1)c2nc3N(Cc4ccccc4)C(═O)NC(═O)c3n2C | Scaffold-3 |
| SJ000403937-1 | 69.9301 | CCCN1C(═O)NC(═O)c2c1nc(c3ccc(cc3)C#N)n2CCN4CCOCC4 | Scaffold-3 |
| SJ000404631-1 | 69.9301 | Cn1c(nc2N(Cc3ccccc3)C(═O)NC(═O)c12)c4ccc(cc4)C(F)(F)F | Scaffold-3 |
| SJ000406218-1 | 69.9301 | CCCN1C(═O)NC(═O)c2c1nc(c3ccc(NC(═O)C)cc3)n2CCN4CCOCC4 | Scaffold-3 |
| SJ000411586-1 | 69.9301 | COCCCn1c(nc2N(Cc3ccccc3)C(═O)NC(═O)c12)c4cc(Br)ccc4F | Scaffold-3 |
| SJ000414469-1 | 69.9301 | CCCCN1C(═O)NC(═O)c2c1nc(c3cccc(F)c3)n2CC | Scaffold-3 |
| SJ000415940-1 | 69.9301 | CCCCN1C(═O)NC(═O)c2c1nc(c3cccc(F)c3)n2C | Scaffold-3 |
| SJ000416762-1 | 69.9301 | CCCN1C(═O)NC(═O)c2c1nc(c3ccc(SC)cc3)n2CCOC | Scaffold-3 |
| SJ000420075-1 | 69.9301 | COCCCn1c(nc2N(Cc3ccccc3)C(═O)NC(═O)c12)c4ccc(cc4)C#N | Scaffold-3 |
| SJ000420085-1 | 69.9301 | Cn1c(nc2N(Cc3ccccc3)C(═O)NC(═O)c12)c4ccc(OCC═C)c(Br)c4 | Scaffold-3 |
| SJ000420129-1 | 69.9301 | CCCn1c(nc2N(Cc3ccccc3)C(═O)NC(═O)c12)c4ccc(cc4)C(═O)O | Scaffold-3 |
| SJ000420179-1 | 69.9301 | CCCn1c(nc2N(Cc3ccccc3)C(═O)NC(═O)c12)c4ccc(cc4)C(═O)OC | Scaffold-3 |
| SJ000420343-1 | 69.9301 | CCc1ccc(cc1)c2nc3N(CC(C)C)C(═O)NC(═O)c3n2CCOC | Scaffold-3 |
| SJ000421295-1 | 69.9301 | CCCCN1C(═O)NC(═O)c2c1nc(c3ccc(cc3)C#N)n2C | Scaffold-3 |
| SJ000421396-1 | 69.9301 | COCCn1c(nc2N(CC(C)C)C(═O)NC(═O)c12)c3ccc(C)cc3 | Scaffold-3 |
| SJ000421862-1 | 69.9301 | CCCCN1C(═O)NC(═O)c2c1nc(c3ccc(F)cc3)n2CCOC | Scaffold-3 |
| SJ000422648-1 | 69.9301 | COCCn1c(nc2N(CC(C)C)C(═O)NC(═O)c12)c3ccc(F)cc3 | Scaffold-3 |
| SJ000423271-1 | 69.9301 | CCCN1C(═O)NC(═O)c2c1nc(c3ccc(OC)cc3)n2CCOC | Scaffold-3 |
| SJ000423281-1 | 69.9301 | CCCN1C(═O)NC(═O)c2c1nc(c3cccc(C)c3)n2CCOC | Scaffold-3 |
| SJ000424475-1 | 69.9301 | CCCN1C(═O)NC(═O)c2c1nc(c3ccc(Cl)cc3)n2CCOC | Scaffold-3 |
| SJ000425093-1 | 69.9301 | CCCCN1C(═O)NC(═O)c2c1nc(c3ccc(C)cc3)n2C | Scaffold-3 |
| SJ000425187-1 | 69.9301 | CCCN1C(═O)NC(═O)c2c1nc(c3ccc(OC)c(C)c3)n2CCOC | Scaffold-3 |
| SJ000425224-1 | 69.9301 | COCCn1c(nc2N(CC(C)C)C(═O)NC(═O)c12)c3ccc(OC)cc3 | Scaffold-3 |
| SJ000425256-1 | 69.9301 | CCCCN1C(═O)NC(═O)c2c1nc(c3ccc(OC)c(C)c3)n2C | Scaffold-3 |
| SJ000425260-1 | 69.9301 | CCCCN1C(═O)NC(═O)c2c1nc(c3ccc(OC)cc3)n2C | Scaffold-3 |
| SJ000426389-1 | 69.9301 | COCCn1c(nc2N(CC(C)C)C(═O)NC(═O)c12)c3cccc(C)c3 | Scaffold-3 |
| SJ000427207-1 | 69.9301 | CCCN1C(═O)NC(═O)c2c1nc(c3cccc(OC)c3)n2CCOC | Scaffold-3 |
| SJ000427387-1 | 69.9301 | CCCCN1C(═O)NC(═O)c2c1nc(c3ccc(OCC)c(OC)c3)n2CCOC | Scaffold-3 |
| SJ000429070-1 | 69.9301 | CCCCN1C(═O)NC(═O)c2c1nc(c3ccc(O)c(OC)c3)n2CCC | Scaffold-3 |
| SJ000430392-1 | 69.9301 | CCn1c(nc2N(Cc3ccccc3)C(═O)NC(═O)c12)c4ccc(O)c(OC)c4 | Scaffold-3 |
| SJ000430813-1 | 69.9301 | COc1cc(ccc1O)c2nc3N(Cc4ccccc4)C(═O)NC(═O)c3n2C | Scaffold-3 |
| SJ000431347-1 | 69.9301 | CCCCN1C(═O)NC(═O)c2c1nc(c3ccc(OCC(═O)O)cc3)n2CCC(C)C | Scaffold-3 |
| SJ000431389-1 | 69.9301 | CCCN1C(═O)NC(═O)c2c1nc(c3cccc(Br)c3)n2CCOC | Scaffold-3 |
| SJ000437843-1 | 69.9301 | CCCn1c(nc2N(Cc3ccccc3)C(═O)NC(═O)c12)c4cc(Br)ccc4F | Scaffold-3 |
| SJ000441071-1 | 69.9301 | Cn1c(nc2N(Cc3ccccc3)C(═O)NC(═O)c12)c4ccc(OCC(═O)O)cc4 | Scaffold-3 |
| SJ000451928-1 | 69.9301 | CCCCCn1c(nc2N(CCCC)C(═O)NC(═O)c12)c3cccc(OC)c3 | Scaffold-3 |
| SJ000452204-1 | 69.9301 | CCCn1c(nc2N(Cc3ccccc3)C(═O)NC(═O)c12)c4ccc(OC)cc4 | Scaffold-3 |
| SJ000456724-1 | 69.9301 | CCCn1c(nc2N(Cc3ccccc3)C(═O)NC(═O)c12)c4ccc(F)cc4 | Scaffold-3 |
| SJ000459257-1 | 69.9301 | CCCN1C(═O)NC(═O)c2c1nc(c3ccc(OC(F)F)c(OC)c3)n2CCOC | Scaffold-3 |
| SJ000460165-1 | 69.9301 | CCCCCn1c(nc2N(CCCC)C(═O)NC(═O)c12)c3cccc(F)c3 | Scaffold-3 |
| SJ000460758-1 | 69.9301 | CCCN1C(═O)NC(═O)c2c1nc(c3ccc(F)c(F)c3)n2CCOC | Scaffold-3 |
| SJ000517919-1 | 69.9301 | CN1C(═O)NC(═O)c2c1nc(c3ccc(Cl)c(C)c3)n2C | Scaffold-3 |
| SJ000518479-1 | 69.9301 | CN1C(═O)NC(═O)c2c1nc(c3ccc4OCOc4c3)n2C | Scaffold-3 |
| SJ000832251-1 | 72.951 | CCCN1C(═O)N(CCC)c2[nH]c(nc2C1═O)c3ccccc3 | Scaffold-3 |
| SJ000287614-4 | 75.0839 | CN1C(═O)N(CC═C)C(═O)c2c1nc(c3ccc(cc3)S(═O)(═O)O)n2C | Scaffold-3 |
| SJ000832270-1 | 85.8042 | CN1C(═O)N(CC═C)C(═O)c2c1nc(c3ccccc3)n2C | Scaffold-3 |
| SJ000521054-1 | 0.3969 | FC(F)(F)c1ccc2c(NCCCNCc3ccc4[nH]ccc4c3)ccnc2c1 | Scaffold-4 |
| SJ000311289-1 | 4.6177 | COc1ccc(F)cc1CNCCCNc2ccnc3cc(ccc23)c4ccc(cc4)C(C)(C)C | Scaffold-4 |
| SJ000311287-1 | 4.6707 | COc1cccc(CNCCCNc2ccnc3cc(ccc23)c4ccc(cc4)C(C)(C)C)c1O | Scaffold-4 |
| SJ000311233-1 | 5.6335 | COc1ccc(F)cc1CNCCCNc2ccnc3cc(Oc4cccc(c4)N(C)C)ccc23 | Scaffold-4 |
| SJ000311237-1 | 6.2948 | COc1ccc(F)cc1CNCCCNc2ccnc3cc(Oc4ccc(cc4)C(C)(C)C)ccc23 | Scaffold-4 |
| SJ000311288-1 | 6.8705 | CC(C)(C)c1ccc(cc1)c2ccc3c(NCCCNCc4cccc5OCOc45)ccnc3c2 | Scaffold-4 |
| SJ000311235-1 | 7.6478 | COc1cccc(CNCCCNc2ccnc3cc(Oc4ccc(cc4)C(C)(C)C)ccc23)c1O | Scaffold-4 |
| SJ000311273-1 | 7.6851 | COc1ccc(F)cc1CNCCCNc2ccnc3cc(ccc23)c4cc(cc(c4)C(F)(F)F)C(F)(F)F | Scaffold-4 |
| SJ000311236-1 | 8.6715 | CC(C)(C)c1ccc(Oc2ccc3c(NCCCNCc4cccc5OCOc45)ccnc3c2)cc1 | Scaffold-4 |
| SJ000311272-1 | 9.017 | FC(F)(F)c1cc(cc(c1)C(F)(F)F)c2ccc3c(NCCCNCc4cccc5OCOc45)ccnc3c2 | Scaffold-4 |
| SJ000311321-1 | 9.0403 | COc1ccc(F)cc1CNCCCNc2ccnc3cc(Cc4cc(C)cc(C)c4)ccc23 | Scaffold-4 |
| SJ000311279-1 | 9.5166 | COc1cccc(CNCCCNc2ccnc3cc(ccc23)c4ccc(cc4)C(F)(F)F)c1O | Scaffold-4 |
| SJ000311267-1 | 9.9542 | CCC(CC)c1ccc2c(NCCCNCc3cccc(OC)c3O)ccnc2c1 | Scaffold-4 |
| SJ000311277-1 | 10.2711 | COc1ccc(F)cc1CNCCCNc2ccnc3cc(ccc23)c4cccc5ccccc45 | Scaffold-4 |
| SJ000311316-1 | 11.0363 | Clc1cccc(Cl)c1Cc2ccc3c(NCCCNCc4cccc5OCOc45)ccnc3c2 | Scaffold-4 |
| SJ000311317-1 | 12.2308 | COc1ccc(F)cc1CNCCCNc2ccnc3cc(Cc4c(Cl)cccc4Cl)ccc23 | Scaffold-4 |
| SJ000311300-1 | 12.792 | FC(F)(F)c1cccc(Cc2ccc3c(NCCCNCc4cccc5OCOc45)ccnc3c2)c1 | Scaffold-4 |
| SJ000311281-1 | 13.3814 | COc1ccc(F)cc1CNCCCNc2ccnc3cc(ccc23)c4ccc(cc4)C(F)(F)F | Scaffold-4 |
| SJ000311315-1 | 13.8323 | COc1cccc(CNCCCNc2ccnc3cc(Cc4c(Cl)cccc4Cl)ccc23)c1O | Scaffold-4 |
| SJ000311280-1 | 15.7052 | FC(F)(F)c1ccc(cc1)c2ccc3c(NCCCNCc4cccc5OCOc45)ccnc3c2 | Scaffold-4 |
| SJ000311275-1 | 16.0049 | COc1cccc(CNCCCNc2ccnc3cc(ccc23)c4cccc5ccccc45)c1O | Scaffold-4 |
| SJ000311252-1 | 16.0341 | C(CNCc1cccc2OCOc12)CNc3ccnc4cc(CC5CCCCC5)ccc34 | Scaffold-4 |
| SJ000311320-1 | 16.259 | Cc1cc(C)cc(Cc2ccc3c(NCCCNCc4cccc5OCOc45)ccnc3c2)c1 | Scaffold-4 |
| SJ000311323-1 | 17.6036 | COc1cccc(CNCCCNc2ccnc3cc(Cc4ccc(F)cc4Cl)ccc23)c1O | Scaffold-4 |
| SJ000311325-1 | 17.6063 | COc1ccc(F)cc1CNCCCNc2ccnc3cc(Cc4ccc(F)cc4Cl)ccc23 | Scaffold-4 |
| SJ000311228-1 | 17.6847 | Clc1ccc(Oc2ccc3c(NCCCNCc4cccc5OCOc45)ccnc3c2)cc1 | Scaffold-4 |
| SJ000311309-1 | 18.2808 | COc1ccc(F)cc1CNCCCNc2ccnc3cc(Cc4ccccc4)ccc23 | Scaffold-4 |
| SJ000311229-1 | 19.6597 | COc1ccc(F)cc1CNCCCNc2ccnc3cc(Oc4ccc(Cl)cc4)ccc23 | Scaffold-4 |
| SJ000311324-1 | 20.6175 | Fc1ccc(Cc2ccc3c(NCCCNCc4cccc5OCOc45)ccnc3c2)c(Cl)c1 | Scaffold-4 |
| SJ000311227-1 | 20.7039 | COc1cccc(CNCCCNc2ccnc3cc(Oc4ccc(Cl)cc4)ccc23)c1O | Scaffold-4 |
| SJ000311301-2 | 22.1465 | COc1ccc(F)cc1CNCCCNc2ccnc3cc(Cc4cccc(c4)C(F)(F)F)ccc23 | Scaffold-4 |
| SJ000311284-1 | 22.2392 | C(CNCc1cccc2OCOc12)CNc3ccnc4cc(ccc34)c5ccccc5 | Scaffold-4 |
| SJ000311319-1 | 23.6134 | COc1cccc(CNCCCNc2ccnc3cc(Cc4cc(C)cc(C)c4)ccc23)c1O | Scaffold-4 |
| SJ000311264-1 | 23.7397 | C(CNCc1cccc2OCOc12)CNc3ccnc4cc(ccc34)C5CCCCC5 | Scaffold-4 |
| SJ000311283-1 | 24.123 | COc1cccc(CNCCCNc2ccnc3cc(ccc23)c4ccccc4)c1O | Scaffold-4 |
| SJ000311265-1 | 24.1611 | COc1ccc(F)cc1CNCCCNc2ccnc3cc(ccc23)C4CCCCC4 | Scaffold-4 |
| SJ000311291-1 | 24.5885 | COc1cccc(CNCCCNc2ccnc3cc(ccc23)c4ccc5OCOc5c4)c1O | Scaffold-4 |
| SJ000311240-1 | 24.9735 | Fc1ccc(cc1)c2ccc3c(NCCCNCc4cccc5OCOc45)ccnc3c2 | Scaffold-4 |
| SJ000311253-1 | 26.2563 | COc1ccc(F)cc1CNCCCNc2ccnc3cc(CC4CCCCC4)ccc23 | Scaffold-4 |
| SJ000311255-1 | 26.5454 | COc1cccc(CNCCCNc2ccnc3cc(CCc4ccccc4)ccc23)c1O | Scaffold-4 |
| SJ000311299-1 | 27.471 | COc1cccc(CNCCCNc2ccnc3cc(Cc4cccc(c4)C(F)(F)F)ccc23)c1O | Scaffold-4 |
| SJ000311248-1 | 27.6895 | CC(C)CCc1ccc2c(NCCCNCc3cccc4OCOc34)ccnc2c1 | Scaffold-4 |
| SJ000311285-1 | 28.2291 | COc1ccc(F)cc1CNCCCNc2ccnc3cc(ccc23)c4ccccc4 | Scaffold-4 |
| SJ000311268-1 | 28.938 | CCC(CC)c1ccc2c(NCCCNCc3cccc4OCOc34)ccnc2c1 | Scaffold-4 |
| SJ000311209-1 | 28.9587 | COc1ccc(F)cc1CNCCCNc2ccnc3cc(Oc4ccccc4)ccc23 | Scaffold-4 |
| SJ000311219-1 | 29.046 | COc1ccc(Oc2ccc3c(NCCCNCc4cccc(OC)c4O)ccnc3c2)cc1 | Scaffold-4 |
| SJ000311251-1 | 29.238 | COc1cccc(CNCCCNc2ccnc3cc(CC4CCCCC4)ccc23)c1O | Scaffold-4 |
| SJ000311293-1 | 29.8651 | COc1ccc(F)cc1CNCCCNc2ccnc3cc(ccc23)c4ccc5OCOc5c4 | Scaffold-4 |
| SJ000311208-1 | 30.0953 | C(CNCc1cccc2OCOc12)CNc3ccnc4cc(Oc5ccccc5)ccc34 | Scaffold-4 |
| SJ000311193-1 | 30.449 | ClC1═CC2═NC═CC(NCCCNCC3═C(O)C═CC(C(C)(C)C)═C3)═C2C═C1 | Scaffold-4 |
| SJ000311292-1 | 30.4912 | C(CNCc1cccc2OCOc12)CNc3ccnc4cc(ccc34)c5ccc6OCOc6c5 | Scaffold-4 |
| SJ000311276-1 | 31.1901 | C(CNCc1cccc2OCOc12)CNc3ccnc4cc(ccc34)c5cccc6ccccc56 | Scaffold-4 |
| SJ000311312-1 | 31.2742 | COc1cc(Cc2ccc3c(NCCCNCc4cccc5OCOc45)ccnc3c2)cc(OC)c1 | Scaffold-4 |
| SJ000311263-1 | 32.3955 | COc1cccc(CNCCCNc2ccnc3cc(ccc23)C4CCCCC4)c1O | Scaffold-4 |
| SJ000311239-1 | 33.2974 | COc1cccc(CNCCCNc2ccnc3cc(ccc23)c4ccc(F)cc4)c1O | Scaffold-4 |
| SJ000311311-1 | 33.6057 | COc1cc(Cc2ccc3c(NCCCNCc4cccc(OC)c4O)ccnc3c2)cc(OC)c1 | Scaffold-4 |
| SJ000311313-1 | 33.7701 | COc1cc(Cc2ccc3c(NCCCNCc4cc(F)ccc4OC)ccnc3c2)cc(OC)c1 | Scaffold-4 |
| SJ000523064-1 | 34.8574 | Clc1ccc2c(NCCCNCc3ccc(cc3)c4ccccc4)ccnc2c1 | Scaffold-4 |
| SJ000311245-1 | 35.052 | COc1ccc(F)cc1CNCCCNc2ccnc3cc(Cc4ccc(F)cc4)ccc23 | Scaffold-4 |
| SJ000311217-1 | 36.7893 | COc1cccc(Oc2ccc3c(NCCCNCc4cc(F)ccc4OC)ccnc3c2)c1 | Scaffold-4 |
| SJ000311216-1 | 36.9006 | COc1cccc(Oc2ccc3c(NCCCNCc4cccc5OCOc45)ccnc3c2)c1 | Scaffold-4 |
| SJ000311256-1 | 37.0782 | COc1ccc(F)cc1CNCCCNc2ccnc3cc(CCc4ccccc4)ccc23 | Scaffold-4 |
| SJ000311241-1 | 38.8868 | COc1ccc(F)cc1CNCCCNc2ccnc3cc(ccc23)c4ccc(F)cc4 | Scaffold-4 |
| SJ000521067-1 | 40.8529 | CCc1ccc(CNCCCNc2ccnc3cc(ccc23)C(F)(F)F)cc1 | Scaffold-4 |
| SJ000311244-1 | 42.2755 | Fc1ccc(Cc2ccc3c(NCCCNCc4cccc5OCOc45)ccnc3c2)cc1 | Scaffold-4 |
| SJ000311269-1 | 42.9599 | CCC(CC)c1ccc2c(NCCCNCc3cc(F)ccc3OC)ccnc2c1 | Scaffold-4 |
| SJ000311243-1 | 43.553 | COc1cccc(CNCCCNc2ccnc3cc(Cc4ccc(F)cc4)ccc23)c1O | Scaffold-4 |
| SJ000311220-1 | 43.7389 | COc1ccc(Oc(cc2)cc3c2c(NCCCNCc4c5c(OCO5)ccc4)ccn3)cc1 | Scaffold-4 |
| SJ000311308-1 | 46.1014 | C(CNCc1cccc2OCOc12)CNc3ccnc4cc(Cc5ccccc5)ccc34 | Scaffold-4 |
| SJ000311221-1 | 46.446 | COc1ccc(Oc2ccc3c(NCCCNCc4cc(F)ccc4OC)ccnc3c2)cc1 | Scaffold-4 |
| SJ000311303-1 | 50.9933 | COc1cccc(CNCCCNc2ccnc3cc(Cc4ccc(cc4)C#N)ccc23)c1O | Scaffold-4 |
| SJ000311224-1 | 59.5758 | Fc1ccc(Oc2ccc3c(NCCCNCc4cccc5OCOc45)ccnc3c2)cc1 | Scaffold-4 |
| SJ000311223-1 | 61.8706 | COc1cccc(CNCCCNc2ccnc3cc(Oc4ccc(F)cc4)ccc23)c1O | Scaffold-4 |
| SJ000311257-1 | 62.259 | C(CNCc1cccc2OCOc12)CNc3ccnc4cc(CCc5ccccc5)ccc34 | Scaffold-4 |
| SJ000311304-1 | 65.975 | N#Cc1ccc(Cc2ccc3c(NCCCNCc4cccc5OCOc45)ccnc3c2)cc1 | Scaffold-4 |
| SJ000520909-2 | 67.1033 | Clc1ccc2c(NCCCNCc3ccc4[nH]ccc4c3)ccnc2c1 | Scaffold-4 |
| SJ000520911-2 | 68.1168 | Cc1cc(C)c(CNCCCNc2ccnc3cc(Cl)ccc23)c(C)c1 | Scaffold-4 |
| SJ000311259-1 | 68.501 | COc1cccc(CNCCCNc2ccnc3cc(CC(C)C)ccc23)c1O | Scaffold-4 |
| SJ000521064-1 | 69.1347 | COc1ccc(cc1CNCCCNc2ccnc3cc(ccc23)C(F)(F)F)C(F)(F)F | Scaffold-4 |
| SJ000000500-3 | 69.9301 | COc1ccc(F)cc1CNCCCNc2ccnc3cc(Cl)ccc23 | Scaffold-4 |
| SJ000000501-3 | 69.9301 | Clc1ccc2c(NCCCNCc3cccc4OCOc34)ccnc2c1 | Scaffold-4 |
| SJ000311185-1 | 69.9301 | ClC1═CC2═NC═CC(NCCCNCC3═C(OC)C═CC═C3)═C2C═C1 | Scaffold-4 |
| SJ000311186-1 | 69.9301 | ClC1═CC2═NC═CC(NCCCNCC3═C(O)C═CC(Br)═C3)═C2C═C1 | Scaffold-4 |
| SJ000311187-1 | 69.9301 | ClC1═CC2═NC═CC(NCCCNCC3═C(O)C═CC═C3)═C2C═C1 | Scaffold-4 |
| SJ000311188-1 | 69.9301 | ClC1═CC2═NC═CC(NCCCNC(C)C3═C(O)C═CC(OC)═C3)═C2C═C1 | Scaffold-4 |
| SJ000311191-1 | 69.9301 | ClC1═CC2═NC═CC(NCCCNCC3═CC═C([N+]([O−])═O)C(O)═C3)═C2C═C1 | Scaffold-4 |
| SJ000311192-1 | 69.9301 | ClC1═CC2═NC═CC(NCCCNCC3═CC═C(OC)C(O)═C3)═C2C═C1 | Scaffold-4 |
| SJ000311194-1 | 69.9301 | ClC1═CC2═NC═CC(NCCCNCC3═C(Br)C═CC(O)═C3)═C2C═C1 | Scaffold-4 |
| SJ000311196-1 | 69.9301 | ClC1═CC2═NC═CC(NCCCCN(CCC)CC3═CC(Br)═CC═C3O)═C2C═C1 | Scaffold-4 |
| SJ000311207-1 | 69.9301 | COc1cccc(CNCCCNc2ccnc3cc(Oc4ccccc4)ccc23)c1O | Scaffold-4 |
| SJ000311211-1 | 69.9301 | COc1cccc(CNCCCNc2ccnc3cc(Oc4ccccc4OC)ccc23)c1O | Scaffold-4 |
| SJ000311212-1 | 69.9301 | COc1ccccc1Oc2ccc3c(NCCCNCc4cccc5OCOc45)ccnc3c2 | Scaffold-4 |
| SJ000311213-1 | 69.9301 | COc1ccc(F)cc1CNCCCNc2ccnc3cc(Oc4ccccc4OC)ccc23 | Scaffold-4 |
| SJ000311215-1 | 69.9301 | COc1cccc(Oc2ccc3c(NCCCNCc4cccc(OC)c4O)ccnc3c2)c1 | Scaffold-4 |
| SJ000311225-1 | 69.9301 | COc1ccc(F)cc1CNCCCNc2ccnc3cc(Oc4ccc(F)cc4)ccc23 | Scaffold-4 |
| SJ000311231-1 | 69.9301 | COc1cccc(CNCCCNc2ccnc3cc(Oc4cccc(c4)N(C)C)ccc23)c1O | Scaffold-4 |
| SJ000311232-1 | 69.9301 | CN(C)c1cccc(Oc2ccc3c(NCCCNCc4cccc5OCOc45)ccnc3c2)c1 | Scaffold-4 |
| SJ000311247-1 | 69.9301 | COc1cccc(CNCCCNc2ccnc3cc(CCC(C)C)ccc23)c1O | Scaffold-4 |
| SJ000311249-1 | 69.9301 | COc1ccc(F)cc1CNCCCNc2ccnc3cc(CCC(C)C)ccc23 | Scaffold-4 |
| SJ000311260-1 | 69.9301 | CC(C)Cc1ccc2c(NCCCNCc3cccc4OCOc34)ccnc2c1 | Scaffold-4 |
| SJ000311261-1 | 69.9301 | COc1ccc(F)cc1CNCCCNc2ccnc3cc(CC(C)C)ccc23 | Scaffold-4 |
| SJ000311271-1 | 69.9301 | COc1cccc(CNCCCNc2ccnc3cc(ccc23)c4cc(cc(c4)C(F)(F)F)C(F)(F)F)c1O | Scaffold-4 |
| SJ000311295-1 | 69.9301 | COc1ccc(cc1)c2ccc3c(NCCCNCc4cccc(OC)c4O)ccnc3c2 | Scaffold-4 |
| SJ000311296-1 | 69.9301 | COc1ccc(cc1)c2ccc3c(NCCCNCc4cccc5OCOc45)ccnc3c2 | Scaffold-4 |
| SJ000311297-1 | 69.9301 | COc1ccc(cc1)c2ccc3c(NCCCNCc4cc(F)ccc4OC)ccnc3c2 | Scaffold-4 |
| SJ000311305-1 | 69.9301 | COc1ccc(F)cc1CNCCCNc2ccnc3cc(Cc4ccc(cc4)C#N)ccc23 | Scaffold-4 |
| SJ000311307-1 | 69.9301 | COc1cccc(CNCCCNc2ccnc3cc(Cc4ccccc4)ccc23)c1O | Scaffold-4 |
| SJ000311328-2 | 69.9301 | COc1cccc(F)c1CNCCCNc2ccnc3cc(Cl)ccc23 | Scaffold-4 |
| SJ000361100-1 | 69.9301 | Clc1ccc2c(NCCCNCc3ccccc3)ccnc2c1 | Scaffold-4 |
| SJ000361106-1 | 69.9301 | FC(F)(F)c1ccc2c(NCCCNCc3ccc4OCOc4c3)ccnc2c1 | Scaffold-4 |
| SJ000361107-1 | 69.9301 | FC(F)(F)c1ccc2c(NCCCNCc3ccccc3)ccnc2c1 | Scaffold-4 |
| SJ000361122-2 | 69.9301 | Clc1ccc2c(NCCCNCc3c4ccccc4cc5ccccc35)ccnc2c1 | Scaffold-4 |
| SJ000361126-1 | 69.9301 | FC(F)(F)c1ccc2c(NCCCNCc3c4ccccc4cc5ccccc35)ccnc2c1 | Scaffold-4 |
| SJ000520908-2 | 69.9301 | FC(F)(F)c1ccccc1CNCCCNc2ccnc3cc(Cl)ccc23 | Scaffold-4 |
| SJ000520910-1 | 69.9301 | Fc1c(F)c(F)c(CNCCCNc2ccnc3cc(Cl)ccc23)c(F)c1F | Scaffold-4 |
| SJ000521022-2 | 69.9301 | FC(F)(F)Oc1cccc(CNCCCNc2ccnc3cc(Cl)ccc23)c1 | Scaffold-4 |
| SJ000521024-1 | 69.9301 | FC(F)(F)c1cccc(CNCCCNc2ccnc3cc(Cl)ccc23)c1 | Scaffold-4 |
| SJ000521025-1 | 69.9301 | FC(F)(F)Oc1ccc(CNCCCNc2ccnc3cc(Cl)ccc23)cc1 | Scaffold-4 |
| SJ000521030-1 | 69.9301 | COc1ccc(F)c(CNCCCNc2ccnc3cc(Cl)ccc23)c1 | Scaffold-4 |
| SJ000521031-1 | 69.9301 | Fc1cccc(c1CNCCCNc2ccnc3cc(Cl)ccc23)C(F)(F)F | Scaffold-4 |
| SJ000521035-1 | 69.9301 | COc1ccc(OC(F)(F)F)cc1CNCCCNc2ccnc3cc(Cl)ccc23 | Scaffold-4 |
| SJ000521036-1 | 69.9301 | COc1ccc(cc1CNCCCNc2ccnc3cc(Cl)ccc23)C(F)(F)F | Scaffold-4 |
| SJ000521039-1 | 69.9301 | CCc1ccc(CNCCCNc2ccnc3cc(Cl)ccc23)cc1 | Scaffold-4 |
| SJ000521045-2 | 69.9301 | FC(F)(F)Oc1cccc(CNCCCNc2ccnc3cc(ccc23)C(F)(F)F)c1 | Scaffold-4 |
| SJ000521047-1 | 69.9301 | FC(F)(F)c1cccc(CNCCCNc2ccnc3cc(ccc23)C(F)(F)F)c1 | Scaffold-4 |
| SJ000521048-1 | 69.9301 | FC(F)(F)c1ccc2c(NCCCNCc3cccc4OCOc34)ccnc2c1 | Scaffold-4 |
| SJ000521049-1 | 69.9301 | FC(F)(F)c1ccc2c(NCCCNCc3ccccc3C(F)(F)F)ccnc2c1 | Scaffold-4 |
| SJ000521050-1 | 69.9301 | FC(F)(F)Oc1ccc(CNCCCNc2ccnc3cc(ccc23)C(F)(F)F)cc1 | Scaffold-4 |
| SJ000521051-1 | 69.9301 | COc1cccc(F)c1CNCCCNc2ccnc3cc(ccc23)C(F)(F)F | Scaffold-4 |
| SJ000521052-1 | 69.9301 | COc1ccc(F)cc1CNCCCNc2ccnc3cc(ccc23)C(F)(F)F | Scaffold-4 |
| SJ000521058-1 | 69.9301 | COc1ccc(F)c(CNCCCNc2ccnc3cc(ccc23)C(F)(F)F)c1 | Scaffold-4 |
| SJ000521059-1 | 69.9301 | Fc1cccc(c1CNCCCNc2ccnc3cc(ccc23)C(F)(F)F)C(F)(F)F | Scaffold-4 |
| SJ000521063-1 | 69.9301 | COc1ccc(OC(F)(F)F)cc1CNCCCNc2ccnc3cc(ccc23)C(F)(F)F | Scaffold-4 |
| SJ000521066-1 | 69.9301 | Fc1cccc(CNCCCNc2ccnc3cc(ccc23)C(F)(F)F)c1 | Scaffold-4 |
| SJ000523054-1 | 69.9301 | CN(C)c1ccc(CNCCCNc2ccnc3cc(Cl)ccc23)c4ccccc14 | Scaffold-4 |
| SJ000523055-1 | 69.9301 | Nc1ccccc1CNCCCNc2ccnc3cc(Cl)ccc23 | Scaffold-4 |
| SJ000523056-1 | 69.9301 | Clc1ccc2c(NCCCNCc3ccc4OCOc4c3)ccnc2c1 | Scaffold-4 |
| SJ000523059-1 | 69.9301 | COc1ccc(CNCCCNc2ccnc3cc(Cl)ccc23)cc1 | Scaffold-4 |
| SJ000523060-1 | 69.9301 | Fc1ccccc1CNCCCNc2ccnc3cc(Cl)ccc23 | Scaffold-4 |
| SJ000523061-1 | 69.9301 | Fc1cccc(CNCCCNc2ccnc3cc(Cl)ccc23)c1 | Scaffold-4 |
| SJ000523062-1 | 69.9301 | Fc1ccc(CNCCCNc2ccnc3cc(Cl)ccc23)cc1 | Scaffold-4 |
| SJ000523063-1 | 69.9301 | Clc1ccc2c(NCCCNCc3cccc(c3)C#N)ccnc2c1 | Scaffold-4 |
| SJ000523065-1 | 69.9301 | Clc1ccc2c(NCCCNCc3ccc4ccccc4c3)ccnc2c1 | Scaffold-4 |
| SJ000523066-1 | 69.9301 | Cc1cccc(CNCCCNc2ccnc3cc(Cl)ccc23)c1 | Scaffold-4 |
| SJ000523067-1 | 69.9301 | COC(═O)c1ccc(CNCCCNc2ccnc3cc(Cl)ccc23)cc1 | Scaffold-4 |
| SJ000456069-1 | 0.8605 | CC(═C)CSC1═Nc2cc(sc2C(═O)N1CCN3CCOCC3)c4ccccc4 | Scaffold-5 |
| SJ000296471-1 | 69.9301 | CC1═Nc2cc(sc2C(═O)N1N)c3ccccc3 | Scaffold-5 |
| SJ000296726-1 | 69.9301 | COc1ccc(cc1)c2cc3N═CNC(═O)c3s2 | Scaffold-5 |
| SJ000395304-1 | 69.9301 | COCCNC(═O)CN1C═Nc2cc(sc2C1═O)c3ccc(Cl)cc3 | Scaffold-5 |
| SJ000397503-1 | 69.9301 | COc1ccc(cc1)c2cc3N═CN(CC(═O)O)C(═O)c3s2 | Scaffold-5 |
| SJ000397713-1 | 69.9301 | Brc1ccc(cc1)c2cc3N═CN(CCC#N)C(═O)c3s2 | Scaffold-5 |
| SJ000399852-1 | 69.9301 | CCCCOC(═O)CN1C═Nc2cc(sc2C1═O)c3ccc(OC)cc3 | Scaffold-5 |
| SJ000399862-1 | 69.9301 | CCOC(═O)CN1C═Nc2cc(sc2C1═O)c3ccc(OC)cc3 | Scaffold-5 |
| SJ000405932-1 | 69.9301 | CSC1═Nc2cc(sc2C(═O)N1CCN3CCOCC3)c4ccccc4 | Scaffold-5 |
| SJ000418500-1 | 69.9301 | COC(═O)CN1C═Nc2cc(sc2C1═O)c3ccc(Cl)cc3 | Scaffold-5 |
| SJ000425388-1 | 69.9301 | CC(C)NC(═O)NC(═O)CN1C═Nc2cc(sc2C1═O)c3ccccc3 | Scaffold-5 |
| SJ000425603-1 | 69.9301 | CN(C)C(═O)CN1C═Nc2cc(sc2C1═O)c3ccccc3 | Scaffold-5 |
| SJ000425613-1 | 69.9301 | CCC(C)(C)NC(═O)CN1C═Nc2cc(sc2C1═O)c3ccccc3 | Scaffold-5 |
| SJ000425666-1 | 69.9301 | CCN(CC)C(═O)CN1C═Nc2cc(sc2C1═O)c3ccccc3 | Scaffold-5 |
| SJ000426777-1 | 69.9301 | C═CCNC(═O)NC(═O)CN1C═Nc2cc(sc2C1═O)c3ccccc3 | Scaffold-5 |
| SJ000427828-1 | 69.9301 | COCC(C)NC(═O)CN1C═Nc2cc(sc2C1═O)c3ccc(Br)cc3 | Scaffold-5 |
| SJ000427956-1 | 69.9301 | CC(C)(C)NC(═O)NC(═O)CN1C═Nc2cc(sc2C1═O)c3ccccc3 | Scaffold-5 |
| SJ000429044-1 | 69.9301 | CCN(CC(═C)C)C(═O)CN1C═Nc2cc(sc2C1═O)c3ccc(OC)cc3 | Scaffold-5 |
| SJ000431447-1 | 69.9301 | COc1ccc(cc1)c2cc3N═CN(CC(═O)N(CC═C)CC═C)C(═O)c3s2 | Scaffold-5 |
| SJ000441316-1 | 69.9301 | COc1ccc(CNC(═O)CN2C═Nc3cc(sc3C2═O)c4ccc(OC)cc4)cc1 | Scaffold-5 |
| SJ000443969-1 | 69.9301 | COc1ccc(cc1)c2cc3N═CN(CC(═O)NCc4ccc(F)cc4)C(═O)c3s2 | Scaffold-5 |
| SJ000444020-1 | 69.9301 | Fc1ccc(CNC(═O)CN2C═Nc3cc(sc3C2═O)c4ccc(Cl)cc4)cc1 | Scaffold-5 |
| SJ000444040-1 | 69.9301 | COc1ccccc1CNC(═O)CN2C═Nc3cc(sc3C2═O)c4ccc(Cl)cc4 | Scaffold-5 |
| SJ000445272-1 | 69.9301 | Cc1ccc(CNC(═O)CN2C═Nc3cc(sc3C2═O)c4ccc(Cl)cc4)cc1 | Scaffold-5 |
| SJ000445282-1 | 69.9301 | COc1ccc(cc1)c2cc3N═CN(CC(═O)NCc4ccc(C)cc4)C(═O)c3s2 | Scaffold-5 |
| SJ000448314-1 | 69.9301 | CC(═O)CN1C═Nc2cc(sc2C1═O)c3ccccc3 | Scaffold-5 |
| SJ000449696-1 | 69.9301 | COc1ccc(cc1)c2cc3N═CN(CC(═O)NCc4ccccc4F)C(═O)c3s2 | Scaffold-5 |
| SJ000449845-1 | 69.9301 | COc1ccc(cc1)c2cc3N═CN(CC(═O)NCc4ccccc4Cl)C(═O)c3s2 | Scaffold-5 |
| SJ000451551-1 | 69.9301 | COC(═O)CSC1═Nc2cc(sc2C(═O)N1CCN3CCOCC3)c4ccccc4 | Scaffold-5 |
| SJ000451590-1 | 69.9301 | CC(C)(C)C(═O)CSC1═Nc2cc(sc2C(═O)N1CCN3CCOCC3)c4ccccc4 | Scaffold-5 |
| SJ000452125-1 | 69.9301 | COc1ccccc1CNC(═O)CN2C═Nc3cc(sc3C2═O)c4ccccc4 | Scaffold-5 |
| SJ000452142-1 | 69.9301 | CCCN1C═Nc2cc(sc2C1═O)c3ccc(Cl)cc3 | Scaffold-5 |
| SJ000452798-1 | 69.9301 | OCCSC1═Nc2cc(sc2C(═O)N1CCN3CCOCC3)c4ccccc4 | Scaffold-5 |
| SJ000453315-1 | 69.9301 | Cc1ccccc1CNC(═O)CN2C═Nc3cc(sc3C2═O)c4ccccc4 | Scaffold-5 |
| SJ000453419-1 | 69.9301 | CCCSC1═Nc2cc(sc2C(═O)N1CCN3CCOCC3)c4ccccc4 | Scaffold-5 |
| SJ000453564-1 | 69.9301 | C═CCSC1═Nc2cc(sc2C(═O)N1CCN3CCOCC3)c4ccccc4 | Scaffold-5 |
| SJ000455215-1 | 69.9301 | COc1ccc(cc1)c2cc3N═CN(CC(═O)NCc4ccccc4C)C(═O)c3s2 | Scaffold-5 |
| SJ000455225-1 | 69.9301 | Fc1ccccc1CNC(═O)CN2C═Nc3cc(sc3C2═O)c4ccc(Cl)cc4 | Scaffold-5 |
| SJ000455850-1 | 69.9301 | Clc1ccccc1CNC(═O)CN2C═Nc3cc(sc3C2═O)c4ccccc4 | Scaffold-5 |
| SJ000456118-1 | 69.9301 | FC(F)(F)Oc1ccccc1CNC(═O)CN2C═Nc3cc(sc3C2═O)c4ccccc4 | Scaffold-5 |
| SJ000456507-1 | 69.9301 | CC(═C)CN1C═Nc2cc(sc2C1═O)c3ccc(Cl)cc3 | Scaffold-5 |
| SJ000457620-1 | 69.9301 | COc1ccc(CNC(═O)CN2C═Nc3cc(sc3C2═O)c4ccc(Cl)cc4)cc1 | Scaffold-5 |
| SJ000457991-1 | 69.9301 | COc1ccc(cc1)c2cc3N═CN(CC(═O)NCc4ccc(Cl)cc4)C(═O)c3s2 | Scaffold-5 |
| SJ000458551-1 | 69.9301 | Fc1ccc(cc1)c2cc3N═CN(CCC#N)C(═O)c3s2 | Scaffold-5 |
| SJ000167951-1 | 19.1588 | CCCS(═O)(═O)N1CCN(Cc2ccc3c(c2)c4ccccc4n3CC)CC1 | Scaffold-6 |
| SJ000147352-1 | 29.4772 | CCOC(═O)N1CCN(Cc2ccc3c(c2)c4ccccc4n3CC)CC1 | Scaffold-6 |
| SJ000031064-1 | 69.9301 | OCCNC(═O)CNC(═O)c1ccc2[nH]c3ccccc3c2c1 | Scaffold-6 |
| SJ000031065-1 | 69.9301 | OCCNC(═O)c1ccc2[nH]c3ccccc3c2c1 | Scaffold-6 |
| SJ000053810-1 | 69.9301 | OCC(O)CNC(═O)c1ccc2[nH]c3ccccc3c2c1 | Scaffold-6 |
| SJ000053962-1 | 69.9301 | OCCNCCNCc1ccc2[nH]c3ccccc3c2c1 | Scaffold-6 |
| SJ000054243-1 | 69.9301 | CN(C)CCCNCc1ccc2[nH]c3ccccc3c2c1 | Scaffold-6 |
| SJ000111264-1 | 69.9301 | CCn1c2ccccc2c3cc(CN4CCN(Cc5c[nH]c6ccccc56)CC4)ccc13 | Scaffold-6 |
| SJ000111712-1 | 69.9301 | CCn1c2ccccc2c3cc(CNCCc4ccccc4)ccc13 | Scaffold-6 |
| SJ000111875-2 | 69.9301 | CCn1c2ccccc2c3cc(CN4CCN(Cc5cc(OC)ccc5O)CC4)ccc13 | Scaffold-6 |
| SJ000112430-1 | 69.9301 | CCn1c2ccccc2c3cc(CNCc4ccc(OC)cc4)ccc13 | Scaffold-6 |
| SJ000112458-1 | 69.9301 | CCn1c2ccccc2c3cc(CNCCc4ccc(Cl)cc4)ccc13 | Scaffold-6 |
| SJ000112461-1 | 69.9301 | CCn1c2ccccc2c3cc(CNCCc4ccc5OCOc5c4)ccc13 | Scaffold-6 |
| SJ000112860-1 | 69.9301 | CCn1c2ccccc2c3cc(CN4CCC(CC4)N5CCN(CC5)c6ccccc6)ccc13 | Scaffold-6 |
| SJ000123281-1 | 69.9301 | CN(C)CCCNC(═O)c1ccc2[nH]c3ccccc3c2c1 | Scaffold-6 |
| SJ000142875-1 | 69.9301 | CCN1CCN(Cc2ccc3c(c2)c4ccccc4n3CC)CC1 | Scaffold-6 |
| SJ000142908-1 | 69.9301 | CCn1c2ccccc2c3cc(CN4CCN(C)CC4)ccc13 | Scaffold-6 |
| SJ000145592-1 | 69.9301 | CCn1c2ccccc2c3cc(CN4CCN(CCO)CC4)ccc13 | Scaffold-6 |
| SJ000168261-1 | 69.9301 | CCn1c2ccccc2c3cc(CN4CCN(CC4)S(═O)(═O)C)ccc13 | Scaffold-6 |
| SJ000168321-1 | 69.9301 | CCn1c2ccccc2c3cc(CN4CCN(CC4)S(═O)(═O)CC)ccc13 | Scaffold-6 |
| SJ000288408-1 | 69.9301 | Cn1c2ccccc2c3cc(ccc13)C4Nc5ccccc5C(═O)N4Cc6ccccc6 | Scaffold-6 |
| SJ000298589-1 | 69.9301 | CCn1c2ccccc2c3cc(CN4CCN(CC4)C(═O)c5ccc(OC)c(OC)c5)ccc13 | Scaffold-6 |
| SJ000307416-1 | 69.9301 | Cn1c2ccccc2c3cc(ccc13)C(═O)N4CCc5nncn5CC4 | Scaffold-6 |
| SJ000307753-1 | 69.9301 | CCn1c2ccccc2c3cc(CN4CCc5nnc(CNS(═O)(═O)C(C)C)n5CC4)ccc13 | Scaffold-6 |
| SJ000307915-1 | 69.9301 | CCn1c2ccccc2c3cc(CN4CCc5nnc(C(C)NC(═O)CCOC)n5CC4)ccc13 | Scaffold-6 |
| SJ000307948-1 | 69.9301 | CCn1c2ccccc2c3cc(CN4CCc5nnc(CNC(═O)c6ccccc6)n5CC4)ccc13 | Scaffold-6 |
| SJ000308489-1 | 69.9301 | Cn1c2ccccc2c3cc(ccc13)C(═O)NCC4CCC5(CCN(Cc6ccccn6)CC5)O4 | Scaffold-6 |
| SJ000461804-1 | 69.9301 | CC(NC═O)c1ccc2c(c1)c3ccccc3n2C | Scaffold-6 |
| SJ000461805-1 | 69.9301 | CCn1c2ccccc2c3cc(ccc13)C(C)NC═O | Scaffold-6 |
| SJ000505727-1 | 69.9301 | CCn1c2ccccc2c3cc(CNCC(C)O)ccc13 | Scaffold-6 |
| SJ000516830-1 | 69.9301 | CN(Cc1oncc1)C(═O)c2ccc3c(c2)c4ccccc4n3C | Scaffold-6 |
| SJ000550865-1 | 69.9301 | CCn1c2ccccc2c3cc(CNC4CCCCC4)ccc13 | Scaffold-6 |
| SJ000550874-1 | 69.9301 | CCn1c2ccccc2c3cc(CN4CCCC(O)C4)ccc13 | Scaffold-6 |
| SJ000230856-1 | 4.337 | Cc1ccc(cc1)S(═O)(═O)c2nc(oc2N3CCOCC3)c4ccccc4Cl | Scaffold-8 |
| SJ000038124-1 | 7.4311 | COc1cccc(c1)c2oc(N3CCOCC3)c(n2)S(═O)(═O)c4ccc(Cl)cc4 | Scaffold-8 |
| SJ000244320-1 | 8.6307 | Brc1ccc(cc1)c2oc(N3CCOCC3)c(n2)S(═O)(═O)c4ccccc4 | Scaffold-8 |
| SJ000027481-1 | 9.2039 | Clc1ccc(cc1)S(═O)(═O)c2nc(oc2N3CCOCC3)c4cccc(Br)c4 | Scaffold-8 |
| SJ000027482-1 | 9.538 | Cc1ccc(cc1)S(═O)(═O)c2nc(oc2N3CCOCC3)c4cccc(c4)[N+](═O)[O−] | Scaffold-8 |
| SJ000027480-1 | 11.2321 | Cc1ccc(cc1)S(═O)(═O)c2nc(oc2N3CCOCC3)c4cccc(C)c4 | Scaffold-8 |
| SJ000230854-1 | 11.6729 | Cc1ccc(cc1)S(═O)(═O)c2nc(oc2N3CCOCC3)c4ccc(F)cc4 | Scaffold-8 |
| SJ000329041-1 | 11.8298 | CC1CN(CC(C)O1)c2oc(nc2S(═O)(═O)c3ccc(C)cc3)c4ccccc4C | Scaffold-8 |
| SJ000329066-1 | 13.0083 | CC1CN(CC(C)O1)c2oc(nc2S(═O)(═O)c3ccc(C)cc3)c4ccccc4Cl | Scaffold-8 |
| SJ000090185-1 | 17.3583 | COP(═O)(OC)c1nc(oc1N2CCOCC2)c3ccc(Cl)cc3 | Scaffold-8 |
| SJ000038123-1 | 17.4113 | COc1cccc(c1)c2oc(N3CCOCC3)c(n2)S(═O)(═O)c4ccc(C)cc4 | Scaffold-8 |
| SJ000329067-1 | 27.2645 | CC1CN(CC(C)O1)c2oc(nc2S(═O)(═O)c3ccc(F)cc3)c4ccccc4F | Scaffold-8 |
| SJ000010022-1 | 69.9301 | Clc1ccc(cc1)c2oc(N3CCOCC3)c(n2)C#N | Scaffold-8 |
| SJ000011204-1 | 69.9301 | Fc1ccc(cc1)c2oc(N3CCOCC3)c(n2)C#N | Scaffold-8 |
| SJ000041896-1 | 69.9301 | CCOc1ccc(cc1)c2oc(N3CCOCC3)c(n2)C#N | Scaffold-8 |
| SJ000051527-1 | 69.9301 | COc1ccc(cc1)c2oc(N3CCOCC3)c(n2)C#N | Scaffold-8 |
| SJ000090156-1 | 69.9301 | COP(═O)(OC)c1nc(oc1N2CCOCC2)c3ccc(C)cc3 | Scaffold-8 |
| SJ000090176-1 | 69.9301 | COP(═O)(OC)c1nc(oc1N2CCOCC2)c3ccc(Br)cc3 | Scaffold-8 |
| SJ000090230-1 | 69.9301 | CCOP(═O)(OCC)c1nc(oc1N2CCOCC2)c3ccc(Cl)cc3 | Scaffold-8 |
| SJ000092444-1 | 69.9301 | COP(═O)(OC)c1nc(oc1N2CCOCC2)c3ccc(cc3)[N+](═O)[O−] | Scaffold-8 |
| SJ000092459-1 | 69.9301 | CCOP(═O)(OCC)c1nc(oc1N2CCOCC2)c3ccc(cc3)[N+](═O)[O−] | Scaffold-8 |
| SJ000124835-1 | 69.9301 | COP(═O)(OC)c1nc(oc1N2CCOCC2)c3ccccc3 | Scaffold-8 |
| SJ000140851-1 | 69.9301 | Cc1ccc(cc1)c2oc(N3CCOCC3)c(n2)S(═O)(═O)c4ccc(Cl)cc4 | Scaffold-8 |
| SJ000141574-1 | 69.9301 | Clc1ccc(cc1)S(═O)(═O)c2nc(oc2N3CCOCC3)c4ccccc4Cl | Scaffold-8 |
| SJ000159484-1 | 69.9301 | Cc1ccc(cc1)c2oc(N3CCOCC3)c(n2)S(═O)(═O)c4ccccc4 | Scaffold-8 |
| SJ000184129-1 | 69.9301 | Clc1ccc(cc1)S(═O)(═O)c2nc(oc2N3CCOCC3)c4ccccc4Br | Scaffold-8 |
| SJ000185415-1 | 69.9301 | Cc1ccc(cc1)S(═O)(═O)c2nc(oc2N3CCOCC3)c4ccccc4Br | Scaffold-8 |
| SJ000198209-1 | 69.9301 | CCOc1ccc(cc1)c2oc(N3CCOCC3)c(n2)S(═O)(═O)c4ccccc4 | Scaffold-8 |
| SJ000230691-1 | 69.9301 | Clc1ccc(cc1)c2oc(N3CCOCC3)c(n2)S(═O)(═O)c4ccccc4 | Scaffold-8 |
| SJ000230692-1 | 69.9301 | Clc1ccc(cc1)c2oc(N3CCOCC3)c(n2)S(═O)(═O)c4ccc(Cl)cc4 | Scaffold-8 |
| SJ000230693-1 | 69.9301 | Cc1ccc(cc1)S(═O)(═O)c2nc(oc2N3CCOCC3)c4ccc(Cl)cc4 | Scaffold-8 |
| SJ000230855-1 | 69.9301 | Cc1ccc(cc1)S(═O)(═O)c2nc(oc2N3CCOCC3)c4ccc(Br)cc4 | Scaffold-8 |
| SJ000245271-1 | 69.9301 | COc1ccc(cc1)c2oc(N3CCOCC3)c(n2)S(═O)(═O)c4ccc(C)cc4 | Scaffold-8 |
| SJ000329031-1 | 69.9301 | CC1CN(CC(C)O1)c2oc(nc2S(═O)(═O)c3ccc(F)cc3)c4ccccc4C | Scaffold-8 |
| SJ000329051-1 | 69.9301 | CC1CN(CC(C)O1)c2oc(nc2S(═O)(═O)c3ccc(Cl)cc3)c4ccccc4C | Scaffold-8 |
| SJ000478204-1 | 69.9301 | CC1CN(CC(C)O1)c2oc(nc2C#N)c3ccc(cc3)[N+](═O)[O−] | Scaffold-8 |
| SJ000478208-1 | 69.9301 | CC1CN(CC(C)O1)c2oc(nc2C#N)c3ccc(Cl)cc3 | Scaffold-8 |
| SJ000478212-1 | 69.9301 | COc1ccc(cc1)c2oc(N3CC(C)OC(C)C3)c(n2)C#N | Scaffold-8 |
| SJ000478356-1 | 69.9301 | CCOc1ccc(cc1)c2oc(N3CC(C)OC(C)C3)c(n2)C#N | Scaffold-8 |
| SJ000478358-1 | 69.9301 | CC1CN(CC(C)O1)c2oc(nc2C#N)c3ccc(C)cc3 | Scaffold-8 |
| SJ000478453-1 | 69.9301 | CC1CN(CC(C)O1)c2oc(nc2C#N)c3ccc(F)cc3 | Scaffold-8 |

TABLE 4
Single point TR-FRET screening of 797 quinoline compounds against MAGE-A11:PCF11.

| | TR-FRET | Se- | |
|---|---|---|---|
| SAMPLE | signal | ries* | MOLSMILES |
| SJ000522746 | 96.84498178 | Q-1 | CCCN(CCCNc1ccnc2cc(Cl)ccc12)Cc3ccc4[nH]ccc4c3 |
| SJ000311286 | 73.49903728 | Q-1 | CC(C)(C)c1ccc(cc1)c2ccc3c(NCCCNCc4occc4)ccnc3c2 |
| SJ000520430 | 66.42744618 | Q-1 | Clc1ccc2c(NCCCN(Cc3ccccc3)Cc4ccc5[nH]ccc5c4)ccnc2c1 |
| SJ000311306 | 64.53702083 | Q-1 | C(CNCc1occc1)CNc2ccnc3cc(Cc4ccccc4)ccc23 |
| SJ000311230 | 63.41676877 | Q-1 | CN(C)c1cccc(Oc2ccc3c(NCCCNCc4occc4)ccnc3c2)c1 |
| SJ000311270 | 59.70593384 | Q-1 | FC(F)(F)c1cc(cc(c1)C(F)(F)F)c2ccc3c(NCCCNCc4occc4)ccnc3c2 |
| SJ000311234 | 57.53544548 | Q-1 | CC(C)(C)c1ccc(Oc2ccc3c(NCCCNCc4occc4)ccnc3c2)cc1 |
| SJ000311278 | 56.20514616 | Q-1 | FC(F)(F)c1ccc(cc1)c2ccc3c(NCCCNCc4occc4)ccnc3c2 |
| SJ000575192 | 52.99174398 | Q-1 | Clc1ccc2c(NCCCNCc3ccc[nH]3)ccnc2c1 |
| SJ000361266 | 48.15336204 | Q-1 | CCCN(CCCNc1ccnc2cc(ccc12)C(F)(F)F)CC3CC3 |
| SJ000521061 | 41.85988738 | Q-1 | FC(F)(F)c1ccc2c(NCCCNCc3csc4ccccc34)ccnc2c1 |
| SJ000361105 | 41.68865435 | Q-1 | FC(F)(F)c1ccc2c(NCCCNCc3ccc[nH]3)ccnc2c1 |
| SJ000311274 | 41.57185367 | Q-1 | C(CNCc1occc1)CNc2ccnc3cc(ccc23)c4cccc5ccccc45 |
| SJ000311250 | 40.66164887 | Q-1 | C(CNCc1occc1)CNc2ccnc3cc(CC4CCCCC4)ccc23 |
| SJ000520433 | 40.19065452 | Q-1 | Clc1ccc2c(NCCCN(Cc3ccccc3)Cc4c[nH]c5ccccc45)ccnc2c1 |
| SJ000523021 | 40.00496853 | Q-1 | CCCN(CCCNc1c2ccc(Cl)cc2nc3ccc(OC)cc13)Cc4c(Cl)ccc(Cl)c4Cl |
| SJ000522732 | 39.47138106 | Q-1 | CCCN(CCCNc1c2ccc(Cl)cc2nc3ccc(OC)cc13)Cc4ccc5[nH]ccc5c4 |
| SJ000311318 | 39.30547281 | Q-1 | Cc1cc(C)cc(Cc2ccc3c(NCCCNCc4occc4)ccnc3c2)c1 |
| SJ000521021 | 39.12130229 | Q-1 | COC(═O)CCCCCNCCCNc1ccnc2cc(Cl)ccc12 |
| SJ000311226 | 37.65097147 | Q-1 | Clc1ccc(Oc2ccc3c(NCCCNCc4occc4)ccnc3c2)cc1 |
| SJ000311218 | 36.2506564 | Q-1 | COc1ccc(Oc2ccc3c(NCCCNCc4occc4)ccnc3c2)cc1 |
| SJ000521060 | 35.63265982 | Q-1 | FC(F)(F)c1ccc2c(NCCCNCc3cc4ccccc4s3)ccnc2c1 |
| SJ000522749 | 35.43391852 | Q-1 | CCCN(CCCNc1ccnc2cc(Cl)ccc12)Cc3c(C)cc(C)cc3C |
| SJ000523083 | 34.94765512 | Q-1 | CCCCCCN(CCC)CCCNc1ccnc2cc(ccc12)C(F)(F)F |
| SJ000523069 | 33.51275257 | Q-1 | Clc1cccc(c1)c2oc(CNCCCNc3ccnc4cc(Cl)ccc34)cc2 |
| SJ000521033 | 33.38025836 | Q-1 | Clc1ccc2c(NCCCNCc3csc4ccccc34)ccnc2c1 |
| SJ000361268 | 33.31401126 | Q-1 | CCCN(CCCNc1ccnc2cc(ccc12)C(F)(F)F)CC3CCCCC3 |
| SJ000311262 | 32.95991598 | Q-1 | C(CNCc1occc1)CNc2ccnc3cc(ccc23)C4CCCCC4 |
| SJ000575171 | 32.88990023 | Q-1 | CCCN(CCCNc1ccnc2cc(ccc12)C(F)(F)F)Cc3ccc4[nH]ccc4c3 |
| SJ000523040 | 32.85028155 | Q-1 | CCCN(CCCNc1ccnc2cc(Cl)ccc12)Cc3ccc4ccccc4c3 |
| SJ000521056 | 32.71778735 | Q-1 | FC(F)(F)c1ccc2c(NCCCNCc3cc(Br)cs3)ccnc2c1 |
| SJ000523072 | 32.60983721 | Q-1 | CCCN(CCCNc1ccnc2cc(ccc12)C(F)(F)F)Cc3ccccc3OC |
| SJ000311314 | 32.39978995 | Q-1 | Clc1cccc(Cl)c1Cc2ccc3c(NCCCNCc4occc4)ccnc3c2 |
| SJ000520476 | 32.3297742 | Q-1 | CCCN(CCCNc1ccnc2cc(ccc12)C(F)(F)F)CCC(C)(C)C |
| SJ000521009 | 31.76964817 | Q-1 | CCCN(CCCNc1ccnc2cc(ccc12)C(F)(F)F)Cc3ccc(CC)cc3 |
| SJ000361124 | 31.61120095 | Q-1 | CCCCCCNCCCNc1ccnc2cc(ccc12)C(F)(F)F |
| SJ000361169 | 31.19410401 | Q-1 | Cc1cc(C)c(CN(CCCNc2ccnc3cc(Cl)ccc23)Cc4ccccc4)c(C)c1 |
| SJ000361271 | 30.93029194 | Q-1 | CCCCCN(CCC)CCCNc1ccnc2cc(ccc12)C(F)(F)F |
| SJ000311290 | 30.78942762 | Q-1 | C(CNCc1occc1)CNc2ccnc3cc(ccc23)c4ccc5OCOc5c4 |
| SJ000361255 | 30.66412719 | Q-1 | CCCN(CCCNc1ccnc2cc(ccc12)C(F)(F)F)Cc3ccc[nH]3 |
| SJ000361269 | 30.66412719 | Q-1 | CCCN(CCCNc1ccnc2cc(ccc12)C(F)(F)F)CC(CC)CC |
| SJ000523012 | 30.39913879 | Q-1 | CCCN(CCCNc1ccnc2cc(Cl)ccc12)Cc3cc(ccc3OC)C(F)(F)F |
| SJ000523039 | 30.22930159 | Q-1 | CCCN(CCCNc1ccnc2cc(Cl)ccc12)Cc3ccc(cc3)c4ccccc4 |
| SJ000521032 | 30.13415038 | Q-1 | Clc1ccc2c(NCCCNCc3cc4ccccc4s3)ccnc2c1 |
| SJ000523001 | 30.13415038 | Q-1 | CCCN(CCCNc1ccnc2cc(Cl)ccc12)Cc3ccc(CC)cc3 |
| SJ000520458 | 29.77274662 | Q-1 | Cc1ccc(F)cc1CN(CCCNc2ccnc3cc(Cl)ccc23)Cc4ccccc4 |
| SJ000361186 | 29.59915981 | Q-1 | CCC(C)CN(CCCNc1ccnc2cc(Cl)ccc12)Cc3ccccc3 |
| SJ000522992 | 29.33918516 | Q-1 | CCCN(CCCNc1ccnc2cc(Cl)ccc12)Cc3c[nH]c4ccc(F)cc34 |
| SJ000361262 | 29.22801941 | Q-1 | CCCN(CCCNc1ccnc2cc(ccc12)C(F)(F)F)Cc3c(F)cccc3OC |
| SJ000311214 | 29.10904954 | Q-1 | COc1cccc(Oc2ccc3c(NCCCNCc4occc4)ccnc3c2)c1 |
| SJ000521043 | 29.10904954 | Q-1 | Cn1cc(CNCCCNc2ccnc3cc(ccc23)C(F)(F)F)c4ccccc14 |
| SJ000361254 | 28.87545545 | Q-1 | CCCN(CCCNc1ccnc2cc(ccc12)C(F)(F)F)Cc3ccsc3 |
| SJ000361256 | 28.819474 | Q-1 | CCCN(CCCNc1ccnc2cc(ccc12)C(F)(F)F)Cc3ccccc3 |
| SJ000311282 | 28.68895501 | Q-1 | C(CNCc1occc1)CNc2ccnc3cc(ccc23)c4ccccc4 |
| SJ000361267 | 28.54892351 | Q-1 | CCCN(CCCNc1ccnc2cc(ccc12)C(F)(F)F)Cc3cocc3 |
| SJ000523041 | 28.54892351 | Q-1 | CCCN(CCCNc1ccnc2cc(Cl)ccc12)Cc3cccc(C)c3 |
| SJ000578380 | 28.47890775 | Q-1 | Clc1ccc2c(NCCCNC(═O)C[C@@H]3CC[C@@]4(CC3)OO[C@@]5(O4)C6CC7CC(CC5C7)C6)ccnc2c1 |
| SJ000361171 | 28.21298443 | Q-1 | Clc1ccc2c(NCCCN(CC3CCCCC3)Cc4ccccc4)ccnc2c1 |
| SJ000520454 | 28.13856498 | Q-1 | Clc1ccc2c(NCCCN(Cc3ccccc3)Cc4csc5ccccc45)ccnc2c1 |
| SJ000523080 | 28.00238318 | Q-1 | CCCN(CCCNc1ccnc2cc(ccc12)C(F)(F)F)CCCc3ccccc3 |
| SJ000521008 | 27.84876597 | Q-1 | CCCN(CCCNc1ccnc2cc(ccc12)C(F)(F)F)Cc3cccc(F)c3 |
| SJ000520464 | 27.77875022 | Q-1 | FC(F)(F)Oc1cccc(CN(CCCNc2ccnc3cc(Cl)ccc23)Cc4ccccc4)c1 |
| SJ000357504 | 27.63871871 | Q-1 | Oc1c(F)cc(F)cc1CN(CCCNc2ccnc3cc(Cl)ccc23)Cc4ccccc4 |
| SJ000361174 | 27.55051342 | Q-1 | Fc1ccc(CN(CCCNc2ccnc3cc(Cl)ccc23)Cc4ccccc4)cc1F |
| SJ000357461 | 27.4986872 | Q-1 | Clc1ccc2c(NCCCN(Cc3ccccc3)Cc4ccc(Br)s4)ccnc2c1 |
| SJ000521057 | 27.48426631 | Q-1 | FC(F)(F)c1ccc2c(NCCCNCc3oc4ccccc4c3)ccnc2c1 |
| SJ000311238 | 27.42867145 | Q-1 | Fc1ccc(cc1)c2ccc3c(NCCCNCc4occc4)ccnc3c2 |
| SJ000522758 | 27.41801921 | Q-1 | CCCN(CCCNc1ccnc2cc(Cl)ccc12)Cc3cccc(F)c3 |
| SJ000521053 | 27.3586557 | Q-1 | CC(C)(C)CCNCCCNc1ccnc2cc(ccc12)C(F)(F)F |
| SJ000361110 | 27.11720146 | Q-1 | FC(F)(F)c1ccc2c(NCCCNCC3CCCCC3)ccnc2c1 |
| SJ000311298 | 27.00857693 | Q-1 | FC(F)(F)c1cccc(Cc2ccc3c(NCCCNCc4occc4)ccnc3c2)c1 |
| SJ000522996 | 26.65849816 | Q-1 | CCCN(CCCNc1ccnc2cc(Cl)ccc12)Cc3cc(OC)ccc3F |
| SJ000361173 | 26.55680689 | Q-1 | CCCCCN(CCCNc1ccnc2cc(Cl)ccc12)Cc3ccccc3 |
| SJ000361257 | 26.50438335 | Q-1 | CCCN(CCCNc1ccnc2cc(ccc12)C(F)(F)F)Cc3ccccc3O |
| SJ000523070 | 26.43629245 | Q-1 | CCCN(CCCNc1ccnc2cc(ccc12)C(F)(F)F)Cc3ccc(F)cc3 |
| SJ000361181 | 26.22557138 | Q-1 | Clc1ccc2c(NCCCN(Cc3ccccc3)Cc4cccc5ccccc45)ccnc2c1 |
| SJ000523007 | 26.22557138 | Q-1 | CCCN(CCCNc1ccnc2cc(Cl)ccc12)Cc3c[nH]c4ccccc34 |
| SJ000521011 | 25.82347434 | Q-1 | CCCN(CCCNc1ccnc2cc(ccc12)C(F)(F)F)CCOCc3ccccc3 |
| SJ000523057 | 25.81830912 | Q-1 | CCCCCNCCCNc1ccnc2cc(Cl)ccc12 |
| SJ000357578 | 25.68729254 | Q-1 | CC(C)(C)CCN(CCCNc1ccnc2cc(Cl)ccc12)Cc3ccccc3 |
| SJ000520469 | 25.53824611 | Q-1 | CCCN(CCCNc1ccnc2cc(ccc12)C(F)(F)F)Cc3cn(C)c4ccccc34 |
| SJ000361184 | 25.49685326 | Q-1 | Clc1ccc(CN(CCCNc2ccnc3cc(Cl)ccc23)Cc4ccccc4)c(Cl)c1 |
| SJ000361263 | 25.25818309 | Q-1 | CCCN(CCCNc1ccnc2cc(ccc12)C(F)(F)F)Cc3cc(OC)ccc3O |
| SJ000520460 | 25.04813583 | Q-1 | FC(F)(F)Oc1ccc(CN(CCCNc2ccnc3cc(Cl)ccc23)Cc4ccccc4)cc1 |
| SJ000357573 | 24.97812008 | Q-1 | CCCN(CCCNc1ccnc2cc(Cl)ccc12)CCC(C)c3ccccc3 |
| SJ000311322 | 24.76807282 | Q-1 | Fc1ccc(Cc2ccc3c(NCCCNCc4occc4)ccnc3c2)c(Cl)c1 |
| SJ000311210 | 24.55802556 | Q-1 | COc1ccccc1Oc2ccc3c(NCCCNCc4occc4)ccnc3c2 |
| SJ000311266 | 24.55802556 | Q-1 | CCC(CC)c1ccc2c(NCCCNCc3occc3)ccnc2c1 |
| SJ000361125 | 24.46165631 | Q-1 | CSC(C)CCNCCCNc1ccnc2cc(ccc12)C(F)(F)F |
| SJ000361111 | 24.32547451 | Q-1 | CCC(CC)CNCCCNc1ccnc2cc(ccc12)C(F)(F)F |
| SJ000361265 | 24.10566413 | Q-1 | CCCN(CCCNc1ccnc2cc(ccc12)C(F)(F)F)Cc3occc3 |
| SJ000029259 | 24.0531109 | Q-1 | CN(C)CCCNc1c2CCCc2nc3ccccc13 |
| SJ000361261 | 23.90692282 | Q-1 | CCCN(CCCNc1ccnc2cc(ccc12)C(F)(F)F)Cc3oc(C)cc3 |
| SJ000311310 | 23.78785227 | Q-1 | COc1cc(Cc2ccc3c(NCCCNCc4occc4)ccnc3c2)cc(OC)c1 |
| SJ000523077 | 23.7126564 | Q-1 | CCCN(CCCNc1ccnc2cc(ccc12)C(F)(F)F)Cc3ccc(cc3)N(C)C |
| SJ000520437 | 23.64782076 | Q-1 | COc1ccc(CN(CCCNc2ccnc3cc(Cl)ccc23)Cc4ccccc4)cc1F |
| SJ000520477 | 23.57780501 | Q-1 | CCCCN(CCC)CCCNc1ccnc2cc(ccc12)C(F)(F)F |
| SJ000520450 | 23.1782047 | Q-1 | COc1ccc(cc1CN(CCCNc2ccnc3cc(Cl)ccc23)Cc4ccccc4)C(F)(F)F |
| SJ000522968 | 23.08769473 | Q-1 | CCCN(CCCNc1c2ccc(Cl)cc2nc3ccc(OC)cc13)Cc4c(F)cccc4C(F)(F)F |
| SJ000361172 | 22.9132163 | Q-1 | CCC(CC)CN(CCCNc1ccnc2cc(Cl)ccc12)Cc3ccccc3 |
| SJ000361273 | 22.89556558 | Q-1 | CCCN(CCCNc1ccnc2cc(ccc12)C(F)(F)F)Cc3ccc(OC)cc3 |
| SJ000361108 | 22.80763172 | Q-1 | FC(F)(F)c1ccc2c(NCCCNCC3CC3)ccnc2c1 |
| SJ000522999 | 22.73761596 | Q-1 | CCCN(CCCNc1ccnc2cc(Cl)ccc12)Cc3ccc(OC(F)(F)F)cc3 |
| SJ000523014 | 22.71447499 | Q-1 | CCCN(CCCNc1ccnc2cc(Cl)ccc12)Cc3cccn3C |
| SJ000522961 | 22.69129288 | Q-1 | CCCN(CCCNc1c2ccc(Cl)cc2nc3ccc(OC)cc13)Cc4ccccc4OC(F)(F)F |
| SJ000523081 | 22.28274747 | Q-1 | CCCN(CCCNc1ccnc2cc(ccc12)C(F)(F)F)CC(C)CC |
| SJ000361120 | 22.25074528 | Q-1 | CCCCCCNCCCNc1ccnc2cc(Cl)ccc12 |
| SJ000521028 | 22.10747418 | Q-1 | Clc1ccc2c(NCCCNCc3cc(Br)cs3)ccnc2c1 |
| SJ000361182 | 22.05200397 | Q-1 | Clc1ccc2c(NCCCN(Cc3ccccc3)Cc4ccc5ccccc5c4)ccnc2c1 |
| SJ000520467 | 22.01038386 | Q-1 | Fc1cccc(c1CN(CCCNc2ccnc3cc(Cl)ccc23)Cc4ccccc4)C(F)(F)F |
| SJ000522757 | 21.98575687 | Q-1 | CCCN(CCCNc1ccnc2cc(Cl)ccc12)Cc3ccccc3F |
| SJ000521013 | 21.75739541 | Q-1 | CCCN(CCCNc1ccnc2cc(ccc12)C(F)(F)F)Cc3ccc(OC)c(F)c3 |
| SJ000523082 | 21.73802026 | Q-1 | CCCN(CCCNc1ccnc2cc(ccc12)C(F)(F)F)Cc3cc(C)ccc3C |
| SJ000523068 | 21.65452136 | Q-1 | [O−][N+](═O)c1ccccc1c2oc(CNCCCNc3ccnc4cc(Cl)ccc34)cc2 |
| SJ000357483 | 21.05829745 | Q-1 | Clc1ccc2c(NCCCN(CC3CC3)Cc4ccccc4)ccnc2c1 |
| SJ000522759 | 20.79330904 | Q-1 | CCCN(CCCNc1ccnc2cc(Cl)ccc12)Cc3ccc(F)cc3 |
| SJ000521014 | 20.63714336 | Q-1 | CCCN(CCCNc1ccnc2cc(ccc12)C(F)(F)F)Cc3cc(F)ccc3C |
| SJ000522717 | 20.46207353 | Q-1 | Clc1ccc2c(NCCCN(Cc3ccccc3)Cc4ccc[nH]4)ccnc2c1 |
| SJ000520452 | 20.44429313 | Q-1 | COc1ccc(OC(F)(F)F)cc1CN(CCCNc2ccnc3cc(Cl)ccc23)Cc4ccccc4 |
| SJ000311302 | 20.4270961 | Q-1 | N#Cc1ccc(Cc2ccc3c(NCCCNCc4occc4)ccnc3c2)cc1 |
| SJ000361176 | 20.35708034 | Q-1 | Fc1cccc(CN(CCCNc2ccnc3cc(Cl)ccc23)Cc4ccccc4)c1 |
| SJ000361187 | 20.21704884 | Q-1 | Cc1ccc(C)c(CN(CCCNc2ccnc3cc(Cl)ccc23)Cc4ccccc4)c1 |
| SJ000361179 | 20.19708513 | Q-1 | [O−][N+](═O)c1ccc(CN(CCCNc2ccnc3cc(Cl)ccc23)Cc4ccccc4)cc1 |
| SJ000523076 | 20.17192953 | Q-1 | CCCN(CCCNc1ccnc2cc(ccc12)C(F)(F)F)Cc3cccc(C)c3 |
| SJ000357437 | 20.14703308 | Q-1 | CCCN(CCCNc1ccnc2cc(Cl)ccc12)Cc3oc(C)cc3 |
| SJ000044383 | 20.10383862 | Q-1 | NC(═O)CC(Nc1c2ccccc2nc3ccccc13)C(═O)O |
| SJ000361264 | 19.93209672 | Q-1 | CCCN(CCCNc1ccnc2cc(ccc12)C(F)(F)F)Cc3c(C)cc(C)cc3C |
| SJ000522748 | 19.86584962 | Q-1 | CCCN(CCCNc1ccnc2cc(Cl)ccc12)Cc3c(F)c(F)c(F)c(F)c3F |
| SJ000357599 | 19.76338412 | Q-1 | FC(F)(F)Oc1ccccc1CN(CCCNc2ccnc3cc(Cl)ccc23)Cc4ccccc4 |
| SJ000357439 | 19.72693856 | Q-1 | CCCN(CCCNc1ccnc2cc(Cl)ccc12)Cc3sccc3C |
| SJ000521044 | 19.58690705 | Q-1 | COC(═O)CCCCCNCCCNc1ccnc2cc(ccc12)C(F)(F)F |
| SJ000361253 | 19.53461411 | Q-1 | CCCN(CCCNc1ccnc2cc(ccc12)C(F)(F)F)Cc3cccs3 |
| SJ000520483 | 19.44687555 | Q-1 | CCCN(CCCNc1ccnc2cc(ccc12)C(F)(F)F)Cc3csc4ccccc34 |
| SJ000357499 | 19.40211991 | Q-1 | CCCN(CCCNc1ccnc2cc(Cl)ccc12)Cc3c(F)cccc3OC |
| SJ000357579 | 19.30684404 | Q-1 | CCCCN(CCCNc1ccnc2cc(Cl)ccc12)Cc3ccccc3 |
| SJ000361167 | 19.2696257 | Q-1 | Oc1ccccc1CN(CCCNc2ccnc3cc(Cl)ccc23)Cc4ccccc4 |
| SJ000361274 | 19.21865691 | Q-1 | CCCN(CCCNc1ccnc2cc(ccc12)C(F)(F)F)Cc3ccccc3F |
| SJ000357431 | 19.1371315 | Q-1 | CCCN(CCCNc1ccnc2cc(Cl)ccc12)Cc3ccccc3 |
| SJ000522756 | 19.1371315 | Q-1 | CCCN(CCCNc1ccnc2cc(Cl)ccc12)Cc3ccc(OC)cc3 |
| SJ000361258 | 19.0824751 | Q-1 | CCCN(CCCNc1ccnc2cc(ccc12)C(F)(F)F)Cc3ccc(C)s3 |
| SJ000523053 | 19.0143842 | Q-1 | CC(CCNCCCNc1ccnc2cc(Cl)ccc12)c3ccccc3 |
| SJ000521019 | 18.95676527 | Q-1 | Cn1cccc1CNCCCNc2ccnc3cc(Cl)ccc23 |
| SJ000520442 | 18.88674952 | Q-1 | CCc1ccc(CN(CCCNc2ccnc3cc(Cl)ccc23)Cc4ccccc4)cc1 |
| SJ000357468 | 18.74671801 | Q-1 | CC(C)CN(CCCNc1ccnc2cc(Cl)ccc12)Cc3ccccc3 |
| SJ000522753 | 18.74671801 | Q-1 | CCCN(CCCNc1ccnc2cc(Cl)ccc12)Cc3ccc4OCOc4c3 |
| SJ000521055 | 18.53667075 | Q-1 | CCCCNCCCNc1ccnc2cc(ccc12)C(F)(F)F |
| SJ000361112 | 18.46965699 | Q-1 | CCC(C)CNCCCNc1ccnc2cc(ccc12)C(F)(F)F |
| SJ000521020 | 18.466655 | Q-1 | Cn1cc(CNCCCNc2ccnc3cc(Cl)ccc23)c4ccccc14 |
| SJ000357594 | 18.14342498 | Q-1 | CCCN(CCCNc1ccnc2cc(Cl)ccc12)Cc3cccc4OCOc34 |
| SJ000522721 | 18.07717787 | Q-1 | Fc1ccc(CN(CCCNc2ccnc3cc(Cl)ccc23)Cc4ccccc4)cc1 |
| SJ000361113 | 18.04656048 | Q-1 | FC(F)(F)c1ccc2c(NCCCNCc3occc3)ccnc2c1 |
| SJ000521029 | 17.90652897 | Q-1 | Clc1ccc2c(NCCCNCc3oc4ccccc4c3)ccnc2c1 |
| SJ000523046 | 17.74594237 | Q-1 | CCCN(CCCNc1ccnc2cc(Cl)ccc12)Cc3c(Cl)ccc(Cl)c3Cl |
| SJ000361251 | 17.48643445 | Q-1 | CCCN(CCCNc1ccnc2cc(ccc12)C(F)(F)F)Cc3ccncc3 |
| SJ000523013 | 17.48095396 | Q-1 | CCCN(CCCNc1ccnc2cc(Cl)ccc12)Cc3cccc(c3)C(F)(F)F |
| SJ000520474 | 17.38020257 | Q-1 | CCCN(CCCNc1ccnc2cc(ccc12)C(F)(F)F)Cc3ccc(OC(F)(F)F)cc3 |
| SJ000311184 | 17.34640294 | Q-1 | ClC1═CC2═NC═CC(NCCCN(CCC(C)C3═CC═CC═C3)CC4═CC═CC═C4)═C2C═C1 |
| SJ000520429 | 17.13635568 | Q-1 | COC(═O)CCCCCN(CCCNc1ccnc2cc(Cl)ccc12)Cc3ccccc3 |
| SJ000357494 | 17.08347135 | Q-1 | CCCN(CCCNc1ccnc2cc(Cl)ccc12)Cc3cc(F)ccc3OC |
| SJ000361178 | 17.01722425 | Q-1 | [O−][N+](═O)c1cccc(CN(CCCNc2ccnc3cc(Cl)ccc23)Cc4ccccc4)c1 |
| SJ000522761 | 16.95097714 | Q-1 | CCCN(CCCNc1ccnc2cc(Cl)ccc12)Cc3ccc(Cl)c(c3)[N+](═O)[O−] |
| SJ000523073 | 16.85629267 | Q-1 | CCCN(CCCNc1ccnc2cc(ccc12)C(F)(F)F)Cc3ccccc3C(═O)O |
| SJ000044378 | 16.76738446 | Q-1 | CN(C)CCCNc1c2ccccc2nc3ccccc13 |
| SJ000523042 | 16.71626116 | Q-1 | CCCN(CCCNc1ccnc2cc(Cl)ccc12)Cc3ccc(cc3)C(═O)OC |
| SJ000361103 | 16.55349453 | Q-1 | CCC(CC)CNCCCNc1ccnc2cc(Cl)ccc12 |
| SJ000357433 | 16.5062139 | Q-1 | CCCN(CCCNc1ccnc2cc(Cl)ccc12)Cc3ccc(C)s3 |
| SJ000361177 | 16.42100033 | Q-1 | [O−][N+](═O)c1ccccc1CN(CCCNc2ccnc3cc(Cl)ccc23)Cc4ccccc4 |
| SJ000522974 | 16.35883905 | Q-1 | CCCN(CCCNc1c2ccc(Cl)cc2nc3ccc(OC)cc13)Cc4ccc(Cl)c(c4)C(F)(F)F |
| SJ000522754 | 16.28850613 | Q-1 | CCCCCN(CCC)CCCNc1ccnc2cc(Cl)ccc12 |
| SJ000523045 | 16.22615088 | Q-1 | CCCN(CCCNc1ccnc2cc(Cl)ccc12)Cc3oc(cc3)c4cccc(Cl)c4 |
| SJ000520468 | 16.15613513 | Q-1 | CCCN(CCCNc1ccnc2cc(ccc12)C(F)(F)F)Cc3c[nH]c4ccccc34 |
| SJ000522986 | 16.15456635 | Q-1 | CCCN(CCCNc1c2ccc(Cl)cc2nc3ccc(OC)cc13)Cc4cccc(c4)C(F)(F)F |
| SJ000311182 | 16.08647544 | Q-1 | ClC1═CC2═NC═CC(NCCCN(CC3CCC═CC3)CC4═CC═CC═C4)═C2C═C1 |
| SJ000357597 | 15.95727062 | Q-1 | CCCN(CCCNc1ccnc2cc(Cl)ccc12)Cc3cc(F)cc(F)c3O |
| SJ000311254 | 15.94608787 | Q-1 | C(CNCc1occc1)CNc2ccnc3cc(CCc4ccccc4)ccc23 |
| SJ000520475 | 15.74602094 | Q-1 | CCCN(CCCNc1ccnc2cc(ccc12)C(F)(F)F)Cc3cc(F)ccc3OC |
| SJ000361250 | 15.67793004 | Q-1 | CCCN(CCC)CCCNc1ccnc2cc(ccc12)C(F)(F)F |
| SJ000575182 | 15.67793004 | Q-1 | CCCN(CCCNc1ccnc2cc(ccc12)C(F)(F)F)Cc3cnn(C)c3 |
| SJ000523011 | 15.66602486 | Q-1 | CCCN(CCCNc1ccnc2cc(Cl)ccc12)Cc3cc(OC(F)(F)F)ccc3OC |
| SJ000361102 | 15.62603511 | Q-1 | Clc1ccc2c(NCCCNCC3CCCCC3)ccnc2c1 |
| SJ000522751 | 15.62603511 | Q-1 | CCCN(CCCNc1ccnc2cc(Cl)ccc12)CC3CCCCC3 |
| SJ000521065 | 15.55978801 | Q-1 | FC(F)(F)c1ccc2c(NCCCNCC3CCOC3)ccnc2c1 |
| SJ000361260 | 15.49354091 | Q-1 | CCCN(CCCNc1ccnc2cc(ccc12)C(F)(F)F)Cc3cccc(OC)c3O |
| SJ000522762 | 15.49354091 | Q-1 | CCCN(CCCNc1ccnc2cc(Cl)ccc12)Cc3c4ccccc4cc5ccccc35 |
| SJ000522747 | 15.42729381 | Q-1 | CCCN(CCCNc1ccnc2cc(Cl)ccc12)Cc3ccccc3N |
| SJ000520431 | 15.3610467 | Q-1 | Cn1ccnc1CN(CCCNc2ccnc3cc(Cl)ccc23)Cc4ccccc4 |
| SJ000575193 | 15.33747553 | Q-1 | Clc1ccc2c(NCCCNCc3ccc(Br)s3)ccnc2c1 |
| SJ000523052 | 15.24593033 | Q-1 | Clc1ccc2c(NCCCNCc3ccsc3)ccnc2c1 |
| SJ000361252 | 15.1623054 | Q-1 | CCCN(CCCNc1ccnc2cc(ccc12)C(F)(F)F)Cc3ccccn3 |
| SJ000522994 | 15.1623054 | Q-1 | CCCN(CCCNc1ccnc2cc(Cl)ccc12)Cc3ccc(OC)c(F)c3 |
| SJ000521042 | 15.03588307 | Q-1 | FC(F)(F)c1ccc2c(NCCCNCc3cccnc3)ccnc2c1 |
| SJ000521012 | 14.99702102 | Q-1 | CCCN(CCCNc1ccnc2cc(ccc12)C(F)(F)F)Cc3cc(F)cc(F)c30 |
| SJ000523051 | 14.96586732 | Q-1 | CSCCCNCCCNc1ccnc2cc(Cl)ccc12 |
| SJ000037397 | 14.92893012 | Q-1 | CCOC(═O)c1cnc2c(C)cc(C)cc2c1NCCCN(C)C |
| SJ000361166 | 14.76482279 | Q-1 | Clc1ccc2c(NCCCN(Cc3ccccc3)Cc4ccccc4)ccnc2c1 |
| SJ000522741 | 14.76482279 | Q-1 | CN(C)c1ccc(CN(CCCNc2ccnc3cc(Cl)ccc23)Cc4ccccc4)cc1 |
| SJ000523079 | 14.65656652 | Q-1 | CCCN(CCCNc1ccnc2cc(ccc12)C(F)(F)F)Cc3ccc(OC)c(OC)c3 |
| SJ000520472 | 14.61578855 | Q-1 | CCCN(CCCNc1ccnc2cc(ccc12)C(F)(F)F)Cc3cccc4OCOc34 |
| SJ000311206 | 14.58847561 | Q-1 | C(CNCc1occc1)CNc2ccnc3cc(Oc4ccccc4)ccc23 |
| SJ000311195 | 14.47575705 | Q-1 | ClC1═CC2═NC═CC(NCCCN(CCC)CC3═CC═C(Br)O3)═C2C═C1 |
| SJ000522967 | 14.47575705 | Q-1 | CCCN(CCCNc1c2ccc(Cl)cc2nc3ccc(OC)cc13)Cc4cccc(OC(F)(F)F)c4 |
| SJ000361109 | 14.45229381 | Q-1 | Cc1oc(CNCCCNc2ccnc3cc(ccc23)C(F)(F)F)cc1 |
| SJ000521062 | 14.43358728 | Q-1 | FC(F)(F)c1ccc2c(NCCCNCc3nc4ccccc4s3)ccnc2c1 |
| SJ000357580 | 14.40574129 | Q-1 | Clc1ccc2c(NCCCN(CCc3ccccc3)Cc4ccccc4)ccnc2c1 |
| SJ000523074 | 14.31611201 | Q-1 | CCCN(CCCNc1ccnc2cc(ccc12)C(F)(F)F)Cc3cccc4ccccc34 |
| SJ000523017 | 14.30109308 | Q-1 | CCCN(CCCCCC(═O)OC)CCCNc1ccnc2cc(Cl)ccc12 |
| SJ000134254 | 14.26570978 | Q-1 | CCOC(═O)c1cnc2c(C)c(C)ccc2c1NCCCN3CCOCC3 |
| SJ000357463 | 14.16859887 | Q-1 | COc1ccc(O)c(CN(CCCNc2ccnc3cc(Cl)ccc23)Cc4ccccc4)c1 |
| SJ000520928 | 14.16859887 | Q-1 | CCCN(CCCNc1c2ccc(Cl)cc2nc3ccc(OC)cc13)Cc4ccccc4C(F)(F)F |
| SJ000522752 | 14.16859887 | Q-1 | CCCN(CCCNc1ccnc2cc(Cl)ccc12)CC(CC)CC |
| SJ000361272 | 13.8394757 | Q-1 | CCCN(CCCNc1ccnc2cc(ccc12)C(F)(F)F)Cc3ccc(F)c(F)c3 |
| SJ000522755 | 13.63862206 | Q-1 | CCCN(CCCNc1ccnc2cc(Cl)ccc12)CC3═C(C)N(C)N(C3═O)c4ccccc4 |
| SJ000361121 | 13.49902119 | Q-1 | CSC(C)CCNCCCNc1ccnc2cc(Cl)ccc12 |
| SJ000523006 | 13.30738655 | Q-1 | CCCN(CCCNc1ccnc2cc(Cl)ccc12)Cc3cccnc3Br |
| SJ000357583 | 13.24113945 | Q-1 | Clc1ccc2c(NCCCN(CCOCc3ccccc3)Cc4ccccc4)ccnc2c1 |
| SJ000357465 | 13.17489235 | Q-1 | COc1cccc(CN(CCCNc2ccnc3cc(Cl)ccc23)Cc4ccccc4)c1O |
| SJ000520471 | 13.02238488 | Q-1 | CCCN(CCCNc1ccnc2cc(ccc12)C(F)(F)F)Cc3cccc(c3)C(F)(F)F |
| SJ000311246 | 13.00542622 | Q-1 | CC(C)CCc1ccc2c(NCCCNCc3occc3)ccnc2c1 |
| SJ000520482 | 13.00542622 | Q-1 | CCCN(CCCNc1ccnc2cc(ccc12)C(F)(F)F)Cc3cc4ccccc4s3 |
| SJ000522763 | 12.90990394 | Q-1 | CCCN(CCCNc1ccnc2cc(Cl)ccc12)Cc3cccc(c3)C#N |
| SJ000361170 | 12.84365684 | Q-1 | Clc1ccc2c(NCCCN(Cc3cocc3)Cc4ccccc4)ccnc2c1 |
| SJ000361123 | 12.81811218 | Q-1 | CCCNCCCNc1ccnc2cc(ccc12)C(F)(F)F |
| SJ000522760 | 12.79537896 | Q-1 | CCCN(CCCNc1ccnc2cc(Cl)ccc12)Cc3cc4ccccc4nc3Cl |
| SJ000357571 | 12.77740974 | Q-1 | CCCCN(CCC)CCCNc1ccnc2cc(Cl)ccc12 |
| SJ000523000 | 12.44617423 | Q-1 | CCCN(CCCNc1ccnc2cc(Cl)ccc12)Cc3ccccc3C#C |
| SJ000357570 | 12.11493872 | Q-1 | CCCN(CCCNc1ccnc2cc(Cl)ccc12)CCC(C)(C)C |
| SJ000523043 | 12.09522142 | Q-1 | CCCN(CCCNc1ccnc2cc(Cl)ccc12)Cc3ccc(Cl)cc3Cl |
| SJ000523010 | 12.04869162 | Q-1 | CCCN(CCCNc1ccnc2cc(Cl)ccc12)Cc3[nH]cnc3C |
| SJ000357454 | 11.95518992 | Q-1 | CCCN(CCCNc1ccnc2cc(Cl)ccc12)Cc3nccs3 |
| SJ000520480 | 11.95518992 | Q-1 | CCCN(CCCNc1ccnc2cc(ccc12)C(F)(F)F)Cc3cc(OC)ccc3F |
| SJ000523005 | 11.84995031 | Q-1 | CCCN(CCCNc1ccnc2cc(Cl)ccc12)Cc3scnc3C |
| SJ000522720 | 11.78370321 | Q-1 | COc1ccc(CN(CCCNc2ccnc3cc(Cl)ccc23)Cc4ccccc4)cc1 |
| SJ000361101 | 11.46507964 | Q-1 | Clc1ccc2c(NCCCNCC3CC3)ccnc2c1 |
| SJ000520481 | 11.46507964 | Q-1 | CCCN(CCCNc1ccnc2cc(ccc12)C(F)(F)F)Cc3c(F)cccc3C(F)(F)F |
| SJ000357568 | 11.4524677 | Q-1 | CCCN(CCCNc1ccnc2cc(Cl)ccc12)CC(C)C |
| SJ000523075 | 11.38820325 | Q-1 | CCCN(CCCNc1ccnc2cc(ccc12)C(F)(F)F)Cc3ccc4ccccc4c3 |
| SJ000176583 | 11.25503238 | Q-1 | CCOC(═O)c1ccc2ncc(C(═O)OCC)c(NCCCN3CCOCC3)c2c1 |
| SJ000521015 | 11.04498512 | Q-1 | CCCN(CCCNc1ccnc2cc(ccc12)C(F)(F)F)Cc3ccc(Cl)c(c3)C(F)(F)F |
| SJ000357440 | 10.90495361 | Q-1 | Cc1ccsc1CN(CCCNc2ccnc3cc(Cl)ccc23)Cc4ccccc4 |
| SJ000311190 | 10.83493786 | Q-1 | ClC1═CC2═NC═CC(NCCCNCC3═CC═C(C)S3)═C2C═C1 |
| SJ000279665 | 10.70729424 | Q-1 | CCOC(═O)c1cnc2ccc(cc2c1NCCCN3CCOCC3)C(═O)OC |
| SJ000357445 | 10.34482759 | Q-1 | CCCN(CCCNc1ccnc2cc(Cl)ccc12)Cc3ccc[nH]3 |
| SJ000311326 | 10.20479608 | Q-1 | ClC1═CC═C2C(N═CC═C2NCCCNCC3═CC═C(C)O3)═C1 |
| SJ000521068 | 10.19377277 | Q-1 | Cn1ccnc1CNCCCNc2ccnc3cc(ccc23)C(F)(F)F |
| SJ000131358 | 10.06127857 | Q-1 | CCOC(═O)c1cnc2c(cccc2c1NCCCN3CCOCC3)C(F)(F)F |
| SJ000523009 | 10.06127857 | Q-1 | CCCN(CCCNc1ccnc2cc(Cl)ccc12)Cc3cnn(C)c3 |
| SJ000361104 | 9.82211252 | Q-1 | CCC(C)CNCCCNc1ccnc2cc(Cl)ccc12 |
| SJ000361119 | 9.784701558 | Q-1 | CCCNCCCNc1ccnc2cc(Cl)ccc12 |
| SJ000520462 | 9.714685804 | Q-1 | Clc1ccc2c(NCCCN(Cc3ccccc3)Cc4cccnc4Br)ccnc2c1 |
| SJ000523016 | 9.663795959 | Q-1 | CCCN(CCCNc1ccnc2cc(Cl)ccc12)CC3CCOC3 |
| SJ000312675 | 9.597548857 | Q-1 | Clc1ccc2c(NCCCN(Cc3ccccc3)Cc4ccsc4)ccnc2c1 |
| SJ000361180 | 9.597548857 | Q-1 | OC(═O)c1ccccc1CN(CCCNc2ccnc3cc(Cl)ccc23)Cc4ccccc4 |
| SJ000520956 | 9.597548857 | Q-1 | CCCN(CCCNc1c2ccc(Cl)cc2nc3ccc(OC)cc13)Cc4ccc(Cl)cc4Cl |
| SJ000523047 | 9.574654297 | Q-1 | CCCN(CCCNc1ccnc2cc(Cl)ccc12)Cc3oc(cc3)[N+](═O)[O−] |
| SJ000311222 | 9.364607037 | Q-1 | Fc1ccc(Oc2ccc3c(NCCCNCc4occc4)ccnc3c2)cc1 |
| SJ000575181 | 9.345476211 | Q-1 | CCCN(CCCNc1ccnc2cc(ccc12)C(F)(F)F)Cc3ncc[nH]3 |
| SJ000357449 | 9.294591283 | Q-1 | CCCN(CCCNc1ccnc2cc(Cl)ccc12)Cc3ccncc3 |
| SJ000522991 | 9.294591283 | Q-1 | CCCN(CCCNc1ccnc2cc(Cl)ccc12)Cc3oc4ccccc4c3 |
| SJ000523044 | 9.266313349 | Q-1 | CCCN(CCCNc1ccnc2cc(Cl)ccc12)Cc3oc(cc3)c4ccccc4[N+](═O)[O−] |
| SJ000521026 | 9.224575529 | Q-1 | CC(C)(C)CCNCCCNc1ccnc2cc(Cl)ccc12 |
| SJ000522750 | 9.133819145 | Q-1 | CCCN(CCCNc1ccnc2cc(Cl)ccc12)Cc3ccc(s3)[N+](═O)[O−] |
| SJ000520473 | 9.084544022 | Q-1 | CCCN(CCCNc1ccnc2cc(ccc12)C(F)(F)F)Cc3cccc(OC(F)(F)F)c3 |
| SJ000522719 | 9.001324942 | Q-1 | Clc1ccc2c(NCCCN(Cc3ccccc3)Cc4ccc5OCOc5c4)ccnc2c1 |
| SJ000311258 | 8.874496762 | Q-1 | CC(C)Cc1ccc2c(NCCCNCc3occc3)ccnc2c1 |
| SJ000575189 | 8.664567197 | Q-1 | CCCN(CCCNc1ccnc2cc(ccc12)C(F)(F)F)Cc3nccs3 |
| SJ000521027 | 8.454402241 | Q-1 | CCCCNCCCNc1ccnc2cc(Cl)ccc12 |
| SJ000520486 | 8.187930888 | Q-1 | CCCN(CCCNc1ccnc2cc(ccc12)C(F)(F)F)Cc3cc(ccc3OC)C(F)(F)F |
| SJ000311294 | 7.824260459 | Q-1 | COc1ccc(cc1)c2ccc3c(NCCCNCc4occc4)ccnc3c2 |
| SJ000357576 | 7.824260459 | Q-1 | CCCN(CCCNc1ccnc2cc(Cl)ccc12)CCCSC |
| SJ000522744 | 7.824260459 | Q-1 | CCCN(CCCNc1ccnc2cc(Cl)ccc12)Cc3ccsc3 |
| SJ000357446 | 7.808877112 | Q-1 | CCCN(CCCNc1ccnc2cc(Cl)ccc12)Cc3cccnc3 |
| SJ000521046 | 7.77938548 | Q-1 | FC(F)(F)c1ccc2c(NCCCNCc3nccs3)ccnc2c1 |
| SJ000520484 | 7.404165937 | Q-1 | CCCN(CCCNc1ccnc2cc(ccc12)C(F)(F)F)Cc3nc4ccccc4s3 |
| SJ000575170 | 7.124102923 | Q-1 | CCCN(CCCNc1ccnc2cc(ccc12)C(F)(F)F)Cc3nccn3C |
| SJ000521037 | 7.013911891 | Q-1 | Clc1ccc2c(NCCCNCc3cncc(Br)c3)ccnc2c1 |
| SJ000357495 | 6.682676383 | Q-1 | COc1ccc(F)cc1CN(CCCNc2ccnc3cc(Cl)ccc23)Cc4ccccc4 |
| SJ000311189 | 6.553749255 | Q-1 | ClC1═CC2═NC═CC(NCCCNCC3═C(C)C═CS3)═C2C═C1 |
| SJ000173725 | 6.493961141 | Q-1 | CCOC(═O)c1cnc2c(C)cc(C)cc2c1NCCCN3CCOCC3 |
| SJ000523002 | 6.351440874 | Q-1 | CCCN(CCCNc1ccnc2cc(Cl)ccc12)Cc3nccn3C |
| SJ000000502 | 6.349476551 | Q-1 | ClC1═CC═C2C(N═CC═C2NCCCNCC3═CC═CO3)═C1 |
| SJ000520912 | 6.283913881 | Q-1 | [O−][N+](═O)c1ccc(CNCCCNc2ccnc3cc(Cl)ccc23)s1 |
| SJ000357442 | 6.213898127 | Q-1 | CCCN(CCCNc1ccnc2cc(Cl)ccc12)Cc3cocc3 |
| SJ000523049 | 6.077112946 | Q-1 | Clc1ccc2c(NCCCNCc3ccncc3)ccnc2c1 |
| SJ000361185 | 6.07386662 | Q-1 | Clc1ccc2c(NCCCN(CCCc3ccccc3)Cc4ccccc4)ccnc2c1 |
| SJ000361183 | 6.020205366 | Q-1 | Cc1cccc(CN(CCCNc2ccnc3cc(Cl)ccc23)Cc4ccccc4)c1 |
| SJ000520470 | 5.933835113 | Q-1 | CCCN(CCCNc1ccnc2cc(ccc12)C(F)(F)F)Cc3ccccc3OC(F)(F)F |
| SJ000522993 | 5.863819359 | Q-1 | CCCN(CCCNc1ccnc2cc(Cl)ccc12)Cc3cc4ccccc4s3 |
| SJ000361270 | 5.622722756 | Q-1 | CCCN(CCCNc1ccnc2cc(ccc12)C(F)(F)F)Cc3ccc4OCOc4c3 |
| SJ000520461 | 5.600476636 | Q-1 | Clc1ccc2c(NCCCN(Cc3oc4ccccc4c3)Cc5ccccc5)ccnc2c1 |
| SJ000520485 | 5.600476636 | Q-1 | CCCN(CCCNc1ccnc2cc(ccc12)C(F)(F)F)Cc3cc(OC(F)(F)F)ccc3OC |
| SJ000575191 | 5.600476636 | Q-1 | CCCN(CCCCCC(═O)OC)CCCNc1ccnc2cc(ccc12)C(F)(F)F |
| SJ000523058 | 5.583756345 | Q-1 | CN1N(C(═O)C(═C1C)CNCCCNc2ccnc3cc(Cl)ccc23)c4ccccc4 |
| SJ000279525 | 5.532385735 | Q-1 | CCOC(═O)c1cnc2ccc(OCC)cc2c1NCCCN3CCOCC3 |
| SJ000575179 | 5.163661824 | Q-1 | CCCN(CCCNc1ccnc2cc(ccc12)C(F)(F)F)Cc3scnc3C |
| SJ000575180 | 5.163661824 | Q-1 | CCCN(CCCNc1ccnc2cc(ccc12)C(F)(F)F)Cc3cccnc3Br |
| SJ000361259 | 4.987658524 | Q-1 | CCCN(CCCNc1ccnc2cc(ccc12)C(F)(F)F)Cc3ccccc3C(F)(F)F |
| SJ000521023 | 4.987658524 | Q-1 | Clc1ccc2c(NCCCNCc3nccs3)ccnc2c1 |
| SJ000357574 | 4.88359881 | Q-1 | CCCN(CCCNc1ccnc2cc(Cl)ccc12)CCc3ccccc3 |
| SJ000522990 | 4.827757536 | Q-1 | CCCN(CCCNc1ccnc2cc(Cl)ccc12)Cc3c[nH]c(C)n3 |
| SJ000523004 | 4.827757536 | Q-1 | CCCN(CCCNc1ccnc2cc(Cl)ccc12)Cc3cncc(Br)c3 |
| SJ000520445 | 4.761510434 | Q-1 | COc1ccc(F)c(CN(CCCNc2ccnc3cc(Cl)ccc23)Cc4ccccc4)c1 |
| SJ000312672 | 4.743567303 | Q-1 | Clc1ccc2c(NCCCN(Cc3ccccc3)Cc4cccs4)ccnc2c1 |
| SJ000523050 | 4.743567303 | Q-1 | Clc1ccc2c(NCCCNCc3ccccn3)ccnc2c1 |
| SJ000521034 | 4.695263332 | Q-1 | Clc1ccc2c(NCCCNCc3nc4ccccc4s3)ccnc2c1 |
| SJ000312673 | 4.629016231 | Q-1 | Cc1ccc(CN(CCCNc2ccnc3cc(Cl)ccc23)Cc4ccccc4)s1 |
| SJ000522722 | 4.430274925 | Q-1 | COc1ccccc1CN(CCCNc2ccnc3cc(Cl)ccc23)Cc4ccccc4 |
| SJ000521007 | 4.323472781 | Q-1 | CCCN(CCCNc1ccnc2cc(ccc12)C(F)(F)F)Cc3cncc(Br)c3 |
| SJ000523008 | 4.165286519 | Q-1 | CCCN(CCCNc1ccnc2cc(Cl)ccc12)Cc3ncc[nH]3 |
| SJ000279431 | 4.102476807 | Q-1 | CCOC(═O)c1cnc2c(Cl)cccc2c1NCCCN3CCOCC3 |
| SJ000523078 | 3.830113201 | Q-1 | CCCN(CCCNc1ccnc2cc(ccc12)C(F)(F)F)Cc3ccc(Cl)cc3Cl |
| SJ000180455 | 3.635309705 | Q-1 | CCOC(═O)c1cnc2c(OC)cccc2c1NCCCN3CCOCC3 |
| SJ000357598 | 3.489658694 | Q-1 | Clc1ccc2c(NCCCN(Cc3ccccc3)Cc4cccc5OCOc45)ccnc2c1 |
| SJ000357458 | 3.483283739 | Q-1 | CCCN(CCCNc1ccnc2cc(Cl)ccc12)Cc3occc3 |
| SJ000522997 | 3.413267985 | Q-1 | CCCN(CCCNc1ccnc2cc(Cl)ccc12)Cc3nc(C)cs3 |
| SJ000279636 | 3.353476892 | Q-1 | CCOC(═O)c1cnc2c(C)cccc2c1NCCCN3CCOCC3 |
| SJ000127046 | 2.944931484 | Q-1 | CCOC(═O)c1cnc2ccc(OC)cc2c1NCCCN3CCOCC3 |
| SJ000312669 | 2.840344485 | Q-1 | Cc1oc(CN(CCCNc2ccnc3cc(Cl)ccc23)Cc4ccccc4)cc1 |
| SJ000311183 | 2.672567878 | Q-1 | ClC1═CC2═NC═CC(NCCCN(CCC(C)C)CC3═CC═CC═C3)═C2C═C1 |
| SJ000361168 | 2.509108976 | Q-1 | FC(F)(F)c1ccccc1CN(CCCNc2ccnc3cc(Cl)ccc23)Cc4ccccc4 |
| SJ000522982 | 2.045379265 | Q-1 | CCCN(CCCNc1c2ccc(Cl)cc2nc3ccc(OC)cc13)Cc4cc(F)cc(F)c4O |
| SJ000520931 | 1.84663796 | Q-1 | CCCN(CCCNc1c2ccc(Cl)cc2nc3ccc(OC)cc13)Cc4cc(F)ccc4OC |
| SJ000575190 | 1.787386161 | Q-1 | CCCN(CCCNc1ccnc2cc(ccc12)C(F)(F)F)CC3CCOC3 |
| SJ000521040 | 1.449155349 | Q-1 | Cn1ccnc1CNCCCNc2ccnc3cc(Cl)ccc23 |
| SJ000523048 | 1.316661146 | Q-1 | CCCCCCN(CCC)CCCNc1ccnc2cc(Cl)ccc12 |
| SJ000522743 | 1.312795379 | Q-1 | CCCN(CCCNc1ccnc2cc(Cl)ccc12)Cc3ccccn3 |
| SJ000361084 | 0.985425638 | Q-1 | COc1ccc2nc3cc(Cl)ccc3c(NCCCNCc4ccc[nH]4)c2c1 |
| SJ000520932 | 0.985425638 | Q-1 | CCCN(CCCNc1c2ccc(Cl)cc2nc3ccc(OC)cc13)Cc4oc(C)cc4 |
| SJ000520942 | 0.970295344 | Q-1 | CCCN(CCCNc1c2ccc(Cl)cc2nc3ccc(OC)cc13)Cc4ccc(F)c(F)c4 |
| SJ000361175 | 0.962716611 | Q-1 | Fc1ccccc1CN(CCCNc2ccnc3cc(Cl)ccc23)Cc4ccccc4 |
| SJ000521010 | 0.892700858 | Q-1 | CCCN(CCCNc1ccnc2cc(ccc12)C(F)(F)F)Cc3nc(C)cs3 |
| SJ000522730 | 0.786684333 | Q-1 | CCCN(CCCNc1c2ccc(Cl)cc2nc3ccc(OC)cc13)CCCSC |
| SJ000312668 | 0.521695926 | Q-1 | Clc1ccc2c(NCCCN(Cc3occc3)Cc4ccccc4)ccnc2c1 |
| SJ000521038 | 0.402590583 | Q-1 | Clc1ccc2c(NCCCNCC3CCOC3)ccnc2c1 |
| SJ000522977 | 0.256707519 | Q-1 | CCCN(CCCNc1c2ccc(Cl)cc2nc3ccc(OC)cc13)Cc4cccnc4Br |
| SJ000279864 | −0.391522683 | Q-1 | CCOC(═O)c1cnc2ccc(F)cc2c1NCCCN3CCOCC3 |
| SJ000522742 | −0.538257701 | Q-1 | COc1ccc(CN(CCCNc2ccnc3cc(Cl)ccc23)Cc4ccccc4)cc1OC |
| SJ000522995 | −1.531964227 | Q-1 | CCCN(CCCNc1ccnc2cc(Cl)ccc12)Cc3cn(C)c4ccccc34 |
| SJ000523028 | −1.531964227 | Q-1 | COc1ccc2nc3cc(Cl)ccc3c(NCCCNCc4ccc(N(C)C)c5ccccc45)c2c1 |
| SJ000312671 | −1.796952633 | Q-1 | Clc1ccc2c(NCCCN(Cc3oc(Br)cc3)Cc4ccccc4)ccnc2c1 |
| SJ000520918 | −1.796952633 | Q-1 | CCCN(CCCNc1c2ccc(Cl)cc2nc3ccc(OC)cc13)Cc4ccncc4 |
| SJ000176476 | −2.117976545 | Q-1 | CCOC(═O)c1cnc2c(Cl)cc(Cl)cc2c1NCCCN3CCOCC3 |
| SJ000522734 | −2.258008052 | Q-1 | CCCN(CCCNc1c2ccc(Cl)cc2nc3ccc(OC)cc13)Cc4c(F)c(F)c(F)c(F)c4F |
| SJ000357500 | −2.85690626 | Q-1 | COc1cccc(F)c1CN(CCCNc2ccnc3cc(Cl)ccc23)Cc4ccccc4 |
| SJ000361099 | −3.728338876 | Q-1 | COc1ccc2nc3cc(Cl)ccc3c(NCCCNCc4c5ccccc5cc6ccccc46)c2c1 |
| SJ000312678 | −3.850612786 | Q-1 | Clc1ccc2c(NCCCN(Cc3ccccc3)Cc4ccccn4)ccnc2c1 |
| SJ000312676 | −4.148433398 | Q-1 | Clc1ccc2c(NCCCN(Cc3ccccc3)Cc4nccs4)ccnc2c1 |
| SJ000185196 | −4.498512165 | Q-1 | CCOC(═O)c1cnc2c(CC)cccc2c1NCCCN3CCOCC3 |
| SJ000312679 | −5.57303743 | Q-1 | Clc1ccc2c(NCCCN(Cc3ccccc3)Cc4ccncc4)ccnc2c1 |
| SJ000521041 | −6.879047786 | Q-1 | Cn1cccc1CNCCCNc2ccnc3cc(ccc23)C(F)(F)F |
| SJ000522956 | −7.089095046 | Q-1 | COc1ccc2nc3cc(Cl)ccc3c(NCCCNCc4cccc(C)c4)c2c1 |
| SJ000523071 | −7.745340029 | Q-1 | CCCN(CCCNc1ccnc2cc(ccc12)C(F)(F)F)Cc3ccccc3[N+](═O)[O−] |
| SJ000520949 | −9.039067155 | Q-1 | CCCN(CCCNc1c2ccc(Cl)cc2nc3ccc(OC)cc13)Cc4ccc(cc4)[N+](═O)[O−] |
| SJ000361188 | −9.049536146 | Q-1 | COc1cccc(CN(CCCNc2ccnc3cc(Cl)ccc23)Cc4ccccc4)c1 |
| SJ000520478 | −9.243339859 | Q-1 | CCCN(CCCNc1ccnc2cc(ccc12)C(F)(F)F)Cc3cc(Br)cs3 |
| SJ000520439 | −9.31143076 | Q-1 | Cn1cc(CN(CCCNc2ccnc3cc(Cl)ccc23)Cc4ccccc4)c5ccccc15 |
| SJ000520955 | −10.06043067 | Q-1 | CCCN(CCCNc1c2ccc(Cl)cc2nc3ccc(OC)cc13)Cc4ccc(cc4)N(C)C |
| SJ000520463 | −10.46897608 | Q-1 | Cc1csc(CN(CCCNc2ccnc3cc(Cl)ccc23)Cc4ccccc4)n1 |
| SJ000174094 | −10.51986697 | Q-1 | CCOC(═O)c1cnc2c(C)cc(Cl)cc2c1NCCCN3CCOCC3 |
| SJ000520948 | −10.74133969 | Q-1 | CCCN(CCCNc1c2ccc(Cl)cc2nc3ccc(OC)cc13)Cc4cccc(c4)[N+](═O)[O−] |
| SJ000520913 | −11.15000875 | Q-1 | Clc1ccc2c(NCCCNCc3cocc3)ccnc2c1 |
| SJ000357577 | −13.1804656 | Q-1 | CCCN(CCCNc1ccnc2cc(Cl)ccc12)CCOCc3ccccc3 |
| SJ000520479 | −20.41024768 | Q-1 | CCCN(CCCNc1ccnc2cc(ccc12)C(F)(F)F)Cc3oc4ccccc4c3 |
| SJ000575150 | −21.37230877 | Q-1 | CCCN(CCCNc1c2ccc(Cl)cc2nc3ccc(OC)cc13)CC(C)C |
| SJ000522962 | −21.58235603 | Q-1 | CCCN(CCCNc1c2ccc(Cl)cc2nc3ccc(OC)cc13)Cc4cc5ccccc5s4 |
| SJ000357595 | −21.86982445 | Q-1 | CCCN(CCCNc1ccnc2cc(Cl)ccc12)Cc3ccccc3OC(F)(F)F |
| SJ000176507 | −22.00245055 | Q-1 | CCOC(═O)c1cnc2ccc(Br)cc2c1NCCCN3CCOCC3 |
| SJ000129912 | −23.26101358 | Q-1 | CCOC(═O)c1cnc2c(C)c(Cl)ccc2c1NCCCN3CCOCC3 |
| SJ000575152 | −23.40276562 | Q-1 | CCCN(CCCNc1c2ccc(Cl)cc2nc3ccc(OC)cc13)Cc4cccnc4 |
| SJ000575149 | −35.93558551 | Q-1 | CCCN(CCCNc1c2ccc(Cl)cc2nc3ccc(OC)cc13)CC4CCOC4 |
| SJ000522984 | −36.7057588 | Q-1 | CCCN(CCCNc1c2ccc(Cl)cc2nc3ccc(OC)cc13)Cc4cc(OC(F)(F)F)ccc4OC |
| SJ000575151 | −37.40591633 | Q-1 | CCCN(CCCCCC(═O)OC)CCCNc1c2ccc(Cl)cc2nc3ccc(OC)cc13 |
| SJ000522745 | −38.03411726 | Q-1 | CCCN(CCCNc1ccnc2cc(Cl)ccc12)Cc3ccccc3C(F)(F)F |
| SJ000361189 | −40.75024843 | Q-1 | CCCCCCN(CCCNc1ccnc2cc(Cl)ccc12)Cc3ccccc3 |
| SJ000520952 | −40.7666725 | Q-1 | CCCN(CCCNc1c2ccc(Cl)cc2nc3ccc(OC)cc13)Cc4cccc5ccccc45 |
| SJ000522985 | −46.01785402 | Q-1 | CCCN(CCCNc1c2ccc(Cl)cc2nc3ccc(OC)cc13)Cc4cc(ccc4OC)C(F)(F)F |
| SJ000520934 | −46.31500497 | Q-1 | CCCN(CCCNc1c2ccc(Cl)cc2nc3ccc(OC)cc13)Cc4c(C)cc(C)cc4C |
| SJ000522959 | −47.41816909 | Q-1 | COc1ccc2nc3cc(Cl)ccc3c(NCCCNCc4oc(cc4)c5cccc(Cl)c5)c2c1 |
| SJ000522970 | −51.68913005 | Q-1 | CCCN(CCCNc1c2ccc(Cl)cc2nc3ccc(OC)cc13)Cc4csc5ccccc45 |
| SJ000520924 | −56.4502013 | Q-1 | CCCN(CCCNc1c2ccc(Cl)cc2nc3ccc(OC)cc13)Cc4ccc(Br)s4 |
| SJ000522989 | −56.7026981 | Q-1 | CCCN(CCCNc1c2ccc(Cl)cc2nc3ccc(OC)cc13)Cc4cc(Br)cs4 |
| SJ000523031 | −57.15035883 | Q-1 | COc1ccc2nc3cc(Cl)ccc3c(NCCCNCc4ccccc4N)c2c1 |
| SJ000522969 | −68.77297392 | Q-1 | CCCN(CCCNc1c2ccc(Cl)cc2nc3ccc(OC)cc13)Cc4nc5ccccc5s4 |
| SJ000522738 | −71.0251739 | Q-1 | CCCN(CCCNc1c2ccc(Cl)cc2nc3ccc(OC)cc13)Cc4ccc(Cl)c(c4)[N+](═O)[O−] |
| SJ000520988 | −73.54256376 | Q-1 | COc1ccc2nc3cc(Cl)ccc3c(NCCCNCc4cccc(OC(F)(F)F)c4)c2c1 |
| SJ000522740 | −74.00629347 | Q-1 | CCCN(CCCNc1c2ccc(Cl)cc2nc3ccc(OC)cc13)Cc4ccc(cc4)c5ccccc5 |
| SJ000522965 | −81.23577805 | Q-1 | CCCN(CCCNc1c2ccc(Cl)cc2nc3ccc(OC)cc13)Cc4cc(OC)ccc4F |
| SJ000522960 | −81.49221597 | Q-1 | COc1ccc2nc3cc(Cl)ccc3c(NCCCNCc4c(Cl)ccc(Cl)c4Cl)c2c1 |
| SJ000520930 | −81.95594568 | Q-1 | CCCN(CCCNc1c2ccc(Cl)cc2nc3ccc(OC)cc13)Cc4cccc(OC)c4O |
| SJ000522966 | −82.14598285 | Q-1 | CCCN(CCCNc1c2ccc(Cl)cc2nc3ccc(OC)cc13)Cc4nc(C)cs4 |
| SJ000522737 | −83.74461742 | Q-1 | CCCN(CCCNc1c2ccc(Cl)cc2nc3ccc(OC)cc13)Cc4cc5ccccc5nc4Cl |
| SJ000520921 | −83.81086452 | Q-1 | CCCN(CCCNc1c2ccc(Cl)cc2nc3ccc(OC)cc13)Cc4sccc4C |
| SJ000522975 | −89.07754245 | Q-1 | CCCN(CCCNc1c2ccc(Cl)cc2nc3ccc(OC)cc13)Cc4cncc(Br)c4 |
| SJ000520945 | −90.12777875 | Q-1 | CCCN(CCCNc1c2ccc(Cl)cc2nc3ccc(OC)cc13)Cc4cccc(F)c4 |
| SJ000520926 | −91.8376032 | Q-1 | CCCN(CCCNc1c2ccc(Cl)cc2nc3ccc(OC)cc13)Cc4ccccc4O |
| SJ000522739 | −94.542895 | Q-1 | CCCN(CCCNc1c2ccc(Cl)cc2nc3ccc(OC)cc13)Cc4cccc(c4)C#N |
| SJ000523036 | −95.37896027 | Q-1 | COc1ccc2nc3cc(Cl)ccc3c(NCCCNCc4ccc(Cl)c(c4)[N+](═O)[O−])c2c1 |
| SJ000522958 | −97.82951164 | Q-1 | COc1ccc2nc3cc(Cl)ccc3c(NCCCNCc4ccc(Cl)cc4Cl)c2c1 |
| SJ000523019 | −102.7575356 | Q-1 | CCCN(CCCNc1c2ccc(Cl)cc2nc3ccc(OC)cc13)Cc4oc(cc4)c5ccccc5[N+](═O)[O−] |
| SJ000522972 | −105.0761842 | Q-1 | CCCN(CCCNc1c2ccc(Cl)cc2nc3ccc(OC)cc13)Cc4ccc(CC)cc4 |
| SJ000522979 | −107.9910566 | Q-1 | CCCN(CCCNc1c2ccc(Cl)cc2nc3ccc(OC)cc13)Cc4cccc5OCOc45 |
| SJ000523038 | −109.4521267 | Q-1 | COc1ccc2nc3cc(Cl)ccc3c(NCCCNCc4ccc5ccccc5c4)c2c1 |
| SJ000520999 | −116.7337651 | Q-1 | COc1ccc2nc3cc(Cl)ccc3c(NCCCNCc4c(F)cccc4C(F)(F)F)c2c1 |
| SJ000522988 | −117.5306393 | Q-1 | CCCN(CCCNc1c2ccc(Cl)cc2nc3ccc(OC)cc13)Cc4nccs4 |
| SJ000520944 | −118.5541747 | Q-1 | CCCN(CCCNc1c2ccc(Cl)cc2nc3ccc(OC)cc13)Cc4ccccc4F |
| SJ000523018 | −118.6241904 | Q-1 | CCCN(CCCNc1c2ccc(Cl)cc2nc3ccc(OC)cc13)Cc4ccc(cc4)C(═O)OC |
| SJ000520960 | −119.324348 | Q-1 | CCCN(CCCNc1c2ccc(Cl)cc2nc3ccc(OC)cc13)Cc4cc(C)ccc4C |
| SJ000522731 | −120.9754886 | Q-1 | CCCN(CCCNc1c2ccc(Cl)cc2nc3ccc(OC)cc13)CCC(C)c4ccccc4 |
| SJ000520927 | −124.8178205 | Q-1 | CCCN(CCCNc1c2ccc(Cl)cc2nc3ccc(OC)cc13)Cc4ccc(C)s4 |
| SJ000522976 | −127.0260809 | Q-1 | CCCN(CCCNc1c2ccc(Cl)cc2nc3ccc(OC)cc13)Cc4scnc4C |
| SJ000523023 | −127.5862069 | Q-1 | COc1ccc2nc3cc(Cl)ccc3c(NCCCNCc4ccncc4)c2c1 |
| SJ000520925 | −127.9257809 | Q-1 | CCCN(CCCNc1c2ccc(Cl)cc2nc3ccc(OC)cc13)Cc4ccccc4 |
| SJ000520953 | −129.1238821 | Q-1 | CCCN(CCCNc1c2ccc(Cl)cc2nc3ccc(OC)cc13)Cc4ccc5ccccc5c4 |
| SJ000522957 | −133.0324611 | Q-1 | COC(═O)c1ccc(CNCCCNc2c3ccc(Cl)cc3nc4ccc(OC)cc24)cc1 |
| SJ000520929 | −137.3183966 | Q-1 | CCCN(CCCNc1c2ccc(Cl)cc2nc3ccc(OC)cc13)Cc4oc(Br)cc4 |
| SJ000520946 | −141.6593734 | Q-1 | CCCN(CCCNc1c2ccc(Cl)cc2nc3ccc(OC)cc13)Cc4ccc(F)cc4 |
| SJ000520919 | −146.2103973 | Q-1 | CCCN(CCCNc1c2ccc(Cl)cc2nc3ccc(OC)cc13)Cc4ccccn4 |
| SJ000520997 | −146.7005076 | Q-1 | COc1ccc2nc3cc(Cl)ccc3c(NCCCNCc4oc5ccccc5c4)c2c1 |
| SJ000520933 | −150.8314371 | Q-1 | CCCN(CCCNc1c2ccc(Cl)cc2nc3ccc(OC)cc13)Cc4cc(OC)ccc4O |
| SJ000520963 | −153.7015568 | Q-1 | CCCCCCN(CCC)CCCNc1c2ccc(Cl)cc2nc3ccc(OC)cc13 |
| SJ000522735 | −154.4302749 | Q-1 | CCCN(CCCNc1c2ccc(Cl)cc2nc3ccc(OC)cc13)Cc4ccc(s4)[N+](═O)[O−] |
| SJ000520954 | −157.8088771 | Q-1 | CCCN(CCCNc1c2ccc(Cl)cc2nc3ccc(OC)cc13)Cc4cccc(C)c4 |
| SJ000522973 | −159.8634693 | Q-1 | CCCN(CCCNc1c2ccc(Cl)cc2nc3ccc(OC)cc13)Cc4nccn4C |
| SJ000520939 | −160.2835638 | Q-1 | CCCN(CCCNc1c2ccc(Cl)cc2nc3ccc(OC)cc13)CC(CC)CC |
| SJ000522964 | −162.2440049 | Q-1 | CCCN(CCCNc1c2ccc(Cl)cc2nc3ccc(OC)cc13)Cc4cn(C)c5ccccc45 |
| SJ000520938 | −163.9036105 | Q-1 | CCCN(CCCNc1c2ccc(Cl)cc2nc3ccc(OC)cc13)CC4CCCCC4 |
| SJ000520920 | −164.1344302 | Q-1 | CCCN(CCCNc1c2ccc(Cl)cc2nc3ccc(OC)cc13)Cc4cccs4 |
| SJ000523032 | −165.3246981 | Q-1 | COc1ccc2nc3cc(Cl)ccc3c(NCCCNCc4occc4)c2c1 |
| SJ000522978 | −166.2349029 | Q-1 | CCCN(CCCNc1c2ccc(Cl)cc2nc3ccc(OC)cc13)Cc4c[nH]c5ccccc45 |
| SJ000361095 | −169.468367 | Q-1 | COc1ccc2nc3cc(Cl)ccc3c(NCCCNCc4ccccc4C(F)(F)F)c2c1 |
| SJ000523020 | −169.7356905 | Q-1 | CCCN(CCCNc1c2ccc(Cl)cc2nc3ccc(OC)cc13)Cc4oc(cc4)c5cccc(Cl)c5 |
| SJ000520957 | −175.4532301 | Q-1 | CCCN(CCCNc1c2ccc(Cl)cc2nc3ccc(OC)cc13)Cc4ccc(OC)c(OC)c4 |
| SJ000522963 | −177.5074392 | Q-1 | CCCN(CCCNc1c2ccc(Cl)cc2nc3ccc(OC)cc13)Cc4ccc(OC)c(F)c4 |
| SJ000520914 | −184.2289515 | Q-1 | COc1ccc2nc3cc(Cl)ccc3c(NCCCNCc4ccc5[nH]ccc5c4)c2c1 |
| SJ000522733 | −187.0900961 | Q-1 | CCCN(CCCNc1c2ccc(Cl)cc2nc3ccc(OC)cc13)Cc4c(F)cccc4OC |
| SJ000522736 | −192.3507789 | Q-1 | CCCN(CCCNc1c2ccc(Cl)cc2nc3ccc(OC)cc13)Cc4cn(C(═O)C)c5ccccc45 |
| SJ000520940 | −193.5410467 | Q-1 | CCCN(CCCNc1c2ccc(Cl)cc2nc3ccc(OC)cc13)Cc4ccc5OCOc5c4 |
| SJ000520992 | −200.9627166 | Q-1 | COc1ccc2nc3cc(Cl)ccc3c(NCCCNCc4ccc(OC(F)(F)F)cc4)c2c1 |
| SJ000520935 | −202.6430947 | Q-1 | CCCN(CCCNc1c2ccc(Cl)cc2nc3ccc(OC)cc13)Cc4occc4 |
| SJ000520941 | −210.3448276 | Q-1 | CCCCCN(CCC)CCCNc1c2ccc(Cl)cc2nc3ccc(OC)cc13 |
| SJ000522981 | −212.3964889 | Q-1 | CCCN(CCCNc1c2ccc(Cl)cc2nc3ccc(OC)cc13)CCOCc4ccccc4 |
| SJ000523030 | −219.4186817 | Q-1 | COc1ccc2nc3cc(Cl)ccc3c(NCCCNCc4oc(Br)cc4)c2c1 |
| SJ000521000 | −220.3570803 | Q-1 | COc1ccc2nc3cc(Cl)ccc3c(NCCCNCc4cc5ccccc5s4)c2c1 |
| SJ000520947 | −228.3388762 | Q-1 | CCCN(CCCNc1c2ccc(Cl)cc2nc3ccc(OC)cc13)Cc4ccccc4[N+](═O)[O−] |
| SJ000361089 | −229.0245114 | Q-1 | COc1ccc2nc3cc(Cl)ccc3c(NCCCNCc4oc(C)cc4)c2c1 |
| SJ000521001 | −231.8396639 | Q-1 | COc1ccc2nc3cc(Cl)ccc3c(NCCCNCc4cc(OC(F)(F)F)ccc4OC)c2c1 |
| SJ000522983 | −233.7300893 | Q-1 | CCCN(CCCNc1c2ccc(Cl)cc2nc3ccc(OC)cc13)CCC(C)(C)C |
| SJ000520943 | −241.4318222 | Q-1 | CCCN(CCCNc1c2ccc(Cl)cc2nc3ccc(OC)cc13)Cc4ccc(OC)cc4 |
| SJ000520961 | −244.5825311 | Q-1 | CCCN(CCCNc1c2ccc(Cl)cc2nc3ccc(OC)cc13)Cc4cccc(OC)c4 |
| SJ000520987 | −252.3542797 | Q-1 | COc1ccc2nc3cc(Cl)ccc3c(NCCCNCc4cn(C)c5ccccc45)c2c1 |
| SJ000523024 | −255.2583637 | Q-1 | COc1ccc2nc3cc(Cl)ccc3c(NCCCNCc4ccccn4)c2c1 |
| SJ000520991 | −257.115351 | Q-1 | COc1ccc2nc3cc(Cl)ccc3c(NCCCNCc4cccc5OCOc45)c2c1 |
| SJ000520922 | −258.0255558 | Q-1 | CCCN(CCCNc1c2ccc(Cl)cc2nc3ccc(OC)cc13)Cc4ccsc4 |
| SJ000520951 | −258.5922206 | Q-1 | CCCN(CCCNc1c2ccc(Cl)cc2nc3ccc(OC)cc13)Cc4ccccc4C(═O)O |
| SJ000520959 | −264.8170838 | Q-1 | CCCN(CCCNc1c2ccc(Cl)cc2nc3ccc(OC)cc13)CC(C)CC |
| SJ000520958 | −267.2397651 | Q-1 | CCCN(CCCNc1c2ccc(Cl)cc2nc3ccc(OC)cc13)CCCc4ccccc4 |
| SJ000520923 | −271.0484859 | Q-1 | CCCN(CCCNc1c2ccc(Cl)cc2nc3ccc(OC)cc13)Cc4ccc[nH]4 |
| SJ000520950 | −271.1209465 | Q-1 | CCCN(CCCNc1c2ccc(Cl)cc2nc3ccc(OC)cc13)Cc4ccccc4OC |
| SJ000520936 | −271.3564094 | Q-1 | CCCN(CCCNc1c2ccc(Cl)cc2nc3ccc(OC)cc13)CC4CC4 |
| SJ000522980 | −276.6497462 | Q-1 | CCCN(CCCNc1c2ccc(Cl)cc2nc3ccc(OC)cc13)Cc4cnn(C)c4 |
| SJ000521006 | −279.9046727 | Q-1 | COc1ccc2nc3cc(Cl)ccc3c(NCCCNCc4nccn4C)c2c1 |
| SJ000361097 | −281.6909573 | Q-1 | CCCCCCNCCCNc1c2ccc(Cl)cc2nc3ccc(OC)cc13 |
| SJ000522971 | −286.942062 | Q-1 | CCCCN(CCC)CCCNc1c2ccc(Cl)cc2nc3ccc(OC)cc13 |
| SJ000520996 | −287.6422195 | Q-1 | COc1ccc2nc3cc(Cl)ccc3c(NCCCNCc4cc(Br)cs4)c2c1 |
| SJ000520917 | −288.2023455 | Q-1 | CCCN(CCC)CCCNc1c2ccc(Cl)cc2nc3ccc(OC)cc13 |
| SJ000521002 | −290.1627866 | Q-1 | COc1ccc2nc3cc(Cl)ccc3c(NCCCNCc4cc(ccc4OC)C(F)(F)F)c2c1 |
| SJ000520915 | −298.7747243 | Q-1 | COc1ccc2nc3cc(Cl)ccc3c(NCCCNCc4c(F)cccc4OC)c2c1 |
| SJ000520916 | −299.4048661 | Q-1 | COc1ccc2nc3cc(Cl)ccc3c(NCCCNCc4cocc4)c2c1 |
| SJ000522987 | −302.6255908 | Q-1 | CCCN(CCCNc1c2ccc(Cl)cc2nc3ccc(OC)cc13)Cc4cccn4C |
| SJ000523029 | −304.7960791 | Q-1 | COc1ccc2nc3cc(Cl)ccc3c(NCCCNCc4ccc(C)s4)c2c1 |
| SJ000361094 | −321.319797 | Q-1 | COc1ccc2nc3cc(Cl)ccc3c(NCCCNCc4sccc4C)c2c1 |
| SJ000361091 | −327.2011203 | Q-1 | COc1ccc2nc3cc(Cl)ccc3c(NCCCNCc4cc(F)ccc4F)c2c1 |
| SJ000523022 | −329.6538589 | Q-1 | CCCN(CCCNc1c2ccc(Cl)cc2nc3ccc(OC)cc13)Cc4oc(cc4)[N+](═O)[O−] |
| SJ000523037 | −333.1524593 | Q-1 | COc1ccc2nc3cc(Cl)ccc3c(NCCCNCc4cccc(c4)C#N)c2c1 |
| SJ000523025 | −336.8632942 | Q-1 | COc1ccc2nc3cc(Cl)ccc3c(NCCCNCCCSC)c2c1 |
| SJ000523033 | −338.862206 | Q-1 | COc1ccc(CNCCCNc2c3ccc(Cl)cc3nc4ccc(OC)cc24)cc1 |
| SJ000523027 | −340.2533952 | Q-1 | COc1ccc2nc3cc(Cl)ccc3c(NCCCNCCC(C)c4ccccc4)c2c1 |
| SJ000523026 | −353.834051 | Q-1 | COc1ccc2nc3cc(Cl)ccc3c(NCCCNCc4ccsc4)c2c1 |
| SJ000523034 | −353.834051 | Q-1 | COc1ccc2nc3cc(Cl)ccc3c(NCCCNCc4ccccc4F)c2c1 |
| SJ000520998 | −354.9973744 | Q-1 | COc1ccc(F)c(CNCCCNc2c3ccc(Cl)cc3nc4ccc(OC)cc24)c1 |
| SJ000520989 | −355.2074217 | Q-1 | COc1ccc2nc3cc(Cl)ccc3c(NCCCNCc4nccs4)c2c1 |
| SJ000361085 | −363.3073866 | Q-1 | COc1ccc2nc3cc(Cl)ccc3c(NCCCNCc4ccc5OCOc5c4)c2c1 |
| SJ000520993 | −365.3597059 | Q-1 | COc1ccc2nc3cc(Cl)ccc3c(NCCCNCc4cc(F)ccc4OC)c2c1 |
| SJ000521004 | −366.222259 | Q-1 | COc1ccc2nc3cc(Cl)ccc3c(NCCCNCc4cccc(F)c4)c2c1 |
| SJ000521005 | −390.3553299 | Q-1 | CCc1ccc(CNCCCNc2c3ccc(Cl)cc3nc4ccc(OC)cc24)cc1 |
| SJ000520937 | −394.5760185 | Q-1 | CCCN(CCCNc1c2ccc(Cl)cc2nc3ccc(OC)cc13)Cc4cocc4 |
| SJ000523035 | −398.2196091 | Q-1 | COc1ccc2nc3cc(Cl)ccc3c(NCCCNCc4ccc(F)cc4)c2c1 |
| SJ000520985 | −402.4680553 | Q-1 | COc1ccc2nc3cc(Cl)ccc3c(NCCCNCc4cccn4C)c2c1 |
| SJ000520995 | −405.5487485 | Q-1 | CCCCNCCCNc1c2ccc(Cl)cc2nc3ccc(OC)cc13 |
| SJ000520990 | −408.1393313 | Q-1 | COc1ccc2nc3cc(Cl)ccc3c(NCCCNCc4cccc(c4)C(F)(F)F)c2c1 |
| SJ000520986 | −408.9795204 | Q-1 | COc1ccc2nc3cc(Cl)ccc3c(NCCCNCc4cccnc4)c2c1 |
| SJ000361090 | −411.1500088 | Q-1 | COc1ccc2nc3cc(Cl)ccc3c(NCCCNCC4CCCCC4)c2c1 |
| SJ000361092 | −427.3683339 | Q-1 | CCC(CC)CNCCCNc1c2ccc(Cl)cc2nc3ccc(OC)cc13 |
| SJ000361086 | −432.4347978 | Q-1 | COc1ccc2nc3cc(Cl)ccc3c(NCCCNCc4ccccc4)c2c1 |
| SJ000521003 | −442.3070191 | Q-1 | COc1ccc2nc3cc(Cl)ccc3c(NCCCNCC4CCOC4)c2c1 |
| SJ000361093 | −451.4937441 | Q-1 | CCC(C)CNCCCNc1c2ccc(Cl)cc2nc3ccc(OC)cc13 |
| SJ000361088 | −453.5364712 | Q-1 | COc1ccc2nc3cc(Cl)ccc3c(NCCCNCc4cccs4)c2c1 |
| SJ000520994 | −456.3101698 | Q-1 | COc1ccc2nc3cc(Cl)ccc3c(NCCCNCCC(C)(C)C)c2c1 |
| SJ000361096 | −498.4764661 | Q-1 | CCCNCCCNc1c2ccc(Cl)cc2nc3ccc(OC)cc13 |
| SJ000361087 | −502.3576475 | Q-1 | COc1ccc2nc3cc(Cl)ccc3c(NCCCNCC4CC4)c2c1 |
| SJ000361098 | −514.5459188 | Q-1 | COc1ccc2nc3cc(Cl)ccc3c(NCCCNCCC(C)SC)c2c1 |
| SJ000520962 | −591.7273932 | Q-1 | CCCN(CCCNc1c2ccc(Cl)cc2nc3ccc(OC)cc13)CCc4ccccc4 |
| SJ000578361 | 28.01424313 | Q-2 | Clc1ccc2c(NCCNC(═O)C[C@@H]3CC[C@@]4(CC3)OO[C@@]5(04)C6CC7CC(CC5C7)C6)ccnc2c1 |
| SJ000016433 | 22.71447499 | Q-2 | CC(Nc1c2ccccc2nc3ccccc13)C(═O)NC(CC(═O)N)C(═O)O |
| SJ000180435 | 18.67340179 | Q-2 | CCOC(═O)c1cnc2c(OC)cccc2c1NCCN3CCOCC3 |
| SJ000578381 | 18.39663924 | Q-2 | Clc1ccc2c(NCCNCC[C@@H]3CC[C@@]4(CC3)OO[c@@]5(04)C6CC7CC(CC5C7)C6)ccnc2c1 |
| SJ000332030 | 17.03974806 | Q-2 | CN(C)CC(═O)Nc1c2CCN(C)c2nc3ccccc13 |
| SJ000111684 | 11.84995031 | Q-2 | O═C(CN1CCN(CC1)C(c2ccccc2)c3ccccc3)Nc4c5CCCc5nc6ccccc46 |
| SJ000331537 | 11.78370321 | Q-2 | CCN(CC)CC(═O)Nc1c2CCN(C)c2nc3ccccc13 |
| SJ000027288 | 10.65750248 | Q-2 | CCOC(═O)c1cnc2ccc(OCC)cc2c1NCCN(C)C |
| SJ000111678 | 10.6248906 | Q-2 | CN(C)c1cc(NC(═O)CNC(C)(C)CC(C)(C)C)c2ccccc2n1 |
| SJ000016432 | 9.597548857 | Q-2 | OC(═O)CCCNC(═O)CNc1c2ccccc2nc3ccccc13 |
| SJ000172983 | 8.868830739 | Q-2 | CCOC(═O)c1cnc2c(C)cc(C)cc2c1NCCN3CCOCC3 |
| SJ000134078 | 8.53759523 | Q-2 | CCOC(═O)c1cnc2c(C)c(C)ccc2c1NCCN(C)C |
| SJ000111681 | 6.894203762 | Q-2 | CCc1nc2ccccc2c(NC(═O)CN3CCN(CC3)c4ccccc4)c1C |
| SJ000019487 | 6.417567453 | Q-2 | CCOC(═O)c1cnc2c(ccc3ccccc23)c1NCCN(C)C |
| SJ000027289 | 5.291487247 | Q-2 | CCOC(═O)c1cnc2ccc(OCC)cc2c1NCCN3CCOCC3 |
| SJ000111677 | 4.987658524 | Q-2 | CN(C)c1cc(NC(═O)CN2CCOCC2)c3ccccc3n1 |
| SJ000129560 | 4.960251739 | Q-2 | CCOC(═O)c1cnc2c(C)cc(Cl)cc2c1NCCN3CCOCC3 |
| SJ000037396 | 4.919567623 | Q-2 | CCOC(═O)c1cnc2c(C)cc(C)cc2c1NCCN(C)C |
| SJ000111680 | 4.647204017 | Q-2 | CN1CCN(CC(═O)Nc2cc(C)nc3ccccc23)CC1 |
| SJ000832405 | 4.364027824 | Q-2 | Cc1cc(NC(═O)CN[C@H]2CCN(Cc3ccccc3)C2)c4ccccc4n1 |
| SJ000184799 | 4.043409767 | Q-2 | CCOC(═O)c1cnc2c(cccc2c1NCCN3CCOCC3)C(═O)OC |
| SJ000027210 | 3.83405101 | Q-2 | CCOC(═O)c1cnc2c(C)cc(Cl)cc2c1NCCN(C)C |
| SJ000013622 | 3.28538599 | Q-2 | CN(C)CCNc1c2CCCc2nc3ccccc13 |
| SJ000019523 | 2.740658779 | Q-2 | CCN(CC)CC(═O)Nc1c2CCCCc2nc3ccccc13 |
| SJ000027266 | 2.575356078 | Q-2 | CCOC(═O)c1cnc2c(Cl)c(Cl)ccc2c1NCCN(C)C |
| SJ000331523 | 2.503063189 | Q-2 | CCCCN(CCCC)CC(═O)Nc1c2CCN(C)c2nc3ccccc13 |
| SJ000111682 | 1.592858393 | Q-2 | CCc1nc2ccccc2c(NC(═O)CN3CCCCC3)c1C |
| SJ000281260 | 0.834113542 | Q-2 | CCOC(═O)c1cnc2c(cccc2c1NCCN3CCOCC3)C(F)(F)F |
| SJ000031347 | 0.654190129 | Q-2 | CCOC(═O)c1ccc2ncc(C(═O)OCC)c(NCCN3CCOCC3)c2c1 |
| SJ000049655 | 0.654190129 | Q-2 | CCN(CC)CC(═O)Nc1c2CCCc2nc3ccccc13 |
| SJ000027268 | −0.391522683 | Q-2 | CCOC(═O)c1cnc2ccc(C)cc2c1NCCN3CCOCC3 |
| SJ000049110 | −0.604504803 | Q-2 | CCc1nc2ccccc2c(NC(═O)CNC(C)(C)CC(C)(C)C)c1C |
| SJ000111679 | −1.072431696 | Q-2 | Cc1cc(NC(═O)CN2CCN(CC2)C(═O)c3occc3)c4ccccc4n1 |
| SJ000175495 | −1.137755995 | Q-2 | CCOC(═O)c1cnc2c(C)cccc2c1NCCN3CCOCC3 |
| SJ000832581 | −1.277787502 | Q-2 | Cc1cc(NC(═O)CN[C@H]2CCN(Cc3cccc(F)c3)C2)c4ccccc4n1 |
| SJ000037393 | −1.333222921 | Q-2 | CCOC(═O)c1cnc2c(C)c(Cl)ccc2c1NCCN(C)C |
| SJ000279714 | −6.103014243 | Q-2 | CCOC(═O)c1cnc2c(CC)cccc2c1NCCN3CCOCC3 |
| SJ000163436 | −6.528969018 | Q-2 | CCOC(═O)c1cnc2c(Cl)cccc2c1NCCN3CCOCC3 |
| SJ000127079 | −7.81343093 | Q-2 | CCOC(═O)c1cnc2ccc(cc2c1NCCN3CCOCC3)C(═O)OC |
| SJ000175864 | −13.05895992 | Q-2 | CCOC(═O)c1cnc2c(Cl)cc(Cl)cc2c1NCCN3CCOCC3 |
| SJ000031339 | −47.24246439 | Q-2 | CCOC(═O)c1cnc2ccc(Br)cc2c1NCCN3CCOCC3 |
| SJ000487033 | −68.07281638 | Q-2 | CCCCCN(CCCCC)CC(═O)Nc1c2CCCc2nc3ccccc13 |
| SJ000111683 | −264.3269736 | Q-2 | Clc1cccc(c1)N2CCc3c(NC(═O)CN4CCN(CC4)C(═O)c5ccccc5)c6ccccc6nc23 |
| SJ000520434 | 69.43812358 | Q-3 | Clc1ccc2c(NCCCCN(Cc3ccccc3)Cc4ccc5[nH]ccc5c4)ccnc2c1 |
| SJ000520449 | 42.32361709 | Q-3 | Cc1ccc(F)cc1CN(CCCCNc2ccnc3cc(Cl)ccc23)Cc4ccccc4 |
| SJ000361194 | 41.92613448 | Q-3 | Clc1ccc2c(NCCCCN(CC3CCCCC3)Cc4ccccc4)ccnc2c1 |
| SJ000520451 | 39.87247433 | Q-3 | FC(F)(F)Oc1ccc(CN(CCCCNc2ccnc3cc(Cl)ccc23)Cc4ccccc4)cc1 |
| SJ000361207 | 35.43391852 | Q-3 | Clc1ccc(CN(CCCCNc2ccnc3cc(Cl)ccc23)Cc4ccccc4)c(Cl)c1 |
| SJ000361208 | 35.36767141 | Q-3 | CCC(C)CN(CCCCNc1ccnc2cc(Cl)ccc12)Cc3ccccc3 |
| SJ000357537 | 34.53910971 | Q-3 | CC(C)(C)CCN(CCCCNc1ccnc2cc(Cl)ccc12)Cc3ccccc3 |
| SJ000520446 | 33.97648228 | Q-3 | Clc1ccc2c(NCCCCN(Cc3ccccc3)Cc4csc5ccccc45)ccnc2c1 |
| SJ000520457 | 33.31401126 | Q-3 | FC(F)(F)Oc1cccc(CN(CCCCNc2ccnc3cc(Cl)ccc23)Cc4ccccc4)c1 |
| SJ000361209 | 32.65154025 | Q-3 | Cc1ccc(C)c(CN(CCCCNc2ccnc3cc(Cl)ccc23)Cc4ccccc4)c1 |
| SJ000520444 | 31.81547366 | Q-3 | COc1ccc(cc1CN(CCCCNc2ccnc3cc(Cl)ccc23)Cc4ccccc4)C(F)(F)F |
| SJ000520459 | 30.85944337 | Q-3 | FC(F)(F)Oc1ccccc1CN(CCCCNc2ccnc3cc(Cl)ccc23)Cc4ccccc4 |
| SJ000357506 | 30.64939611 | Q-3 | Oc1c(F)cc(F)cc1CN(CCCCNc2ccnc3cc(Cl)ccc23)Cc4ccccc4 |
| SJ000522725 | 30.20039748 | Q-3 | Fc1ccc(CN(CCCCNc2ccnc3cc(Cl)ccc23)Cc4ccccc4)cc1 |
| SJ000520443 | 29.90892842 | Q-3 | CCc1ccc(CN(CCCCNc2ccnc3cc(Cl)ccc23)Cc4ccccc4)cc1 |
| SJ000361206 | 29.53792647 | Q-3 | Cc1cccc(CN(CCCCNc2ccnc3cc(Cl)ccc23)Cc4ccccc4)c1 |
| SJ000520465 | 28.7513831 | Q-3 | Fc1cccc(c1CN(CCCCNc2ccnc3cc(Cl)ccc23)Cc4ccccc4)C(F)(F)F |
| SJ000361199 | 28.33887625 | Q-3 | Fc1cccc(CN(CCCCNc2ccnc3cc(Cl)ccc23)Cc4ccccc4)c1 |
| SJ000312692 | 28.27923153 | Q-3 | Clc1ccc2c(NCCCCN(Cc3ccccc3)Cc4ccc[nH]4)ccnc2c1 |
| SJ000361192 | 27.81550182 | Q-3 | FC(F)(F)c1ccccc1CN(CCCCNc2ccnc3cc(Cl)ccc23)Cc4ccccc4 |
| SJ000522729 | 27.81550182 | Q-3 | Clc1ccc2c(NCCCCN(CCCc3ccccc3)Cc4ccccc4)ccnc2c1 |
| SJ000357538 | 27.63871871 | Q-3 | CCCCN(CCCCNc1ccnc2cc(Cl)ccc12)Cc3ccccc3 |
| SJ000520447 | 26.65849816 | Q-3 | COc1ccc(OC(F)(F)F)cc1CN(CCCCNc2ccnc3cc(Cl)ccc23)Cc4ccccc4 |
| SJ000361204 | 26.49055979 | Q-3 | Clc1ccc2c(NCCCCN(Cc3ccccc3)Cc4cccc5ccccc45)ccnc2c1 |
| SJ000520455 | 26.37843515 | Q-3 | FC(F)(F)c1cccc(CN(CCCCNc2ccnc3cc(Cl)ccc23)Cc4ccccc4)c1 |
| SJ000361197 | 26.09307718 | Q-3 | Fc1ccc(CN(CCCCNc2ccnc3cc(Cl)ccc23)Cc4ccccc4)cc1F |
| SJ000361193 | 26.02683008 | Q-3 | Cc1cc(C)c(CN(CCCCNc2ccnc3cc(Cl)ccc23)Cc4ccccc4)c(C)c1 |
| SJ000361195 | 25.53824611 | Q-3 | CCC(CC)CN(CCCCNc1ccnc2cc(Cl)ccc12)Cc3ccccc3 |
| SJ000357470 | 25.46823035 | Q-3 | CC(C)CN(CCCCNc1ccnc2cc(Cl)ccc12)Cc3ccccc3 |
| SJ000520466 | 25.27874713 | Q-3 | FC(F)(F)c1cc(CN(CCCCNc2ccnc3cc(Cl)ccc23)Cc4ccccc4)ccc1Cl |
| SJ000578384 | 25.11815158 | Q-3 | Clc1ccc2c(NCCCCNC(═O)C[C@@H]3CC[C@@]4(CC3)OO[C@@]5(O4)C6CC7CC(CC5C7)C6)ccnc2c1 |
| SJ000312686 | 25.04813583 | Q-3 | Clc1ccc2c(NCCCCN(Cc3ccccc3)Cc4cccs4)ccnc2c1 |
| SJ000312697 | 24.83438225 | Q-3 | Oc1ccccc1CN(CCCCNc2ccnc3cc(Cl)ccc23)Cc4ccccc4 |
| SJ000361205 | 24.63564094 | Q-3 | Clc1ccc2c(NCCCCN(Cc3ccccc3)Cc4ccc5ccccc5c4)ccnc2c1 |
| SJ000361201 | 24.37065253 | Q-3 | [O−][N+](═O)c1cccc(CN(CCCCNc2ccnc3cc(Cl)ccc23)Cc4ccccc4)c1 |
| SJ000361212 | 23.57780501 | Q-3 | CCCCCCN(CCCCNc1ccnc2cc(Cl)ccc12)Cc3ccccc3 |
| SJ000520432 | 23.57568731 | Q-3 | COC(═O)CCCCCN(CCCCNc1ccnc2cc(Cl)ccc12)Cc3ccccc3 |
| SJ000361191 | 23.4377735 | Q-3 | Cc1ccsc1CN(CCCCNc2ccnc3cc(Cl)ccc23)Cc4ccccc4 |
| SJ000361211 | 23.37694601 | Q-3 | Clc1ccc2c(NCCCCN(CCc3ccccc3)Cc4ccccc4)ccnc2c1 |
| SJ000312688 | 22.03745843 | Q-3 | Clc1ccc2c(NCCCCN(Cc3ccccc3)Cc4ccc(Br)s4)ccnc2c1 |
| SJ000520436 | 21.65452136 | Q-3 | Clc1ccc2c(NCCCCN(Cc3ccccc3)Cc4cccc5OCOc45)ccnc2c1 |
| SJ000522727 | 21.52202716 | Q-3 | CN(C)c1ccc(CN(CCCCNc2ccnc3cc(Cl)ccc23)Cc4ccccc4)cc1 |
| SJ000357496 | 20.77717486 | Q-3 | COc1ccc(F)cc1CN(CCCCNc2ccnc3cc(Cl)ccc23)Cc4ccccc4 |
| SJ000361198 | 20.77717486 | Q-3 | Fc1ccccc1CN(CCCCNc2ccnc3cc(Cl)ccc23)Cc4ccccc4 |
| SJ000312687 | 20.70715911 | Q-3 | Cc1ccc(CN(CCCCNc2ccnc3cc(Cl)ccc23)Cc4ccccc4)s1 |
| SJ000522726 | 19.40211991 | Q-3 | COc1ccccc1CN(CCCCNc2ccnc3cc(Cl)ccc23)Cc4ccccc4 |
| SJ000361196 | 19.09679678 | Q-3 | CCCCCN(CCCCNc1ccnc2cc(Cl)ccc12)Cc3ccccc3 |
| SJ000357485 | 18.34216628 | Q-3 | Clc1ccc2c(NCCCCN(CC3CC3)Cc4ccccc4)ccnc2c1 |
| SJ000522718 | 17.34640294 | Q-3 | Clc1ccc2c(NCCCCN(Cc3ccccc3)Cc4ccccc4)ccnc2c1 |
| SJ000361200 | 17.20637143 | Q-3 | [O−][N+](═O)c1ccccc1CN(CCCCNc2ccnc3cc(Cl)ccc23)Cc4ccccc4 |
| SJ000522723 | 16.5062139 | Q-3 | Clc1ccc2c(NCCCCN(Cc3ccccc3)Cc4ccc5OCOc5c4)ccnc2c1 |
| SJ000312689 | 16.29616664 | Q-3 | Clc1ccc2c(NCCCCN(Cc3ccccc3)Cc4ccsc4)ccnc2c1 |
| SJ000361202 | 15.24593033 | Q-3 | [O−][N+](═O)c1ccc(CN(CCCCNc2ccnc3cc(Cl)ccc23)Cc4ccccc4)cc1 |
| SJ000520428 | 15.10589883 | Q-3 | Cc1[nH]cnc1CN(CCCCNc2ccnc3cc(Cl)ccc23)Cc4ccccc4 |
| SJ000312684 | 14.96356409 | Q-3 | Clc1ccc2c(NCCCCN(Cc3oc(Br)cc3)Cc4ccccc4)ccnc2c1 |
| SJ000312685 | 14.89731699 | Q-3 | Clc1ccc2c(NCCCCN(Cc3cocc3)Cc4ccccc4)ccnc2c1 |
| SJ000520441 | 14.5457728 | Q-3 | COc1ccc(F)c(CN(CCCCNc2ccnc3cc(Cl)ccc23)Cc4ccccc4)c1 |
| SJ000520438 | 13.14545773 | Q-3 | COc1ccc(CN(CCCCNc2ccnc3cc(Cl)ccc23)Cc4ccccc4)cc1F |
| SJ000311200 | 12.30526869 | Q-3 | ClC1═CC2═NC═CC(NCCCCN(CCC)CC3═COC═C3)═C2C═C1 |
| SJ000312681 | 12.04869162 | Q-3 | Clc1ccc2c(NCCCCN(Cc3occc3)Cc4ccccc4)ccnc2c1 |
| SJ000361210 | 11.71745611 | Q-3 | COc1cccc(CN(CCCCNc2ccnc3cc(Cl)ccc23)Cc4ccccc4)c1 |
| SJ000312690 | 9.531301756 | Q-3 | Clc1ccc2c(NCCCCN(Cc3ccccc3)Cc4ccccn4)ccnc2c1 |
| SJ000311205 | 7.684228952 | Q-3 | ClC1═CC2═NC═CC(NCCCCN(CCC)CC3═CC═CC═C3)═C2C═C1 |
| SJ000312694 | 7.676382908 | Q-3 | COc1cccc(CN(CCCCNc2ccnc3cc(Cl)ccc23)Cc4ccccc4)c1O |
| SJ000311203 | 6.844039909 | Q-3 | ClC1═CC2═NC═CC(NCCCCN(CCC)CC3═C(F)C═CC═C3OC)═C2C═C1 |
| SJ000522724 | 6.682676383 | Q-3 | COc1ccc(CN(CCCCNc2ccnc3cc(Cl)ccc23)Cc4ccccc4)cc1 |
| SJ000312696 | 6.633992648 | Q-3 | COc1ccc(O)c(CN(CCCCNc2ccnc3cc(Cl)ccc23)Cc4ccccc4)c1 |
| SJ000311199 | 6.493961141 | Q-3 | ClC1═CC2═NC═CC(NCCCCN(CCC)CC3═C(C)C═CS3)═C2C═C1 |
| SJ000520435 | 5.887711163 | Q-3 | Cn1ccnc1CN(CCCCNc2ccnc3cc(Cl)ccc23)Cc4ccccc4 |
| SJ000311196 | 5.723787852 | Q-3 | ClC1═CC2═NC═CC(NCCCCN(CCC)CC3═CC(Br)═CC═C3O)═C2C═C1 |
| SJ000311202 | 5.396203932 | Q-3 | ClC1═CC2═NC═CC(NCCCCN(CCC)CC3═CC(F)═CC═C3OC)═C2C═C1 |
| SJ000357501 | 5.09364607 | Q-3 | COc1cccc(F)c1CN(CCCCNc2ccnc3cc(Cl)ccc23)Cc4ccccc4 |
| SJ000361203 | 4.099039417 | Q-3 | OC(═O)c1ccccc1CN(CCCCNc2ccnc3cc(Cl)ccc23)Cc4ccccc4 |
| SJ000311197 | 3.90337826 | Q-3 | ClC1═CC2═NC═CC(NCCCCN(CCC)CCCSC)═C2C═C1 |
| SJ000286026 | 3.701556807 | Q-3 | CCN(CCO)CCCC(C)Nc1ccnc2cc(Cl)ccc12 |
| SJ000520453 | 2.376614773 | Q-3 | Clc1ccc2c(NCCCCN(Cc3ccccc3)Cc4cccnc4Br)ccnc2c1 |
| SJ000312682 | 2.012952914 | Q-3 | Cc1oc(CN(CCCCNc2ccnc3cc(Cl)ccc23)Cc4ccccc4)cc1 |
| SJ000522728 | 0.057966214 | Q-3 | COc1ccc(CN(CCCCNc2ccnc3cc(Cl)ccc23)Cc4ccccc4)cc1OC |
| SJ000311198 | −0.997724488 | Q-3 | ClC1═CC2═NC═CC(NCCCCN(CCC)CC3═CC═CN3)═C2C═C1 |
| SJ000520448 | −1.697882023 | Q-3 | Clc1ccc2c(NCCCCN(Cc3ccccc3)Cc4nccs4)ccnc2c1 |
| SJ000285185 | −2.591917854 | Q-3 | CCN(CC)CCCC(C)Nc1ccnc2cc(Cl)ccc12 |
| SJ000311204 | −3.378260109 | Q-3 | ClC1═CC2═NC═CC(NCCCCN(CCC)CC3═CNC4═C3C═CC═C4)═C2C═C1 |
| SJ000361190 | −9.945346141 | Q-3 | Clc1ccc2c(NCCCCN(Cc3ccccc3)Cc4ccncc4)ccnc2c1 |
| SJ000520456 | −12.86021862 | Q-3 | Cc1csc(CN(CCCCNc2ccnc3cc(Cl)ccc23)Cc4ccccc4)n1 |
| SJ000520440 | −14.35015746 | Q-3 | Cn1cc(CN(CCCCNc2ccnc3cc(Cl)ccc23)Cc4ccccc4)c5ccccc15 |
| SJ000357543 | −15.91624819 | Q-3 | Clc1ccc2c(NCCCCN(CCOCc3ccccc3)Cc4ccccc4)ccnc2c1 |
| SJ000285202 | −265.6736744 | Q-3 | CCN(CC)CCCC(C)Nc1c2ccc(Cl)cc2nc3ccc(OC)cc13 |
| SJ000523108 | 29.29611031 | Q-4 | CCCCCCN1CCC(CC1)Nc2ccnc3cc(Cl)ccc23 |
| SJ000523090 | 23.71783651 | Q-4 | COc1ccc(CN2CCC(CC2)Nc3ccnc4cc(Cl)ccc34)cc1 |
| SJ000357529 | 22.14656567 | Q-4 | Oc1ccccc1CN2CCC(CC2)Nc3ccnc4cc(Cl)ccc34 |
| SJ000523104 | 20.64856584 | Q-4 | Clc1ccc2c(NC3CCN(CCCc4ccccc4)CC3)ccnc2c1 |
| SJ000523106 | 18.67670226 | Q-4 | Cc1ccc(C)c(CN2CCC(CC2)Nc3ccnc4cc(Cl)ccc34)c1 |
| SJ000523101 | 18.53774789 | Q-4 | Cc1cccc(CN2CCC(CC2)Nc3ccnc4cc(Cl)ccc34)c1 |
| SJ000523085 | 15.42729381 | Q-4 | Clc1ccc2c(NC3CCN(CC4CCCCC4)CC3)ccnc2c1 |
| SJ000523102 | 13.09047579 | Q-4 | CN(C)c1ccc(CN2CCC(CC2)Nc3ccnc4cc(Cl)ccc34)cc1 |
| SJ000523088 | 12.16523718 | Q-4 | CCCCCN1CCC(CC1)Nc2ccnc3cc(Cl)ccc23 |
| SJ000523084 | 11.74514266 | Q-4 | Cc1cc(C)c(CN2CCC(CC2)Nc3ccnc4cc(Cl)ccc34)c(C)c1 |
| SJ000523103 | 11.74514266 | Q-4 | COc1ccc(CN2CCC(CC2)Nc3ccnc4cc(Cl)ccc34)cc1OC |
| SJ000523105 | 11.18501663 | Q-4 | CCC(C)CN1CCC(CC1)Nc2ccnc3cc(Cl)ccc23 |
| SJ000523093 | 10.65750248 | Q-4 | Fc1ccc(CN2CCC(CC2)Nc3ccnc4cc(Cl)ccc34)cc1 |
| SJ000357498 | 10.50302153 | Q-4 | COc1cccc(F)c1CN2CCC(CC2)Nc3ccnc4cc(Cl)ccc34 |
| SJ000357525 | 9.994748818 | Q-4 | COc1cccc(CN2CCC(CC2)Nc3ccnc4cc(Cl)ccc34)c1O |
| SJ000523086 | 9.754021619 | Q-4 | CCC(CC)CN1CCC(CC1)Nc2ccnc3cc(Cl)ccc23 |
| SJ000523097 | 8.596476296 | Q-4 | COc1ccccc1CN2CCC(CC2)Nc3ccnc4cc(Cl)ccc34 |
| SJ000357527 | 7.77938548 | Q-4 | COc1ccc(O)c(CN2CCC(CC2)Nc3ccnc4cc(Cl)ccc34)c1 |
| SJ000523087 | 7.643203677 | Q-4 | Clc1ccc2c(NC3CCN(Cc4ccc5OCOc5c4)CC3)ccnc2c1 |
| SJ000523092 | 6.621840157 | Q-4 | Fc1cccc(CN2CCC(CC2)Nc3ccnc4cc(Cl)ccc34)c1 |
| SJ000357524 | 6.553749255 | Q-4 | Clc1ccc2c(NC3CCN(Cc4ccccc4)CC3)ccnc2c1 |
| SJ000357493 | 6.152699569 | Q-4 | COc1ccc(F)cc1CN2CCC(CC2)Nc3ccnc4cc(Cl)ccc34 |
| SJ000523100 | 4.170567708 | Q-4 | Clc1ccc2c(NC3CCN(Cc4ccc5ccccc5c4)CC3)ccnc2c1 |
| SJ000357481 | 3.973394014 | Q-4 | Clc1ccc2c(NC3CCN(CC4CC4)CC3)ccnc2c1 |
| SJ000523107 | 3.149204188 | Q-4 | COc1cccc(CN2CCC(CC2)Nc3ccnc4cc(Cl)ccc34)c1 |
| SJ000523095 | 0.892700858 | Q-4 | [O−][N+](═O)c1cccc(CN2CCC(CC2)Nc3ccnc4cc(Cl)ccc34)c1 |
| SJ000311180 | 0.682653597 | Q-4 | ClC1═CC2═NC═CC(NC3CCN(CC4═CC═C(Cl)O4)CC3)═C2C═C1 |
| SJ000523091 | −0.997724488 | Q-4 | Fc1ccccc1CN2CCC(CC2)Nc3ccnc4cc(Cl)ccc34 |
| SJ000311181 | −1.208613499 | Q-4 | ClC1═CC2═NC═CC(NC3CCN(CC4═CC(Br)═CC═C4O)CC3)═C2C═C1 |
| SJ000523089 | −1.957613414 | Q-4 | Fc1ccc(CN2CCC(CC2)Nc3ccnc4cc(Cl)ccc34)cc1F |
| SJ000523094 | −2.025704315 | Q-4 | [O−][N+](═O)c1ccccc1CN2CCC(CC2)Nc3ccnc4cc(Cl)ccc34 |
| SJ000523098 | −2.194435243 | Q-4 | OC(═O)c1ccccc1CN2CCC(CC2)Nc3ccnc4cc(Cl)ccc34 |
| SJ000357512 | −4.380589599 | Q-4 | Clc1ccc2c(NC3CCN(Cc4oc(Br)cc4)CC3)ccnc2c1 |
| SJ000523096 | −4.953613073 | Q-4 | [O−][N+](═O)c1ccc(CN2CCC(CC2)Nc3ccnc4cc(Cl)ccc34)cc1 |
| SJ000523099 | −6.17889025 | Q-4 | Clc1ccc2c(NC3CCN(Cc4cccc5ccccc45)CC3)ccnc2c1 |
| SJ000278939 | 11.58496191 | Q-5 | CCc1ccc(cc1)S(═O)(═O)c2cnc3ccccc3c2N4CCN(C)CC4 |
| SJ000083972 | 7.411394501 | Q-5 | CCN1CCN(CC1)c2c(cnc3cc(OC)c(OC)cc23)S(═O)(═O)c4ccc(OC)cc4 |
| SJ000083965 | 7.146406095 | Q-5 | CCN1CCN(CC1)c2c(cnc3cc(OC)c(OC)cc23)S(═O)(═O)c4ccc(C)cc4 |
| SJ000227655 | 6.55018218 | Q-5 | Cc1cc(N2CCN(CC(O)COCC#C)CC2)c3ccccc3n1 |
| SJ000117076 | 6.020205366 | Q-5 | COc1ccc2ncc(c(N3CCN(CC3)c4ccccc4F)c2c1)S(═O)(═O)c5ccc(C)cc5 |
| SJ000117077 | 5.158993044 | Q-5 | CCOc1ccc2ncc(c(N3CCN(CC3)c4ccccc4F)c2c1)S(═O)(═O)c5ccc(C)cc5 |
| SJ000083953 | 4.673551549 | Q-5 | COc1cc2ncc(c(N3CCN(C)CC3)c2cc1OC)S(═O)(═O)c4ccc(Cl)cc4 |
| SJ000083959 | 4.533520042 | Q-5 | CCOc1ccc2ncc(c(N3CCN(CC)CC3)c2c1)S(═O)(═O)c4ccccc4 |
| SJ000083960 | 4.430274925 | Q-5 | CCN1CCN(CC1)c2c(cnc3ccc(Cl)cc23)S(═O)(═O)c4ccccc4 |
| SJ000247282 | 3.900298112 | Q-5 | CCN1CCN(CC1)c2c(cnc3ccc(OC)cc23)C(═O)c4ccc(C)cc4 |
| SJ000516812 | 3.502815502 | Q-5 | COC(═O)CN1CCN(C(C)C1═O)c2cc(C)nc3ccccc23 |
| SJ000083957 | 3.237827095 | Q-5 | CCN1CCN(CC1)c2c(cnc3ccc(CC)cc23)S(═O)(═O)c4ccccc4 |
| SJ000117080 | 3.171579993 | Q-5 | Cc1ccc(cc1)S(═O)(═O)c2cnc3ccccc3c2N4CCN(CC4)c5ccc(C)cc5C |
| SJ000247332 | 2.972838688 | Q-5 | CCc1ccc2ncc(c(N3CCN(C)CC3)c2c1)S(═O)(═O)c4ccc(C)c(C)c4 |
| SJ000247256 | 2.672567878 | Q-5 | Cc1ccc2ncc(c(N3CCN(Cc4ccccc4)CC3)c2c1)S(═O)(═O)c5ccccc5 |
| SJ000117084 | 2.442861875 | Q-5 | Cc1ccc(cc1)S(═O)(═O)c2cnc3ccccc3c2N4CCN(CC4)c5cc(C)ccc5C |
| SJ000083962 | 2.376614773 | Q-5 | CCN1CCN(CC1)c2c(cnc3ccc(CC)cc23)S(═O)(═O)c4ccc(C)cc4 |
| SJ000117055 | 2.111626366 | Q-5 | Fc1ccc2ncc(c(N3CCN(CC3)c4ccccc4)c2c1)S(═O)(═O)c5ccc(Cl)cc5 |
| SJ000083951 | 1.312795379 | Q-5 | COc1cc2ncc(c(N3CCN(C)CC3)c2cc1OC)S(═O)(═O)c4ccc(C)cc4 |
| SJ000247345 | 1.117919841 | Q-5 | CCN1CCN(CC1)c2c(cnc3ccc(F)cc23)S(═O)(═O)c4ccc(C)c(C)c4 |
| SJ000083955 | 0.822685104 | Q-5 | COc1ccc(cc1)S(═O)(═O)c2cnc3cc(OC)c(OC)cc3c2N4CCN(C)CC4 |
| SJ000083961 | 0.221295429 | Q-5 | CCN1CCN(CC1)c2c(cnc3ccc(C)cc23)S(═O)(═O)c4ccc(C)cc4 |
| SJ000083958 | 0.124213316 | Q-5 | CCN1CCN(CC1)c2c(cnc3ccc(OC)cc23)S(═O)(═O)c4ccccc4 |
| SJ000227652 | −0.207022193 | Q-5 | Cc1cc(N2CCN(CC(O)COCC═C)CC2)c3ccccc3n1 |
| SJ000247283 | −0.273269294 | Q-5 | CCOc1ccc2ncc(C(═O)c3ccccc3)c(N4CCN(CC)CC4)c2c1 |
| SJ000247365 | −0.273269294 | Q-5 | CCN1CCN(CC1)c2c(cnc3ccc(OC)cc23)C(═O)c4ccccc4 |
| SJ000280902 | −0.595795387 | Q-5 | CCOC(═O)c1cnc2ccc(cc2c1N3CCN(CCO)CC3)C(═O)O |
| SJ000187322 | −0.927708734 | Q-5 | CCOC(═O)c1cnc2ccc(Cl)cc2c1N3CCN(C)CC3 |
| SJ000083947 | −2.093795217 | Q-5 | COc1ccc2ncc(c(N3CCN(C)CC3)c2c1)S(═O)(═O)c4ccccc4 |
| SJ000228017 | −2.128188142 | Q-5 | OC(COCC═C)CN1CCN(CC1)c2cc(nc3ccccc23)C(F)(F)F |
| SJ000247281 | −2.260682345 | Q-5 | CCN1CCN(CC1)c2c(cnc3ccc(CC)cc23)C(═O)c4ccccc4 |
| SJ000247367 | −2.260682345 | Q-5 | CCN1CCN(CC1)c2c(cnc3ccccc23)S(═O)(═O)c4ccc(Cl)cc4 |
| SJ000247245 | −2.393176549 | Q-5 | COc1ccc2ncc(C(═O)c3ccccc3)c(N4CCN(C)CC4)c2c1 |
| SJ000117056 | −2.790659159 | Q-5 | CCc1ccc2ncc(c(N3CCN(CC3)c4ccccc4)c2c1)S(═O)(═O)c5ccc(OC)cc5 |
| SJ000279507 | −2.790659159 | Q-5 | CCOC(═O)c1cnc2ccc(OC)cc2c1N3CCN(C)CC3 |
| SJ000518980 | −2.989400464 | Q-5 | Clc1ccc2c(ccnc2c1)N3CCN(CCCN4CCN(CC4)c5ccnc6cc(Cl)ccc56)CC3 |
| SJ000083967 | −3.098197094 | Q-5 | CCN1CCN(CC1)c2c(cnc3ccc(C)cc23)S(═O)(═O)c4ccc(Cl)cc4 |
| SJ000117075 | −3.121894667 | Q-5 | COc1ccc(cc1)N2CCN(CC2)c3c(cnc4ccc(C)cc34)S(═O)(═O)c5ccc(Cl)cc5 |
| SJ000227654 | −3.25134054 | Q-5 | CC(C)COCC(O)CN1CCN(CC1)c2cc(C)nc3ccccc23 |
| SJ000117050 | −3.320635972 | Q-5 | Cc1ccc(cc1)S(═O)(═O)c2cnc3ccc(Cl)cc3c2N4CCN(CC4)c5ccccc5 |
| SJ000083956 | −3.448275862 | Q-5 | CCN1CCN(CC1)c2c(cnc3ccc(C)cc23)S(═O)(═O)c4ccccc4 |
| SJ000083952 | −3.658323123 | Q-5 | COc1ccc2ncc(c(N3CCN(C)CC3)c2c1)S(═O)(═O)c4ccc(Cl)cc4 |
| SJ000083963 | −3.916859887 | Q-5 | CCN1CCN(CC1)c2c(cnc3ccc(OC)cc23)S(═O)(═O)c4ccc(C)cc4 |
| SJ000083948 | −3.938386137 | Q-5 | CCOc1ccc2ncc(c(N3CCN(C)CC3)c2c1)S(═O)(═O)c4ccccc4 |
| SJ000117058 | −4.068431356 | Q-5 | Cc1ccc(cc1)S(═O)(═O)c2cnc3ccc(C)cc3c2N4CCN(CC4)c5ccc(F)cc5 |
| SJ000117063 | −4.204613159 | Q-5 | Fc1ccc(cc1)N2CCN(CC2)c3c(cnc4ccc(Cl)cc34)S(═O)(═O)c5ccc(F)cc5 |
| SJ000083966 | −4.358480658 | Q-5 | CCN1CCN(CC1)c2c(cnc3ccc(Cl)cc23)S(═O)(═O)c4ccc(C)cc4 |
| SJ000247335 | −4.579330904 | Q-5 | CCN1CCN(CC1)c2c(cnc3ccc(OC)cc23)S(═O)(═O)c4ccc(C)c(C)c4 |
| SJ000247334 | −4.711825108 | Q-5 | CCN1CCN(CC1)c2c(cnc3ccc(CC)cc23)S(═O)(═O)c4ccc(C)c(C)c4 |
| SJ000227653 | −4.953613073 | Q-5 | Cc1cc(N2CCN(CC(O)CO)CC2)c3ccccc3n1 |
| SJ000083969 | −4.98862244 | Q-5 | CCOc1ccc2ncc(c(N3CCN(CC)CC3)c2c1)S(═O)(═O)c4ccc(Cl)cc4 |
| SJ000083950 | −5.374296125 | Q-5 | CN1CCN(CC1)c2c(cnc3ccc(Cl)cc23)S(═O)(═O)c4ccccc4 |
| SJ000315176 | −5.440543226 | Q-5 | Fc1ccccc1N2CCN(CC2)c3c(cnc4ccc(Cl)cc34)S(═O)(═O)c5ccccc5 |
| SJ000247338 | −5.57303743 | Q-5 | CN1CCN(CC1)c2c(cnc3ccc(Cl)cc23)C(═O)c4ccccc4 |
| SJ000117068 | −5.705531633 | Q-5 | COc1ccccc1N2CCN(CC2)c3c(cnc4ccccc34)S(═O)(═O)c5ccc(C)c(C)c5 |
| SJ000117072 | −5.838025836 | Q-5 | COc1ccc(cc1)N2CCN(CC2)c3c(cnc4ccc(Cl)cc34)S(═O)(═O)c5ccc(C)cc5 |
| SJ000083964 | −5.898827236 | Q-5 | CCOc1ccc2ncc(c(N3CCN(CC)CC3)c2c1)S(═O)(═O)c4ccc(C)cc4 |
| SJ000083954 | −5.96884299 | Q-5 | CCc1ccc2ncc(c(N3CCN(C)CC3)c2c1)S(═O)(═O)c4ccc(OC)cc4 |
| SJ000083946 | −6.248906004 | Q-5 | CCc1ccc2ncc(c(N3CCN(C)CC3)c2c1)S(═O)(═O)c4ccccc4 |
| SJ000083971 | −6.388937511 | Q-5 | CCOc1ccc2ncc(c(N3CCN(CC)CC3)c2c1)S(═O)(═O)c4ccc(OC)cc4 |
| SJ000117047 | −6.566743955 | Q-5 | Cc1ccc(cc1)S(═O)(═O)c2cnc3ccccc3c2N4CCN(CC4)c5ccccc5 |
| SJ000083970 | −6.809032032 | Q-5 | CCN1CCN(CC1)c2c(cnc3cc(OC)c(OC)cc23)S(═O)(═O)c4ccc(Cl)cc4 |
| SJ000279526 | −7.064431015 | Q-5 | CCOC(═O)c1cnc2ccc(OCC)cc2c1N3CCN(C)CC3 |
| SJ000247344 | −7.626697582 | Q-5 | CN1CCN(CC1)c2c(cnc3ccc(F)cc23)S(═O)(═O)c4ccc(C)c(C)c4 |
| SJ000117078 | −8.289168599 | Q-5 | COc1ccc2ncc(c(N3CCN(CC3)c4ccccc4F)c2c1)S(═O)(═O)c5ccc(C)c(C)c5 |
| SJ000083945 | −8.419394364 | Q-5 | CN1CCN(CC1)c2c(cnc3ccc(C)cc23)S(═O)(═O)c4ccccc4 |
| SJ000247333 | −8.554157006 | Q-5 | COc1ccc2ncc(c(N3CCN(C)CC3)c2c1)S(═O)(═O)c4ccc(C)c(C)c4 |
| SJ000117065 | −8.686651209 | Q-5 | COc1ccccc1N2CCN(CC2)c3c(cnc4ccccc34)S(═O)(═O)c5ccc(C)cc5 |
| SJ000117064 | −9.084133819 | Q-5 | CCc1ccc2ncc(c(N3CCN(CC3)c4ccc(F)cc4)c2c1)S(═O)(═O)c5ccc(OC)cc5 |
| SJ000247243 | −9.084133819 | Q-5 | CCOc1ccc2ncc(C(═O)c3ccc(OC)cc3)c(N4CCN(CC)CC4)c2c1 |
| SJ000228018 | −9.515703464 | Q-5 | OC(COCC#C)CN1CCN(CC1)c2cc(nc3ccccc23)C(F)(F)F |
| SJ000247306 | −10.14408745 | Q-5 | CN1CCN(CC1)c2c(cnc3ccccc23)S(═O)(═O)c4ccc(Cl)cc4 |
| SJ000247254 | −10.60781716 | Q-5 | CCOc1ccc2ncc(C(═O)c3ccc(C)cc3)c(N4CCN(C)CC4)c2c1 |
| SJ000247339 | −10.80655846 | Q-5 | CCOc1ccc(cc1)C(═O)c2cnc3ccc(C)cc3c2N4CCN(CC)CC4 |
| SJ000117060 | −11.07154687 | Q-5 | CCOc1ccc2ncc(c(N3CCN(CC3)c4ccc(F)cc4)c2c1)S(═O)(═O)c5ccc(C)cc5 |
| SJ000083980 | −11.079993 | Q-5 | Fc1ccc(cc1)N2CCN(CC2)c3c(cnc4ccc(F)cc34)S(═O)(═O)c5ccccc5 |
| SJ000117074 | −11.20404107 | Q-5 | CCc1ccc(cc1)S(═O)(═O)c2cnc3ccccc3c2N4CCN(CC4)c5ccc(OC)cc5 |
| SJ000083949 | −11.50008752 | Q-5 | CN1CCN(CC1)c2c(cnc3ccc(F)cc23)S(═O)(═O)c4ccccc4 |
| SJ000083975 | −12.06021355 | Q-5 | CCOc1ccc2ncc(c(N3CCN(CC3)c4ccccc4)c2c1)S(═O)(═O)c5ccccc5 |
| SJ000117067 | −12.86021862 | Q-5 | COc1ccccc1N2CCN(CC2)c3c(cnc4ccc(Cl)cc34)S(═O)(═O)c5ccc(C)cc5 |
| SJ000117081 | −13.12520702 | Q-5 | Cc1cccc(N2CCN(CC2)c3c(cnc4ccccc34)S(═O)(═O)c5ccccc5)c1C |
| SJ000117073 | −13.25770123 | Q-5 | COc1ccc(cc1)N2CCN(CC2)c3c(cnc4ccc(F)cc34)S(═O)(═O)c5ccc(C)c(C)c5 |
| SJ000247355 | −13.39019543 | Q-5 | CCOc1ccc2ncc(c(N3CCN(CC)CC3)c2c1)S(═O)(═O)c4ccc(C)c(C)c4 |
| SJ000247268 | −13.58893673 | Q-5 | CN1CCN(CC1)c2c(cnc3ccccc23)S(═O)(═O)c4ccc(C)cc4 |
| SJ000247354 | −14.05266645 | Q-5 | CCOc1ccc2ncc(c(N3CCN(C)CC3)c2c1)S(═O)(═O)c4ccc(C)c(C)c4 |
| SJ000247284 | −14.25140775 | Q-5 | CCOc1ccc2ncc(C(═O)c3ccc(C)cc3)c(N4CCN(CC)CC4)c2c1 |
| SJ000117070 | −15.37152098 | Q-5 | COc1ccccc1N2CCN(CC2)c3c(cnc4ccc(C)cc34)S(═O)(═O)c5ccc(Cl)cc5 |
| SJ000117083 | −15.37760848 | Q-5 | Cc1ccc(C)c(c1)N2CCN(CC2)c3c(cnc4ccc(F)cc34)S(═O)(═O)c5ccccc5 |
| SJ000083977 | −15.70103273 | Q-5 | Clc1ccc2ncc(c(N3CCN(CC3)c4ccccc4)c2c1)S(═O)(═O)c5ccccc5 |
| SJ000247249 | −15.90758529 | Q-5 | CCOc1ccc2ncc(c(N3CCN(Cc4ccccc4)CC3)c2c1)S(═O)(═O)c5ccccc5 |
| SJ000193862 | −16.19114301 | Q-5 | CC(═O)Nc1ccc(cc1)S(═O)(═O)N2CCN(CC2)c3ccnc4cc(Cl)ccc34 |
| SJ000201754 | −16.75126904 | Q-5 | Clc1ccc2c(ccnc2c1)N3CCN(CC3)S(═O)(═O)c4ccc(Br)cc4 |
| SJ000117052 | −16.90129182 | Q-5 | Cc1ccc(cc1C)S(═O)(═O)c2cnc3ccc(F)cc3c2N4CCN(CC4)c5ccccc5 |
| SJ000083983 | −17.94153685 | Q-5 | COc1ccc(cc1)N2CCN(CC2)c3c(cnc4ccc(Cl)cc34)S(═O)(═O)c5ccccc5 |
| SJ000247277 | −18.95495197 | Q-5 | CCOc1ccc2ncc(C(═O)c3ccc(OC)cc3)c(N4CCN(C)CC4)c2c1 |
| SJ000083968 | −19.13180466 | Q-5 | CCN1CCN(CC1)c2c(cnc3ccc(CC)cc23)S(═O)(═O)c4ccc(Cl)cc4 |
| SJ000315081 | −19.55117589 | Q-5 | COc1ccccc1N2CCN(CC2)c3c(cnc4ccc(C)cc34)S(═O)(═O)c5ccccc5 |
| SJ000117057 | −20.61112951 | Q-5 | Cc1ccc(cc1)S(═O)(═O)c2cnc3ccccc3c2N4CCN(CC4)c5ccc(F)cc5 |
| SJ000117053 | −21.20735343 | Q-5 | CCc1ccc(cc1)S(═O)(═O)c2cnc3ccccc3c2N4CCN(CC4)c5ccccc5 |
| SJ000083973 | −29.70418344 | Q-5 | Cc1ccc2ncc(c(N3CCN(CC3)c4ccccc4)c2c1)S(═O)(═O)c5ccccc5 |
| SJ000247242 | −30.28320636 | Q-5 | Clc1ccc2ncc(c(N3CCN(Cc4ccccc4)CC3)c2c1)S(═O)(═O)c5ccccc5 |
| SJ000083976 | −31.10449851 | Q-5 | Fc1ccc2ncc(c(N3CCN(CC3)c4ccccc4)c2c1)S(═O)(═O)c5ccccc5 |
| SJ000199955 | −32.07187811 | Q-5 | COc1ccc(cc1)S(═O)(═O)N2CCN(CC2)c3ccnc4cc(Cl)ccc34 |
| SJ000117071 | −32.66810202 | Q-5 | COc1ccc(cc1)N2CCN(CC2)c3c(cnc4ccc(OC)cc34)S(═O)(═O)c5ccccc5 |
| SJ000083982 | −32.85489235 | Q-5 | COc1ccc(cc1)N2CCN(CC2)c3c(cnc4ccc(F)cc34)S(═O)(═O)c5ccccc5 |
| SJ000315092 | −33.48370074 | Q-5 | Cc1ccc2ncc(c(N3CCN(CC3)c4ccccc4F)c2c1)S(═O)(═O)c5ccccc5 |
| SJ000204656 | −34.39052666 | Q-5 | Clc1ccc(cc1)S(═O)(═O)N2CCN(CC2)c3ccnc4cc(Cl)ccc34 |
| SJ000083981 | −37.96604236 | Q-5 | COc1ccc(cc1)N2CCN(CC2)c3c(cnc4ccc(C)cc34)S(═O)(═O)c5ccccc5 |
| SJ000192519 | −38.69658827 | Q-5 | Cc1ccc(cc1)S(═O)(═O)N2CCN(CC2)c3ccnc4cc(Cl)ccc34 |
| SJ000117049 | −45.32129844 | Q-5 | CCOc1ccc2ncc(c(N3CCN(CC3)c4ccccc4)c2c1)S(═O)(═O)c5ccc(C)cc5 |
| SJ000117069 | −47.44233552 | Q-5 | CCc1ccc(cc1)S(═O)(═O)c2cnc3ccccc3c2N4CCN(CC4)c5ccccc5OC |
| SJ000203833 | −50.64260788 | Q-5 | Fc1ccc(cc1)S(═O)(═O)N2CCN(CC2)c3ccnc4cc(Cl)ccc34 |
| SJ000117051 | −52.47598543 | Q-5 | COc1ccc2ncc(c(N3CCN(CC3)c4ccccc4)c2c1)S(═O)(═O)c5ccc(C)c(C)c5 |
| SJ000114009 | −53.43433484 | Q-5 | COc1ccc(cc1OC)c2csc(n2)N3CCN(CC3)c4cc(C)nc5ccccc45 |
| SJ000117062 | −54.86424377 | Q-5 | Cc1ccc(cc1C)S(═O)(═O)c2cnc3ccccc3c2N4CCN(CC4)c5ccc(F)cc5 |
| SJ000117086 | −55.2583637 | Q-5 | CCc1ccc(cc1)S(═O)(═O)c2cnc3ccccc3c2N4CCN(Cc5ccccc5)CC4 |
| SJ000117079 | −56.58330573 | Q-5 | CCc1ccc(cc1)S(═O)(═O)c2cnc3ccccc3c2N4CCN(CC4)c5ccccc5F |
| SJ000247257 | −62.28055648 | Q-5 | Fc1ccc2ncc(c(N3CCN(Cc4ccccc4)CC3)c2c1)S(═O)(═O)c5ccccc5 |
| SJ000117061 | −70.49519709 | Q-5 | Cc1ccc(cc1)S(═O)(═O)c2cnc3ccc(F)cc3c2N4CCN(CC4)c5ccc(F)cc5 |
| SJ000247248 | −71.1576681 | Q-5 | COc1ccc2ncc(c(N3CCN(Cc4ccccc4)CC3)c2c1)S(═O)(═O)c5ccccc5 |
| SJ000117085 | −82.22093408 | Q-5 | COc1ccc2ncc(c(N3CCN(Cc4ccccc4)CC3)c2c1)S(═O)(═O)c5ccc(C)cc5 |
| SJ000117048 | −85.57324028 | Q-5 | COc1ccc2ncc(c(N3CCN(CC3)c4ccccc4)c2c1)S(═O)(═O)c5ccc(C)cc5 |
| SJ000117066 | −92.88671746 | Q-5 | COc1ccccc1N2CCN(CC2)c3c(cnc4ccc(C)cc34)S(═O)(═O)c5ccc(C)cc5 |
| SJ000315166 | −99.05523874 | Q-5 | Fc1ccc2ncc(c(N3CCN(CC3)c4ccccc4F)c2c1)S(═O)(═O)c5ccccc5 |
| SJ000117059 | −118.5243458 | Q-5 | COc1ccc2ncc(c(N3CCN(CC3)c4ccc(F)cc4)c2c1)S(═O)(═O)c5ccc(C)cc5 |
| SJ000083974 | −132.9774199 | Q-5 | COc1ccc2ncc(c(N3CCN(CC3)c4ccccc4)c2c1)S(═O)(═O)c5ccccc5 |
| SJ000083978 | −133.1174514 | Q-5 | COc1ccc2ncc(c(N3CCN(CC3)c4ccc(F)cc4)c2c1)S(═O)(═O)c5ccccc5 |
| SJ000083979 | −181.2882899 | Q-5 | CCOc1ccc2ncc(c(N3CCN(CC3)c4ccc(F)cc4)c2c1)S(═O)(═O)c5ccccc5 |
| SJ000117082 | −241.8102021 | Q-5 | COc1ccc2ncc(c(N3CCN(CC3)c4cccc(C)c4C)c2c1)S(═O)(═O)c5ccccc5 |
| SJ000315263 | −252.4640395 | Q-5 | COc1ccc2ncc(c(N3CCN(CC3)c4ccccc4F)c2c1)S(═O)(═O)c5ccccc5 |
| SJ000117054 | −446.9112289 | Q-5 | CCOc1ccc2ncc(c(N3CCN(CC3)c4ccccc4)c2c1)S(═O)(═O)c5ccc(Cl)cc5 |

TABLE 5
Dose response TR-FRET screening of 59 quinoline compounds against comounds against MAGE-11:PCF-11.

| | TR-FRET | | Compound Structure |
|---|---|---|---|
| SAMPLE | IC50 (uM) | IC50 Cl | (SMILES format) |
| SJ000311270-1 | 0.58 | 0.4340-0.8623 | FC(F)(F)c1cc(cc(cc(c1)C(F)(F)F)c2ccc3c(NCCCNCc40ccc4)ccnc3c2 |
| SJ000311286-1 | 1.16 | 1.0435-1.6665 | CC(C)(C)c1ccc(cc1)c2ccc3c(NCCCNCc4occc4)ccnc3c2 |
| SJ000522746-2 | 2.80 | 2.0839-3.6648 | CCCN(CCCNc1ccnc2cc(Cl)ccc12)Cc3ccc4[nH]ccc4c3 |
| SJ000311278-1 | 22.68 | 16.0357-26.8320 | FC(F)(F)c1ccc(cc1)c2ccc3c(NCCCNCc4occc4)ccnc3c2 |
| SJ000575192-1 | 22.75 | 21.3134-26.5078 | Clc1ccc2c(NCCCNCc3ccc[nH]3)ccnc2c1 |
| SJ000520434-1 | 23.44 | 20.4575-28.2250 | Clc1ccc2c(NCCCCN(Cc3ccccc3)Cc4ccc5[nH]ccc5c4)ccnc2c1 |
| SJ000521021-1 | 31.35 | 27.2520-35.8585 | COC(═O)CCCCCNCCCNc1ccnc2cc(Cl)ccc12 |
| SJ000311234-1 | 32.15 | UNDEFINED | CC(C)(C)c1ccc(Oc2ccc3c(NCCCNCc4occc4)ccnc3c2)cc1 |
| SJ000311306-2 | 32.24 | 29.7140-35.9595 | C(CNCc1occc1)CNc2ccnc3cc(Cc4ccccc4)ccc23 |
| SJ000361105-1 | 32.98 | 28.8870-39.9693 | FC(F)(F)c1ccc2c(NCCCNCc3ccc[nH]3)ccnc2c1 |
| SJ000575171-1 | 33.20 | 29.6281-36.3496 | CCCN(CCCNc1ccnc2cc(ccc12)C(F)(F)F)Cc3ccc4[nH]ccc4c3 |
| SJ000311314-1 | 38.36 | 34.1383-44.3800 | Clc1cccc(Cl)c1Cc2ccc3c(NCCCNCc4occc4)ccnc3c2 |
| SJ000520430-1 | 41.71 | 36.8262-48.8449 | Clc1ccc2c(NCCCN(Cc3ccccc3)Cc4ccc5[nH]ccc5c4)ccnc2c1 |
| SJ000311250-1 | 54.90 | 47.7215-62.2736 | C(CNCc1occc1)CNc2ccnc3cc(CC4CCCCC4)ccc23 |
| SJ000361124-1 | 57.63 | 47.8394-72.1804 | CCCCCCNCCCNc1ccnc2cc(ccc12)C(F)(F)F |
| SJ000311218-1 | >70 | UNDEFINED | COc1ccc(Oc2ccc3c(NCCCNCc4occc4)ccnc3c2)cc1 |
| SJ000311226-1 | >70 | 66.1725-86.8582 | Clc1ccc(Oc2ccc3c(NCCCNCc4occc4)ccnc3c2)cc1 |
| SJ000311262-1 | >70 | 61.6173-84.2183 | C(CNCc1occc1)CNc2ccnc3cc(ccc23)C4CCCCC4 |
| SJ000311274-1 | >70 | 25.8667-128.8723 | C(CNCc1occc1)CNc2ccnc3cc(ccc23)c4cccc5ccccc45 |
| SJ000311290-1 | >70 | 82.4277-122.7627 | C(CNCc1occc1)CNc2ccnc3cc(ccc23)c4ccc5OCOc5c4 |
| SJ000311318-2 | >70 | UNDEFINED | Cc1cc(C)cc(Cc2ccc3c(NCCCNCc4occc4)ccnc3c2)c1 |
| SJ000357506-2 | >70 | UNDEFINED | Oc1c(F)cc(F)cc1CN(CCCCNc2ccnc(Cl)ccc23)Cc4ccccc4 |
| SJ000357537-2 | >70 | UNDEFINED | CC(C)(C)CCN(CCCCNc1ccnc2cc(Cl)ccc12)Cc3ccccc3 |
| SJ000361169-2 | >70 | UNDEFINED | Cc1cc(C)cc(CN(CCCNc2ccnc3cc(Cl)ccc23)Cc4ccccc4)c(C)c1 |
| SJ000361194-2 | >70 | 0.3441-1.3005 | Clc1ccc2c(NCCCCN(CC3CCCCC3)Cc4ccccc4)ccnc2c1 |
| SJ000361207-2 | >70 | UNDEFINED | Clc1ccc(CN(CCCCNc2ccnc3cc(Cl)ccc23)Cc4ccccc4)c(Cl)c1 |
| SJ000361208-2 | >70 | 0.7324-0.8837 | CCC(C)CN(CCCCNc1ccnc2cc(Cl)ccc12)Cc3ccccc3 |
| SJ000361209-2 | >70 | UNDEFINED | Cc1ccc(C)1(CN(CCCCNc2ccnc3cc(Cl)ccc23)Cc4ccccc4)c1 |
| SJ000361255-1 | >70 | UNDEFINED | CCCN(CCNc1ccnc2cc(ccc12)C(F)(F)F)Cc3ccc[nH]3 |
| SJ000361266-1 | >70 | UNDEFINED | CCCN(CCCNc1ccnc2cc(ccc12)C(F)(F)F)CC3CC3 |
| SJ000361268-1 | >70 | UNDEFINED | CCCN(CCCNc1ccnc2cc(ccc12)C(F)(F)F)CC3CCCCC3 |
| SJ000361269-1 | >70 | UNDEFINED | CCCN(CCCNc1ccnc2cc(ccc12)C(F)(F)F)CC(CC)CC |
| SJ000361271-1 | >70 | 62.2262-90.4812 | CCCCCN(CCC)CCCNc1ccnc2cc(ccc12)C(F)(F)F |
| SJ000520433-1 | >70 | UNDEFINED | Clc1ccc2c(NCCCN(Cc3ccccc3)Cc4c[nH]c5ccccc45)ccnc2c1 |
| SJ000520444-1 | >70 | UNDEFINED | COc1ccc(c1CN(CCCCNc2ccnc3cc(Cl)ccc23)Cc4ccccc4)C(F)(F)F |
| SJ000520446-1 | >70 | UNDEFINED | Clc1ccc2c(NCCCCN(Cc3ccccc3)Cc4csc5ccccc45)ccnc2c1 |
| SJ000520449-1 | >70 | UNDEFINED | Cc1ccc(F)cc1CN(CCCCNc2ccnc3cc(Cl)ccc23)Cc4ccccc4 |
| SJ000520451-1 | >70 | UNDEFINED | FC(F)(F)Oc1ccc(CN(CCCCNc2ccnc3cc(Cl)ccc23)Cc4ccccc4)cc1 |
| SJ000520457-1 | >70 | UNDEFINED | FC(F)(F)Oc1cccc(CN(CCCCNc2ccnc3cc(Cl)ccc23)Cc4ccccc4)c1 |
| SJ000520476-1 | >70 | 114.7351-189.0376 | CCCN(CCCNc1ccnc2cc(ccc12)C(F)(F)F)CCC(C)(C)C |
| SJ000521009-2 | >70 | UNDEFINED | CCCN(CCCNc1ccnc2cc(ccc12)C(F)(F)F)Cc3ccc(CC)cc3 |
| SJ000521032-1 | >70 | UNDEFINED | Clc1ccc2c(NCCCNCc3cc4ccccc4s3)ccnc2c1 |
| SJ000521033-1 | >70 | 125.2880-288.8645 | Clc1ccc2c(NCCCNCc3csc4ccccc34)ccnc2c1 |
| SJ000521056-1 | >70 | UNDEFINED | FC(F)(F)c1ccc2c(NCCCNCc3cc(Br)cs3)ccnc2c1 |
| SJ000521060-1 | >70 | 42.8056-151.8022 | FC(F)(F)c1ccc2c(NCCCNCc3cc4333334s3)ccnc2c1 |
| SJ000521061-1 | >70 | 54.9933-72.1397 | FC(F)(F)c1ccc2c(NCCCNCc3csc4ccccc34)ccnc2c1 |
| SJ000522725-1 | >70 | UNDEFINED | Fc1ccc(CN(CCCCNc2ccnc3cc(Cl)ccc23)Cc4ccccc4)cc1 |
| SJ000522732-2 | >70 | UNDEFINED | CCCN(CCCNc1c2ccc(Cl)cc2nc3ccc(OC)cc13)Cc4ccc5[nH]ccc5c4 |
| SJ000522749-1 | >70 | UNDEFINED | CCCN(CCCNc1ccnc2cc(Cl)ccc12)Cc3c(C)cc(C)cc3C |
| SJ000523001-1 | >70 | UNDEFINED | CCCN(CCCNc1ccnc2cc(Cl)ccc12)Cc3ccc(CC)cc3 |
| SJ000523012-1 | >70 | UNDEFINED | CCCN(CCCNc1ccnc2cc(Cl)ccc12)Cc3cc(ccc3OC)(c(F)(F)F |
| SJ000523021-1 | >70 | 0.3868-0.6049 | CCCN(CCCNc1c2ccc(Cl)cc2nc3ccc(OC)cc13)Cc4c(Cl)ccc(Cl)c4Cl |
| SJ000523039-1 | >70 | UNDEFINED | CCCN(CCCNc1ccnc2cc(Cl)ccc12)Cc3ccc(cc3)c4ccccc4 |
| SJ000523040-1 | >70 | UNDEFINED | CCCN(CCCNc1ccnc2cc(Cl)ccc12)Cc3ccc4ccccc4c3 |
| SJ000523069-1 | >70 | UNDEFINED | Clc1cccc(c1)c2oc(CNCCCNc3ccnc4cc(Cl)ccc34)cc2 |
| SJ000523072-1 | >70 | UNDEFINED | CCCN(CCCNc1ccnc2cc(ccc12)C(F)(F)F)Cc3ccccc3OC |
| SJ000523083-1 | >70 | 91.5976-192.4126 | CCCCCCN(CCC)CCCNc1ccnc2cc(ccc12)C(F)(F)F |
| SJ000311230-1 | >70 | 66.8540-90.6468 | CN(C)c1cccc(Oc2ccc3c(NCCCNCc4occc4)ccnc3c2)c1 |

TABLE 6
TR-FRET and ADME results of 15 4-amnioquinoline compounds.

| | | | TR- | | | | | |
|---|---|---|---|---|---|---|---|---|
| | | | FRET, | Solu- | Caco-2 | Caco-2 | | |
| | Compound | | IC50, | bility | permeability, | permeability, | | |
| No. | Number | Structure | uM | uM | A/B (nm/sec) | B/A (nm/sec) | cLog P | tPSA |
| 1 | SJ001008068-01 | 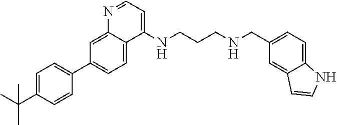 [SMILES: CC(C)(C)c1ccc(-c2ccc3c(NCCCNCc4ccc5[nH]ccc5c4)ccnc3c2)cc1] | 0.13 | 0.67 | 3.68 | 15.36 | 5.73 | 58.39 |
| 2 | SJ001008066-01 | 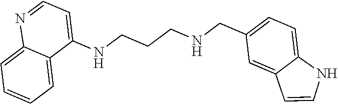 [SMILES: c1ccc2c(NCCCNCc3ccc4[nH]ccc4c3)ccnc2c1] | 0.13 | 37.77 | 81.68 | 80.93 | 2.62 | 48.45 |
| 3 | SJ001008069-01 | 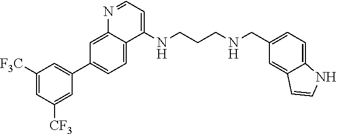 [SMILES: FC(F)(F)c1cc(-c2ccc3c(NCCCNCc4ccc5[nH]ccc5c4)ccnc3c2)cc(C(F)(F)F)c1] | 0.20 | 0.12 | 4.98 | 2.29 | 5.87 | 58.39 |
| 4 | SJ000520909-03 | 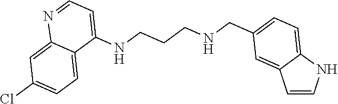 [SMILES: Clc1ccc2c(NCCCNCc3ccc4[nH]ccc4c3)ccnc2c1] | 0.24 | 59.80 | 195.94 | 213.15 | 3.17 | 48.45 |
| 5 | SJ000521054-02 | 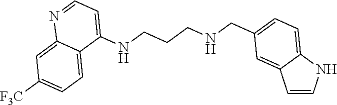 [SMILES: FC(F)(F)c1ccc2c(NCCCNCc3ccc4[nH]ccc4c3)ccnc2c1] | 0.40 | 26.48 | 115.14 | 142.50 | 3.54 | 48.45 |
| 6 | SJ000311270-01 | 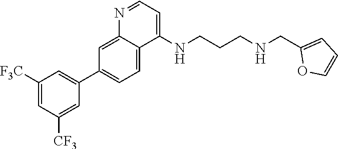 [SMILES: FC(F)(F)c1cc(-c2ccc3c(NCCCNCc4ccco4)ccnc3c2)cc(C(F)(F)F)c1] | 0.58 | 61.08 | 3.26 | 0.79 | 5.21 | 45.65 |
| 7 | SJ001008065-01 | 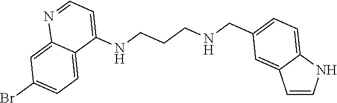 [SMILES: Brc1ccc2c(NCCCNCc3ccc4[nH]ccc4c3)ccnc2c1] | 0.62 | 57.14 | 182.80 | 192.69 | 3.44 | 48.45 |
| 8 | SJ000311286-01 | 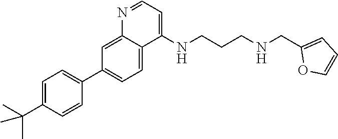 [SMILES: CC(C)(C)c1ccc(-c2ccc3c(NCCCNCc4ccco4)ccnc3c2)cc1] | 1.16 | 56.16 | 5.78 | 1.20 | 5.07 | 45.65 |
| 9 | SJ001008067-01 | 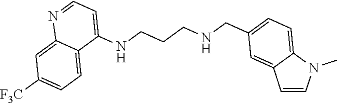 [SMILES: Cn1ccc2cc(CNCCCNc3ccnc4cc(C(F)(F)F)ccc34)ccc21] | 1.16 | 19.40 | 214.41 | 123.40 | 3.77 | 39.66 |
| 10 | SJ001009807-01 | 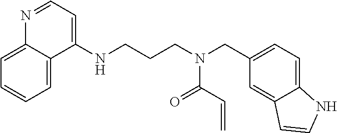 [SMILES: C=CC(=O)N(CCCNc1ccnc2ccccc12)Cc1ccc2[nH]ccc2c1] | 3.50 | 34.77 | 27.31 | 189.32 | 2.95 | 56.73 |
| 11 | SJ000311280-01 | 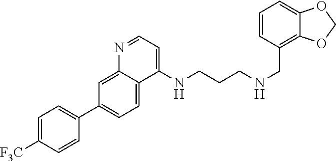 [SMILES: FC(F)(F)c1ccc(-c2ccc3c(NCCCNCc4cccc5c4OCO5)ccnc3c2)cc1] | 4.07 | NT | NT | NT | 5.45 | 54.88 |
| 12 | SJ000311281-01 | 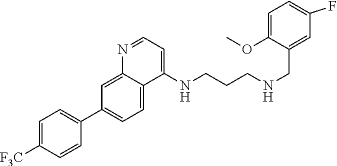 [SMILES: COc1ccc(F)cc1CNCCCNc1ccnc2cc(-c3ccc(C(F)(F)F)cc3)ccc12] | 5.58 | NT | NT | NT | 5.7 | 45.65 |
| 13 | SJ000311284-01 | 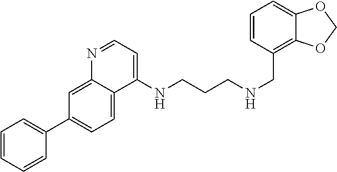 [SMILES: c1ccc(-c2ccc3c(NCCCNCc4cccc5c4OCO5)ccnc3c2)cc1] | 20.60 | NT | NT | NT | 4.53 | 54.88 |
| 14 | SJ000361113-02 | 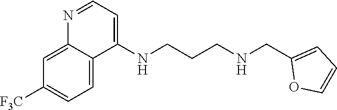 [SMILES: FC(F)(F)c1ccc2c(NCCCNCc3ccco3)ccnc2c1] | >70 | 119.10 | 502.42 | 401.37 | 2.61 | 45.65 |
| 15 | SJ000361106-02 | 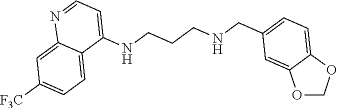 [SMILES: FC(F)(F)c1ccc2c(NCCCNCc3ccc4c(c3)OCO4)ccnc2c1] | >74 | 128.74 | 469.60 | 399.97 | 3.78 | 54.88 |

TABLE 8
Representative Compounds and Activity.

| | Compound Structure | TR-FRET |
|---|---|---|
| Compound ID | (SMILES format) | IC50(uM) |
| SJ000521054 | FC(F)(F)c1ccc2c(NCCCNCc3ccc4[nH]ccc4c3)ccnc2c1 | 0.40 |
| SJ000311289 | COc1ccc(F)cc1CNCCCNc2ccnc3cc(ccc23)c4ccc(cc4)C(C)(C)C | 2.04 |
| SJ000311287 | COc1cccc(CNCCCNc2ccnc3cc(ccc23)c4ccc(cc4)C(C)(C)C)c1O | 4.67 |
| SJ000311233 | COc1ccc(F)cc1CNCCCNc2ccnc3cc(Oc4cccc(c4)N(C)C)ccc23 | 5.63 |
| SJ000311237 | COc1ccc(F)cc1CNCCCNc2ccnc3cc(Oc4ccc(cc4)C(C)(C)C)ccc23 | 6.29 |
| SJ000311288 | CC(C)(C)c1ccc(cc1)c2ccc3c(NCCCNCc4cccc5OCOc45)ccnc3c2 | 6.87 |
| SJ000311235 | COc1cccc(CNCCCNc2ccnc3cc(Oc4ccc)C(C)(C)C)ccc23)c1O | 7.65 |
| SJ000311273 | COc1ccc(F)cc1CNCCCNc2ccnc3cc(ccc23)c4cc(cc(c4)C(F)(F)F)C(F)(F)F | 7.69 |
| SJ000311236 | CC(C)(C)c1ccc(Oc2ccc3c(NCCCNCc4cccc5OCOc45)ccnc3c2)cc1 | 8.67 |
| SJ000311272 | FC(F)(F)c1cc(cc(c1)C(F)(F)F)c2ccc3c(NCCCNCc4cccc5OCOc45)ccnc3c2 | 9.02 |
| SJ000311321 | COc1ccc(F)cc1CNCCCNc2ccnc3cc(Cc4cc(C)cc(C)c4)ccc23 | 9.04 |
| SJ000855285 | CCc1ccc(OCc2ccccc2C(═O)Nc3ccc4nc(C)cc(N)c4c3)cc1 | 9.2905 |
| SJ000311279 | COc1cccc(CNCCCNc2ccnc3cc(ccc23)c4ccc(cc4)C(F)(F)F)c1O | 9.52 |
| SJ000311267 | CCC(CC)c1ccc2c(NCCCNCc3cccc(OC)c3O)ccnc2c1 | 9.95 |
| SJ000311277 | COc1ccc(F)cc1CNCCCNc2ccnc3cc(ccc23)c4cccc5ccccc45 | 10.27 |
| SJ000311316 | Clc1cccc(Cl)c1Cc2ccc3c(NCCCNCc4cccc5OCOc45)ccnc3c2 | 11.04 |
| SJ000311317 | COc1ccc(F)cc1CNCCCNc2ccnc3cc(Cc4c(Cl)cccc4Cl)ccc23 | 12.23 |
| SJ000311300 | FC(F)(F)c1cccc(Cc2ccc3c(NCCCNCc4cccc5OCOc45)ccnc3c2)c1 | 12.79 |
| SJ000311281 | COc1ccc(F)cc1CNCCCNc2ccnc3cc(ccc23)c4ccc(cc4)C(F)(F)F | 13.38 |
| SJ000311315 | COc1cccc(CNCCCNc2ccnc3cc(Cc4c(Cl)cccc4Cl)ccc23)c1O | 13.83 |
| SJ000311280 | FC(F)(F)c1ccc(cc1)c2ccc3c(NCCCNCc4cccc5OCOc45)ccnc3c2 | 15.71 |
| SJ000311275 | COc1cccc(CNCCCNc2ccnc3cc(ccc23)c4cccc5cccccc45)c1O | 16.00 |
| SJ000311252 | C(CNCc1cccc2OCOc12)CNc3ccnc4cc(CC5CCCC5)ccc34 | 16.03 |
| SJ000311320 | Cc1cc(C)cc(Cc2ccc3c(NCCCNCc4cccc5OCOc45)ccnc3c2)c1 | 16.26 |
| SJ000311323 | COc1cccc(CNCCCNc2ccnc3cc(Cc4ccc(F)cc4Cl)ccc23)c1O | 17.60 |
| SJ000311325 | COc1ccc(F)cc1CNCCCNc2ccnc3cc(Cc4ccc(F)cc4Cl)ccc23 | 17.61 |
| SJ000311228 | Clc1ccc(Oc2ccc3c(NCCCNCc4cccc5OCOc45)ccnc3c2)cc1 | 17.68 |
| SJ000311309 | COc1ccc(F)cc1CNCCCNc2ccnc3cc(Cc4ccccc4)ccc23 | 18.28 |
| SJ000285544 | COc1cc(NC(C)CCCN)c2ncccc2c1 | 19.034 |
| SJ000311229 | COc1ccc(F)cc1CNCCCNc2ccnc3cc(Oc4ccc(Cl)cc4)ccc23 | 19.66 |
| SJ000311324 | Fc1ccc(Cc2ccc3c(NCCCNCc4cccc5OCOc45)ccnc3c2)c(Cl)c1 | 20.62 |
| SJ000311227 | COc1cccc(CNCCCNc2ccnc3cc(Oc4ccc(Cl)cc4)ccc23)c1O | 20.70 |
| SJ000311301 | COc1ccc(F)cc1CNCCCNc2ccnc3cc(Cc4cccc(c4)C(F)(F)F)ccc23 | 22.15 |
| SJ000311284 | C(CNCc1cccc2OCOc12)CNc3ccnc4cc(ccc34)c5ccccc5 | 22.24 |
| SJ000311319 | COc1cccc(CNCCCNc2ccnc3cc(Cc4cc(C)cc(C)c4)ccc23)c1O | 23.61 |
| SJ000311264 | C(CNCc1cccc2OCOc12)CNc3ccnc4cc(ccc34)C5CCCCC5 | 23.74 |
| SJ000311283 | COc1cccc(CNCCCNc2ccnc3cc(ccc23)c4ccccc4)c1O | 24.12 |
| SJ000311265 | COc1ccc(F)cc1CNCCCNc2ccnc3cc(ccc23)C4CCCCC4 | 24.16 |
| SJ000311291 | COc1cccc(CNCCCNc2ccnc(ccc23)c4ccc5OCOc5c4)c1O | 24.59 |
| SJ000311240 | Fc1ccc(cc1)c2ccc3c(NCCCNCc4cccc5OCOc45)ccnc3c2 | 24.97 |
| SJ000311253 | COc1ccc(F)cc1CNCCCNc2ccnc3cc(CC4CCCCC4)ccc23 | 26.26 |
| SJ000311255 | COc1cccc(CNCCCNc2ccnc3cc(CCc4ccccc4)ccc23)c1O | 26.55 |
| SJ000311299 | COc1cccc(CNCCCNc2ccnc3cc(Cc4cccc(c4)C(F)(F)F)ccc23)c1O | 27.47 |
| SJ000311248 | CC(C)CCc1ccc2c(NCCCNCc3cccc4OCOc34)ccnc2c1 | 27.69 |
| SJ000311285 | COc1ccc(F)cc1CNCCCNc2ccnc3cc(ccc23)c4ccccc4 | 28.23 |
| SJ000311268 | CCC(CC)c1ccc2c(NCCCNCc3cccc4OCOc34)ccnc2c1 | 28.94 |
| SJ000311209 | COc1ccc(F)cc1CNCCCNc2ccnc3cc(Oc4ccccc4)ccc23 | 28.96 |
| SJ000311219 | COc1ccc(Oc2ccc3c(NCCCNCc4cccc(OC)c4O)ccnc3c2)cc1 | 29.05 |
| SJ000311251 | COc1cccc(CNCCCNc2ccnc3cc(CC4CCCCC4)ccc23)c1O | 29.24 |
| SJ000311293 | COc1ccc(F)cc1CNCCNc2ccnc3cc(ccc23)c4ccc5OCOc5c4 | 29.87 |
| SJ000311208 | C(CNCc1cccc2OCOc12)CNc3ccnc4cc(Oc5ccccc5)ccc34 | 30.10 |
| SJ000311193 | ClC1═CC2═NC═CC(NCCCNCC3═C(O)C═CC(C(C)(C)C)═ | 30.45 |
| | C3)═C2C═C1 | |
| SJ000311292 | C(CNCc1cccc2OCOc12)CNc3ccnc4cc(ccc34)c5ccc6OCOc6c5 | 30.49 |
| SJ000311276 | C(CNCc1cccc2OCOc12)CNc3ccnc4cc(ccc34)c5cccc6ccccc56 | 31.19 |
| SJ000311312 | COc1cc(Cc2ccc3c(NCCCNCc4cccc5OCOc45)ccnc3c2)cc(OC)c1 | 31.27 |
| SJ000311263 | COc1cccc(CNCCCNc2ccnc3cc(ccc23)C4CCCCC4)c1O | 32.40 |
| SJ000311239 | COc1cccc(CNCCCNc2ccnc3cc(ccc23)c4ccc(F)cc4)c1O | 33.30 |
| SJ000311311 | COc1cc(Cc2ccc3c(NCCCNCc4cccc(OC)c4O)ccnc3c2)cc(OC)c1 | 33.61 |
| SJ000311313 | COc1cc(Cc2ccc3c(NCCCNCc4cc(F)ccc4OC)ccnc3c2)cc(OC)c1 | 33.77 |
| SJ000523064 | Clc1ccc2c(NCCCNCc3ccc(cc3)c4ccccc4)ccnc2c1 | 34.86 |
| SJ000311245 | COc1ccc(F)cc1CNCCCNc2ccnc3cc(Cc4ccc(F)cc4)ccc23 | 35.05 |
| SJ000311217 | COc1cccc(Oc2ccc3c(NCCCNCc4cc(F)ccc4OC)ccnc3c2)c1 | 36.79 |
| SJ000311216 | COc1cccc(Oc2ccc3c(NCCCNCc4cccc5OCOc45)ccnc3c2)c1 | 36.90 |
| SJ000311256 | COc1cccc(F)cc1CNCCCNc2ccnc3cc(CCc4ccccc4)ccc23 | 37.08 |
| SJ000311241 | COc1ccc(F)cc1CNCCCNc2ccnc3cc(ccc23)c4ccc(F)cc4 | 38.89 |
| SJ000521067 | CCc1ccc(CNCCCNc2ccnc3cc(ccc23)C(F)(F)F)cc1 | 40.85 |
| SJ000311244 | Fc1ccc(Cc2ccc3c(NCCCNCc4cccc5OCOc45)ccnc3c2)cc1 | 42.28 |
| SJ000311269 | CCC(CC)c1ccc2c(NCCCNCc3cc(F)ccc3OC)ccnc2c1 | 42.96 |
| SJ000311243 | COc1cccc(CNCCCNc2ccnc3cc(Cc4ccc(F)cc4)ccc23)c1O | 43.55 |
| SJ000311220 | COc1ccc(OC(cc2)cc3c2c(NCCCNCc4c5c(OCO5)ccc4)cc23)cc1 | 43.74 |
| SJ000311308 | C(CNCc1cccc2OCOc12)CNc3ccnc4cc(Cc5ccccc5)ccc34 | 46.10 |
| SJ000311221 | COc1ccc(Oc2ccc3c(NCCCNCc4cc(F)ccc4OC)ccnc3c2)cc1 | 46.45 |
| SJ000311303 | COc1cccc(CNCCCNc2ccnc3cc(Cc4ccc(cc4)C#N)ccc23)C1O | 50.99 |
| SJ000311224 | Fc1ccc(Oc2ccc3c(NCCCNCc4cccc5OCOc45)ccnc3c2)cc1 | 59.58 |
| SJ000311223 | COc1cccc(CNCCCNc2ccnc3cc(Oc4ccc(F)cc4)ccc23)c1O | 61.87 |
| SJ000311257 | C(CNCc1cccc2OCOc12)CNc3ccnc4cc(CCc5ccccc5)ccc34 | 62.26 |
| SJ000311304 | N#Cc1ccc(Cc2ccc3c(NCCCNCc4cccc5OCOc45)ccnc3c2)cc1 | 65.98 |
| SJ000520909 | Clc1ccc2c(NCCCNCc3ccc4[nH]ccc4c3)ccnc2c1 | 67.10 |
| SJ000520911 | Cc1cc(C)c(CNCCCNc2ccnc3cc(Cl)ccc23)c(C)c1 | 68.12 |
| SJ000311259 | COc1cccc(CNCCCNc2ccnc3ccc(CC(C)C)ccc23)c1O | 68.50 |
| SJ000521064 | COc1ccc(cc1CNCCCNc2ccnc3cc(ccc23)C(F)(F)F)C(F)(F)F | 69.13 |
| SJ000000500 | COc1ccc(F)cc1CNCCCNc2ccnc3cc(Cl)ccc23 | 69.93 |
| SJ000000501 | Clc1ccc2c(NCCCNCc3cccc4OCOc34)ccnc2c1 | 69.93 |
| SJ000311185 | ClC1═CC2═NC═CC(NCCCNCC3═C(OC)C═CC═C3)═C2C═C1 | 69.93 |
| SJ000311186 | ClC1═CC2═NC═CC(NCCCNCC3═C(O)C═CC(Br)═C3)═C2C═C1 | 69.93 |
| SJ000311187 | ClC1═CC2═NC═CC(NCCCNCC3═C(O)C═CC═C3)═C2C═C1 | 69.93 |
| SJ000311188 | ClC1═CC2═NC═CC(NCCCNC(C)C3═C(O)C═CC(OC)═C3)C2C═C1 | 69.93 |
| SJ000311191 | ClC1═CC2═NC═CC(NCCCNCC3═CC═C([N+]([O—])═O)C(O)═ | 69.93 |
| | C3)═C2C═C1 | |
| SJ000311192 | ClC1═CC2═NC═CC(NCCCNCC3═CC═C(OC)C(O)═C3)═C2C═C1 | 69.93 |
| SJ000311194 | ClC1═CC2═NC═CC(NCCCNCC3═C(Br)C═CC(O)═C3)═C2C═C1 | 69.93 |
| SJ000311196 | ClC1═CC2═NC═CC(NCCCCN(CCC)CC3═CC(Br)═CC═C3O)═ | 69.93 |
| | C2C═C1 | |
| SJ000311207 | COc1cccc(CNCCCNc2ccnc3cc(Oc4ccccc4)ccc23)c1O | 69.93 |
| SJ000311211 | COc1cccc(CNCCCNc2ccnc3cc(Oc4ccccc4OC)ccc23)c1O | 69.93 |
| SJ000311212 | COc1ccccc1Oc2ccc3c(NCCCNCc4cccc5OCOc45)ccnc3c2 | 69.93 |
| SJ000311213 | COc1ccc(F)cc1CNCCCNc2ccnc3cc(Oc4ccccc4OC)ccc23 | 69.93 |
| SJ000311215 | COc1cccc(Oc2ccc3c(NCCCNCc4cccc(OC)c4O)ccnc3c2)c1 | 69.93 |
| SJ000311225 | COc1ccc(F)cc1CNCCCNc2ccnc3cc(Oc4ccc(F)cc4)ccc23 | 69.93 |
| SJ000311231 | COc1cccc(CNCCCNc2ccnc3cc(Oc4cccc(c4)N(C)C)ccc23)c1O | 69.93 |
| SJ000311232 | CN(C)c1cccc(Oc2ccc3c(NCCCNCc4cccc5OCOc45)ccnc3c2)c1 | 69.93 |
| SJ000311247 | COc1cccc(CNCCCNc2ccnc3cc(CCC(C)C)ccc232)c1O | 69.93 |
| SJ000311249 | COc1ccc(F)cc1CNCCCNc2ccnc3cc(CCC(C)C)ccc23 | 69.93 |
| SJ000311260 | CC(C)Cc1ccc2c(NCCCNCc3cccc4OCOc34)ccnc2c1 | 69.93 |
| SJ000311261 | COc1ccc(F)cc1CNCCCNc2ccnc3cc(CC(C)C)ccc23 | 69.93 |
| SJ000311271 | COc1cccc(CNCCCNc2ccnc3cc(ccc23)c4cc(cc(c4)C(F)(F)F)C(F)(F)F)c1O | 69.93 |
| SJ000311295 | COc1ccc(cc1)c2ccc3c(NCCCNCc4cccc(OC)c4O)ccnc3c2 | 69.93 |
| SJ000311296 | COc1ccc(cc1)c2ccc3c(NCCCNCc4cccc5OCOc45)ccnc3c2 | 69.93 |
| SJ000311297 | COc1ccc(cc1)c2ccc3c(NCCCNCc4cc(F)ccc4OC)ccnc3c2 | 69.93 |
| SJ000311305 | COc1ccc(F)cc1CNCCCNc2ccnc3cc(Cc4ccc(cc4)C#N)ccc23 | 69.93 |
| SJ000311307 | COc1cccc(CNCCCNc2ccnc3cc(Cc4ccccc4)ccc23)c1O | 69.93 |
| SJ000311328 | COc1cccc(F)c1CNCCCNc2ccnc3cc(Cl)ccc23 | 69.93 |
| SJ000361100 | Clc1ccc2c(NCCCNCc3ccccc3)ccnc2c1 | 69.93 |
| SJ000361106 | FC(F)(F)c1ccc2c(NCCCNCc3ccc4OCOc4c3)ccnc2c1 | 69.93 |
| SJ000361107 | FC(F)(F)c1ccc2c(NCCCNCc3ccccc3)ccnc2c1 | 69.93 |
| SJ000361122 | Clc1ccc2c(NCCCNCc3c4ccccc4cc5ccccc35)ccnc2c1 | 69.93 |
| SJ000361126 | FC(F)(F)c1ccc2c(NCCCNCc3c4ccccc4cc5ccccc35)ccnc2c1 | 69.93 |
| SJ000520908 | FC(F)(F)c1ccccc1CNCCCNc2ccnc3cc(Cl)ccc23 | 69.93 |
| SJ000520910 | Fc1c(F)c(F)c(CNCCCNc2ccnc333(Cl)ccc23)c(F)c1F | 69.93 |
| SJ000521022 | FC(F)(F)Oc1cccc(CNCCCNc2ccnc3cc(Cl)ccc23)c1 | 69.93 |
| SJ000521024 | FC(F)(F)c1cccc(CNCCCNc2ccnc3cc(Cl)ccc23)c1 | 69.93 |
| SJ000521025 | FC(F)(F)Oc1ccc(CNCCCNc2cc2c3cc(Cl)ccc23)cc1 | 69.93 |
| SJ000521030 | COc1ccc(F)c(CNCCCNc2ccnc3cc(Cl)ccc23)c1 | 69.93 |
| SJ000521031 | Fc1cccc(c1CNCCCNc2ccnc3cc(Cl)ccc23)C(F)(F)F | 69.93 |
| SJ000521035 | COc1ccc(OC(F)(F)F)cc1CNCCCNc2ccnc3cc(Cl)ccc23 | 69.93 |
| SJ000521036 | COc1ccc(cc1CNCCCNc2ccnc3cc(Cl)ccc23)C(F)(F)F | 69.93 |
| SJ000521039 | CCc1ccc(CNCCCNc2ccnc3cc(Cl)ccc23)cc1 | 69.93 |
| SJ000521045 | FC(F)(F)Oc1cccc(CNCCCNc2ccnc3cc(ccc23)C(F)(F)F)c1 | 69.93 |
| SJ000521047 | FC(F)(F)c1cccc(CNCCCNc2ccnc3cc(ccc23)C(F)(F)F)c1 | 69.93 |
| SJ000521048 | FC(F)(F)c1ccc2c(NCCCNCc3cccc4OCOc34)ccnc2c1 | 69.93 |
| SJ000521049 | FC(F)(F)c1ccc2c(NCCCNCc3ccccc3C(F)(F)F)ccnc2c1 | 69.93 |
| SJ000521050 | FC(F)(F)Oc1ccc(CNCCCNc2ccnc3cc(ccc23)C(F)(F)F)cc1 | 69.93 |
| SJ000521051 | COc1cccc(F)c1CNCCCNc2ccnc3cc(ccc23)c(F)(F)F | 69.93 |
| SJ000521052 | COc1ccc(F)cc1CNCCCNc2ccnc3cc(ccc23)C(F)(F)F | 69.93 |
| SJ000521058 | COc1ccc(F)c(CNCCCNc2ccnc3cc(ccc23)C(F)(F)F)c1 | 69.93 |
| SJ000521059 | Fc1cccc(c1CNCCCNc2ccnc3cc(ccc23)C(F)(F)F)C(F)(F)F | 69.93 |
| SJ000521063 | COc1ccc(OC(F)(F)F)cc1CNCCCNc2ccnc3cc(ccc23)C(F)(F)F | 69.93 |
| SJ000521066 | Fc1cccc(CNCCCNc2ccnc3cc(ccc23)C(F)(F)F)c1 | 69.93 |
| SJ000520354 | CN(C)c1ccc(CNCCCNc2ccnc3cc(Cl)ccc23)C4ccccc14 | 69.93 |
| SJ000523055 | Nc1ccccc1CNCCCNc2ccnc3cc(Cl)ccc23 | 69.93 |
| SJ000523056 | Clc1ccc2c(NCCCNCc3ccc4OCOc4c3)ccnc2c1 | 69.93 |
| SJ000523059 | COc1ccc(CNCCCNc2ccnc3cc(Cl)ccc23)cc1 | 69.93 |
| SJ000523060 | Fc1ccccc1CNCCCNc2ccnc3cc(Cl)ccc23 | 69.93 |
| SJ000523061 | Fc1cccc(CNCCCNc2ccnc3cc(Cl)ccc23)c1 | 69.93 |
| SJ000523062 | Fc1ccc(CNCCCNc2ccnc3cc(Cl)ccc23)cc1 | 69.93 |
| SJ000523063 | Clc1ccc2c(NCCCNCc3cccc(c3)C#N)ccnc2c1 | 69.93 |
| SJ000523065 | Clc1ccc2c(NCCCNCc3ccc4ccccc4c3)ccnc2c1 | 69.93 |
| SJ000523066 | Cc1cccc(CNCCCNc2ccnc3cc(Cl)ccc23)c1 | 69.93 |
| SJ000523067 | COC(═O)c1ccc(CNCCCNc2ccnc3cc(Cl)ccc23)cc1 | 69.93 |
| SJ000285410 | CC[C@@]1(O)C(═O)OCC2═C1C═C3N(Cc4cc5ccccc5nc34)C2═O | >55.1818 |
| SJ000312345 | CCc1c2CN3C(═O)C4═C(C═C3c2nc5ccc(OC(═O)N6CCC(CC6) | >58.7413 |
| | N7CCCCC7)cc15)[C@@](O)(CC)C(═O)OC4 | |
| SJ000518991 | COc1cc(C)c2c(Oc3cccc(c3)C(F)(F)F)c(OC)cc(NC(C)CCCN)c2n1 | >65.7343 |
| SJ000834150 | C(CN1CCOCC1)Oc2ccc3c(ccnc3c2)c4c5CCC25nc4c6ccccn6 | >67.3357 |
| SJ000121402 | COc1c(OCC(O)C(C)O)ccc2c(OC)c3ccoc3nc12 | >69.9301 |
| SJ000280708 | COc1ccc(OC)c2c(O)c(CN3CCCCC3C)c(C)nc12 | >69.9301 |
| SJ000284975 | COc1ccc(OC)c2c(O)cc(C)nc12 | >69.9301 |
| SJ000285450 | Nc1c2ccccc2nc3ccccc13 | >75.5245 |

1. A compound having a structure represented by a formula: 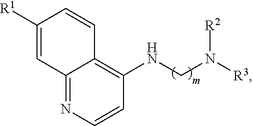
wherein m is selected from 2, 3, and 4;
wherein R¹ is selected from C1-C10 haloalkyl, C1-C10 alkoxy, C1-C10 aminoalkyl, C1-C10 alkylamino, C1-C10 hydoxyalkyl, C1-C10 alkyl, -Q-Ar¹, -Q-Cy¹, —Ar¹, and —Cy¹;
wherein Q is selected from —(CH₂)ₙ— and —O—;
wherein n is selected from 1 and 2
wherein the Ar¹ is selected from phenyl, benzyl, indolyl, benzo[d][1,3]dioxolyl naphthyl, and anthracenyl;
wherein the Ar¹ is substituted with 0, 1, or 2 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl;
wherein the Cy¹ is C3-C8 cycloalkyl; and
wherein the Cy¹ is substituted with 0, 1, 2, 3, 4, or 5 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl;
wherein R² is selected from H and C1-C3 alkyl;
wherein R³ is selected from C1-C3 alkyl and —(CH₂)ₚ—Ar²;
wherein p is selected from 1 and 2;
wherein Ar² is selected from a phenyl, indolyl, benzo[d][1,3]dioxolyl, naphthyl, and anthracenyl
wherein the Ar² is substituted with 0, 1, or 2 groups independently selected from halogen, —SF₅, —CN, —N₃, —NO₂, —OH, —CN, C1-C3 alkoxy, C1-C3 haloalkyl, C1-C3 aminoalkyl, C1-C3 alkylamino, C1-C3 hydoxyalkyl, and C1-C3 alkyl;
or a pharmaceutically acceptable salt thereof.

2. The compound of claim 1, wherein m is selected from 2 and 3.

3. (canceled)

4. The compound of claim 1, wherein R¹ is selected from halogen, C1-C10 haloalkyl, C1-C10 alkoxy, C1-C10 aminoalkyl, C1-C10 alkylamino, C1-C10 hydoxyalkyl, C1-C10 alkyl, —Ar¹, —Cy¹, —O—Ar¹, —O-Cy¹, —(C1-C2 alkanediyl)-Ar¹, and —(C1-C2 alkanediyl)-Cy¹.

5.-6. (canceled)

7. The compound of claim 1, wherein Ar¹ is phenyl substituted with 0, 1, or 2 groups independently selected from halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.

8. (canceled)

9. The compound of claim 1, wherein R² is selected from hydrogen and methyl.

10.-12. (canceled)

13. The compound of claim 1, wherein R³ is —(CH₂)ₙ—Ar².

14. (canceled)

15. The compound of claim 13, wherein Ar² is selected from indolyl, benzo[d][1,3]dioxolyl and phenyl substituted with 0, 1, or 2 groups independently selected from halogen, —SF₅, —CN, —N₃, —NO₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.

16.-21. (canceled)

22. The compound of claim 1, wherein the compound has a structure represented by a formula:

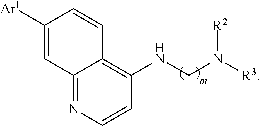

23. The compound of claim 1, wherein the compound has a structure represented by a formula:

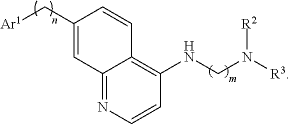

24. (canceled)

25. The compound of claim 1, wherein the compound has a structure represented by a formula:

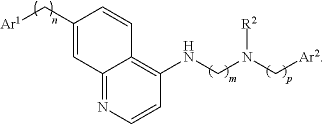

26. The compound of claim 1, having a structure represented by a formula: 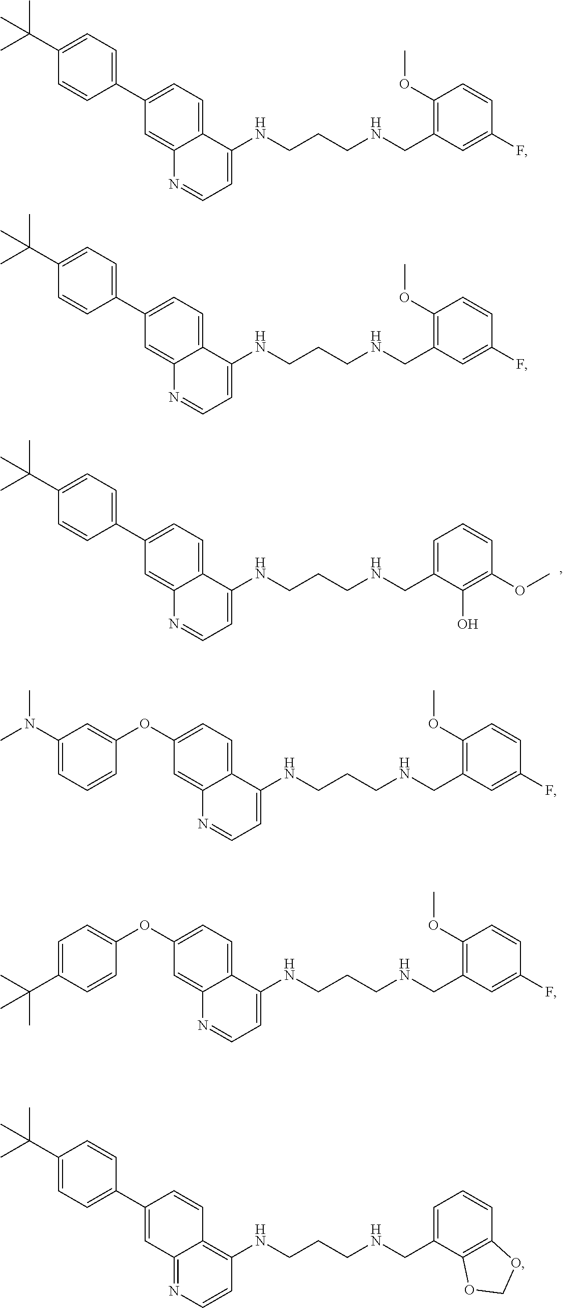 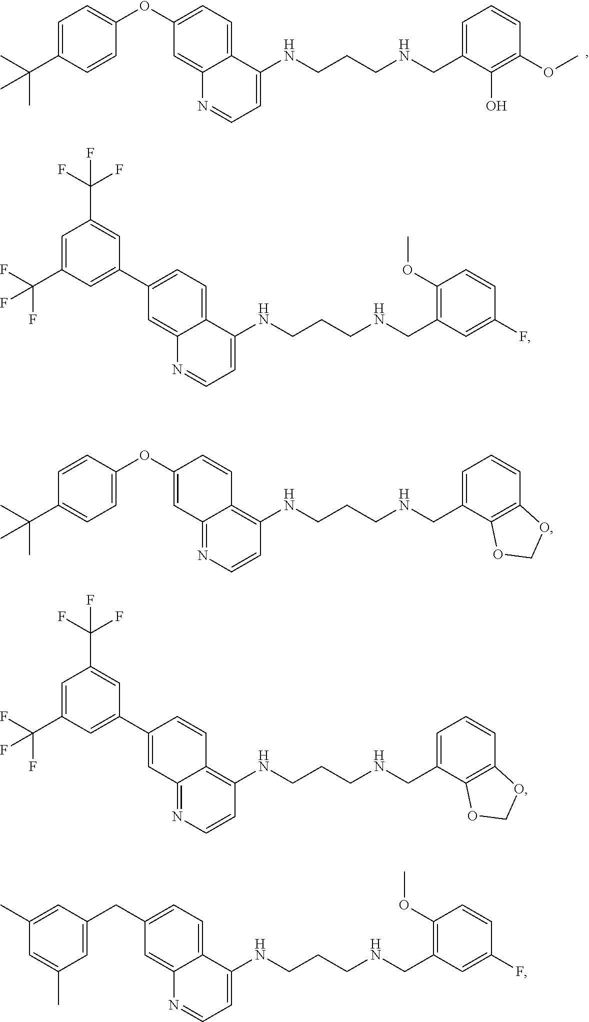 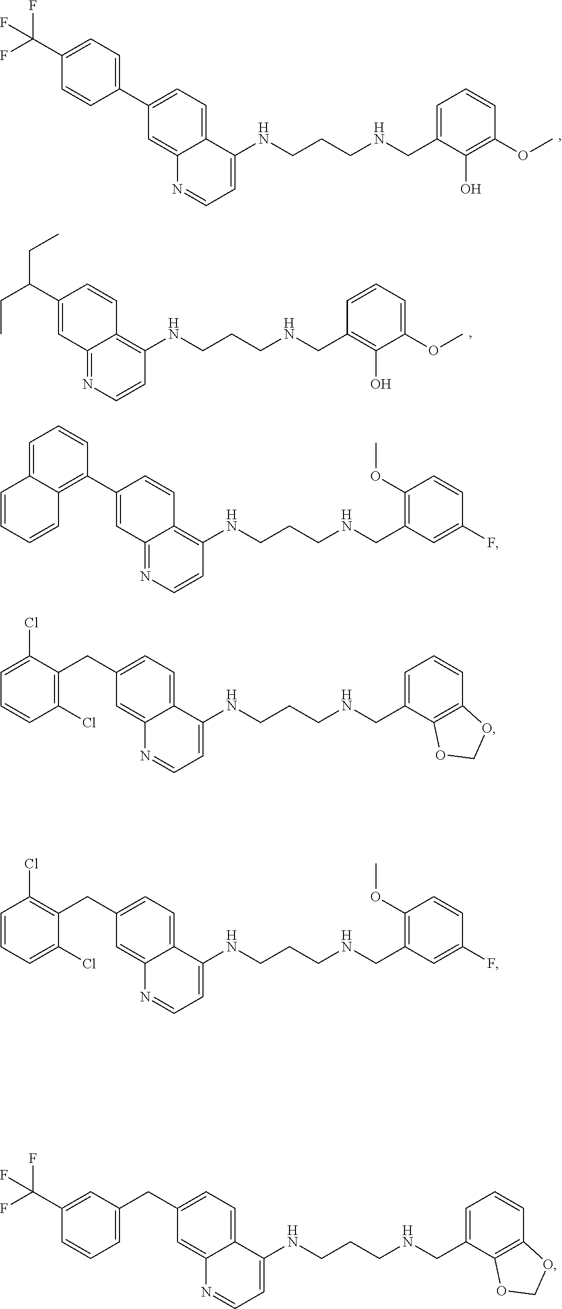 [SMILES: COc1cccc(CNCCCNc2ccnc3cc(-c4ccc(C(F)(F)F)cc4)ccc23)c1O ; CCC(CC)c1ccc2c(NCCCNCc3cccc(OC)c3O)ccnc2c1 ; COc1ccc(F)cc1CNCCCNc1ccnc2cc(-c3cccc4ccccc34)ccc12 ; Clc1cccc(Cl)c1Cc1ccc2c(NCCCNCc3cccc4c3OCO4)ccnc2c1 ; COc1ccc(F)cc1CNCCCNc1ccnc2cc(Cc3c(Cl)cccc3Cl)ccc12 ; FC(F)(F)c1cccc(Cc2ccc3c(NCCCNCc4cccc5c4OCO5)ccnc3c2)c1] 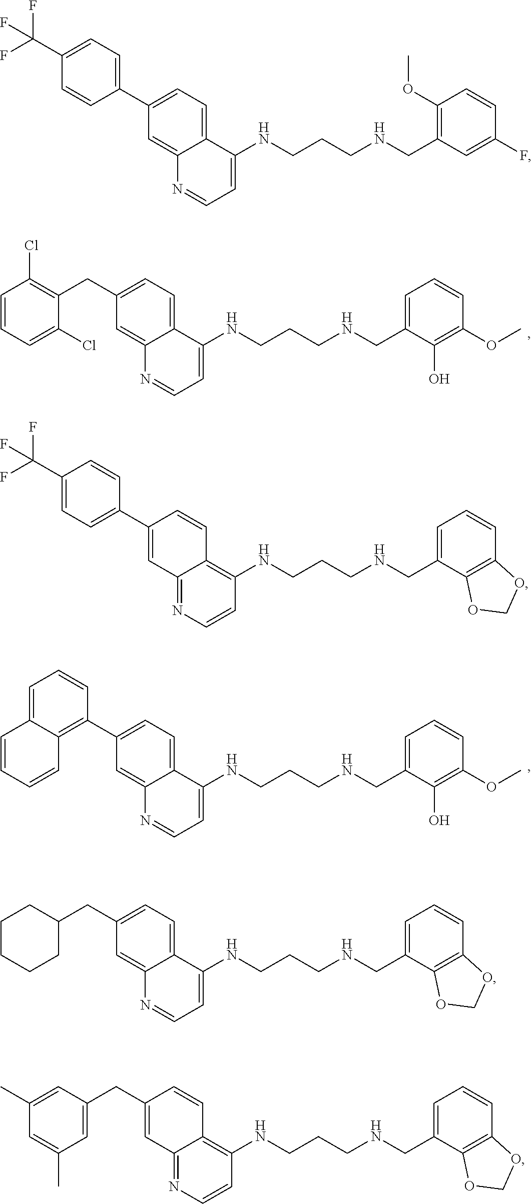 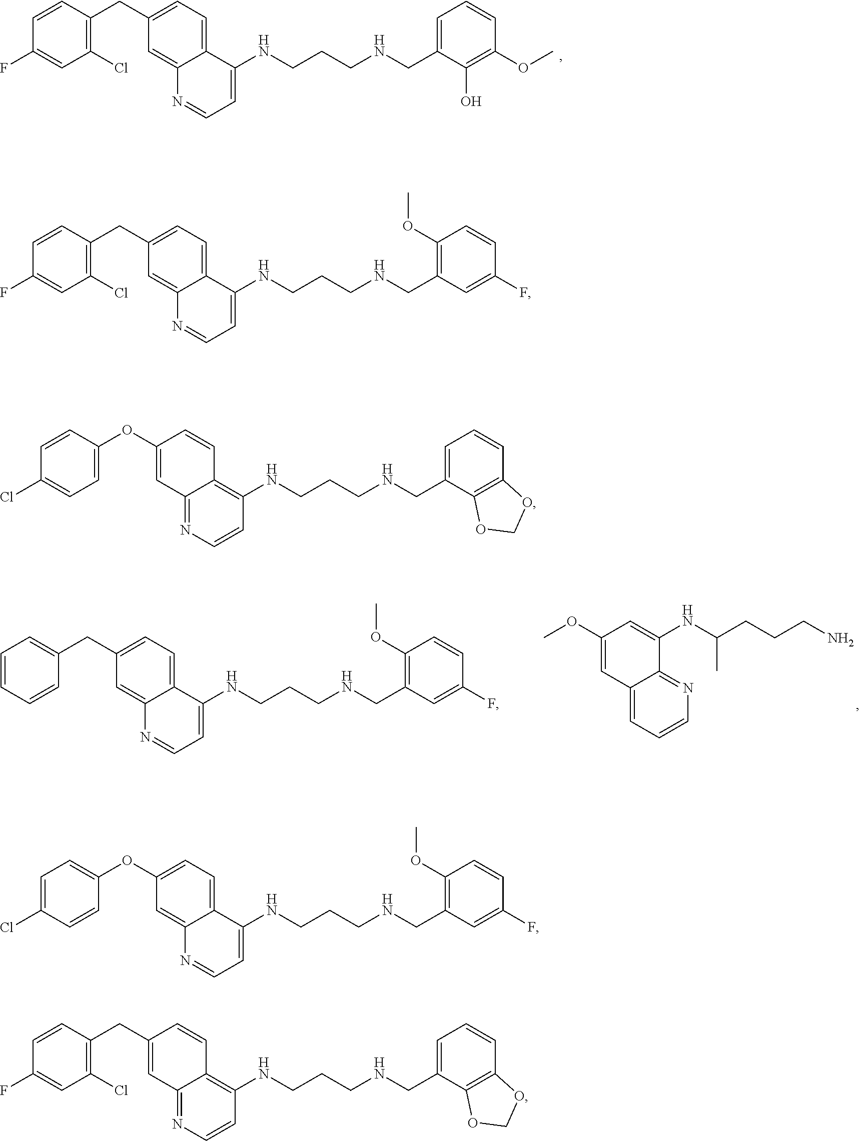 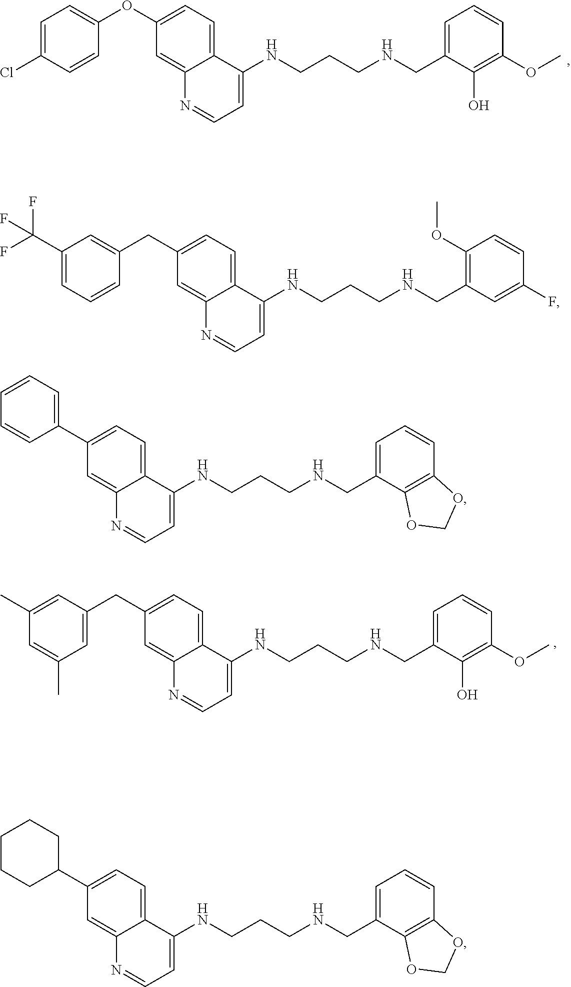 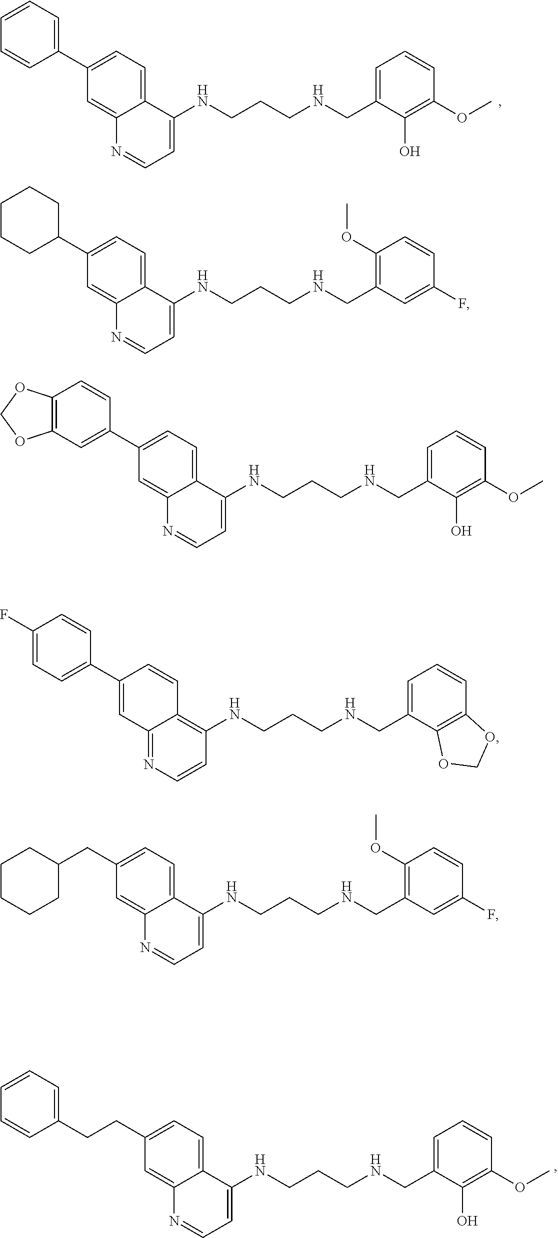 [SMILES: COc1cccc(CNCCCNc2ccnc3cc(-c4ccccc4)ccc23)c1O ; COc1ccc(F)cc1CNCCCNc1ccnc2cc(C3CCCCC3)ccc12 ; COc1cccc(CNCCCNc2ccnc3cc(-c4ccc5c(c4)OCO5)ccc23)c1O ; Fc1ccc(-c2ccc3c(NCCCNCc4cccc5c4OCO5)ccnc3c2)cc1 ; COc1ccc(F)cc1CNCCCNc1ccnc2cc(CC3CCCCC3)ccc12 ; COc1cccc(CNCCCNc2ccnc3cc(CCc4ccccc4)ccc23)c1O] 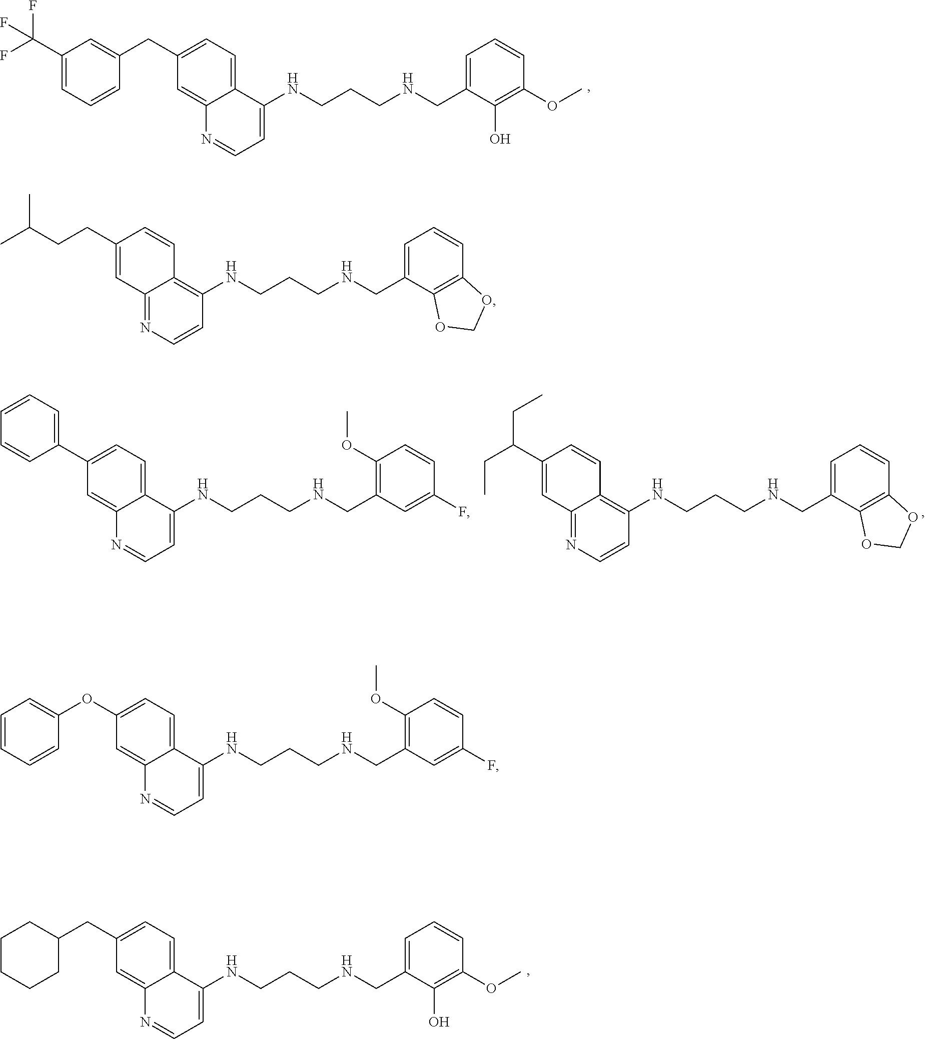 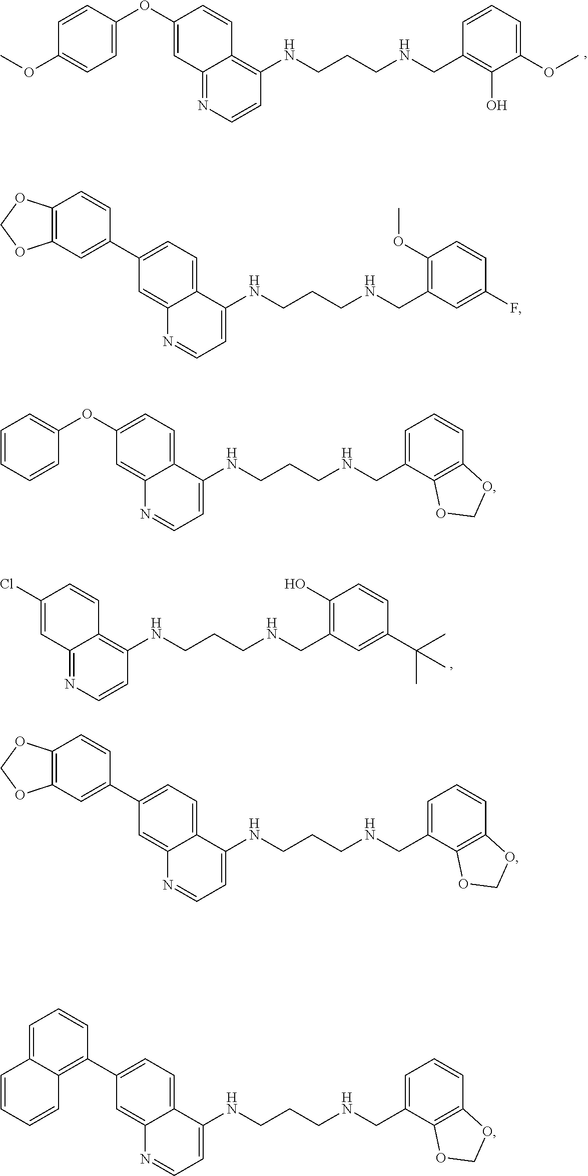 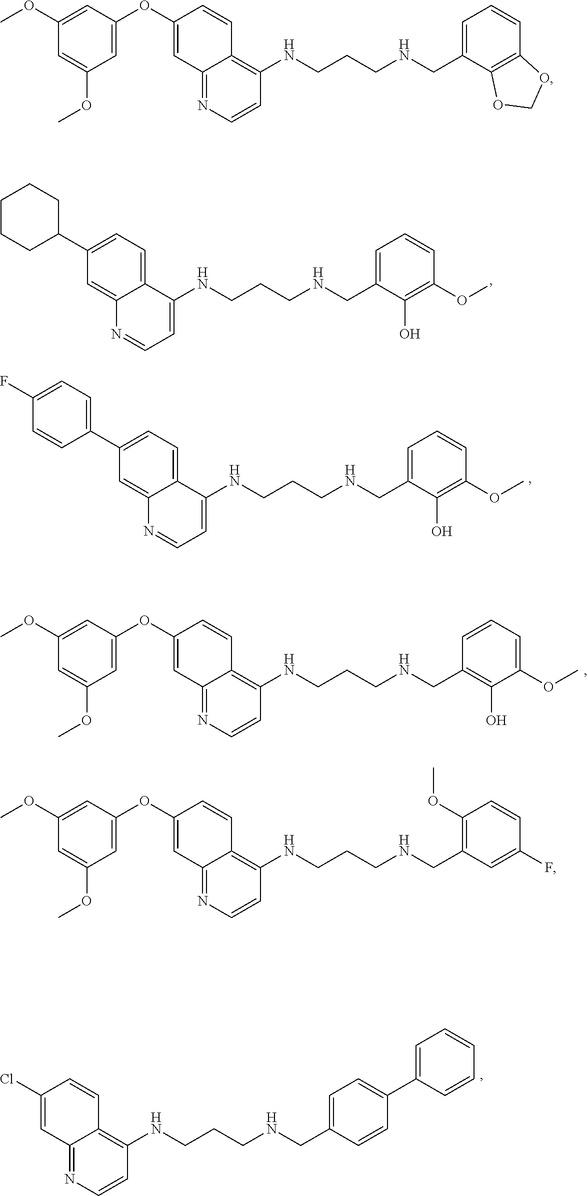 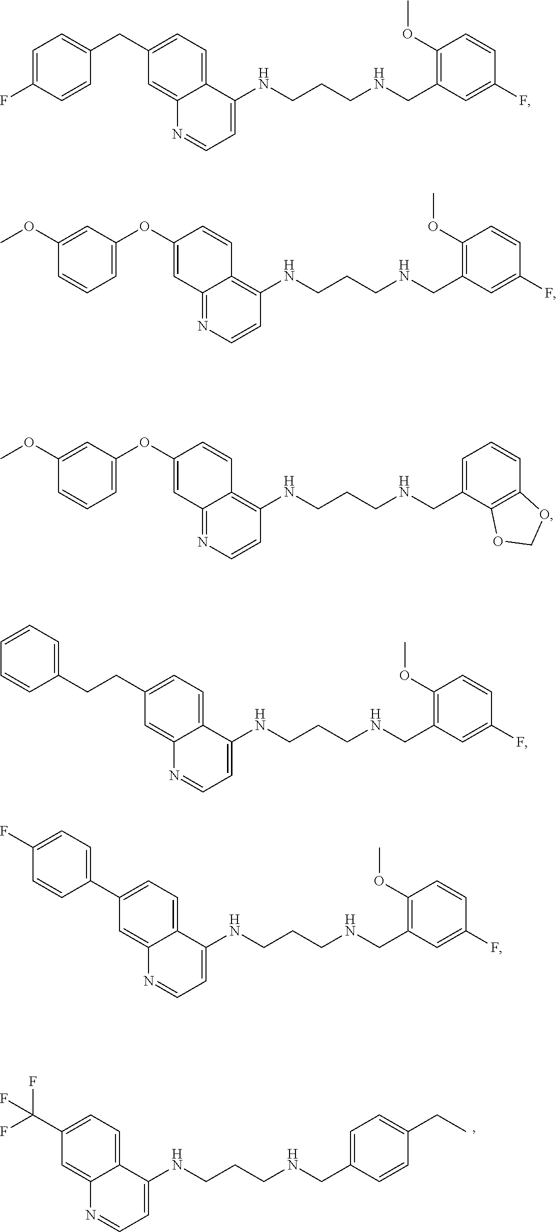 [SMILES: COc1ccc(F)cc1CNCCCNc1ccnc2cc(Cc3ccc(F)cc3)ccc12 ; COc1cccc(Oc2ccc3c(NCCCNCc4cc(F)ccc4OC)ccnc3c2)c1 ; COc1cccc(Oc2ccc3c(NCCCNCc4cccc5c4OCO5)ccnc3c2)c1 ; COc1ccc(F)cc1CNCCCNc1ccnc2cc(CCc3ccccc3)ccc12 ; COc1ccc(F)cc1CNCCCNc1ccnc2cc(-c3ccc(F)cc3)ccc12 ; CCc1ccc(CNCCCNc2ccnc3cc(C(F)(F)F)ccc23)cc1] 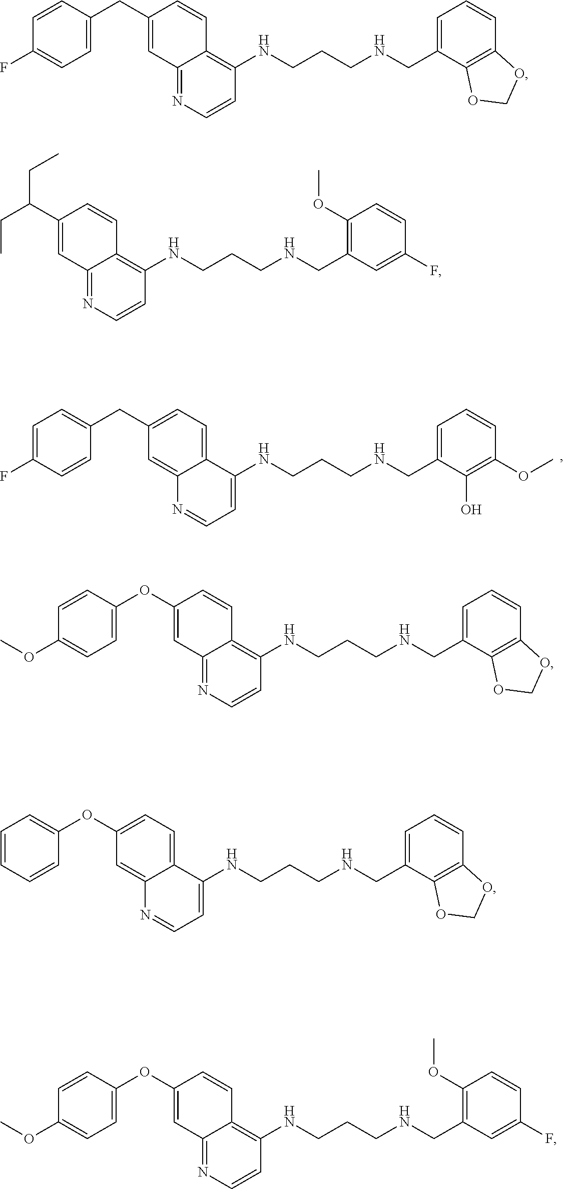 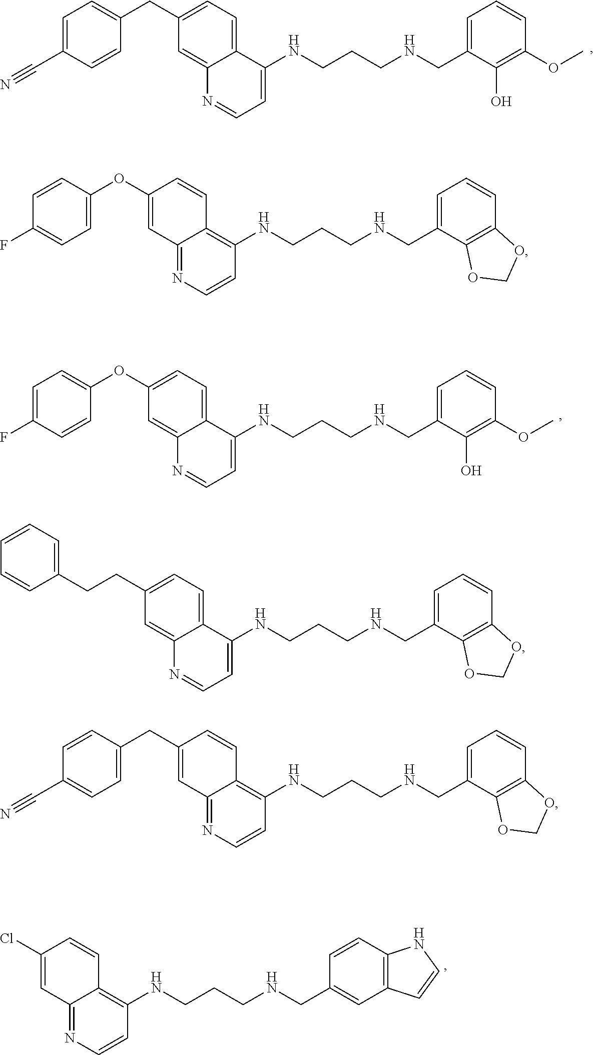 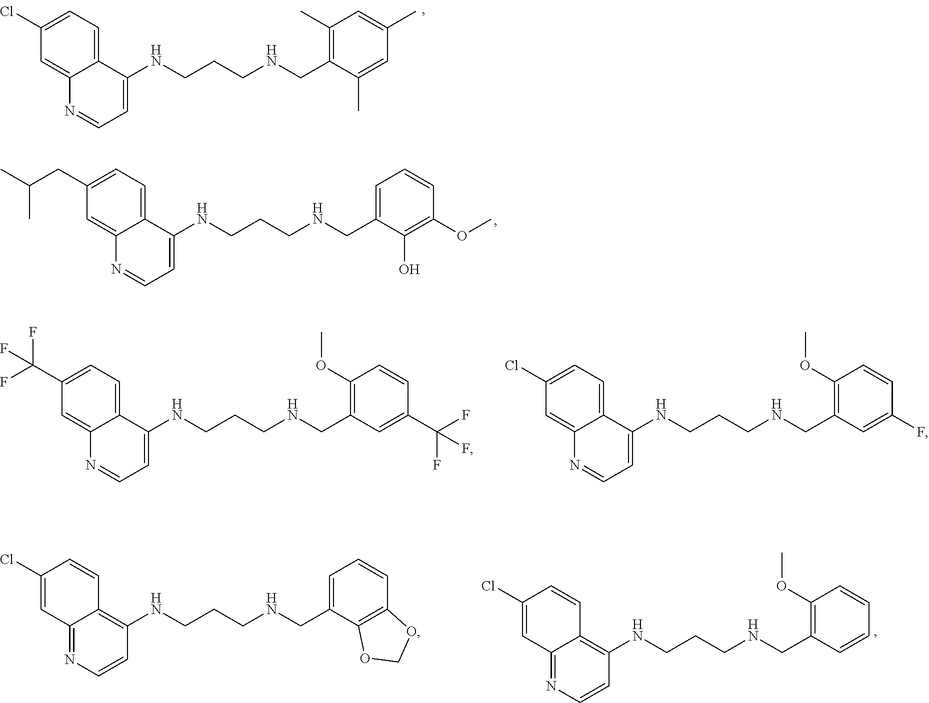 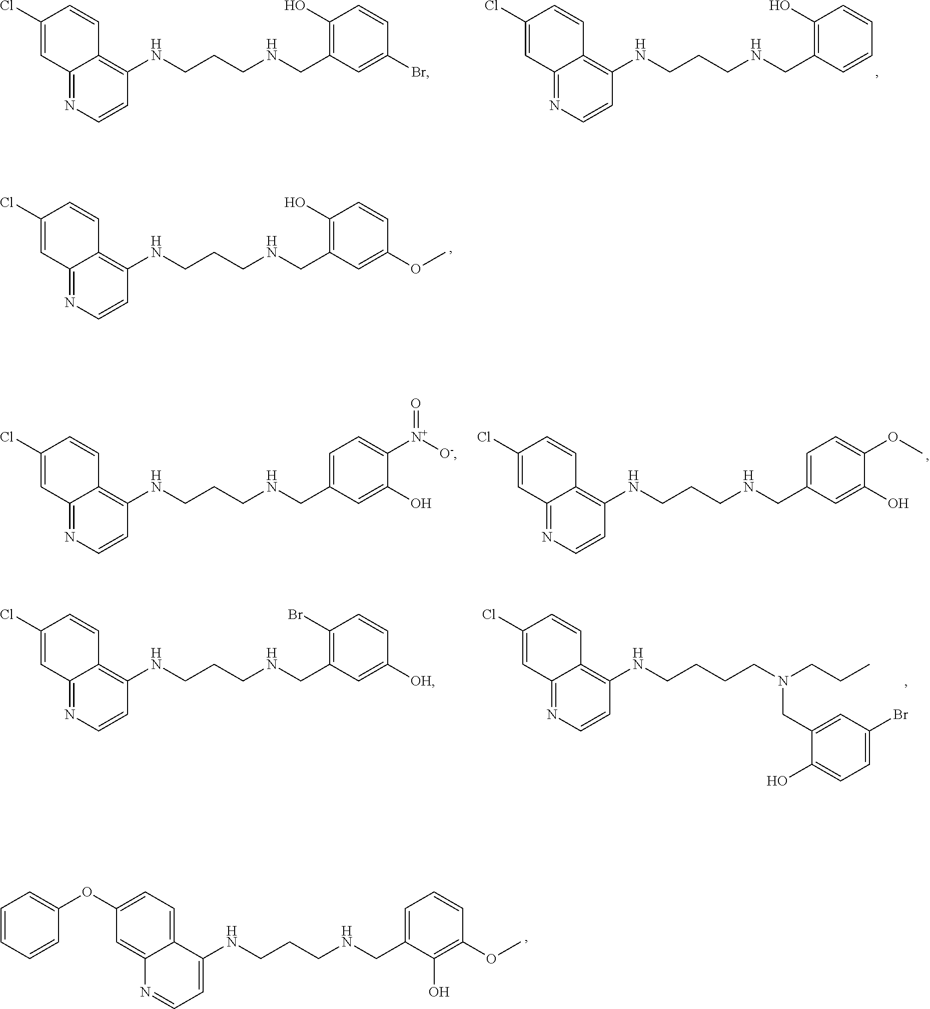 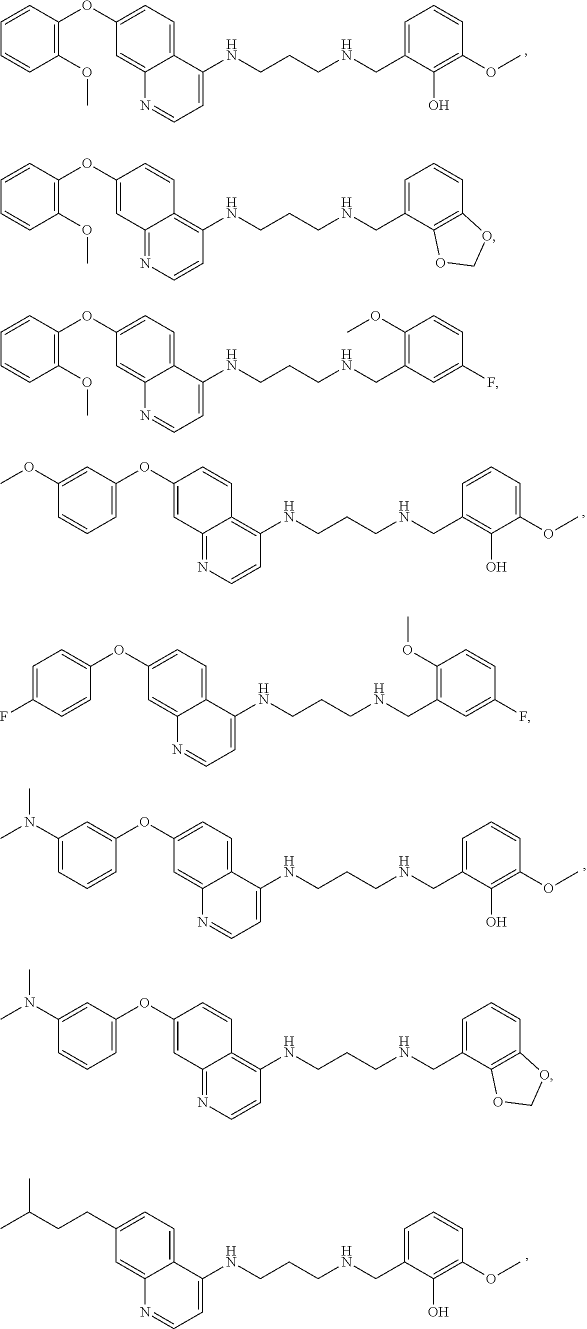 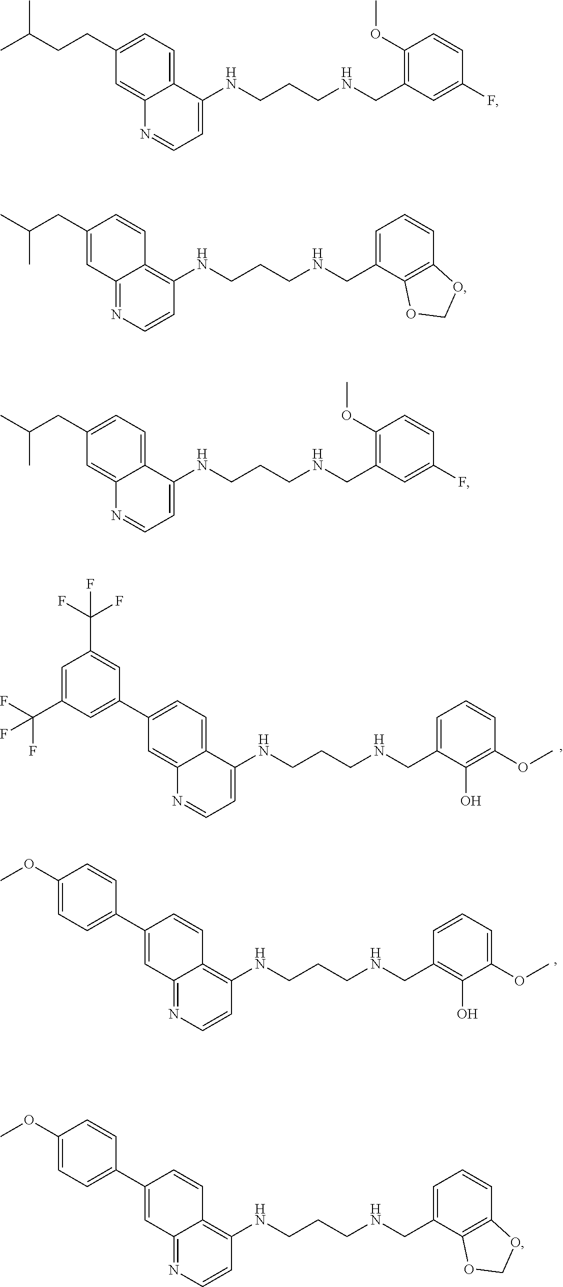 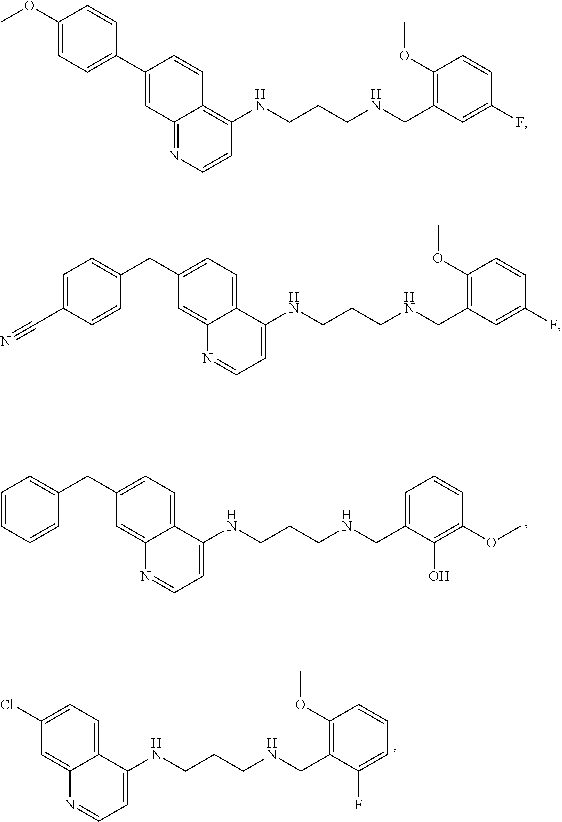 [SMILES: COc1ccc(-c2ccc3c(NCCCNCc4cc(F)ccc4OC)ccnc3c2)cc1 ; COc1ccc(F)cc1CNCCCNc1ccnc2cc(Cc3ccc(C#N)cc3)ccc12 ; COc1cccc(CNCCCNc2ccnc3cc(Cc4ccccc4)ccc23)c1O ; COc1cccc(F)c1CNCCCNc1ccnc2cc(Cl)ccc12] 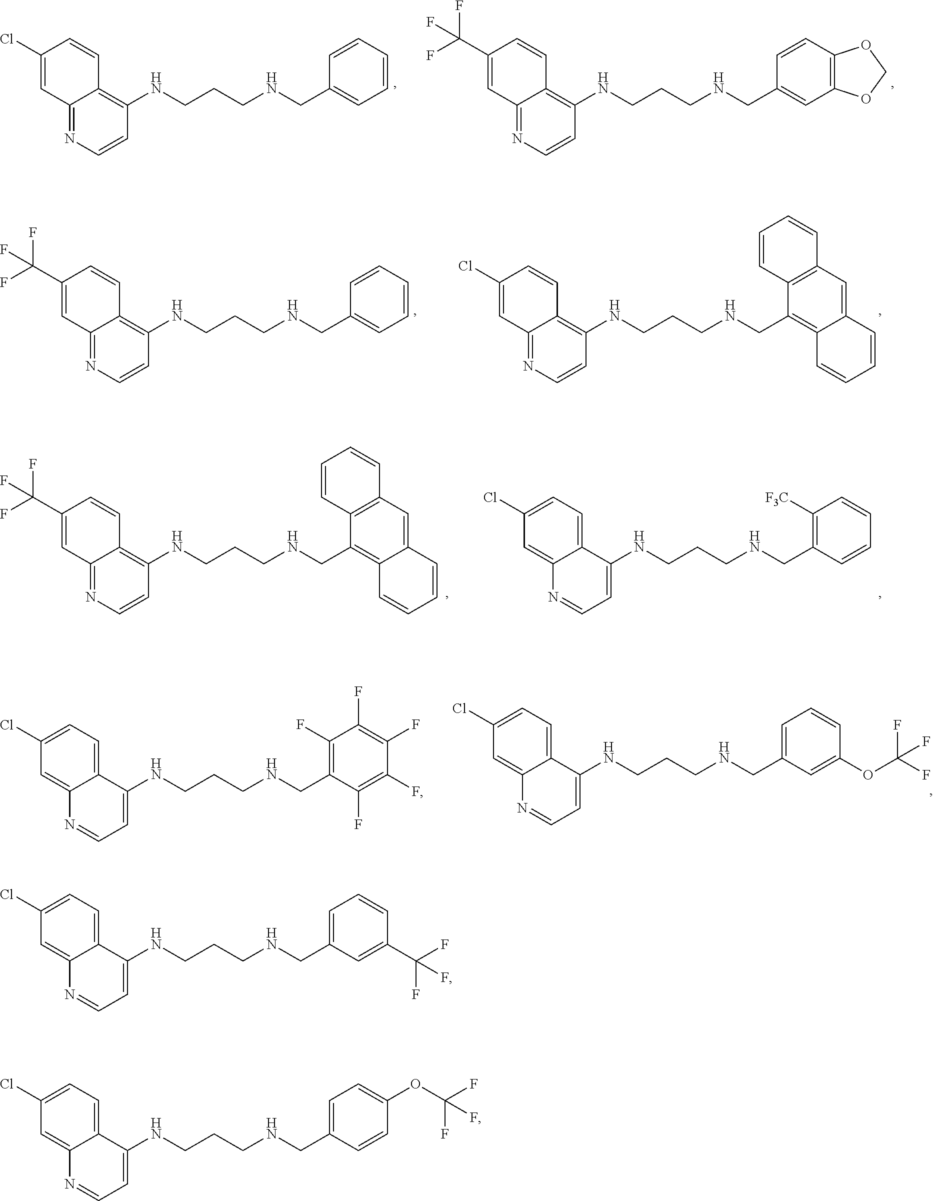 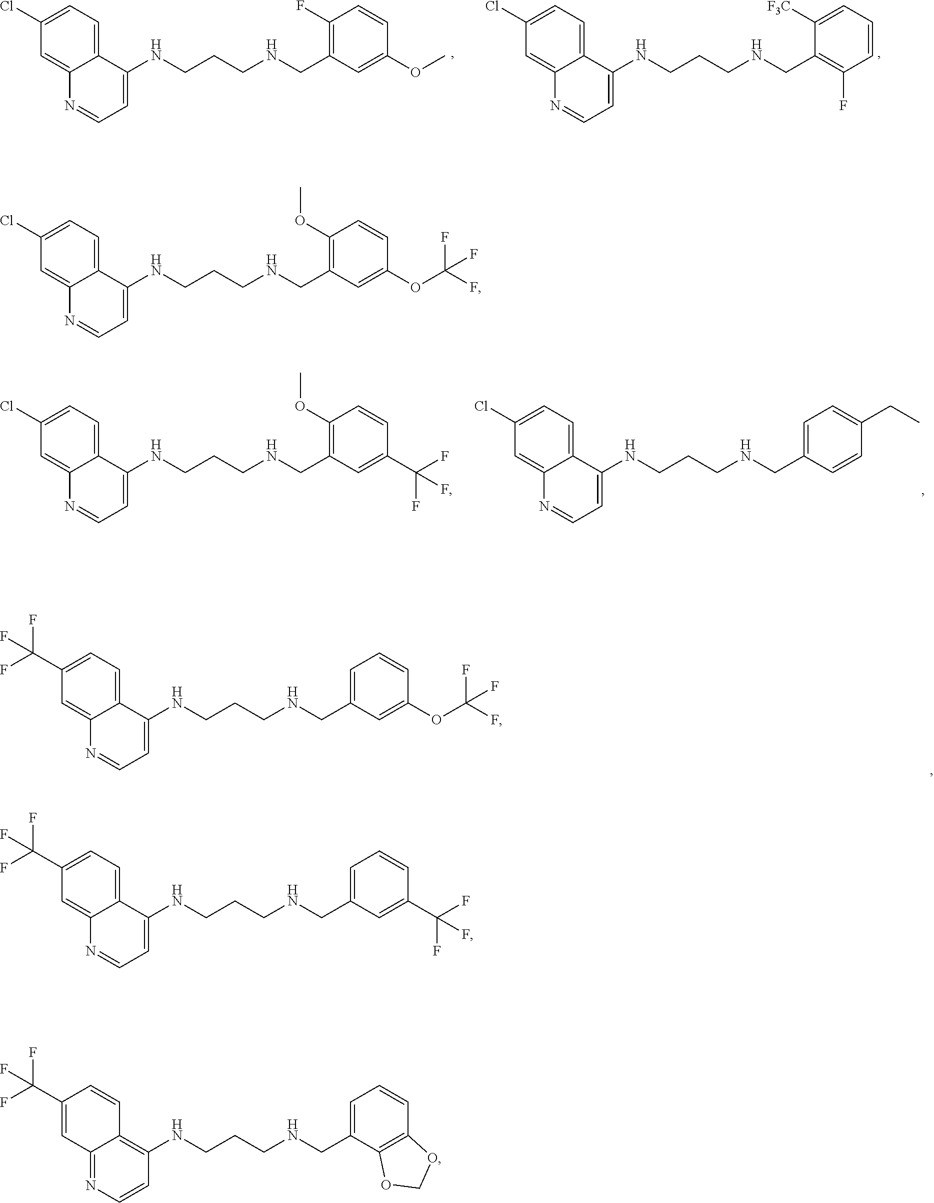 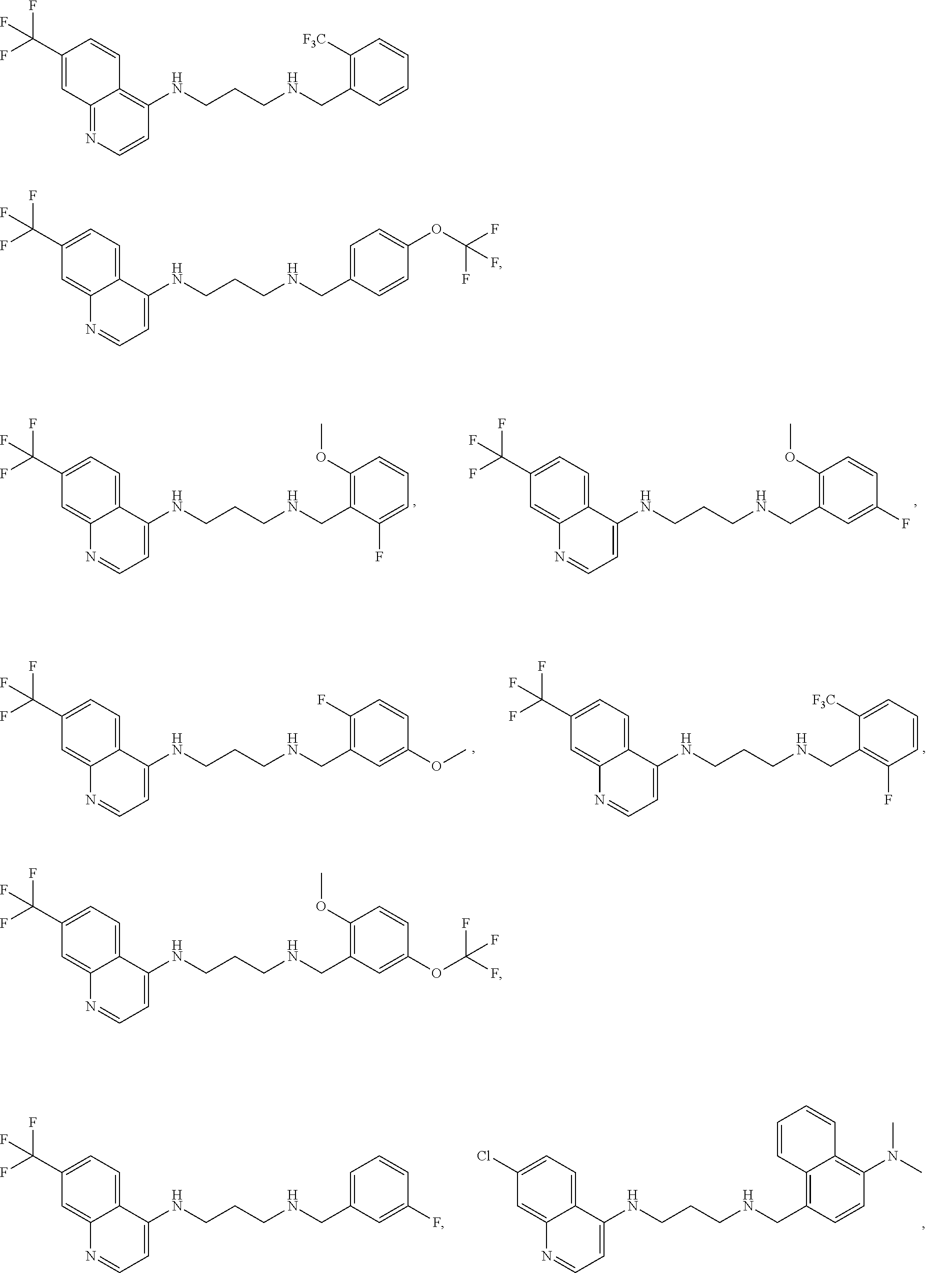 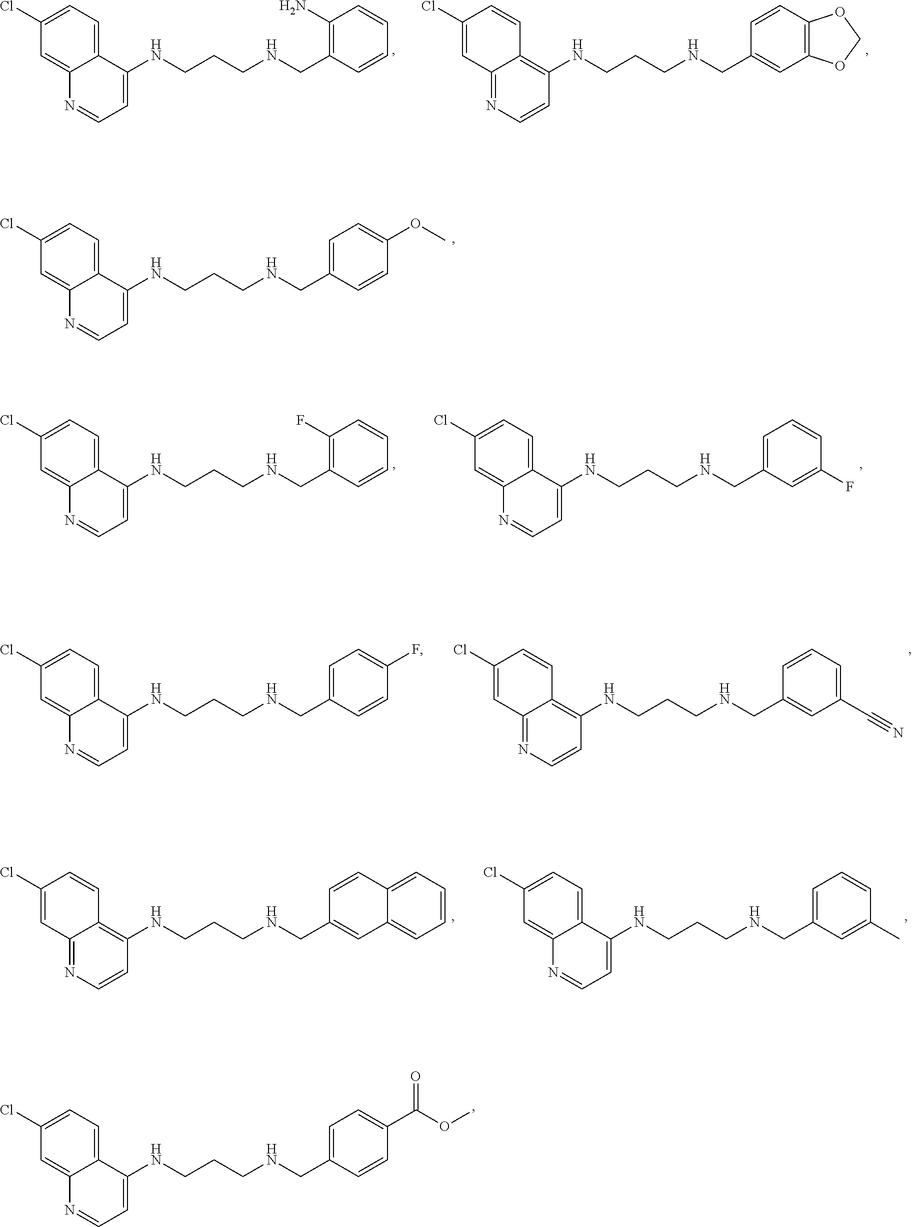
or a subgroup thereof.

27. A peptide having a structure given by the sequence:
FLVVVHQIRQLXQ,
wherein X is a substituted phenylalanine analog having the structure given by the formula: 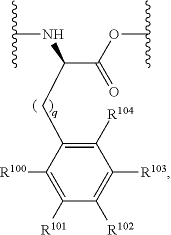
wherein q is selected from 0, 1, and 2;
wherein each of R¹⁰⁰, R¹⁰¹, R¹⁰², R¹⁰³, and R¹⁰⁴ is independently selected from hydrogen, halogen, —SF₅, —CN, —N₃, —NH₂, —OH, —CN, C1-C3 alkoxy, C1-C3 haloalkyl, C1-C3 aminoalkyl, C1-C3 alkylamino, C1-C3 hydoxyalkyl, and C1-C3 alkyl.

28.-41. (canceled)

42. A pharmaceutical composition comprising a therapeutically effective amount of at least one compound of claim 1, or a pharmaceutically acceptable salt.

43. The pharmaceutical composition of claim 42, further comprising at least one agent known to treat a cancer.

44.-51. (canceled)

52. A method for the treatment of a disorder of uncontrolled cellular proliferation associated with a MAGE-A11 dysfunction in a mammal comprising the step of administering to the mammal a therapeutically effective amount of at least one compound of claim 1, or a pharmaceutically acceptable salt thereof.

53.-54. (canceled)

55. The method of claim 52, further comprising the step of identifying a mammal in need of treatment of the disorder of uncontrolled cellular proliferation associated with a MAGE-A11 dysfunction.

56. (canceled)

57. The method of claim 52, wherein the disorder of uncontrolled cellular proliferation is a cancer.

58. The method of claim 57, wherein the cancer is selected from a brain cancer, lung cancer, hematological cancer, bladder cancer, colon cancer, cervical cancer, ovarian cancer, squamous cell cancer, kidney cancer, peritoneal cancer, breast cancer, gastric cancer, colorectal cancer, prostate cancer, pancreatic cancer, genitourinary tract cancer, lymphatic system cancer, stomach cancer, larynx cancer, malignant melanoma, colorectal cancer, endometrial carcinoma, thyroid cancer, rhabdosarcoma, and combinations thereof.

59.-64. (canceled)

65. The method of claim 58, wherein the cancer is hematological cancer; and
wherein the hematological cancer is selected from chronic myeloid leukemia (CML), acute myeloid leukemia (AML), chronic lymphoid leukemia (CLL), acute lymphoid leukemia (ALL), hairy cell leukemia, chronic myelomonocytic leukemia (CMML), juvenile myelomonocyte leukemia (JMML), large granular lymphocytic leukemia (LGL), acute lymphocytic leukemia, acute lymphoblastic leukemia, B-cell lymphoma, T-cell-lymphoma, Hodgkin's lymphoma, non-Hodgkin's lymphoma, hairy cell lymphoma, Burkett's lymphoma, Hodgkin lymphoma, non-Hodgkin lymphoma, and combinations thereof.

66. The method of claim 57, further comprising the step of administering a therapeutically effective amount of at least one agent known to treat a cancer.

67.-127. (canceled)
